SEQUENCE LISTING (215 sequences)

SEQ ID NO: 1 - DNA, 95 bp, Mus musculus
  misc_feature 39, 62: n = a, t, c, or g
agcaagcagggagccagctgcgggccaaggaggaggggngactttcggtaaccgcacagcanccggcgggacagcagcggagtgtagggcagcgc

SEQ ID NO: 2 - DNA, 21 bp, Mus musculus
  misc_feature 6: n = a, t, c, or g
ccgggntttaaaaagcacgcg

SEQ ID NO: 3 - DNA, 193 bp, Mus musculus
  misc_feature 12, 22, 30, 36, 41, 61, 62, 65, 71, 79, 120: n = a, t, c, or g
ctggagagcatnttcagggtgnacagggcnggccgngggcngggtggacaaaggtcagganncantcgatntagcccanatggtccttcagtcacagagccggaacaggcaattctctanccataaacagccactcaggcagccccaaaccacacgcatgcacatgtgaagactctgatgaagtacagctgct

SEQ ID NO: 4 - DNA, 155 bp, Mus musculus
ggagctgtggtcgaggctggtccagcatatccctggagactagaactgtgcagtgggaaatgcggtagactctgagttctggaacttgtttgaatctctgtttgaatctccgtttcctcatctgtaagaggttagtaagttgtctaaggaaaggt

SEQ ID NO: 5 - DNA, 55 bp, Mus musculus
agataagagctaggagacacccacagctggaaaatcaccaagtttctaagaccac

SEQ ID NO: 6 - DNA, 138 bp, Mus musculus
aaaacatgggattaactttataacccaggatcaaactggcttcggtccgctcttgcggtcatcttagacttgtgtttttccttcccttaggaacttcctcagcatgctttttctaaaagcactccagtgtatctgcac

SEQ ID NO: 7 - DNA, 246 bp, Mus musculus
  misc_feature 103,.213: n = a, t, c, or g
agtggaagatgggaattcttagcccaagacctgatcaggctacacttgccctcgttcacctcatccatttgcatggaggtgacttggggtggcttcctgacantatccctcctgcaattcagtccccatagagaaactgccaattgccagtttaagaccttctgttcctccctgcggggcataagtccatgcgctgagcccggtcacgtgacngacctccaacgcctcatcctgctgtctcagtct

SEQ ID NO: 8 - DNA, 119 bp, Mus musculus
  misc_feature 15, 32, 35, 38, 65, 102, 108: n = a, t, c, or g
ccctgacagtatgtngtgtgggttgggtaaanacntancgctgtgggtgtggattggcttagaangtgcatctggtatgtgcctacaggctttctaactgtncctacncgtctatgtac

SEQ ID NO: 9 - DNA, 275 bp, Mus musculus
cacccttgtatcggtctccgccaccaccaccactaccagcatcccccaaagaagaaaatctcctccgaaatgccccgaatgagtgctgctgctggctctgaagccgtgtagaatttcgtaatggaatgtgaactgctcgtccggatctgggctcacgttctatctcttaaccagtaaggaacgagggagggcaaatctgctgagcaaggaaaaataactttcctcctcttttataacccatcacggatgcaccgcggacgagggcagctagcaac

SEQ ID NO: 10 - DNA, 290 bp, Mus musculus
  misc_feature 2, 14, 18, 222, 240: n = a, t, c, or g
tnatggtggccccngacnaggtcccctacctgcttgacctacacttgttcctgggccgctctgtcaccctggcccgtccttgtgaggagccttcaggtgaggccaggctggactgggcttgggtccccatggaccatggagatcatgagcaggctggggtgcagtggtctgaccacaggagatgtctgctgggtctgaccgtacggcctggggtgctgggcntacccttgggctattgtntgccagagtggggggtctggttgcatataatactctagcctgtatctgtt

SEQ ID NO: 11 - DNA, 221 bp, Mus musculus
  misc_feature 45: n = a, t, c, or g
ggagcagtcatcatttggaaaactgagagaagatgtctttaaaangagcccaatctgaggtgtggtgcacttctcttctgctgggcacaccttacccgaactccgcgtgcttgctgctgtctggaccttacttgtcacctctacttcctgttctgtgaggactgccacccagtctcagccaccaccacctctgcccccactgtgatgacacagaactgcgc

SEQ ID NO: 12 - DNA, 122 bp, Mus musculus
  misc_feature 20, 45, 50, 64, 108: n = a, t, c, or g
ctcgtttcagggttgcttanaggattcttaaaaaccagacaattnagcantccatgtttaccangggcagttggaaatccagtttctaaaatcactgtcaactctccnacactttctattgt

SEQ ID NO: 13 - DNA, 331 bp, Mus musculus
  misc_feature 7, 16, 18, 26, 41, 50, 61, 70, 86, 96, 124, 129, 299,306, 329: n = a, t, c, or g
ctccgtngggagccancntggacggngtgtggggaccggtntcccagtcntctccgcaaancggtctccnaggtggtttaaccggngtttggtggnggtcgggtttcttacagttagatgtcanctcanctagtgtgacatcaccccaaaccagtgtgatttttcccccaacatcccaatcacatcccagcgattgggcagcgcagggagacattgactacctgggggatgactctgagggtttagaattctcagtttttacttaaattgtttgctgccatgtcgatttcagggcagcnagggggnatttagatgcctccctgtccttnga

SEQ ID NO: 14 - DNA, 37 bp, Mus musculus
  misc_feature 11: n = a, t, c, or g
acttcaccganatgtagcaagaattcagacggatggg

SEQ ID NO: 15 - DNA, 311 bp, Mus musculus
  misc_feature 178: n = a, t, c, or g
atctcatctcatctcatctcatcttctttcctctccatacttatgttgcctattcaggaatattttggctattgtacctgtggatattcattacaaaggaggcagtggctcaaatgaagccaaagagcctggctctgaaggactgatggccaggtggccagacataggtattcaaaanaagatttgaggcttctgtttacctcttcgctgatggtgccactgctgaagtagtacttctttaccctggcagcattgtctcagtgacagctgtgtcttgtccacggggcctctgtgtcccatgctcttcacaa

SEQ ID NO: 16 - DNA, 189 bp, Mus musculus
  misc_feature 8, 13, 27, 85, 169, 178: n = a, t, c, or g
tcttggangctcnaaagcttgcggggngttggtgtatccatggcagggacttgagttgattatttttaccccgcaaacagggtantgctgacctcgaactctcaatccttttccccaagtgtctggattacaaatgtttgtctacacacccaaacaaattttaatgatncaagaattntccccgtggcc

SEQ ID NO: 17 - DNA, 173 bp, Mus musculus
acccaacactgcccatgcctccccaagccagattaaactcttctctcgattgcctctttatacttctctactctcggataatcccagtcttcaaggccctagagaaggaatgactgtgcgtcccttttaatttttaccctagaactcccctgattttttaactcagtgaccac

SEQ ID NO: 18 - DNA, 95 bp, Mus musculus
  misc_feature 20, 23, 42, 45, 47, 50, 80: n = a, t, c, or g
aaagtgccaacctctgcagntgntcttcactccaccacactnggngnttncctgactggctacagagatggagtctcagnccagctccccgccag

SEQ ID NO: 19 - DNA, 65 bp, Mus musculus
  misc_feature 44,50: n = a, t, c, or g
ttaggactgaaggagctgaaggggtttgcaaccccataggaagnataacnatatcaaccaaccag

SEQ ID NO: 20 - DNA, 56 bp, Mus musculus
  misc_feature 13: n = a, t, c, or g
gagccacactggnaagtctgacaagagtcagtgctgtccatgctgactccaccctg

SEQ ID NO: 21 - DNA, 352 bp, Mus musculus
ctataatgatataccagataaaggtcagaaagggtggtagtctctttatggagtatgtttttggggttaaaaagttttattttgatattagaagagcttcaattcaaaactgacttttaaggctcaaacataacagagatagataaccagtatccttgtaaatgatcaaataatttaatctgttcagaaatatataagaagccatgctaagaactgatgcagttaatttcaagattaagctttatttagtcttctgttgtatattttcaaggtatagtttagagcagataactaaaaacaggtaggtactagccctcaaaccagtcagagatctcctgaatgtggcatttag

SEQ ID NO: 22 - DNA, 168 bp, Mus musculus
  misc_feature 20, 113,.135: n = a, t, c, or g
ctacttggatctgatgatgntgcccaggatacaagaagagacacagtcagccagtcctaagacagacagacttcctaggaagccagtgactctcagcatgaaaggcaccaagnactgggcagccaggactcaggnccctctggcattctggctacctccctgtccccc

SEQ ID NO: 23 - DNA, 193 bp, Mus musculus
  misc_feature 2: n = a, t, c, or g
tnaaaagattgggacaccccctccgcggcccgcccaccgccctcccgccgggaaaccaggcccgcgtcctctagctctcaggccgagggcagaagtccatagtagccccgatcaataattatcccgagcttgctccctggagggaggtttaaaccagggcccctgtcgcactaccccgatgggcacaggcagg

SEQ ID NO: 24 - DNA, 281 bp, Mus musculus
  misc_feature 2, 26,. 32: n = a, t, c, or g
cntctgaccagctctaaatggctctnattacntttcaatggagcatagagtcaaattttgacaagcacataaacttaatagctgatctgcaggcataattaccaccagactgatttgtaactgccagcgaataagcccacgagacggttatccaaagtcttccagttcaaagaccgaagttgtgaggatgaagccactacagccacgttggagctaagcgtctgctgcattcgaggctctagacacaatgcagggaactgagccatctcaaagcatcactc

SEQ ID NO: 25 - DNA, 40 bp, Mus musculus
gtttcaattcagccctgtaaaaaactacacttcctcgtgg

SEQ ID NO: 26 - DNA, 137 bp, Mus musculus
  misc_feature 79, 90: n = a, t, c, or g
tcttaccaaaaccacagctctagggtgattctcacaatattaggccagtgcttcactgattgcatcaaaagctaggggnctccagtgganaacattccagctgtgttttttgcctgatgacacacacacatagatat

SEQ ID NO: 27 - DNA, 143 bp, Mus musculus
aaaggtgcttcttagaggtgctaattgggaagagccaaggtgaaggctgcaggacacaaatgtatctctgtgaaatctgctatggaaacatcgtctgggacctgttggtggaaatcctattggccttgagcaaaaaggcgaaa

SEQ ID NO: 28 - DNA, 369 bp, Mus musculus
ttaaaagaaccctggcttcccaagttctgcctcaggcaaaggagcctgcttacattccaagcaggacttgtgccctccagatagggaaccccaggaagccaccgcccgtcccagaccaattctttccctcccttcagctcggtaggtctttgcatctaggatccccgccccagaccgcctgtgagcagagcaaagcggtcccagcagctctcagatactgctgtgggttctgtgtctgcgaggaaggcagcacagaaactttcagtccccgggtattttgtcagtgtggctcttttatgttaccgcatcccacagggagacacggttatgccatttttattatctctctcccctgctgggagcttcttc

SEQ ID NO: 29 - DNA, 211 bp, Mus musculus
  misc_feature 63, 67, 72, 118, 156, 200: n = a, t, c, or g
acagaaagaagtctggtcacaactggctacagcaaacgagccaggtaccccagggacgactcnccanttccngccagagatctgatctacgtacacctgcgtcatgctgagaccctcnagcctcactaaaagggtccctgcctagttctgtttacnaatctgccttattctgtttttgttcccatgttaaagatagagtnaataccgtatt

SEQ ID NO: 30 - DNA, 198 bp, Mus musculus
  misc_feature 57, 117, 119, 120, 121, 123, 124, 125: n = a, t, c, or g
tgtgagcagagggttaaagacatgaaatctggggctgcagagacagctccatagttngcaacacctgctgctctctaagaggacccagagtttggctcccagcacccacatcaggtngnnnannngcacctgaaaccacagctctaggggtctcaacctcctggggctctgcagcgccagcatatgcacttgcacgcg

SEQ ID NO: 31 - DNA, 71 bp, Mus musculus
  misc_feature 70: n = a, t, c, or g
ggttgcggtcacattcggcgtgtccccagcccgggggacggggccccggggaggccccgcatcgctgcant

SEQ ID NO: 32 - DNA, 76 bp, Mus musculus
  misc_feature 12, 34, 46: n = a, t, c, or g
cttgcaagagtnatttgtgtgctccttctaccancttctaaagatnagacgctggttgtcagcctctgtggcaagc

SEQ ID NO: 33 - DNA, 227 bp, Mus musculus
  misc_feature 4, 5, 10, 116, 134, 172, 224: n = a, t, c, or g
gatnncccantattcactctgatagtgaatatacccaaacatgacaccaccctccgggacaaaggaagcacatgctggcttgctgggaccccttaagtctggccagctctaggtanggacttcctgtcctcatncactggggaaaagaagtgttggagaaacgtgtcaccantaggtgtcgcccgacaacggtctcgatcaaccaaacaaaccaatacagatcnctc

SEQ ID NO: 34 - DNA, 153 bp, Mus musculus
attccacaggtagaaatgtccacatcttacctcatgtgttgctatactaaaatattcatgcattgaaaatactgtatgaagccgggcagtggtggcgcatgcctttaatcccagcactcgggaggcagaggcaggcagatttctctgagtttg

SEQ ID NO: 35 - DNA, 352 bp, Mus musculus; identical to SEQ ID NO: 21

SEQ ID NO: 36 - DNA, 223 bp, Mus musculus
  misc_feature 22, 66, 107, 111, 122, 133, 137, 146, 166, 182: n = a, t, c, or g.
gctgaaaatgctaggctttgtngagctatgagccccgggaatcctcctgtctctacttctccagcngaaggattacaaatctactccaccttgaacatgggtgctgnaggngaacacttaanctcacggaagntcancagcatttnacaaacctgtcatgccttgntttgttttaaagattnatttattcataggcatgattgttttgcctgcatgaatttct

SEQ ID NO: 37 - DNA, 300 bp, Mus musculus
  misc_feature 80: n = a, t, c, or g.
ctttaaccgtcctctcctaaaaaatataagaaatgagtaaatgggtgactggaggaacaagagaaataatagtgtgtaanagggtgagtctccgctgttggtcagcacaacgcacctgcagaggctttctttctcttttatacgttttaataatgctgcttccatctcccagggacgtttgaggctcagcctcaccaatgtttctctcctcttgttctcccctagcctacccatcaccactcacccctgcggcagccacacaggccttcctcagcttctgttcctgaactttgaatcgat

SEQ ID NO: 38 - DNA, 115 bp, Mus musculus
  misc_feature 35, 45: n = a, t, c, or g.
gtctctcctgcttgctgaagtagctgtttgtgtcncctcccccancccaccctcaagctcacacagatcctccgaacatatgaagcagaggaggggcttaggctgcggaactccc

SEQ ID NO: 39 - DNA, 152 bp, Mus musculus
gtctgctcttccttcccgacagtatctaaatataaaagaggactgcaatgccatggcgttctgtgctaaaatgaggagcttcaagaagactgaggtgaagcaggtggtccctgagcctggagtggaggtgactttctatctgttggacaggg

SEQ ID NO: 40 - DNA, 22 bp, Mus musculus
aaatgacaacggggaagatgaa

SEQ ID NO: 41 - DNA, 66 bp, Mus musculus
gggtacgtgggcgaggggctcgcccactggtgaggtctctggacctatcgattcccggctgatgct

SEQ ID NO: 42 - DNA, 308 bp, Mus musculus
ccataagcacacatatgtaaaaggtttgcacacctcataagcttcactttgtgaacgtgtacagcgttagtatgtgcaaaaaatatcatgtcggaagagcagtttctatttgtgctacccaaaaacgggtttgtattttgagaggggagaatcacgctgttaggctttatttatatccaagtgtcctcagccttctgcaaaaaaggcaaaagctttgtgtgtgcgtgtgtgtgttttaatgcagaacaacgaaggactcagacactttcggactctacagaaccagagcatacatcgcgggcctgtgt

SEQ ID NO: 43 - DNA, 61 bp, Mus musculus
  misc_feature 4, 7, 9, 14: n = a, t, c, or g.
cccntcnanaaaanaagaacaaaagctttctcgctcctacatggcaaaacacaaaccacta

SEQ ID NO: 44 - DNA, 57 bp, Mus musculus
ataaaaacccaaggcatgcaaaggtgaaagaaaccagtcaatcaccagacgacggcc

SEQ ID NO: 45 - DNA, 45 bp, Mus musculus
ccaggctggagggcctgcggggaccggtgcgtgaaaggcacctcg

SEQ ID NO: 46 - DNA, 33 bp, Mus musculus
cccctgcctccgccaccaccacctcctccaacg

SEQ ID NO: 47 - DNA, 359 bp, Mus musculus
atattatcactacagaacatgaggatgtcgttgattgcggcaaccactagaccaccactcactggatgaggagctcaggaaagctggccccatttctcactggcagcagcacagtagagctggccctagtggcaggggtgtaggtgagccagccctgagggcatgagtgtgggagaactgtccctgccacaggtatgctgtaggctggtagcatgggcacagagatgattccccctccaccgctccttgtcatctctgtcagtggggaaggctgcctgctggtcctgagcttgggagtgctatccatgatgctgggagtgctatctgtgatgcacacgagcttcaccaggtaggagaac

SEQ ID NO: 48 - DNA, 385 bp, Mus musculus
  misc_feature 30: n = a, t, c, or g.
ttatccccgcgagacagtcgtgcatgctcnaagtcagccttatcgatgtgttaccgtgtctttggtgggggcctggcagcagggtgggagcagcccgcgcgctctgcggctggactgagcgggtctgtaaattaacaagctggacgaccagtggcacatccaggctggctacaaggggtcttctcgggagggaccacagggcctttttccaactcggccgatgggagtgcgcgaggcacactgatgcgagcctccactgctcgggccgaggccatctctcagtgacaggtttgggaggactcgcccacgtgcgggaaacttaagcagaggcctccattctacgatgagtggtgccacctgaggggtcggctcttggcatcaggcc

SEQ ID NO: 49 - DNA, 274 bp, Mus musculus
  misc_feature 66, 71, 138: n = a, t, c, or g.
ggttctttggaagagcagtcagtgctcccaattgctgagatatctttccagcccctatttttaaanatttnagacaggctttcaagggctagcttgaaactcactatgcaatagagaaggacttgaacttctgatccncctgcctctacctcccaagtgctgggattacagcccccacccccacccccaatgccagtttgtatactgtaggcagtggaacccagggctccagcatgctgatgctggtatgcatgggcacttggaccacatcgcc

SEQ ID NO: 50 - DNA, 111 bp, Mus musculus
  misc_feature 106: n = a, t, c, or g.
acagaaaggaaacgcgattcgttccacttggaatttccttgaaatctccgaatctaatccagcgttaactcaccgtgagaagagcgcttgtctcataggaggctgngttaa

SEQ ID NO: 51 - DNA, 249 bp, Mus musculus
aaatgttttttggttttttaaatcgggcagggtgctgcgcacctttaaatcccagaaagaggaaagcagaggcgcgtggctctccaagcaagccaggctagtttcccatccatctgcgggttatccaaccagagagaatttctctcactttggtttccgacatgctttaggcataacctgggaacgagggtaggagggagctccaggctctaaggacaaaggaaccgcaggtgcaggaagctcaaggaa

SEQ ID NO: 52 - DNA, 43 bp, Mus musculus
gtttcaattcagccctgtaaaaaactacacttcctcgtggccg

SEQ ID NO: 53 - DNA, 47 bp, Mus musculus
ttcataaatctgaggccagcgtacagctatagagtgagatcctatct

SEQ ID NO: 54 - DNA, 210 bp, Mus musculus
  misc_feature 20, 21, 27, 44, 131, 167: n = a, t, c, or g.
aaagttctctgagacgtgtnngactcngggcgtgggcaagtgcntgtttgagtggatctgtcaatccgttgtgtgataaactgtcaacaatgaagggatatttatttagcttatagaaagtcctgagccangaactgaagagggaggcacgcactcatggctaggangcagctggctctggctggccttgtcctcatcctactggggact

SEQ ID NO: 55 - DNA, 277 bp, Mus musculus
  misc_feature 190: n = a, t, c, or g.
ccactccccccctttggccctggcgttcccctgtaccggggcacacaaagtctgcgtgtccaatgggcctctctttccagtgatggccgactaggccatcttttgatacatatgcagctagagtcaagagctcaggggtactggttagttcataatgttgttccacctatagggttgaagatccctttanctccttgggtactttctctagctcctccattgggagccctgtgatccatccattagctgactgtgagcatccacttctgtgtttgct

SEQ ID NO: 56 - DNA, 252 bp, Mus musculus
gacggtgatgcagtagaaataaaggtctcagcagtgcactgcagaaaatcaagcaaagcccccttaggagttattcatgtttgccgctttcgtgcaaataggggagggggcttaaggcttaccggaagaccccccacctagctcaggtcttgtacttctgtcttctgggtaaaggcaaaaggagatttggggtgtagttgatggcccatttagggtggtctcgcagactagaaaacctgaaatgcacttaac

SEQ ID NO: 57 - DNA, 35 bp, Mus musculus
agggaatccagagttgtacacagcgaggtctgaac

SEQ ID NO: 58 - DNA, 216 bp, Mus musculus
agaagagtttggtaaactcatagaagcccttgaagtattgtaggtttggtttgccagtttaatcgtaattgctgcttttctacaggtttttgctggtgtgaaatgactgagtacaaactggtggtggttggagcaggtggtgttgggaaaagcgccttgacgatccagctaatccagaaccactttgtggatgaatatgatcccaccatagaggtg

SEQ ID NO: 59 - DNA, 176 bp, Mus musculus
  misc_feature 17, 158: n = a, t, c, or g.
ccccccaaaaaaaatanttgttggagcaccagttgataaatatttgcctcaagaaatttgccccgaggacttggagctgacagaaggtcaaagcgaagtgtgtgatttatgttctcctgacaagatactggctgttctacagacacaaggttttgagnctccacggtccacagaca

SEQ ID NO: 60 - DNA, 202 bp, Mus musculus
  misc_feature 32: n = a, t, c, or g.
ctatgttgatctgggatattaattacaatatncaaaacaaaagctgggtatatagcctagtggtaatgtactgacttagcatgcccgaaggcaggcttggtcctttatggaacttacagcctgtcggttttatcaggatcagcacatacagctggtatctgtgtctgtggaactggtaggttgagactcttccccatgggcc

SEQ ID NO: 61 - DNA, 106 bp, Mus musculus
  misc_feature 31, 62: n = a, t, c, or g.
aaaaaagttctaattatcatgtgaggaaganagtaagttatgagcagcctcctggaagcatngcagcgcctcgctctctgctcccctctctctctgtctgggtgag

SEQ ID NO: 62 - DNA, 454 bp, Mus musculus
  misc_feature 9,124, 434: n = a, t, c, or g.
ttctctccnctagacttctggggactgggagactgcagtatgggtcgtgcaggattggagtgatatacttagcaagcctccagcgtgcttgggtctgcagtgaccctgtgcattcctacagtgnttgccagaacaattttgaagtggtttgaggccttgccctgccctctccagagcaaggttatagaaatttcagacaatatggcagacacctgccacgtggataaattacaagccggtaagatttgcaatgctgcactttgggttttttgttttgtttaactgtgtgggatagttctgcacatggtgcagaggcaaataagtcatttcttgttggttttgttttgaggcaaggtttctctgtagttcttgctgtcctggaactcaaaacagaatccactcacctctgcctcctgagtggtggggattaaaantgaagaacccttcataaggc

SEQ ID NO: 63 - DNA, 221 bp, Mus musculus
  misc_feature 7, 9: n = a, t, c, or g.
ctgtttnanattagaagctgaactcccagcaaccacctaaaagccaggggtgaaagatgcatgaccataatggcagcaatggggatgcagacacctgagaatccctggccaatcaggatagcagaatccataagccttagactcaatgagaggccttcagaaaataaggcacagaacaagagaggaagacacccagtgtcaaccttggatctcagcaggtt

SEQ ID NO: 64 - DNA, 199 bp, Mus musculus
  misc_feature 6, 112, 138, 165, 169, 184, 188, 192, 193, 195, 197: n = a, t, c, or g
tttgantcaggatgtgcatagctcaaattggccttaaatttatgatctcccttcctcagcctgccaagtaactaagattatagccctaaaacaccaggccctaggtataagnatttgtttttctttctttttttttcntttttttgggtttgttttgttttgganacantgtttctctttgtancccnggcnntntntt

SEQ ID NO: 65 - DNA, 291 bp, Mus musculus
  misc_feature 128, 129, 130, 131, 132, 133, 134, 135: n = a, t, c, or g.
accaagaagagtaagagtcatgaggggcaattagaacacttgtgttcagcactgggtcgccgaggcttaaacgactgcagtcagctaactagggatgtcgtcagttgtcgcatcggacggcacttccnnnnnnnnctagtttcatcatcattgcagccgacaccccgcccacgcgcggcgccccgcgatgcagacctcgacttaccaggctcccctagatctgtgcagcgcacaagacggagctgaagaggctgggcccgggctcagcatcgctccagaaccgtcaccagc

SEQ ID NO: 66 - DNA, 89 bp, Mus musculus
  misc_feature 10, 31, 38, 45, 49, 53, 61, 65, 72, 75, 87: n = a, t, c, or g.
tgtccagggnattcactcaaagcgctcagtncaagctngtccaanaatnctgnataagcgntcanttcaagnttntccaaaaattcngg

SEQ ID NO: 67 - DNA, 298 bp, Mus musculu
  misc_feature 58, 128, 271: n = a, t, c, or g.
ggacctcagctttcagagtctgttctctcccattctgtgggtcctgtgaactcaagtnagctctcaacaagagcaacaagagcctttacccgcagagccatctcgacaccccatcagtcatttttttnttttattatttggagaaacttaacctgctggtcttggggtgcccttagcctctgggaaaaactcctacaaaaccttcaaaacaactgcaataaggagtggagggattcaaaaagtctcggggcgctgggttgggctggaggcnatgcagtgcggctggtcagtgggtggc

SEQ ID NO: 68 - DNA, 128 bp, Mus musculus
  misc_feature 4, 21, 25, 29, 89, 92: n = a, t, c, or g.
gcanttaggaaggcaaaggcntgtnatcntaagataatgaaggtaaagttagtttatagaaggaagtagtcatgtttgaaagagacggntantttgagcggtagataaagtaagaagagaaagatttg

SEQ ID NO: 69 - DNA, 39 bp, Mus musculus
tgtagttaataacctggtaatccctgctacccccagggc

SEQ ID NO: 70 - DNA, 83 bp, Mus musculus
  misc_feature 16, 18, 37, 74, 80, 81: n = a, t, c, or g.
gaggagaggctgtccncntggatgaggtcggatcatntggggtcgtagacgtgtaggtggagagcacaagtctnattctnngg

SEQ ID NO: 71 - DNA, 168 bp, Mus musculus
  misc_feature 7, 12, 15, 16, 26, 36, 37, 40, 41, 42, 52, 53, 55, 56,60, 61, 63, 76, 97: n = a, t, c, or g.
  misc_feature 108, 109, 110, 115, 116, 118, 121, 122, 123, 124, 125,135, 136, 142, 145 159, 160: 990<223n = a, t, c, or g.
tcttgtnttgtnttnngttgatgatnttgttgagtnngannnggggcctggnntnncganntnctgtctttgattnattggagcgggcgattgaganttcgaggccgnnngagtnnanttnnnnngaggattatnngggganctntgatggtggatatnngggtggtg

SEQ ID NO: 72 - DNA, 122 bp, Mus musculus
  misc_feature 2, 14, 36, 39, 43, 49, 88: n = a, t, c, or g.
tnactgaatggganctggggccagagggcagttggnctnttgnaaagtncgggtctcagctcagagccctaatcccgaaactggcgcnacagtcagccggtggagcgagataaagcgggcaa

SEQ ID NO: 73 - DNA, 120 bp, Mus musculus
  misc_feature 17, 22, 39, 40, 43, 116, 118: n = a, t, c, or g.
tttctggaaactgaatnaaatnttttattcacgtgattnngcntcttctggatctattgatttgagttggtgatactgttggatcacgggattaggcccaatggggacgcggccgncnga

SEQ ID NO: 74 - DNA, 163 bp, Mus musculus
  misc_feature 12, 36, 42, 46, 48, 56, 97, 101, 116, 137, 146, 150,152,158, 161: n = a, t, c, or g.
tgatgctaggcnggctctttgccaactgagccacantccttnaggntnttctgttngggtgccttgggctgtccttgccaaccagggaaatctggantccncgggaggccagctgngctggggacagctccaagtcngagaccacnagcngngatgtngcncg

SEQ ID NO: 75 - DNA, 191 bp, Mus musculus
  misc_feature 3, 4: n = a, t, c, or g.
gtnntcttactataggggttttttattggtaaaaacttcctgacttgaccaatacttgaaatctacagcagtttaatagcacatcagtgtccctgtggtagcatggtcactgtacccctggttctaggcttgggcttgcagatgaatcagcgtgtcttctgattctgcacattctctgacgtgtcaccggc

SEQ ID NO: 76 - DNA, 95 bp, Mus musculus
  misc_feature 23, 31, 48, 59, 68, 69, 73, 84, 86, 92: n = a, t, c, or g.
aaatgttttatttgtgtgatttnggttgttntggatgtattgatttgngttggtgatantgttgggtnngaantggggtgtgcngnagggangtt

SEQ ID NO: 77 - DNA, 504 bp, Mus musculus
  misc_feature 5, 16, 17, 32, 35, 36, 48, 49, 65, 85, 92, 93: n = a, t, c, or g.
  misc_feature 108, 119, 121, 127, 140, 150, 164, 184, 186, 199, 210,211, 215, 235, 245, 254, 257, 259, 266, 268, 271, 291 314, 320,325, 371, 401, 406, 417, 432, 436, 438, 449, 472, 485, 490: n = a, t, c, or g.
caacnattaccgtgcnncaaaaaaattttttnagnnttatgcgggggnnccccaaaaaaaaggtntttagtatggctgttatttnttggganntatttaagttggctnttttggtttgngntattgnaactttttggatntgagtatgtnagtgtgtcttgggntaagttttgatgtgaatttntnttatatgtgtctnacatgtgtagnngatngaataaatggagatttgtangaggagacantgcgatganacnantggtagnanagngtgggtgtttgatttgcatnttgggatggactgattttgagtnagattngggantggtgagtggtggtttagatgctgtggagaatttggggatggtgcnttctttgatgaggatttggattgggttagnaaaangattgttagantttaattgtgttctnttcncngggtggtgatnattggaaagtgtattttggggtnaagatttttggantgaantgtggaaaaaaaat

SEQ ID NO: 78 - DNA, 90 bp, Mus musculus
  misc_feature 2, 22, 26, 31, 45, 48, 68, 79, 88: n = a, t, c, or g.
angtttttgtgaattgatgganatgnttganttgggtgattccgnttnttctggattttttgatttgngttggtgatantgttgggtnag

SEQ ID NO: 79 - DNA, 120 bp, Mus musculus
gcaaggacatacatcggggacgcttcagacttcccactcatacctcacagctcagggacccaaacaggatcctcagaaacacaagtctggtaccctgcctagaatcactacggtgctgtt

SEQ ID NO: 80 - DNA, 220 bp, Mus musculus
tggtgtaccatggtgtgactctagggggcctgtactgtgtaacagggtccttccctccacagtgacctgctgtctgtatagtctgtctgtttctttgggacatgactgtgctgtggagagcaagatcggctggggctctgcctctggccccagcatgtggcagctgtatggctggggacagacacttttgcatccctgtgtttctttcactccaataggc

SEQ ID NO: 81 - DNA, 255 bp, Mus musculus
  misc_feature 237, 251: n = a, t, c, or g.
cactagagaccccgtgtccaggtgactctgcccagggctacagaacctggagcagcccgcctgggaaggtggcttttcctccagatggccatgggctttacgttagcaacaggctttcttgcaatttcgcattgccaatttgtggtggcactcttcaaaacaaaacttctagggctggagagatggctcagctgtttaacggcgctggtggttctagcaacaagaatggaggttccntttctggcacccanactg

SEQ ID NO: 82 - DNA, 256 bp, Mus musculus
  misc_feature 211: n = a, t, c, or g.
atgcttttcaaaaaacaacaaaaatatccaagtgtttattggcctcaccttctgttctctactttattggaaagagatgtactgtggcaccattgacagatgccttttctggtggcaggttctttgtggtctgactctggactcagactcttgcctgtttgccatctgtaatagggatgggcccttcccctcttgcattttttcaaacacngttctccaaggtatgttctgtcatctggcaaatgggcacctggga

SEQ ID NO: 83 - DNA, 223 bp, Mus musculus
  misc_feature 6, 11, 22, 24, 25, 27, 33, 44, 54, 59, 75, 79, 111,113, 121, 123, 153, 161, 163, 168, 169, 180: n = a, t, c, or g.
atgggntattntcgcgtctagngnntntatttncaccaccccanctcctatacnaatantctgctgcaaactggntccncaggggcaaagaggatttgcctcttgtgaaancnactgtggncntggaactgtgtggaggtgtatggggtgtanaccggcananactcnncccggaggacngggtagagcgccccccccgaattcctggacaagctttgactgg

SEQ ID NO: 84 - DNA, 211 bp, Mus musculus
  misc_feature 3, 8, 13, 23, 43, 61, 71, 76, 85, 92, 97, 101, 115,142, 151-152, 177, 196, 203, 205, 207: n = a, t, c, or g
ttntcacnacganttgagtattngtgaactgtattatctggtnttaaaaatatattccgtntcaaaatttngtttnctgaagaantgagtcntattntaanaaaatttgatatcnaaggggggacaaaaatataaaattccnggaaaacanntgacaaatacacaatagaccggggncccccgaattcctggacanacttgantngnacgc

SEQ ID NO: 85 - DNA, 81 bp, Mus musculus
  misc_feature 72: n = a, t, c, or g
actatgcagccagttcaagctagttttgaacttgctgttcgcttgccttggacttcccagtgttcggatganagccacgcg

SEQ ID NO: 86 - DNA, 240 bp, Mus musculus
  misc_feature 3, 5, 8, 26, 30, 53, 56, 64, 124, 147, 155, 169, 220,235: n = a, t, c, or g
gcnanaanaggaaagaatcattattnggtngaggtctcccaccttgtcagacncangtcaccanctttggtgacaagtgcctttacccactgagccatctcactggcccggcctgtgcgtactngtgtgtgtctgtgtgcgcacgcntgtgcacncacagttcactttnagcatgctgtatgtcagctatagtcctgagcccttcgcaggcaggactgtngctgacctttacatnttccg

SEQ ID NO: 87 - DNA, 334 bp, Mus musculus
  misc_feature 235, 237, 300: n = a, t, c, or g
acacaatgccttccccgcgagatggagtggctgtttatccctaagtggctctccaagtatacgtggcagtgagttgctgagcaattttaataaaattccagacatcgtttttcctgcatagacctcatctgcggttgatcaccctctatcactccacacactgagcgggggctcctagataactcattcgttcgtccttccccctttctaaattctgttttccccagccttagananaccctggccgcccgggacgtgcgtgacgcggtccagggtacatggcgtattgtgtggagcgangcagctgttccacctgcggtgactgatatacgca

SEQ ID NO: 88 - DNA, 164 bp, Mus musculus
  misc_feature 26, 30, 32, 35, 109, 125, 158: n = a, t, c, or g
ctctggcagccattgtgtttgttacngcanancanactgctgcaggcctgcctcccctctgaagctgcttgtgctgctgataaactctgccccttagtgctcactgttnctcatactgtgtgcancctgagcaacagcccgggatgaccatccttacngcagcg

SEQ ID NO: 89 - DNA, 107 bp, Mus musculus
gctacagctcgtcaatgcacacgttctttatataatactacacagatcttgtaaacgaagtctggacatcaaagctttatgggaactgctaagtggtctaaggacgc

SEQ ID NO: 90 - DNA, 336 bp, Mus musculus
  misc_feature 258, 263, 266, 268, 271, 283, 292, 299, 302, 307, 310,316, 322: n = a, t, c, or g.
atataataaatctagaaccaatgcacagagcaaaagactcatgtttctggttggttaataagctagattatcgtgtatatataaagtgtgtatgtatacgtttggggattgtacagaatgcacagcgtagtattcaggaaaaaggaaactgggaaattaatgtataaattaaaatcagcttttaattagcttaacacacacatacgaaggcaaaaatgtaacgttactttgatctgatcagggccgactttttttttnaattncananttncaatcccattantaaaagggnaaacctnggnttttnccnggaagnaagggnttaacggtttcctt

SEQ ID NO: 91 - DNA, 310 bp, Mus musculus
  misc_feature 5, 7, 8, 9, 20, 27, 32, 38, 42, 54, 87, 92, 96, 98,106, 108, 130, 152, 182, 195, 210, 219, 225, 230, 280: n = a, t, c, or g
ttagntnnnctggaacttgntatgtanatgangcttgnctcnaactctgatatncacttgtgtctgcctcctgactatgtgaaccanaccantctntnattcaaananactgaggttggaccatccttantcacctgggttgttctattaantgtaactacactcataaattcgaagcaaancaaaccgtaccanctgtgctactttgangcacctgancattcnacaanggatctttttaacctcatgaggcccagtcctgctaatccaggttggctcnatcctgcaatcccctgctcacaacacctgt

SEQ ID NO: 92 - DNA, 359 bp, Mus musculus
  misc_feature 116, 257, 301: n = a, t, c, or g
gtcaaaatactgagaattagaggctattggatgccaagtcatagagaggacacatatataccaatacttccaaggctcaggaaacatcatggaagaaggggtaggaagaatttaanaaccagaagaaggggggtgaggtatggaatgatgatttccagtcatgacttggctattgagttaacaacagctggatcacctgcacaagatctccacaagagtgggcccattaacactctatcatggaaagaggaggggcntatgaggtaccaccccaccctgaagatttatacacaattaatanttggtgaggtagggagagacatttactttaggggtgcagtcactagtacagtgcctac

SEQ ID NO: 93 - DNA, 151 bp, Mus musculus
ccatctctccagcccccctctctttctaatatgtaggtcccagggaccaggctctagctctcagactttgctatcttcgtgttggaattgttttacatttataaggactttgaagcctcatgtcacctgcaccacccctctgagtctgacc

SEQ ID NO: 94 - DNA, 379 bp, Mus musculus
cagctgcgttgcgtcatccagccagagctcagaacaaactatgaactacaaagttcttcagcaccaaatctcagaggcagaaaacattctaggcctagattagattgtacagaggctaagaggcttctaatagacctaggtttccagagagaggttgtaagccacaaagaccacaattacatcaggcgaatgagttacttttacatatctgtaaaatgagcagagaagagtctggggctcctctgttccccgtggtttccttgctggccctggttttcctgtgagatgtgcctgactccccggatgcccttcaactgatgttggcttagggggctgagcttttaaatgtcagatcttctcatttccgcctctgtccagg

SEQ ID NO: 95 - DNA, 282 bp, Mus musculus
  misc_feature 3, 15, 20, 22, 27, 29, 75, 78, 82, 155, 159, 166, 195,260, 275: n = a, t, c, or g
agnggtacgcggtanagcananactancntaccctttgggcgcctgtggtctccacacagagtgtgtgggtgtanganacangctgatggggactgcctctcggcagccttcacgggcacctgtgagtggcagtctgaagggtggtggccggcanacancctatanagtgatattccaaagcctgaaccattgtngctcccggctgattcctggtctcgcctgatagttttagatgcaccatcttatttgttcttcacangcagttatgctagantggatga

SEQ ID NO: 96 - DNA, 65 bp, Mus musculus
aaacctgtgagctctgcttttgtgctctacccacaggagcacagccagccttaaaactggagcgc

SEQ ID NO: 97 - DNA, 81 bp, Mus musculus
acagcacctatggctgtcctctgacctccacacacatgtgacatatgtccatgtatacatacatgcacacacacacacaca

SEQ ID NO: 98 - DNA, 317 bp, Mus musculus
  misc_feature 11, 56, 103, 111, 112, 120, 123, 143, 167, 230, 239,265, 266, 268, 269: n = a, t, c, or g
gtcttcctggncctcctgagtcccatcacttctccaactctaaatcggcctggggncaacatgctcagccagcagttaagtcccgtgccctcccacctggagnaggtgtannaaataggnggnaaggcccaggcggcctcgancccgaaggcatgaagcccccgggnaccgagcacacactgtccttccccgggtgccgctcaccatctgttgtgacacgggggccgagncctgaaagngcttggcagccccggtgagcgcgaannanncgccaagcagaacccgcaacacgcctaccctgaacgacatagcagcgc

SEQ ID NO: 99 - DNA, 90 bp, Mus musculus
  misc_feature 10, 40: n = a, t, c, or g
ggtaaggaanggctctctctggtttcctcccatgacaggnttctgtgagggccacgcgtcctgtttacagaatggtttccaagtcaccgg

SEQ ID NO: 100 - DNA, 195 bp, Mus musculus
  misc_feature 57, 66, 117, 128, 133: n = a, t, c, or g
gtgtatacaacgccttgttctaaacaacaaaccagtgcagggctgtggcgaagctangtggcagantgcttgcttagccagggtgaggctgggtgccacctaacactgaaaacggangcagtgcagancctantgcacgtgaattatcttctcggaatcattacttcccctgttccgcttgtggtgcgtctatat

SEQ ID NO: 101 - DNA, 146 bp, Mus musculus
  misc_feature 9, 109, 134: n = a, t, c, or g
gtttaatcnagcttcactaaatatcaattcggaagctttctctctgctccatttatttaaaagcaatatttatggaattgagcctgggcatcttagccctagctaagangttttagatgtgtattttaatgtanattaaaaaaacc

SEQ ID NO: 102 - DNA, 195 bp, Mus musculus
  misc_feature 6, 28, 59, 60, 85, 106, 125, 127, 138, 147, 149, 151,155, 167, 172, 181, 188: n = a, t, c, or g
caaganaggacactggcaggctggggangtgactcattctgtaagggcctgtcgcacanncaaaaagacctgaatttgattccanaattcacataaaagtcaagcntggtggggtttgtgatccnancactggggaancagagaaanananatcntgggggtctctngaccngttaattangccaaanaatctat

SEQ ID NO: 103 - DNA, 169 bp, Mus musculus
  misc_feature 114, 158: n = a, t, c, or g
cacatatacacacatgcacacctgtgtacacatatatacacatgtgtatgcacacacatataagcacatgcatgcatgcacacacatgcacatgtgtgtacacatacccacacntgtatacacacacccacacatgtgtgtacatacacatacacacntgcgtatatac

SEQ ID NO: 104 - DNA, 222 bp, Mus musculus
ctgggaagtccgggttttccccaaccccccaattcatggcatattctcgcgtctagcgccttgattttccccaccccagctcctaaaccagagtctgctgcaaactggctccacaggggcaaagaggatttgcctcttgtgaaaaccgactgtggccctggaactgtgtggaggtgtatggggtgtagaccggcagagactcctcccggaggagccgggtag

SEQ ID NO: 105 - DNA, 68 bp, Mus musculus
  misc_feature 7: n = a, t, c, or g
gtggaanacgccttttaccctagcagaggcagaagcagaggtagacggatctctgtaaacctgaggcc

SEQ ID NO: 106 - DNA, 118 bp, Mus musculus
  misc_feature 4, 5, 6, 14, 21, 27, 34, 37, 40, 42, 48, 51, 61, 64,81, 83, 86, 90, 94, 95, 96, 101, 105, 107, 110: n = a, t, c, or g
ttannnaaagtgtntatgtanacgtcnggggatngtncanantgcacnccntaatattcanganaaaggaaactgggaaantnatntatnaatnnnaatcncctntnaantagcttaa

SEQ ID NO: 107 - DNA, 212 bp, Mus musculus
  misc_feature 5, 14, 31, 32, 56, 64, 94: n = a, t, c, or g
ttatnactccacanactgagcgggggctccnngataactcattcgttcgtccttcnccctttcnaaattctgttttccccagccttagagagacncctggccgcccgggacgtgcgtgacgcggtccagggtacatggcgtattgtgtggagcgaggcagctgttccacctgcggtgactgatatacgcagggcaagaacacagttcagccg

SEQ ID NO: 108 - DNA, 109 bp, Mus musculus
  misc_feature 59,.71: n = a, t, c, or g
ggtacagtcaaaccattgggtttccagttgtataaaagcaagcacatacaattatgtanagcacacaggtngtgtgtgtgtgtgtgtgtgtgtgtgtgtgtgtgtgtgt

SEQ ID NO: 109 - DNA, 91 bp, Mus musculus
ggtccgcgtgaaggtccatgtgtaatgtgtcagatgtggggctataggtgtgactccagtctcagaattgggggctatgcagctgcaccgg

SEQ ID NO: 110 - DNA, 170 bp, Mus musculus
  misc_feature 2, 45, 46, 47, 49, 80, 82, 107, 119, 131, 168: n = a, t, c, or g
anatcatcagatgcattctgtggaaaggacctggagcatgaatgnnnangcagccccagtctgcaacactactgggcatnangcttcaacaagggaaacataatggnggtttcccctcnaaagcaattatnggatactggtctcttttctaatctctttacttcctantt

SEQ ID NO: 111 - DNA, 155 bp, Mus musculus
  misc_feature 4, 31, 46, 50, 54, 79, 83, 84, 99, 109, 118, 147, 153: n = a, t, c, or g
ctanaacgttctggagagctcaaaaggacanattatcacccactantaanctantaagaaaatccatgatgtgtctacncatnngcacatgtagcttcntggctgcgcntcctggaantctgcacagttctcccacaccactcatangtacanca

SEQ ID NO: 112 - DNA, 132 bp, Mus musculus
  misc_feature 9, 59, 96, 117, 124: n = a, t, c, or g
caaaaaatnaagaaacgtaaaaaactaaagtgagctctccagtcctctaagaaaaaacnaacttctcagtgctgttgtgtcatctgctttacacanaggaaaaccgtggcagagcanaacgcancacaggcc

SEQ ID NO: 113 - DNA, 294 bp, Mus musculus
  misc_feature 3, 7, 9, 10, 109, 123, 152, 166, 176, 223, 224, 231, 233,236, 238, 249, 265: n = a, t, c, or g
cantgangnnggctcaaatggttagtcctggtgtatgttgcaaagggcactcatagtttactctggctttggggctttggttccccaggagggaaacagacccatccantgtgcccctccacnaggtcggctttgtttaaaaataccctgcngcattccagatcanctgagaaccnctgaaaaagacttttttgttcccttcccctttccagggtagacggcnnagtcaancnttncntcattaacaanactgccaccggctatngctttgccgagccctacaacctgtacagc

SEQ ID NO: 114 - DNA, 212 bp, Mus musculus
  misc_feature 3, 7, 26, 50, 98, 106, 110, 116, 147: n = a, t, c, or g.
agnaccngttcgccaagaggactcangccaagaaagaacgcgtggccaanaatgagctgaaccgtctgcggaacctggctcgcgcgcacaatatgcanatgcccanctcngccggnctgcaccctactggacaccagagtaaggaanagctgggccgcgccatgcaagtggccaaggtttccaccgcttcggtgggacgcttccaggagcgc

SEQ ID NO: 115 - DNA, 179 bp, Mus musculus
  misc_feature 39, 75, 77, 121, 144, 146, 148, 150: n = a, t, c, or g
ttccctttcagctgctttcaggcatgcccacccatccancactccccccaaccccaccccgtgaatacacagagngngacaaactctgtgtgtgtgtgtgtgtgtgtgtgtgtgtgtgtgngagagagagagagagagagagananananagagagagagagagagagagagagagaga

SEQ ID NO: 116 - DNA, 254 bp, Mus musculus
  misc_feature 14, 33, 47, 71, 100, 120, 140, 172, 174, 175, 178,179, 180, 185, 187, 190, 193, 194, 195, 197, 211, 213, 216,220, 223, 224, 228, 230, 236, 241, 243, 248, 252: n = a, t, c, or g.
agtgtatgtatacntttggggattgtacagaangcacagcgtagtantcaggaaaaaggaaactgggaaantaatgtataaattaaaatcagcttttaantagcttaacacacacatacnaaggcaaaaatgtaacgttnctttgatctgatcagggccgactttttttttnanntgnnnaattncnatnccnnnantaaaaggggaaagntnggntttntcnngggngnaagggnttaangnttttntttntt

SEQ ID NO: 117 - DNA, 144 bp, Mus musculus
aatcctttctgtactgagtgcctggggaggcagagagcagaagtctccagcccagtgaatactcttctcaccactagaccccagctcctgcctcagcctccccagcctggctatcagagcttagccccactctatttcccaggc

SEQ ID NO: 118 - DNA, 203 bp, Mus musculus
  misc_feature 25, 153: n = a, t, c, or g.
agtcaacataactgtacgaccaaangcaaaatacacaatgccttccccgcgagatggagtggctgtttatccctaagtggctctccaagtatacgtggcagtgagttgctgagcaattttaataaaattccagacatcgtttttcctgcatanacctcatctgcggttgatcaccctctatcactccacacactgagcggggg

SEQ ID NO: 119 - DNA, 199 bp, Mus musculus
  misc_feature 5, 26, 32, 34, 35, 48, 55, 57, 58, 59, 65, 86, 112,118, 124,131, 152, 161, 164, 168, 170, 184, 186, 190: n = a, t, c, or g.
ttatntctccatggctccaactgganggagangnngagggacacttanaattcgncnnngcaacnttgaatttttccagaaaagantgctttcacgccatgcaacatggganaagganatggangtgaaantttccatggacagaaagtaanaacactcanacntctnanttgagggcctgaantntgcntccattata

SEQ ID NO: 120 - DNA, 89 bp, Mus musculus
  misc_feature 3: n = a, t, c, or g.
tgngcatacacaccttagccgaagggtgcctgaaatccgctcagggtaacctaggcggagcagccgtgtagcacgtgggctgccacgcg

SEQ ID NO: 121 - DNA, 203 bp, Mus musculus
  misc_feature 25, 28, 68, 99, 163, 173: n = a, t, c, or g.
cccccaattcatggcatattctcgngtntagcgccttgattttccccaccccagctcctaaaccagantctgctgcaaactggctccacaggggcaaanaggatttgcctcttgtgaaaaccgactgtggccctggaactgtgtggaggtgtatggggtgtanaccggcaganactcctcccggaggagccgggtagagcgcc

SEQ ID NO: 122 - DNA, 291 bp, Mus musculus
  misc_feature 4, 133: n = a, t, c, or g.
ctgntgccagcttaaagctcaaagcttttccactccagtgcaaagagatgagattttgaatcaacagaatttgttggacttaaatgtcattttaattttttaactgatctagaaaagcacaaaggtgcacgtntttctggggcagcatgtgtgtgtcaatatgcaaacctgggctaattagaccacttcacttcactgaaacagaaaccactagattccctgtgaatccctctcttcaggaggccatgggggcagggagcacccctacatctgtgggggcactggaccccc

SEQ ID NO: 123 - DNA, 108 bp, Mus musculus
  misc_feature 32: n = a, t, c, or g.
ctcctattcagtcacaccctgctgccccatanatctctacttgaaagaggggagttaaccagcaagcctcaggataagaggacagaagtcacaaaagccacaggaggc

SEQ ID NO: 124 - DNA, 339 bp, Mus musculus
  misc_feature 58, 205, 214, 279, 317, 319: n = a, t, c, or g.
tggtgaaactggcccaggctggtcgggagggcaaggaaggaatacaggacgatctgcncatcgtattgcttccaacctgaaaaaggagcagtgtggcaacaggctgcttttttacaggctgggatgcatttcgtccccctacctgcctcgacagccctgcgcactgcaggaaggagacgaaagcattgaccaccccgaaccgccnagggagaangggcggctgggagcggacaagaccgaagacagcacccagcttcagcctttctaagcccggcgagntcaggaaccccacagacaagggccgcagcgactcgtgnanctgccgctgggaggctgtag

SEQ ID NO: 125 - DNA, 139 bp, Mus musculus
  misc_feature 5, 6, 7, 9, 10, 11, 14, 24, 25, 33, 50, 53, 54, 68,78, 80, 82, 83, 108, 110, 113, 122: n = a, t, c, or g.
atctnnncnnnctntgacctgttnngctctacntctattctccaaaaacnaanncctagaccaaggtntctgtttcancntnnacttttaagtgaaaccaaattaaancnggngacactggnagaggggagtcactgac

SEQ ID NO: 126 - DNA, 52 bp, Mus musculus
gtatggagagtgcaatgcttggtggcttcctgggtgcacccatgcccagcgc

SEQ ID NO: 127 - DNA, 149 bp, Mus musculus
  misc_feature 44, 108: n = a, t, c, or g.
ctcaaactccctcctcttgctctcctcacccacttgcgtttatntcgaaagctctcttactcatctttccccttttctgtccttcgatgtctctgattctttctccanctctgttccctcctcttttcccggtgtctctgtctccggct

SEQ ID NO: 128 - DNA, 427 bp, Mus musculus
cggccagggactcccctccaggctcctcagagagcaacaggcgaagagaactaaactgttttgccctcttcaagatcaataaccctcatataccccagggatgaaggatgctaagcccaatcctgctgcctttgtcacccctctccctgttgtgggacccaggaaagggccttggagcatcttaccccacaggggactcttaagatcactgccatcccttctctaagacaaaaccttccctaactatcacacattttaagtgtgccattccagagggctctacaaggtcattttacctttccttagacaacttactaacctcttacagatgaggaaacggagattcaaacagagattcaaacaagttccagaactcagagtctaccgcatttcccactgcacagttctagtctccagggatatgctg

SEQ ID NO: 129 - DNA, 642 bp, Mus musculus
actagaggcagtaaagtttattacattaaaactcaatgctgggtcagaggcatccacacggccctgatctctgaatcctgaaggtgtggaaccagaagccgctgtgacttgcagggtcaggacttgggtctgcctgctttgcatagctagactcctatgcatcctttcagaggtcacccaatgtcccagtcaaaagcagctgttgctctgtggccatatggcactactcctcacagagcagcgcctgtggaaggatcttccaacagcacatggacatagtccctgacgtccacacccggggctaccaggaagccccagggctgcgtctggctcctcacatccttttcctcatcttgcccttcctggagggagcaccccggccaaaggcgccctggcgcagctcctgggctcggcgtcggttgcttgggtccttgctggaggcattgatctcaaagatggttgtgcgcgtgcgatagttcttgatgctgtccaccagcctcaggcgttggagctctccctcctcaaagcatgagctgaagagtgggtgcaagcccagctctgccaggtccagctccttggctctcttgatggactcaggcgagggcgctggccgtgagcgcacatactgctgctgagcgttgt

SEQ ID NO: 130 - DNA, 7594 bp, Mus musculus
ccgccaccaaacgccggttaaaccacctcggagactgctgtgcggagaggactgggaaaccggtccccacacactgtccacgctggctccccacggaggcccacccacacccgcggcccggggcaagatgcagtgatctcagccctcccgctcctccgcacttccgcctcagtatggcctcacagctgcaggtgttttcgcccccatcagtgtcgtcgagtgccttctgcagtgcaaagaaactgaaaatagagccctctggctgggatgtttcaggacagagcagcaacgacaaatactatacccacagcaaaaccctcccagctacacaagggcaagccagctcctctcaccaggtagcaaatttcaatcttcctgcttacgaccagggcctccttctcccagctcctgccgtggagcatattgtggtaacagctgctgatagctcaggcagcgccgctacagcaaccttccaaagcagccagaccctgactcacaggagcaacgtttctttgcttgagccatatcaaaaatgtggattgaagagaaagagtgaggaagtggagagcaacggtagcgtgcagatcatagaagaacacccccctctcatgctgcagaacagaaccgtggtgggtgctgctgccacgaccaccactgtgaccaccaagagtagcagttccagtggagaaggggattaccagctggtccagcatgagatcctttgctctatgaccaacagctatgaagtcctggagttcctaggccgggggacatttggacaggtggcaaagtgctggaagcggagcaccaaggaaattgtggccattaagatcttgaagaaccacccctcctatgccagacaaggacagattgaagtgagcatcctttcccgcctaagcagtgaaaatgctgatgagtataactttgtccgttcttatgagtgttttcagcacaagaatcatacctgccttgtgtttgagatgttggagcagaacttgtacgattttctaaagcagaacaagtttagcccactgccactcaagtacataagaccaatcttgcagcaggtggccacagccctgatgaagctgaagagtcttggtctgattcatgctgaccttaaacctgaaaacataatgctagtcgatccagttcgccaaccctaccgagtgaaggtcattgactttggttctgctagtcatgtttccaaagccgtgtgttcaacctacctgcaatcacgctactacagagctcctgaaattatccttggattaccattctgtgaagctattgacatgtggtcactgggctgtgtaatagctgagctgttcctgggatggcctctttatcctggtgcttcagaatacgatcagattcgctatatttcacaaacacaaggcctgccagctgagtatcttctcagtgccggaacaaaaacaaccaggttttttaacagagatcctaatttggggtacccactgtggaggcttaagacacctgaagaacatgaattggaaactggaataaagtcaaaagaagctcggaagtacatttttaactgtttagatgacatggctcaggtaaatatgtctacagacttagaggggacagatatgttagcagagaaagcagatcggagagagtatattgatcttctaaagaaaatgctgacgattgatgcagataagagaatcacgcctctgaagactcttaaccaccaatttgtgacgatgagtcacctcctggactttcctcacagcagccacgttaagtcctgtttccagaacatggagatctgcaagcggagggttcacatgtatgacacagtgagtcagatcaagagtcccttcactacacatgtcgctccaaatacaagcacaaatctaaccatgagcttcagcaaccagctcaacacagtgcacaatcaggccagtgttctagcttccagctctactgcagcagcagctaccctttctctggctaattcagatgtctcgctgctaaactaccaatcggctttgtacccatcgtcggcagcgccagttcctggagttgcccagcagggtgtttccttacaacctggaaccacccagatctgcactcagacagatccattccagcaaacatttatagtatgcccacctgcttttcagactggactacaagcaacaacaaagcattctggattccctgtgaggatggataatgctgtgccaattgtaccccaggcgcctgctgctcagccgctgcagatccagtcaggagtactcacacagggaagctgtacaccactaatggtagcaactctccaccctcaagtagccaccatcacgccgcagtatgcggtgccctttaccctgagctgcgcagcaggccggccggcgctggttgaacagactgctgctgtactgcaagcctggcctggaggaacccaacaaattctcctgccttcagcctggcagcagctgcccggggtagctctgcacaactctgtccagcctgctgcagtgattccagaggccatggggagcagccaacagctagctgactggaggaatgcccactctcatggcaaccagtacagcactattatgcagcagccatctttgctgaccaaccatgtgaccttggccactgctcagcctctgaatgttggtgttgcccatgttgtcagacaacaacagtctagttccctcccttcaaagaagaataagcagtctgctccagtttcatccaaatcctctctggaagtcctgccttctcaagtttattctctggttgggagtagtcctcttcgtaccacatcttcttataattccctagttcctgtccaagaccagcatcagccaatcatcattccagatacccccagccctcctgtgagtgtcatcactatccgtagtgacactgatgaagaagaggacaacaaatacaagcccaatagctcgagcctgaaggcgaggtctaatgtcatcagttatgtcactgtcaatgattctccagactctgactcctccctgagcagcccacatcccacagacactctgagtgctctgcggggcaacagtgggacccttctggagggacctggcagacctgcagcagatggcattggcacccgtactatcattgtgcctcctttgaaaacacagcttggcgactgcactgtagcaacacaggcctcaggtctccttagcagtaagaccaagccagtggcctcagtgagtgggcagtcatctggatgctgtatcactcccacggggtaccgggctcagcgagggggagccagcgcggtgcagccactcaaccttagccagaaccagcagtcatcgtcagcttcaacctcgcaggaaagaagcagcaaccctgctccccgcagacagcaggcatttgtggccccgctctcccaagccccctacgccttccagcatggcagcccactgcactcgacggggcacccacacttggccccagcccctgctcacctgccaagccagcctcacctgtatacgtacgctgcccccacttctgctgctgcattgggctccaccagttccattgctcatctgttctccccccagggttcctcaaggcatgctgcagcttataccacacaccctagcactctggtgcatcaggttcctgtcagtgtcgggcccagcctcctcacttctgccagtgtggcccctgctcagtaccaacaccagtttgccactcagtcctacatcgggtcttcccgaggctcaacaatttacactggatacccgctgagtcctaccaagatcagtcagtattcttacttgtagttgatgagcacgaggagggctccgtggctgcctgctaagtagccctgagttcttaatgggctctggagagcacctccattatctcctcttgaaagttcctagccagcagcgcgttctgcggggcccactgaagcagaaggcttttccctgggaacagctctcggtgttgactgcattgttgcagtctcccaagtctgccctgtttttttaattctttattcttgtgacagcatttttggacgttggaagagctcagaagcccatcttctgcagttaccaaggaagaaagatcgttctgaagttaccctctgtcatacatttggtctctttgacttggtttctataaatgtttttaaaatgaagtaaagctcttctttacgaggggaaatgctgacttgaaatcctgtagcagatgagaaagagtcattactttttgtttgcttaaaaaactaaaacacaagacttccttgtcttttattttgaaagcagcttagcaagggtgtgcttatggcgtatggaaacagaatgatttcattttcatgtcgtgctgtccttactgggcagttgttagagttttagtacaacgagtcactgaaacctgtgcagctgctgctgagctgctcgcagagcagcactgaacaggcagccagcgctgctgggaaggaaggtgagggtgaggactgtgcccaccaggattcattctaaatgaagaccatgagttcaagtcctcctcctctctctagtttaacttaaattctccttatagaaaagccagtgaggtggtaagtgtatggtggtggtttgcatacaatagtatgcaaaatctctctctagaatgagatactggcactgataaacattgcctaagatttctatgaatttcaataatacacgtctgtgttttcctcatctctcccttctgtttcatgtgacttatttgaggggaaaactaaagaaactaaaaccagataagttgtgtatagcttttatactttaaagtagcttcctttgtatgccaacagcaaattgaatgctctcttactaagacttatgtaataagtgcatgtaggaattgcagaaaatattttaaaagtttattactgaatttaaaaatattttagaagttttgtaatggtggtgttttaatattttgcataattaaatatgtacatattgattagaagaaatataacaatttttcctctaacccaaaatgttatttgtaatcaaatgtgtagtgattacacttgaattgtgtatttagtgtgtatctgatcctccagtgttaccccggagatggattatgtctccattgtatttaaaccaaaatgaactgatacttgttggaatgtatgtgaactaattgcaattctattagagcatattactgtagtgctgagagagcaggggcattgcctgcagagaggagaccttgggattgttttgcacaggtgtgtctggtgaggagttgttcagtgtgtgtcttttccttcctcctctcctctctccccttattgtagtgccttatatgataatgtagtggttaatagagtttacagtgagcttgccttaggatgaccagcaagccccagtgaccccaagctgttcgctgggatttaacagagcaggttgagtagctgtgttgtgtaaatgcgttcgtgttctcagtctccctaccgacagtgacaagtcaaagccgcagctttcctccttaactgccacctctgtcccgttccattttggatcttcagctcagttctcacagaagcattccctaacgtggctctctcactgtgccttgctacctggcttctgtgagagttcaggaagcaggcgagaagagtgacgccagtgctaaatatgcatatttgaaggtttgtgcattacttagggtgggattccttttctctcctccatgtgatatgatagtcctttctgcatagctgtcgtttcctggtaaactttgcttggttttttttttttttgtttgttgttttttttttaaagcatgtaacagatgtgtttataccaaagagcctgttgtattgcttaatatgtcccatactacgagaagggttttgtagaactactggtgacaagaagctcacagaaaggtttcttaattagtgacgaatatgaaaaagaaagcaaaacctcttgaatctgaacaattcctgaggtttctttgggacaacatgttgttcttggggccctgcacactgtaaaattgtcctagtattcaacccctccatggatttgggtcaagttgaaggtactaggggtggggacattcttgcccatgagggatttgtggggagaaggttaaccctaagctacagagtggtccacctgaattaaattatatcagagtggtaattctaggattggttctgtgtaggtggtgtcaggaggtgcaggatggagatgggagatttcatggaacccgttcaggaaagctctgaaccaggtggaacaccgaggggctgtcaacgaacttggagtttcttcatcatggggaggaagagtttccagggcagggcaggtagtcagtttagcctgccggcaacgtggtgtgtgttgtcttttctttaatcattatattaagctgtgcgttcagcagtctgttggttgagataaccacgcatcattgtgtagtttgtcactagtgttataccgtttatgtcattctgtgtgtgatctttgtgtttcctttcccccaagcattctgggtttttcctatttaaatacagttctagtttctaggcaaacattttttttaaccttttctctataagggacaagatttattgtttttataggaatgagatgcagggaaaaaacaaaccaaccctgtccccactcctcacctccctaatccaataagcagttattgaagatgggagtcttaaatttatgggaaaagaggatgcctaggagtttgcatcgttacctgagacatctggctagcagtgtgactttacagactttgaggttgtcactctgcaaactgacatttcagattttcctagataacccatctgtgtctgctgaatgtgtatgcgccagacatagttttacattcattctggcctggggcttaacattgactgcttgccctgatggcatggaggagagccctacgaacatagcgctgactaggtcagcattgcctgaccttggaacagcttaaggctttaaaccttctcttagaacgtgcatttccagtttctcccttcccaggtgagagaggaactggaagggttgcataggcacacaccaggacacttagtcactccagagtccccagttgcaactaggaggtggttaccctgttaaccccaggaagaagaaccccatttcaaacagttccggccattgagagcctgcttttgtggttgctcatccgtcatcatccgctagaggggcttagccaggccagcacagtactggctgtcctattctgcattagtatgcaggaatttactagttgagatggtttgttttaggataggagatgaaattgcctttcggtgacaggaatggccaagcctgctttgtgtttttttttaaatgatggatggtgcagcatgtttccaagtttccatggttgtttgttgctaaaatttatataatgtgtggtttcaattcaattcagcttgaaaaataatttcactatatgtagcagtacattatatgtacattatatgtaatgttagtatttttgctttgaatccttgatattgcaatggaattcctaatttattaaatgtatttgatatgctaaaaaa

SEQ ID NO: 131 - DNA, 6543 bp, Mus musculus
ccggtcacatgctttctttgtgatgaccatcgtgatgggttccgtagaggtgggagcagcagctaaagtcaagagcatttgtgagtatgactctagcagctggacacacagagaaatgtgcatcccagctataactaaatcaagaaaggcctggctgtggaattcacaggggtccttactggattcacaggctttgatataccttgaagaagtgacacttttttccccccttggctctcagcctttcttccaggctaattcatatttacttagatggctctagatattctctcactaacctgaacctttggcatcaacacaggcttaaaggacatacttagggtctctagtgtcaattgaatggcagcatcctgactttggtcttcaaagcaaagatgacactgaagtctgccccttccaaacaagggctaccctgcctgcttccagaagcaaagcacgccttaccatctgcttaggacttcacagttcataaagttctttccatcccgtctgctttctttttattgcacaagtgtttactttttattgctcagtatttactgagataccgcagatgccactgtgcagggcgcctgcggtccttgaggaagagctgttgttcccatgcctaggcaattcagaaggccatggctggaatctgggggcaattgcatagcctgaaatcaggctgctagctgtagtggctttcccaagagaacacggggcttctgtttctggacctgtctgatgaggacaccctttcctgtctcctgccttcttctccagcagggttccccctcctttcctattcccccacgtcttctcatccccttcccgtctccacttaccccctcctaccagctcatttcttctgaagatgagccggattctttctacagtacttttgtgggatgtgaatctgactatgcagagctgggcctgggatttgtgtaacttcccttgagagcatagccttagctcttattctgttattcattatttgtaatgaatgcaggatgctccagtgccctccttgtcctcaactcttctgtgtcaagtcaggtgctatagcaggttgaggttctagctatatataagctactatctctatcattaaaatatttcaggttgttggtggcacatgcctttaatctcagcatttaggaggcagaggaaaaaggatctcttgagtttgagactagcctggctggtctacagagtgagtttcaggacagctacagccacacagaaaaaccttgtcttgggggttggggtggggaatctagatatattagtcaggattgtcttgaacgatagagccaatgtgcaatgaaagatagacatgtatctcaatatctgtgtctatatggagaaggatttatttttcataaggcattgacagagattatcatggagcttgtgaagttctgatggtctgctgtgtatacctggaaactagagaagctggctgtgtgcatagacagaattatgaaagagtgtctcagcgcaagtgcccaggcagagaaagaatgaacttgcttctcctgcttccttattcagctttctaggcatccttgagttctgatcctcagtgggctggatgatgttcacccatactgatgtaagctactcaccacactcactcactttccctcccttctctggaaacaccatcatcaatcctccttagaaatgtccttaactggttccctttgtagctcttggcccagccaaattgacacactgagtagacacaatgtatctaaccatcaattgagacactggggagacacaatgtattcaattgtctgaatcagctggctgacatccacctcaggccacaagctgaacgcacttagactgctgagggcacaaaagcactcccttccaatccaagttttgcaacaaggtagaccaaatcgagtcatcataagttattgtccttatctggctatgccctgctttgatgtttacccaatacagaacccccactgattgatgatatttgcttcctcatcactacaacttggcctgtaatgagcactgctgtttttacagcatcaggctgctaggactatgtatagagagagagctttggctttgctctggtcttataccttgtgacccattgaacacctcactttcaagacctgatggggattcatctaggactctggtccttccttcagatgtgtgtatgttgtatcagtccctcagtcccttctcctgaatcctgctaggagacctcacagcacagtattctatctgctaaaggagtttgctttccttcaatgatgctgtagtgatgctgctggaggagtagctggttctagtaatgttggtgttgaggaagataataataatactggggacattgcttttgaattaggggactagctcaagtatattatttttcatatctcatctcatctcatctcatctcatctcatctcatctcatctcatctcatctcatcttctttcctctccatacttatgttgcctattcaggaatattttggctattgtacctgtggatattcattacaaaggaggcagtggctcaaatgaagccaaagagcctggctctgaaggactgatgccaggtggccagacataggtattcaaaagaagatttgaggcttctgtttacctcttcgctgatggtgccactgctgaagtagtacttctttaccctggcagcattgtctcagtgacagctgtgtcttgtccacggggcctctgtgtcccatgctcttcacaagcttcatctccatcctctcaatgctgcagaaggccctgggctcctcagttctgcacctactactttgcttcttcccattccgaggtggtgtatttgcctcagttgctgctcctcctatcccaccattccctttcttactctctctcaggtttaattcttgtcttgtcctttctcaccattctaagatagccctgtgacgcttcccttgatgagccctaatgagactctgtagcaccaatctctcctttcctgtagtcacacgagctggaatccagattccactttgtcatttggagactcagagtattgccacacacacccctcagcgccacccccccccccataactccctgcagcccccactttctccacggcacctactcccccttgcagcttgtgccgggaagccctgtttcctagctgcagcctattatgttccagtcgacaggccggggggggggggtgtcaccgacagccccagagcctgctgcacatggtgttaagtaaggctttgggttttccatgacattggtcggtccccagggtgggcagggttcatgtgtctgcaggagtatgtgagggcatagactggaaatagccttgtcaaaatagaccaagggcaaatgctgagaggggaaatgaggctgacctggggcggcgtagggcaggtgcttctccaggggctttcctctgtgaggggccctgtagctaaaggctgcctgaaatacttcctgtgaccctctagacctacatgaggcccccatcagacacaagagcttcctgttccctcttcactttccaatacttacagagcaagaagggtttactcagttcttctttctttctcttgtcccctcagctcctgtcttagtgcatttggcctgctctaaggaagtgggactctaggctgtgtggctgtggaacaacaggggttgatttctcctggttctggaggctaggcatccccgactgtgtgccaccgacgtcattagcgcgcggcaagggcctgctttttgactcatggtcccctgtcttccaggtctaacctgggggatgaggtaaggcgcttgctggcatgtcttttctaaggatgcttattgtagttcctgggttctgttcgcatgacatttctcatgaccttggaggttagggattcaacataggaattttatgagggcataaacagcccataatagcctccttgaaatatctcttgagtgcactctccttcctcatcaggcatgtcaacaaaatttcatgtcactgtaaagcagaaataattgtactttctatagttcatattgtgacttgggcttcttcttcaatatgctcaaactgatgaccagttgcatgccaaactcacttttgccggtgtggtaaagtttgtctcctaggcttcttacttagcttcagcctttctgtattccatgaagtgaggagattcattggtggtgtgtgtcaattagtttttttgctgctgtgataaaacaccatgacaaacttgtagccatcatccagagaagtcagggtaggaacctggaggtaggaactgatgcagaggccatcgaggagtgctgcttactcctcctggatcacacagcctgctttctcaacagtaggtaggaccaacagcctaggtggcaccacccacagtgagctgggccttccacatcaatcatcaatcaagaaaaatagcacaaaaccctttcccgaaggccaatctgctggaggcattttctcagttgagattccctcttcccaaatgactgcataaaacttgtgtcatgttgacatgaaactagccagcacagggtgtctgttagtttttcggggctactaaacaatctgaaacacgctagattgctcaaatcctctgggatgcattccggtagctgtggaggcagcaaagctgatatggtgatgcccctacaatccaggggatccatgggaagagcctgccctttttccatgggcttttaatgactactggacgctctaggcatttctcagcttgacggacgcttctctagctgttctcccatggcttacttataggcttatatatttatatataggctcccatggcctatgcctataactttcttcttatatggatcagcttccatgtacgtatgtatctcaaatactatactgtgatagtgtctgtagaacccaggtccaagtcacatcttatttgcaagtactgcaggatacaatagggtatgagaatgaaatgttaactcgggatgagatacacaggtcatcccagctcttgggaagcaggagagggatgatcagaggttcaggactaccttcaattacattgtgagtttaaggctagcctgggctgccagagactttgcctcaacaactctacctttacgagagaaaagaaaaaacaagctctatggcttctctctctctctaagtaaagtatctttggttttatatttgcaatgatgtggacaatcatattgtcttagtgttctatgaagagatgtcatgaacaaggtattcttaagtttcagacgttagcccatgattatggtgacacaaaaaacaacaacaacaacaacaaaaacggacaaggttctggagaaggaactgagagtcttatattctgatctgcacgcagcagaagagggagatactgggtctgtcttgggcttttgaaacctcaaagcccacctccaatgaaacacccctacaataagaccacatctgctaatctaaatccccaagtagtggtattccctgaggactaagcatttgaatatgagcctacaggggccattttcattcaaagaagcatgcatatgtataaagaaaagcaaatacctgcatagatttggcacctgtcagagaagaggtaaattcaaagcagaaaaagcaacctaggctctggtctggtttatggagacactctgttttggcctccgctcattgcaatgacaaattattatccttggcttcagggtaaaattttctcagagttacggataccgagaagttcaaggacaaagtattaacagttcattttctggtgatggtgtctgcttcggtcatggatgtctgtcttcttttgtcatcacagtggggtcaagggttcagtgtgagagcatctaatgaaactcattctcctttaacaaagaaataaatatttatgttccatgtgtgcatgtgtgtgtgtatgggagtatatatggggtcagaacacaacttgtaggacttggatttttccaactaccatgtagattcctggaaactcaggtcttcaggctagatagaccacaagctccatttccaaaaccgtctcaccagccccatccaatgtctcttcttatgggaaacttatgagttcagatctctgccaatgcatgaggtattatgtgttcttcctaacttctatcaatacctcttctccaatatagtctcatggaaatggtggactagagctgataggatgcgcaagcacacgcacgcacgtgtgagcacacacacacacacacacacacacacacacacaccctcacttattagaatgacttataggttgtggtcctgtcttatgacagaagtccaagaacccaatagttaggttacttagatactctcacactgccctcatgctcactggcaagttcatccgtcctggagctgaggcatccttcactgatattaaagcctacctcttcaggattccaacatacattgaatagttcagtagaccagcttgatcccttagttggtcttcggttgtaatcctgaagaagttaaaaa

SEQ ID NO: 132 - DNA, 2940 bp, Mus musculus
cagagttgctctagcctggctgcccaagccaagccgttagaagcaggagcccctggccagtgcctggtcacggagctgagctgtgtttagatgtgttggctgctgcgtggtgaaggaagacccgtctccagaaaagcaatttaggcaaaagggattccgtttgatggcagagtcccagtgctagaaaggtagcgaaggtggacagcttacagtctcaactcatttcgtcgtaaatgtcctcgtaacgacattgattcttctacctggataaccttttgtttgtttgtttgtttgtttttgttttgtttttcccctgtaaccatttttttttctgacaagaaaacattttaattttctaagcaagaagcatttttcaaataccatgtctgtgacccaaagtaaaaatggatgataattcatgtaaatgtgtgcaacatagcaacctgaacctgcacgcgattcgggctctgtaggttgtgaaccatggctatgtggatacaggctcagcagctccagggcgatgcccttcaccagatgcaggccttgtacggccagcatttccccatcgaggtgcgacattatttatcacagtggatcgaaagccaagcctgggactcaatagatcttgataatccacaggagaacattaaggccacccagctcctggagggcctggtgcaggagctgcagaagaaggcggagcaccaggtgggggaagatgggtttttgctgaagatcaagctggggcactatgccacacagctccagagcacgtacgaccgctgccccatggagctggttcgctgtatccggcacattctgtacaacgaacagaggctggttcgcgaagccaacaacggcagctctccagctggaagtcttgctgacgccatgtcccagaagcaccttcagatcaaccaaacgtttgaggagctgcgcctgatcacacaggacacggagaacgagctgaagaagctgcagcagacccaagagtacttcatcatccagtaccaggagagcctgcggatccaagctcagtttgcccagctgggacagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaaccggcagcagatccgcagggctgagcacttgtgccagcagctgcccatcccaggccccgtggaggagatgctggctgaggtcaacgccaccatcacggacatcatctcagccctggtcaccagcacgttcatcatcgagaagcagcctcctcaggtcctgaagacccagaccaagtttgcagccaccgtgcgcctgctggtgggggggaagctgaatgtgcacatgaaccccccgcaggtgaaggcgaccatcatcagcgagcagcaggccaagtccctgctcaagaatgagaacacccgcaatgattacagcggcgagatcctgaacaactgttgcgtcatggagtaccaccaggccactggcacactcagcgcccacttcagaaacatgtccctgaaacgaatcaagaggtctgaccgccgtggggcagggtcagtaacggaagagaagttcacgatcctgtttgactcacagttcagcgtcggtggaaacgagctggtctttcaagtcaagaccttgtcgctcccggtggtggtgattgttcacggcagccaggacaacaatgccacagccactgtcctctgggacaacgcctttgcagagcctggcagggtgccatttgccgtgcctgacaaggtgctgtggccgcagctgtgtgaagcgctcaacatgaaattcaaggctgaagtacagagcaaccggggcttgaccaaggagaacctcgtgttcctggcacagaaactgttcaacatcagcagcaaccacctcgaggactacaacagcatgtccgtgtcctggtcccagttcaaccgggagaatttgccaggacggaattacactttctggcagtggtttgatggcgtgatggaagtattgaaaaaacatctcaagcctcactggaatgatggggctatcctgggtttcgtgaacaagcaacaggcccacgacctgctcatcaacaagccagacgggaccttcctgctgcgcttcagcgactcggaaatcgggggcatcaccattgcttggaagtttgactctcaggagagaatgttttggaatctgatgccttttaccactagagacttctctatccggtccctcgctgaccgcctgggggacctgaattacctcatatatgtgtttcctgatcggccaaaggatgaagtatattctaagtactacacaccggtcccctgtgagcccgcaactgcgaaagcagctgacggatacgtgaagccacagatcaagcaggtggtccccgagtttgcaaatgcatccacagatgctgggagtggcgccacctacatggatcaggctccttccccagtcgtgtgccctcaggctcactacaacatgtacccacccaacccggactccgtccttgataccgatggggacttcgatctggaagacacgatggacgtggcgcggcgggtcgaagagctcttaggccggcccatggacagtcagtggatccctcacgcacagtcatgaccagacctcaccacctgcagcttcatcgccctcgtggaggaacttcctgtggatgttttaattccatgaatcgcttctctttggaaacaatactcg

SEQ ID NO: 133 - DNA, 674 bp, Mus musculus
ctgccttacagcactgttctcggcagcttacaggaaaccttcctttcctgattcccaccttaccacaagacccagggctgtggggtgaggtgtgctaccgaactgaacgccagcaatgatgttccagaaaacattttaatatcttcccttggttccactgctgctaagctggggacggggctggaatagccgctccggtggaggaggcttcccagcaggggagagagataattaaaatggcattaccgtgtctccctgtgggatgcggtgacattaaagagccacactgacaaaatacccgggactggaaggttctgtgctgccttcctcgcagacacagaaccacagcagtatctgagagctgctgggaccgcttgctctgctcacaggcggtctggggcggggatcctagatgcgaagacctaccgagctgaagggagggaaagaatcggtctgggacgggcggggctatcccggggttccctatctggagggcacaagtcctgctgtggatgttagcacgctccttttggcttgaggagaacttgggaaggccggctccatgagggtggcttcccctttgttgtgccggaggtggggttccaacccgggagggtggtaacggctaagggaggcggctaaacaaccggaaggccaaatatttggattggccg

SEQ ID NO: 134 - DNA, 889 bp, Mus musculus
gtaaagatcctaaaggtggttgacccaactccagagcaacttcaggccttcaggaacgaggtggctgttttgcgcaaaacacggcatgttaacatcctgctgttcatggggtacatgacaaaggacaacctggcgattgtgactcagtggtgtgaaggcagcagtctctacaaacacctgcatgtccaggagaccaaattccagatgttccagctaattgacattgcccgacagacagctcagggaatggactatttgcatgcaaagaacatcatccacagagacatgaaatccaacaatatatttctccatgaaggcctcacggtgaaaattggagattttggtttggcaacagtgaagtcacgctggagtttggtcctcagcaggttgaacagcccactgctctgtgctgtggatggccccagaagtaatccggatgcaggatgacaacccgttcagcttccagtccgacgtgtactcgtacggcatcgtgctgtacgagctgatggctggggagcttccctacgcccacatcaacaaccgagaccagatcatcttcatggtaggccgtgggtatgcatcccctgatctcagcaggctctacaagaactgccccaaggcaatgaagaggttggtggctgactgtgtgaagaaagtcacagaagagagacctttgtttcgccagatcctgtcttccatcgagctgcttcagcactctctgccgaaaatccacaggaacgcctctgagctttccctgcatcgggcagctcacactgagggacatcatgcttgcacgctgactacattcccaaggctaccagtctcctaactgatgatgtagcctgtcttaggccacatgggaccaaaagaagtcagcaggaccaatttt

SEQ ID NO: 135 - DNA, 975 bp, Mus musculus
  misc_feature 799: n = a, t, c, or g.
acaagactttgaaaagcggttcctgaagaggattcgtgacttgggagagggtcactttgggaaggttgagctctgcagatatgatcctgagggagacaacacaggggagcaggtagctgtcaagtccctgaagcctgagagtggaggtaaccacatagctgatctgaagaaggagatagagatcttacggaacctctaccatgagaacattgtgaagtacaaaggaatctgcatggaagacggaggcaatggtatcaagctcatcatggagtttctgccttcgggaagcctaaaggagtatctgccaaagaataagaacaaaatcaacctcaaacagcagctaaaaatatgccatccagaattgtaaggggatggactacttgggttctcggcaataagttcaccgggacttagcagccagaatgtccttgttgagagtgagcatccagttgagattggagaccttgggttaacccaagccatttgaaacgattaggagtactacacagttcaggaccaccgggaaaagccagtgttccggtacgctccggaatgtttaatccagtgttaattttaaaacgcctccgatgtccggtcctttggagtgacactgcacgagctgctcaattactgtgactccgaatttagtcccatggccttggtcccgaaaaggtaagcccaactccaggccagaagacaattgaaggcctgtggatcactgaaagaaggaaagccctggcatgtccacccaatgtcctgatgaagttaacagcctatgggaaaattcctggaattcganctactaaccgaacaattttcggaacctatggaagagtttaagcccctttaaatagaagcctggcacactttaatccccatttcaaatctttctccaagcctttaaaaaggtttaaaggaaagttgaatcgggcctaagtcccaaaaaaccgcggtacaattgcaattcacgggtcc

SEQ ID NO: 136 - DNA, 3026 bp, Mus musculus
tggactgggtgcggccggctgcaagactctagtcgtcggcccacgtggctggggcggggactgccgtggcgcctagtgattacgtagcgggtggggcccgaagtgccgctccctggcggggctgttcatggcggtttcggggtctccaacagctcaggttgaagtccaaaagcctcccgaggcgggctgcggagtttgaggtttttgctggtgtgaaatgactgagtacaaactggtggtggttggagcaggtggtgttgggaaaagcgccctgacgatccagctaatccagaaccactttgtggatgaatatgatcccaccatagaggattcttaccgaaagcaagtggtgattgatggtgagacctgcctgctggacatactggacacagctggacaagaggagtacagtgccatgagagaccagtacatgaggacaggcgaagggttcctctgtgtatttgccatcaataatagcaaatcatttgcagatattaacctctacagggagcaaattaagcgtgtgaaagattctgatgatgtccccatggtgctggtaggcaacaagtgtgacttgccaacaaggacagttgacacaaagcaagcccacgaactggccaagagttacggaattccattcattgagacctcagccaagacccgacagggtgtggaggatgccttttacacactggtaagggagatacgccagtaccgattgaaaaagctcaacagcagtgacgatggcactcaaggttgtatggggtcgccctgtgtgctgatgtgtaagacactttgaaagttctgtcatcagaaaagagccactttgaagctgcactgatgccctggttctgacatccctggaggagacctgttcctgctgctctctgcatctcagagaagctcctgcttcctgcttccccgactcagttactgagcacagccatctaacctgagacctcttcagaataactacctcctcactcggctgtctgaccagagaaatggacctgtctctcccggtcgttctctgccctgggttcccctagaaacagacacagcctccagctggctttgtcctctgaaaagcagtttacattgatgcagagaaccaaactagacatgccattctgttgacaacagtttcttatactctaaggtaacaactgctggtgattttcccctgcccccaactgttgaacttggccttgttggtttggggggaaaatgtcataaattactttcttcccaaaatataattagtgttgctgattgatttgtaatgtgatcagctatattccataaactggcatctgctctgtattcataaatgcaaacacgaatactctcaactgcatgcaattaaatccaacattcacaacaaagtgcctttttcctaaaagtgctctgtaggctccattacagtttgtaattggaatagatgtgtcaagaaccattgtataggaaagtgactctgagccatctacctttgagggaaaggtgtatgtacctgatggcagatgctttgtgtatgcacatgaagatagtttccctgtctgggattctcccaggagaaagatggaactgaaacaattacaagtaatttcatttaattctagctaatcttttttttttttttttttttggtagactatcacctataaatatttggaatatcttctagcttactgataatctaataattaatgagcttccattataatgaattggttcataccaggaagccctccatttatagtatagatactgtaaaaattggcatgttgttactttatagctgtgattaatgattcctcagaccttgctgagatatagttattagcagacaggttatatctttgctgcatagtttcttcatggaatatatatctatctgtatgtggagagaacgtggccctcagttcccttctcagcatccctcatctctcagcctagagaagttcgagcatcctagaggggcttgaacagttatctcggttaaaccatggtgctaatggaccgggtcatggtttcaaaacttgaacaagccagttagcatcacagagaaacagtccatccatatttgctccctgcctattattcctgcttacagacttttgcctgatgcctgctgttagtgctacaaggataaagcttgtgtggttctcaccaggactggaagtacctggtgagctctggggtaagcctagatatctttacattttcagacccttattcttagccacgtggaaactgaagccagagtccatacctccatctccttccccccccaaaaaaattagattaatgttctttatatagcttttttaaagtatttaaaacatgtctataagttaggctgccaactaacaaaagctgatgtgtttgttcaaataaagaggtatccttcgctactcgagagaagaatgtaaaatgccattgattgttgtcacttggaggcttgatgtttgccctgataattcattagtgggttttgtttgtcacatgatacctaagatgtaactcagctcagtaattctaatgaaaacataaattggataccttaattgaaaaaagcaaacctaattccaaaatggccattttctcttctgatcttgtaatacctaaaattctgaggtccttgggattcttttgtttataacaggatcttgctgtgtagtcctagctggcctcaaactcacaatactcttcctggatcaatctcccaagtgctgggattacaggcacattccaccacacacacctgactgagctcgttcctaatgagttttcattaagcaaattccccatcaccttgaaactaatcagaagggggaacaaacatttgctatgctcctgagtgctaacactgggctcattcacatggggtttgcattcctaggcaaactaaactgctgccttttacaacaaggctcagtcatcttcctgaagctgctgagaccagcacttggtcttgttttgttttaatatgtctatatgactggtggtggatccgtcgacctgca

SEQ ID NO: 137 - DNA, 3792 bp, Mus musculus
ttataagccgcagtgcccggatgtgaatggattacaatgtatctttcagggaaacctattattatcaatgtgactcctcgggggagtcaatgatggtgttggggaggaggatgatgatgagacgcctctaaacttggaacaagtttaggactttgaaagagaagagaaaaaaaaaatacaaccaacaagaccgaagaacaattataactatccagtgttgattatttttataaacaatacgaaaaagttgtcggatttttttttttaatgattactttttggggggagggaattttgttacagtttgatgatggaaaatgcaaaaaccgagccaggtgcataatcttgtaatctgtggctaaccctggaacaggactgacttctatttaaaatactcttttgggggaacactcatgtgagacactaagttcttgcagaagatttttgtctctctttttaaagtctctttccttggaatattgtgagcatatttgtggccattgaaggtttgtgtgattttgctaaaatgcatcaccaacagcgaatggctgccttagggacggacaaagagctgagtgatttactggatttcagtgcgatgttttcgcctcctgtaagcagtgggaaaaatggaccaacttctttggcgagtggacatttcactggctcaaatgtagaagacagaagtagctcagggtcctggggaactggaggccatccaagcccgtccaggaactatggagatgggactccctatgaccacatgactagcagggatcttgggtcacatgacaatctctctccaccttttgtcaattccagaatacaaagtaaaacagaaaggggctcatactcatcttatgggagagaaaacgttcagggttgccaccagcagagtctcctcggaggggacatggatatgggcaatccaggaaccctttcgcccaccaaacctggctcccagtactatcagtattcaagcaataatgcccgccggaggcctcttcacagtagtgccatggaggtacagacaaagaaagtccgaaaagttcctccgggtttgccgtcttcagtctacgctccttcagccagcactgccgactacaacagggactcgccaggctatccttcctccaagccagcagccagcactttccctagctccttcttcatgcaagatggccatcacagcagcgacccttggagctcctccagcgggatgaatcagcccggctacggagggatgctgggcaattcttctcatatcccacagtccagcagctactgtagcctgcatccacacgaacgtttgagctatccatcccactcctcggcagacatcaactccagtcttcctccgatgtccacgttccatcgtagtggcacaaaccattacagcacctcttcctgcacaccccctgccaacggaacagacagtataatggcaaacagaggaactggggcagcaggcagctcgcagactggagacgctctggggaaagccctagcttcgatctattctcctgaccacacgaacaacagcttttcctccaatccttcaactcctgtgggctcccctccttcactctcagcaggcacagctgtttggtctagaaatggaggacaggcctcgtcatctcccaattatgaaggacccttgcactcactgcaaagccgaatcgaagaccgtttggaaagactggacgatgcgattcatgttctccggaaccacgcagtgggcccgtccacagctgtgcctggtggccatggggacatgcatgggatcatgggaccctcccacaacggagcgatgggtagcctgggctcagggtacggaactagtcttctctcagccaacagacactcgctcatggttggggcccaccgtgaagatggcgtggctctgagaggcagccattctctcctgccaaaccaggttccggtcccacaacttccggtccagtctgcaacttcccctgacttgaacccaccccaagacccttacagagggatgccaccaggcctccagggccagagcgtgtcttctggtagctctgagatcaaatccgatgacgagggcgatgagaacctgcaagacacaaaatcttctgaggacaagaaattagatgacgacaagaaggatatcaaatcaattactaggtcaagatctagcaataacgatgatgaggacctgaccccagagcagaaggctgagcgcgagaaggaacggaggatggccaataatgcccgtgagcgcctgagggtccgagatatcaacgaggctttcaaggagcttggccgtatggtgcagctccacctgaagagcgacaagccccagaccaagctcctgattctccaccaggccgtggctgtcatcctcagcctggagcagcaagttcgagaaaggaatctgaacccgaaagctgcctgtctgaaaagaagggaggaagagaaggtgtcctcagagcctcccccactctccttggctggcccacaccctgggatgggagacgcagcgaatcacatgggacagatgtgaaaaggtccaagttgctaccttgcttcattaaacaagagaccacttccttaacagctgtattaccctaaacccacataaacactgctccttaaccccgtttttttttgtaatataagacaagtctgagtagttatgaatcgcagacgcaagaggtttcagcattcccaattatcaaaaaacagaaaaacaaacaaaaaaatgaatgaaagaaagaaagaaagaaaaaaatgcaacttgagggacgacttctttaacatatcactctgaatgtgcgacggtatgtacaggctgagacacagcccagagactgaatggcaatcctccacactgtggagcaatgcatttgtgcctaaacttcttttggaaaaaaaaaatataattaatttgtaagtctgaaaaaaatatttaatttaaaaaaaattgtaaacttgcaataatgaaaaagtgtacttctgaagaaaacgacatgaacgtttttgttggtattcacgtcagctagtgtttctaattaccggatattgaataggggaagcccggctgccctcgtaacaaaaccagcaaacgtcctgatggcaacgaagtgatgacattagccattccttagggtaggagggacagatggatgttatagacctatgacaaatatatatataaatatatatataaatatatattaaaaatttagtgactatggtaagcttgtgatgtcagcttttctcctgtaaaaatagtactgataactttttaaaagaaagattttactgtaaatatggattttttttttgtctgatttttgtcccttcccccggtttgttatcgtaacctgtagtgccaactctgcttccggaggggcagtgcaggacgaaatgctgaccctgaagttgcttctcattcacaaatagtaaaaagttgtttctccagtcttttgggaacacaggacttaaaagtcacatcatgtgtaggaattacatgcagcattgcccgggcgaggaaaaaagcgtttgtctggcttgtggcgctgcccttgttaccctcccctgggattttcagaggtacacggttagaatgctacaatgttaccactgtgccttccaatgtttatatcatcggaaacataacataatcaaagtggctgtgatttaacaaaaaaaacgattcaagtgttacctacctgtgtagccgaagtagtgtgcagtgaccgagacgtttcagaatacatggtcagattttttttggaaaaaatacaaaaatta

SEQ ID NO: 138 - DNA, 1208 bp, Mus musculus
ctgtccatttcatcaagtcctgaaatatcgaaatggatttagagaaaaattacccgactcctcggaccatcaggacaggacatggaggagtgaatcagcttgggggggtttttgtgaatggacggccactcccagatgtagtccgccaaaggatagtggaacttgcccatcaaggtgtcaggccctgcgacatctccaggcagcttcgggtcagccatggttgtgtcagcaaaattcttggcaggtattatgagacaggaagcatcaagccgggggtgattggaggatccaaaccaaaggttgccactcccaaagtggtggaaaaaatcgctgagtacaaacgccaaaaccctaccatgtttgcctgggagatcagggaccggctgttggcagagcgagtctgtgacaatgacactgtgcccagcgtcagctccatcaacaggatcattcggacaaaagtacagcagccccccaatcagccggtcccagcttccagtcacagcatagtgtctacaggctccgtgacgcaggtgtcatcggtgagcaccgactccgcgggctcctcatactccatcagtggcatcctgggcatcacgtcccccagtgccgacaccaacaaacgcaagagggatgaaggtattcaggagtctccagtgccgaatggccactcacttccgggccgggacttcctccggaagcagatgcggggagacctgttcacacagcagcagctggaggtgctggaccgcgtgtttgagagacagcactactctgacatcttcaccaccacggaacccatcaagccagaacagaccacagagtattcagccatggcttcactggctggaggcctggatgacatgaaagccaacttgacgagccccacccccgctgacatcgggagcagcgttccaggcccacagtcctaccctattgtcacaggccgagacttggcgagcacaaccctcccggggtaccctccacacgtcccccccgctggacagggcagctactctgcaccgacgctgacagggatggtgcctgggagtgaattttctggaagtccctacagccaccctcagtattcttcctacaatgattcttggaggttccccaacccagggctgcttggctccccatactattacagccctgcagcccgaggagcggccccaccggccgcagccactgcgtacgaccgccactga

SEQ ID NO: 139 - DNA, 2655 bp, Mus musculus
gttgagcgcgaagcagccgagatggaaggaagccctaccaccgccactgcggtggaaggaaaagtcccctctccggagagaggggacggatcttccacccagcctgaagcaatggatgccaagccagcccctgctgcccaagccgtctctaccggatctgatgctggagctcctacggattccgcgatgctcacagatagccagagcgatgccggagaagacgggacagccccaggaacgccttcagatctccagtcggatcctgaagaactcgaagaagccccagctgtccgcgccgatcctgacggaggggcagccccagtcgccccagccactcctgccgagtccgagtctgaaggcagcagagatccagccgccgagccagcctccgaggcagtccctgccaccacggccgagtctgcctccggggcagcccctgtcacccaggtggagcccgcagccgcggcagtctctgccaccctggcggagcctgccgcccgggcagcccctatcacccccaaggagcccactacccgggcagtcccctctgctagagcccatccggccgctggagcagtccctggcgccccagcaatgtcagcctctgctagggcagctgccgctagggcagcctatgcaggtccactggtctggggagccaggtcactctcagctactcccgccgctcgggcatcccttcctgcccgcgcagcagctgccgcccgggcagcctctgctgcccgcgcagtcgctgctggccggtcagcctctgccgcgcccagcagggcccatcttagaccccccagccccgagatccaggttgctgacccgcctactccgcggcctcctccgcggccgactgcctggcctgacaagtacgagcggggccgaagctgctgcaggtacgaggcatcgtctggcatctgcgagatcgagtcctccagtgatgagtcggaagaaggggccaccggctgcttccagtggcttctgcggcgaaaccgccgccctggcctgccccggagccacacggtcgggagcaacccagtccgcaacttcttcacccgagccttcggaagctgcttcggtctatccgagtgtacccgatcacgatccctcagccccgggaaggccaaggatcctatggaggagaggcgcaaacagatgcgcaaagaagccattgagatgcgagagcagaagcgcgcagataagaaacgcagcaagctcatcgacaagcaactggaggaggagaagatggactacatgtgtacacaccgcctgctgcttctaggtgctggagagtctggcaaaagcaccattgtgaagcagatgaggatcctgcatgttaatgggtttaacggagatagtgagaaggccactaaagtgcaggacatcaaaaacaacctgaaggaggccattgaaaccattgtggccgccatgagcaacctggtgccccctgtggagctggccaaccctgagaaccagttcagagtggactacattctgagcgtgatgaacgtgccgaactttgacttcccacctgaattctatgagcatgccaaggctctgtgggaggatgagggagtgcgtgcctgctacgagcgctccaatgagtaccagctgattgactgtgcccagtacttcctggacaagattgatgtgatcaagcaggccgactacgtgccaagtgaccaggacctgcttcgctgccgtgtcctgacctctggaatctttgagaccaagttccaggtggacaaagtcaacttccacatgttcgatgtgggcggccagcgcgatgagcgccgcaagtggatccagtgcttcaatgatgtgactgccatcatcttcgtggtggccagcagcagctacaacatggtcattcgggaggacaaccagactaaccgcctgcaggaggctctgaacctcttcaagagcatctggaacaacagatggctgcgcaccatctctgtgattctcttcctcaacaagcaagacctgcttgctgagaaagtcctcgctggcaaatcgaagattgaggactactttccagagttcgctcgctacaccactcctgaggatgcgactcccgagccgggagaggacccacgcgtgacccgggccaagtacttcattcgggatgagtttctgagaatcagcactgctagtggagatgggcgccactactgctaccctcactttacctgcgccgtggacactgagaacatccgccgtgtcttcaacgactgccgtgacatcatccagcgcatgcatctccgccaatacgagctgctctaagaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaatcaaaaatgagcaaaaaaaaaaa

SEQ ID NO: 140 - DNA, 3026 bp, Mus musculus; identical to SEQ ID NO: 136

SEQ ID NO: 141 - DNA, 485 bp, Mus musculus
  misc_feature 383: n = a, t, c, or g.
gctggtgccttcgccgtggcctgctggtgacggtccggagcgatgctgagcccgggcccagcctctcagctccgccttgtgcgctgcacagatctaggggagcctgacgggacgttgacaacgtggaataggagcagtatcatcccaccatgaggttggggatttaagagtggaagatgccaacagctgtgtcctcccatgagggtgtcccctttcaagttctcagaacggatgcaggactgcagatctgtgctggcaacagcagaggctatattcccagaggagtctccagccggcctgaaagcaaatatctatcctaagtgacatgtctgccaatttggttctgggtgggcacatttggtaatcctggtctgtaccacagngatcttctacgccgttttaaaacataaacattgggtttattaaaccaggaaagaacaaacaaaacaaagaaacaacggggggggcgggtctaagaatatccg

SEQ ID NO: 142 - DNA, 2484 bp, Mus musculus
tgctccatgcccttgtcctcgctctggcccttgcctcttgccctagccttttctccgcctctaagttcttgtcccgtccctaggtccttgttccagggggtgggggcggggcggactaaggctggcctgccactccagcgagcaggctatctcctagttctcgctgctcggactagccattgccgccgcctcacctctgctgcaagtagcctcgccgtcggggagccctaccacacggtccgccctcagcatgatggacttggagttgccaccgccagactacagtcccagcaggacatggatttgattgacatcctttggaggcaagacatagatcttggagtaagtcgagaagtgtttgactttagtcagcgacagaaggactatgagctggaaaaacagaaaaaactcgaaaaggaaagacaagagcaactccagaaggaacaggagaaggccttttttgctcagtttcaactggatgaagaaacaggagaattcctcccaattcagccggcccagcacatccagacagacaccagtggatccgccagctactcccaggttgcccacattcccaaacaagatgccttgtactttgaagactgtatgcagcttttggcagagacattcccatttgtagatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacgagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactaggttcgggaggatggagccttttctgagctagtgtttgttttgtactgctaaaacttcctactgtgatgtgaaatgcagaaacactttataagtaactatgcagaattatagccaaagctagtatagcaataatatgaaactttacaaagcattaaagtctcaatgttgaatcagtttcattttaactctcaagttaatttcttaggcaccatttgggagagtttctgtttaagtgtaaatactacagaacttatttatactgttctcacttgttacagtcatagacttatatgacatctggctaaaagcaaactattgaaaactaaccagaccactatacttttttatatactgtatgaacaggaaatgacatttttatattaaattgtttagctcataaaaattaaaaggagctagcactaataaaagaatatcatgact

SEQ ID NO: 143 - DNA, 2300 bp, Mus musculus
tatattccgggggtctgcgcggccgaggacccctgggtgcgctgctctcagctgccgggtccgactcgcctcactcagctcccctcctgcctcctgaagggcagcttcgccgacgcttggcgggaaaaagaagggaggggagggatcctgagtcgcagtataaaagaagcttttcgggcgtttttttctgactcgctgtagtaattccagcgagagacagagggagtgagcggacggttggaagagccgtgtgtgcagagccgcgctccggggcgacctaagaaggcagctctggagtgagaggggctttgcctccgagcctgccgcccactctccccaaccctgcgactgacccaacatcagcggccgcaaccctcgccgccgctgggaaactttgcccattgcagcgggcagacacttctcactggaacttacaatctgcgagccaggacaggactccccaggctccggggagggaatttttgtctatttggggacagtgttctctgcctctgcccgcgatcagctctcctgaaaagagctcctcgagctgtttgaaggctggatttcctttgggcgttggaaaccccgcagacagccacgacgatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataaactgacctaactcgaggaggagctggaatctctcgtgagagtaaggagaacggttccttctgacagaactgatgcgctggaattaaaatgcatgctcaaagcctaacctcacaaccttggctggggctttgggactgtaagcttcagccataattttaactgcctcaaacttaaatagtataaaagaactttttttatgcttcccatcttttttctttttccttttaacagatttgtatttaattgtttttttaaaaaaatcttaaaatctatccaattttcccatgtaaatagggccttgaaatgtaaataactttaataaaacgtttataacagttacaaaagattttaagacatgtaccataatttttttt

SEQ ID NO: 144 - DNA, 2300 bp, Mus musculus; identical to SEQ ID NO: 143

SEQ ID NO: 145 - DNA, 2300 bp, Mus musculus; identical to SEQ ID NO: 143

SEQ ID NO: 146 - DNA, 503 bp, Mus musculus
  misc_feature 476: n = a, t, c, or g.
tttttttttttgcttttttttttctttctttctttttctttttttctttctttttttgagagtatttgggcgacgcattgggcgccctctgcagtacgcgcagcgaagcgcaccgaggctgcggaggcagagctgcatgctgggcgcgtggacaggtgggcgtgaagcaaaaggacatttttgggagtatggggtttgggacgagggtggggagaaaaggcaaaaggagaccacgttagactgaagagctaaaaagggcacggacttggctacgccaagacgaagccagcctgggagagggagtctctgggaccggcggggggagggggggggctcctgaagctggctggttggtgggaaggaggggctcacaaacacagtagggaagtcttgtcactgcgaaggggacgcggcatccgactctcctctggaacttctaaaacgttcagctctggcctagtctccgctggggccgncgcccgcgcctccccgggcgcccccag

SEQ ID NO: 147 - DNA, 673 bp, Mus musculus
gcctttaaaaacgtttattttatgtgcataagtgctttgcatactatgagcatgtctggtgctccaaaaggccaggagagggtgccagatcctctgaaaccagatgtagagggttatgagccgccatgaggatgctgggaactgaacccaggccctttgcacaagcagcaagtgctcctagcgcttcagccacttcttcatcctcagcatgatgaacagagtaaaagccatgaacattgatgaaataaaaacatgagtcatgttaaagaactctggatcttaacggtggacaataggctatactgtctcatttcatttaaaaaaatatgcatctttatataatcatagaaaaagatggcgaggcacagtcacaccaaaacattgagaagattactcatggggcattagaatttggagtggttttagcttctttcccacttacttcctgttttcatgtcacatgaaaagtattaatgctgccctcaaaacagagcaacatagttattaggggagactgaggcctagacaagacagctcttttacactgaatgactgtggacctgacaaagtggtagatggtgtgctgtgactgttcctgccgtggtagctacatggtctgaagactcaattgccgtgtgcaggaggaatcttcttgctcgggcatctgaccgct

SEQ ID NO: 148 - DNA, 2300 bp, Mus musculus; identical to SEQ ID NO: 143

SEQ ID NO: 149 - DNA, 2300 bp, Mus musculus; identical to SEQ ID NO: 143

SEQ ID NO: 150 - DNA, 2300 bp, Mus musculus; identical to SEQ ID NO: 143

SEQ ID NO: 151 - DNA, 3454 bp, Mus musculus
ggcacgagccgagttggaggaagcagcggcagcggcagcggcagcggtagcggtgaggacggctgtgcagccaaggaaccgggacagcgaagcgacggcaggtcgcagctggatcgcaggagcctgggagctgggagcttcagaggccgctgaagcccaggctgggcagaggaaggaagcgagccgacccggaggtgaagctgagagtggagcgtggcagtaaaatcagacgacagatggacagtgtgacaggaacgtcagagaggattgggcctcgctgcgagagtcagcctggagtcaaggtgttgacaagttgctgagaaggacacgtgggaggacggtggcgcgcggagggagagccctgtcttcagtcaccccgttgatggaggacagatggacagcagccggacggccagtcacctctcttaaacctttggatagtggtcctttgtgctctgctggacacctgttggggattttagcccattctctgaactcactttctcttaaaacgtaaactcggacggcagtgtgcgagccagctcctctgtggcagggcactagagctgcagacatgagtgcagagggctaccagtacagagcactgtacgactacaagaaggagcgagaggaagacattgacctacacctgggggacatactgactgtgaataaaggctccttagtggcacttggattcagtgatggccaggaagcccggcctgaagatattggctggttaaatggctacaatgaaaccactggggagaggggagactttccaggaacttacgttgaatacattggaaggaaaagaatttcaccccctactcccaagcctcggccccctcgaccgcttcctgttgctccgggttcttcaaaaactgaagctgacacggagcagcaagcgttgccccttcctgacctggccgagcagtttgcccctcctgatgttgccccgcctctccttataaagctcctggaagccattgagaagaaaggactggaatgttcgactctatacagaacacaaagctccagcaaccctgcagaattacgacagcttcttgattgtgatgccgcgtcagtggacttggagatgatcgacgtacacgtcttagcagatgctttcaaacgctatctcgccgacttaccaaatcctgtcattcctgtagctgtttacaatgagatgatgtctttagcccaagaactacagagccctgaagactgcatccagctgttgaagaagctcattagattgcctaatatacctcatcagtgttggcttacgcttcagtatttgctcaagcattttttcaagctctctcaagcctccagcaaaaaccttttgaatgcaagagtcctctctgagattttcagccccgtgcttttcagatttccagccgccagctctgataatactgaacacctcataaaagcgatagagattttaatctcaacggaatggaatgagagacagccagcaccagcactgccccccaaaccacccaagcccactactgtagccaacaacagcatgaacaacaatatgtccttgcaggatgctgaatggtactggggagacatctcaagggaagaagtgaatgaaaaactccgagacactgctgatgggacctttttggtacgagacgcatctactaaaatgcacggcgattacactcttacacctaggaaaggaggaaataacaaattaatcaaaatctttcaccgtgatggaaaatatggcttctctgatccattaaccttcaactctgtggttgagttaataaaccactaccggaatgagtctttagctcagtacaaccccaagctggatgtgaagttgctctacccagtgtccaaataccagcaggatcaagttgtcaaagaagataatattgaagctgtagggaaaaaattacatgaatataatactcaatttcaagaaaaaagtcgggaatatgatagattatatgaggagtacacccgtacttcccaggaaatccaaatgaaaagaacggctatcgaagcatttaatgaaaccataaaaatatttgaagaacaatgccaaacccaggagcggtacagcaaagaatacatagagaagtttaaacgcgaaggcaacgagaaagaaattcaaaggattatgcataaccatgataagctgaagtcgcgtatcagtgagatcattgacagtaggaggaggttggaagaagacttgaagaagcaggcagctgagtaccgagagatcgacaaacgcatgaacagtattaagccggacctcatccagttgagaaagacaagagaccaatacttgatgtggctgacgcagaaaggtgtgcggcagaagaagctgaacgagtggctggggaatgaaaataccgaagatcaatactccctggtagaagatgatgaggatttgccccaccatgacgagaagacgtggaatgtcgggagcagcaaccgaaacaaagcggagaacctattgcgagggaagcgagacggcactttccttgtccgggagagcagtaagcagggctgctatgcctgctccgtagtggtagacggcgaagtcaagcattgcgtcattaacaagactgccaccggctatggctttgccgagccctacaacctgtacagctccctgaaggagctggtgctacattatcaacacacctccctcgtgcagcacaatgactccctcaatgtcacactagcatacccagtatatgcacaacagaggcgatgaagcgctgccctcggatccagttcctcaccttcaagccacccaaggcctctgagaagcaaagggctcctctccagcccgacctgtgaactgagctgcagaaatgaagccggctgtctgcacatgggactagagctttcttggacaaaaagaagtcggggaagacacgcagcctcggactgttggatgaccagacgtttctaaccttatcctctttctttctttctttctttctttctttctttctttctttctttctttctttctttctttctttctaatttaaagccacaacacacaaccaacacacagagagaaagaaatgcaaaaatctctccgtgcagggacaaagaggcctttaaccatggtgcttgttaacgctttctgaagctttaccagctacaagttgggactttggagaccagaaggtagacagggccgaagagcctgcgcctggggccgcttggtccagcctggtgtagcctgggtgtcgctgggtgtggtgaacccagacacatcacactgtggattatttcctttttaaaagagcgaatgatatgtatcagagagccgcgtctgctcacgcaggacactttgagagaacattgatgcagtctgttcggaggaaaaatgaaacaccagaaaacgtttttgtttaaacttatcaagtcagcaaccaacaacccaccaacagaaaaaaaaaaaaaa

SEQ ID NO: 152 - DNA, 2034 bp, Mus musculus
gttgccggtttagggtgctgctgtagtggcgatacgtcccgccgctgtcccgaagtgagggatccgagccgcagcgagtgccatggagggccagcgcgtggaggagctgctggccaaggcagagcaggaggaggcggagaagctgcagcgcatcacggtgcacaaggagctggagctggagttcgacctgggcaacctgctggcttcggaccgcaaccccccgaccgtgctgcgccaggccgggccgtcgccggaggccgagctgcgggccctggcgcgggacaacacgcagctgctcatcaaccagctgtggcggctgccgaccgagcgcgtggaggaggcggtggtcgcgcgcttgccggagcccgccactcgcctgccccgcgagaagccgctgccccgaccacggccgctcacccgctggcagcagttcgcgcgccttaagggaatccgtcccaagaagaagaccaacctcgtgtgggacgaggctagtggccagtggcggcgccgttggggctacaagcgcgcccgggatgacactaaagaatggctgatcgaggtgcctgggagcgccgaccccatggaagaccagttcgccaagaggactcaggccaagaaagaacgcgtggccaagaatgagctgaaccgtctgcggaacctggctcgcgcgcacaagatgcagatgcccagctcagccggcctgcaccctactggacaccagagtaaggaagagctgggccgcgccatgcaagtggccaaggtttccaccgcttcggtgggacgcttccaggagcgccttcccaaggagaaagctccccggggctccggcaagaagaggaagtttcagcccctctttggggacttcgcagccgagaaaaagaaccagttggagctacttcgagtcatgaacagcaagaaacctcggctggacgtgacgagggccaccaacaagcagatgagggaagaggaccaggaggaggctgccaagaggaggaaaatgagccagaaaggcaagaggaaagggggccggcaaggaccttcgggcaagagaaggggcggcccgccgggtcagggagaaaagaggaaaggaggcttgggaagcaaaaagcattcctggccttctgctttagctggcaagaagaaaggagtgccgccccaaggtgggaagaggaggaagtagcgttctcccctcgggaccagttctgaaaagctgggactgtactaaaagttaacttgggcggtataggtggccgctgccctcagtgacatttgacattaaaaggacgggtttgccttccctcgagtcagtgctggacgagttaatagagacactgactggaaattggtgtattttgagaattatagaaatgatatagccagaaccaggaataagttaaggcctgcctttttatcttgactttggatactgcgttacagtagattggtttcaacatttttgcattatttttataacaaagcttgtgtatttatcaaagcggggagggcggggaaaaattatatctacctgtgatttgcaagtattgtaaatggatgcaggtacctggtgttgcttttaacttttactgtcggtagaggttgcatgtgaagccagtaacctgggcaccaatatggagtgtgcttgagaaaaacaaagtagttacagtggttctaaaaaagaccccttgttttaggaaaactttggccctaactataatattaaaagtatagtgctttttggtgttggttcaggtggtgcatttggccaatggattgctttaagtccagaaatagttgtcattttgtttgtaaccggtggcttttgtttaattggcttgggttttagatattgtcaaaatatctggcattcactatggaaccaaggctgccctggaactcagggccaagtgctgagattataatcgagcagcagatttcatgtttatttctgtcctagatgtttttccctgtttcattgtcttattttgttcttaataaacttatctttgcataaaaaaaaaaaaaaaaaaggccaca

SEQ ID NO: 153 - DNA, 2300 bp, Mus musculus; identical to SEQ ID NO: 143

SEQ ID NO: 154 - DNA, 2300 bp, Mus musculus; identical to SEQ ID NO: 143

SEQ ID NO: 155 - DNA, 2300 bp, Mus musculus; identical to SEQ ID NO: 143

SEQ ID NO: 156 - DNA, 1003 bp, Homo sapiens
gtgtggctggacctcgtgtcgcgagctgccattgcccagtggatggaagaagaaagggctccgcgcaagcgccgatggcgcggcctcccagtgccctgcggcagcgactcggaggacgcgcgagtttgcagatccatgtgctggacagatgactgccctgggcccggaagctgggacctggaagacccctgcccaccttccccacctcggaatgcacctcgcgatgtggagcccggacacccgggcagatggctgcgtgcccagaacaagcaagacagaagaacgtctggcaggcttccagtccatgggccctgagctacccggtgttcaaaggcatcatgacacgaaggggtacaaggtgccaacacccatccagaggaagaccatcccggtgatcttggatggcaaggacgtggtggccatggcccggacgggcagtggcaagacatgctgcttcctcctcccaatgtccgagcggctcaagacccacagttgcccagacccggggccctgtgccctcatcctcttcgccgacccgagagctggcccttgcagaccctgaagttcactacggagctaggccagtcccttggcctcaagactgccctgatcctgggtggcgcccggatgcccacccgcctcgcagcccttgcaccgcaaatcccgacatacttttggcaggcccggaccgttggggcctgtgggctgtggcaattgagcctgcagctcccagttttgcgctccgtggtggtccgcgcaccctgccgcgctcttcgccccgcgttctcgctcatccccttccgtggcgctttccgccggcctccccgcgggggccccaccaccggcgggcgctccctgcgccggcctccccaccctgtcgtgctcggcgattgtccccggctgtgcctccggggggcggtggtcaccccggctgcgggcgactacacccctcgcgcctcagtgcccctcttcccccgggcgggaggacccacgccgcgtcgcc

SEQ ID NO: 157 - DNA, 5761 bp, Homo sapiens
cacaccgcagtatgcggtgccctttactctgagctgcgcagccggccggccggcgctggttgaacagactgccgctgtactggcgtggcctggagggactcagcaaattctcctgccttcaacttggcaacagttgcctggggtagctctacacaactctgtccagcccacagcaatgattccagaggccatggggagtggacagcagctagctgactggaggaatgcccactctcatggcaaccagtacagcactatcatgcagcagccatccttgctgactaaccatgtgacattggccactgctcagcctctgaatgttggtgttgcccatgttgtcagacaacaacaatccagttccctcccttcgaagaagaataagcagtcagctccagtctcttccaagtcctctctagatgttctgccttcccaagtctattctctggttgggagcagtcccctccgcaccacatcttcttataattccttggtccctgtccaagatcagcatcagcccatcatcattccagatactcccagccctcctgtgagtgtcatcactatccgaagtgacactgatgaggaagaggacaacaaatacaagcccagtagctctggactgaagccaaggtctaatgtcatcagttatgtcactgtcaatgattctccagactctgactcttctttgagcagcccttattccactgataccctgagtgctctccgaggcaatagtggatccgttttggaggggcctggcagagttgtggcagatggcactggcacccgcactatcattgtgcctccactgaaaactcagcttggtgactgcactgtagcaacccaggcctcaggtctcctgagcaataagactaagccagtcgcttcagtgagtgggcagtcatctggatgctgtatcacccccacagggtatcgagctcaacgcggggggaccagtgcagcacaaccactcaatcttagccagaaccagcagtcatcggcggctccaacctcacaggagagaagcagcaacccagccccccgcaggcagcaggcgtttgtggcccctctctcccaagccccctacaccttccagcatggcagcccgctacactcgacagggcacccacaccttgccccggcccctgctcacctgccaagccaggctcatctgtatacgtatgctgccccgacttctgctgctgcactgggctcaaccagctccattgctcatcttttctccccacagggttcctcaaggcatgctgcagcctataccactcaccctagcactttggtgcaccaggtccctgtcagtgttgggcccagcctcctcacttctgccagcgtggcccctgctcagtaccaacaccagtttgccacccaatcctacattgggtcttcccgaggctcaacaatttacactggatacccgctgagtcctaccaagatcagccagtattcctacttatagttggtgagcatgagggaggaggaatcatggctaccttctcctggccctgcgttcttaatattgggctatggagagatcctcctttaccctcttgaaatttcttagccagcaacttgttctgcaggggcccactgaagcagaaggtttttctctgggggaacctgtctcagtgttgactgcattgttgtagtcttcccaaagtttgccctatttttaaattcattatttttgtgacagtaattttggtacttggaagagttcagatgcccatcttctgcagttaccaaggaagagagattgttctgaagttaccctctgaaaaatattttgtctctctgacttgatttctataaatgcttttaaaaacaagtgaagcccctctttatttcattttgtgttattgtgattgctggtcaggaaaaatgctgatagaaggagttgaaatctgatgacaaaaaaagaaaaattactttttgtttgtttataaactcagacttgcctattttattttaaaagcggcttacacaatctcccttttgtttattggacatttaaacttacagagtttcagttttgttttaatgtcatattatacttaatgggcaattgttatttttgcaaaactggttacgtattactctgtgttactattgagattctctcaattgctcctgtgtttgttataaagtagtgtttaaaaggcagctcaccatttgctggtaacttaatgtgagagaatccatatctgcgtgaaaacaccaagtattctttttaaatgaagcaccatgaattcttttttaaattattttttaaaagtctttctctctctgattcagcttaaatttttttatcgaaaaagccattaaggtggttattattacatggtggtggtggttttattatatgcaaaatctctgtctattatgagatactggcattgatgagctttgcctaaagattagtatgaattttcagtaatacacctctgttttgctcatctctcccttctgttttatgtgatttgtttggggagaaagctaaaaaaacctgaaaccagataagaacatttcttgtgtatagcttttatacttcaaagtagcttcctttgtatgccagcagcaaattgaatgctctcttattaagacttatataataagtgcatgtaggaattgcaaaaaatattttaaaaatttattactgaatttaaaaatattttagaagttttgtaatggtggtgttttaatattttacataattaaatatgtacatattgattagaaaaatataacaagcaatttttcctgctaacccaaaatgttatttgtaatcaaatgtgtagtgattacacttgaattgtgtacttagtgtgtatgtgatcctccagtgttatcccggagatggattgatgtctccattgtatttaaaccaaaatgaactgatacttgttggaatgtatgtgaactaattgcaattatattagagcatattactgtagtgctgaatgagcaggggcattgcctgcaaggagaggagacccttggaattgttttgcacaggtgtgtctggtgaggagtttttcagtgtgtgtctcttccttccctttcttcctccttcccttattgtagtgccttatatgataatgtagtggttaatagagtttacagtgagcttgccttaggatggaccagcaagcccccgtggaccctaagttgttcaccgggatttatcagaacaggattagtagctgtattgtgtaatgcattgttctcagtttccctgccaacattgaaaaataaaaacagcagcttttctcctttaccaccacctctacccctttccattttggattctcggctgagttctcacagaagcattttccccatgtggctctctcactgtgcgttgctaccttgcttctgtgagaattcaggaagcaggtgagaggagtcaagccaatattaaatatgcattcttttaaagtatgtgcaatcacttttagaatgaatttttttttccttttcccatgtggcagtccttcctgcacatagttgacattcctagtaaaatatttgcttgttgaaaaaaacatgttaacagatgtgtttataccaaagagcctgttgtattgcttaccatgtccccatactatgaggagaagttttgtggtgccgctggtgacaaggaactcacagaaaggtttcttagctggtgaagaatatagagaaggaaccaaagcctgttgagtcattgaggcttttgaggtttcttttttaacagcttgtatagtcttggggcccttcaagctgtgaaattgtccttgtactctcagctcctgcatggatctgggtcaagtagaaggtactggggatggggacattcctgcccataaaggatttggggaaagaagattaatcctaaaatacaggtgtgttccatccgaattgaaaatgatatatttgagatataattttaggactggttctgtgtagatagagatggtgtcaaggaggtgcaggatggagatgggagatttcatggagcctggtcagccagctctgtaccaggttgaacaccgaggagctgtcaaagtatttggagtttcttcattgtaaggagtaagggcttccaagatggggcaggtagtccgtacagcctaccaggaacatgttgtgttttctttattttttaaaatcattatattgagttgtgttttcagcactatattggtcaagatagccaagcagtttgtataatttctgtcactagtgtcatacagttttctggtcaacatgtgtgatctttgtgtctcctttttgccaagcacattctgattttcttgttggaacacaggtctagtttctaaaggacaaattttttgttccttgtcttttttctgtaagggacaagatttgttgtttttgtaagaaatgagatgcaggaaagaaaaccaaatcccattcctgcaccccagtccaataagcagataccacttaagataggagtctaaactccacagaaaaggataataccaagagcttgtattgttaccttagtcacttgcctagcagtgtgtggctttaaaaactagagatttttcagtcttagtctgcaaactggcatttccgattttccagcataaaaatccacctgtgtctgctgaatgtgtatgtatgtgctcactgtggctttagattctgtccctggggttagccctgttggccctgacaggaagggaggaagcctggtgaatttagtgagcagctggcctgggtcacagtgacctgacctcaaaccagcttaaggctttaagtcctctctcagaacttggcatttccaacttcttcctttccgggtgagagaagaagcggagaagggttcagtgtagccactctgggctcatagggacacttggtcactccagagtttttaatagctcccaggaggtgatattattttcagtgctcagctgaaataccaaccccaggaataagaactccatttcaaacagttctggccattctgagcctgcttttgtgattgctcatccattgtcctccactagaggggctaagcttgactgcccttagccaggcaagcacagtaatgtgtgttttgttcagcattattatgcaaaaattcactagttgagatggtttgttttaggataggaaatgaaattgcctctcagtgacaggagtggcccgagcctgcttcctattttgattttttttttttttaactgatagatggtgcagcatgtctacatggttgtttgttgctaaactttatataatgtgtggtttcaattcagcttgaaaaataatctcactacatgtagcagtacattatatgtacattatatgtaatgttagtatttctgctttgaatccttgatattgcaatggaattcctactttattaaatgtatttgatatgctagttattgtgtgcgatttaaactttttttgctttctccctttttttggttgtgcgctttcttttacaacaagcctctagaaacagatagtttctgagaattactgagctatgtttgtaatgcagatgtacttagggagtatgtaaaataatcattttaacaaaagaaatagatatttaaaatttaatactaactatgggaaaagggtccattgtgtaaaacatagtttatctttggattcaatgtttgtctttggttttacaaagtagcttgtattttcagtattttctacataatatggtaaaatgtagagcaattgcaatgcatcaataaaatgggtaaattttctg

SEQ ID NO: 158 - DNA, 2782 bp, Homo sapiens
ggagccgtcaccccgggcggggacccagcgcaggcaactccgcgcggcgcccggccgagggagggagcgagcgggcgggcgggcaagccagacagctgggccggagcagccgccggcgcccgaggggccgagcgagattgtaaaccatggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctgcccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggcggagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactgattaacaagccagatgggaccttcctcctgagattcagtgactcagaaattggcggcatcaccattgcttggaagtttgattctcaggaaagaatgttttggaatctgatgccttttaccaccagagacttctccatcaggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtgaccccgcgacctctccatcttcagcttcttcatcttcaccagaggaatcactcttgtggatgttttaattccatgaatcgcttctcttttgaaacaatactcataatgtgaagtgttaatactagttgtgaccttagtgtttctgtgcatggtggcaccagcgaagggagtgcgagtatgtgtttgtgtgtgtgtgtgtgtgtgtgtgtgtgtgcgttggtgcacgttatggtgtttctccctctcactgtctgagagtttagttgtagcaga

SEQ ID NO: 159 - DNA, 2977 bp, Homo sapiens
ccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcaggagcagtcttccatcatgctgaattttgtcttccaggagctgcccctatggggcgggccgcagggccagcctgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaattt

SEQ ID NO: 160 - DNA, 3541 bp, Homo sapiens
tccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaagacaagaaggatgaggagaaaaacaagatccgggaagagtggaacaatttttcattcttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagtgatgtccttaccacaccatggaaattcaaagttgccaaacagctggccagtgccctgagctacttggaggataaagacctggtccatggaaatgtgtgtactaaaaacctcctcctggcccgtgagggaatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttccagaaaaaaaaaccagccaactgaagtggaccccacacattttgagaagcgcttcctaaagaggatccgtgacttgggagagggccactttgggaaggttgagctctgcaggtatgaccccgaagacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcagcttatgagaaaatgctgggaattccaaccatccaatcggacaagctttcagaaccttattgaaggatttgaagcacttttaaaataagaagcatgaataacatttaaattccacagattatcaa

SEQ ID NO: 161 - DNA, 2436 bp, Homo sapiens
ctgcagcttctaggacccggtttcttttactgatttaaaaacaaaacaaaaaaaaataaaaaagttgtgcctgaaatgaatcttgttttttttttataagtagccgcctggttactgtgtcctgtaaaatacagacattgacccttggtgtagcttctgttcaactttatatcacgggaatggatgggtctgatttcttggccctcttcttgaattggccatatacagggtccctggccagtggactgaaggctttgtctaagatgacaagggtcagctcaggggatgtgggggagggcggttttatcttcccccttgtcgtttgaggttttgatctctgggtaaagaggccgtttatctttgtaaacacgaaacatttttgctttctccagttttctgttaatggcgaaagaatggaagcgaataaagttttactgatttttgagacactagcacctagcgctttcattattgaaacgtcccgtgtgggaggggcgggtctgggtgcggctgccgcatgactcgtggttcggaggcccacgtggccggggcggggactcaggcgcctggcagccgactgattacgtagcgggcggggccggaagtgccgctccttggtgggggctgttcatggcggttccggggtctccaacatttttcccggtctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttcctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctcaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgcctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaacttttcacagtgaagtgcctttttcctagaagtggtttgtagactcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcatatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaatt

SEQ ID NO: 162 - DNA, 2500 bp, Homo sapiens
cggggggatcttggctgtgtgtctgcggatctgtagtggcggcggcggcggcggcggcggggaggcagcaggcgcgggagcgggcgcaggagcaggcggcggcggtggcggcggcggttagacatgaacgccgcctcggcgccggcggtgcacggagagccccttctcgcgcgcgggcggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcggagcctccccctctctccttggccggcccacaccctggaatgggagacgcatcgaatcacatgggacagatgtaaaagggtccaagttgccacattgcttcattaaaacaagagaccacttccttaacagctgtattatcttaaacccacataaacacttctccttaacccccatttttgtaatataagacaagtctgagtagttatgaatcgcagacgcaagaggtttcagcattcccaattatcaaaaaacagaaaaacaaaaaaaagaaagaaaaaagtgcaacttgagggacgactttctttaacatatcattcagaatgtgcaaagcagtatgtacaggctgagacacagcccagagactgaacggc

SEQ ID NO: 163 - DNA, 3277 bp, Homo sapiens
aaaaaaaagaaaaaaaaaggcacaaaaaagtggaaacttttccctgtccattccatcaagtcctgaaaaatcaaaatggatttagagaaaaattatccgactcctcggaccagcaggacaggacatggaggagtgaatcagcttgggggggtttttgtgaatggacggccactcccggatgtagtccgccagaggatagtggaacttgctcatcaaggtgtcaggccctgcgacatctccaggcagcttcgggtcagccatggttgtgtcagcaaaattcttggcaggtattatgagacaggaagcatcaagcctggggtaattggaggatccaaaccaaaggtcgccacacccaaagtggtggaaaaaatcgctgaatataaacgccaaaatcccaccatgtttgcctgggagatcagggaccggctgctggcagagcgggtgtgtgacaatgacaccgtgcctagcgtcagttccatcaacaggatcatccggacaaaagtacagcagccacccaaccaaccagtcccagcttccagtcacagcatagtgtccactggctccgtgacgcaggtgtcctcggtgagcacggattcggccggctcgtcgtactccatcagcggcatcctgggcatcacgtcccccagcgccgacaccaacaagcgcaagagagacgaaggtattcaggagtctccggtgccgaacggccactcgcttccgggcagagacttcctccggaagcagatgcggggagacttgttcacacagcagcagctggaggtgctggaccgcgtgtttgagaggcagcactactcagacatcttcaccaccacagagcccatcaagcccgagcagaccacagagtattcagccatggcctcgctggctggtgggctggacgacatgaaggccaatctggccagccccacccctgctgacatcgggagcagtgtgccaggcccgcagtcctaccccattgtgacaggccgtgacttggcgagcacgaccctccccgggtaccctccacacgtcccccccgctggacagggcagctactcagcaccgacgctgacagggatggtgcctgggagtgagttttccgggagtccctacagccaccctcagtattcctcgtacaacgactcctggaggttccccaacccggggctgcttggctccccctactattatagcgctgccgcccgaggagccgccccacctgcagccgccactgcctatgaccgtcactgacccttggagccaggcgggcaccaaacactgatggcacctattgagggtgacagccacccagccctcctgaagatagccagagagcccatgagaccgtcccccagcatcccccacttgcctgaagctcccctcttcctctcttcctccagggactctggggccctttggtggggccgttggacttctggatgcttgtctatttctaaaagccaatctatgagcttctcccgatggccactgggtctctgcaaaccaatagactgtcctgcaaataaccgcagccccagcccagcctgcctgtcctccagctgtctgactatccatccatcataaccaccccagcctgggaaggagagcttgcttttgttgcttcagcagcacccatgtaaataccttcttgcttttctgtgggcctgaaggtccgactgagaagactgctccacccatgatgcatctcgcactcttggtgcatcaccggacatcttagacctatggcagagcatcctctctgccctgggtgaccctggcaggtgcgctcagagctgtcctcaagatggaggatgctgcccttgggccccagcctcctgctcatccctccttctttagtatctttacgaggagtctcactgggctggttgtgctgcaggctccccctgaggcccctctccaagaggagcacactttggggagatgtcctggtttcctgcctccatttctctgggaccgatgcagtatcagcagctcttttccagatcaaagaactcaaagaaaactgtctgggagattcctcagctacttttccgaagcagaatgtcatccgaggtattgattacattgtggactttgaatgtgagggctggatgggacgcaggagatcatctgatcccagccaaggaggggcctgaggctctccctactccctcagcccctggaacggtgttttctgaggcatgcccaggttcaggtcacttcggacacctgccatggacacttcacccaccctccaggaccccagcaagtggattctgggcaagcctgttccggtgatgtagacaataattaacacagaggactttcccccacacccagatcacaaacagcctacagccagaacttctgagcatcctctcggggcagaccctccccgtcctcgtggagcttagcaggcagctgggcatggaggtgctggggctggggcagatgcctaatttcgcacaatgcatgcccacctgttgatctaaggggccgcgatggtcagggccacggccaagggccacgggaacttggagagggagcttggagaactcactgtgggctagggtggtcagaggaagccagcagggaagatctgggggacagaggaaggcctcctgagggaggggcaggagagcagtgaggagctgctgtgtgacctgggagtgattttgacatgggggtgccaggtgccatcatctctttacctggggccttaattccttgcatagtctctcttgtcaagtcagaacagccaggtagagcccttgtccaaacctgggctgaatgacagtgatgagagggggcttggccttcttaggtgacaatgtcccccatatctgtatgtcaccaggatggcagagagccagggcagagagagactggacttgggatcagcaggccaggcaggtcttgtcctggtcctggccacatgtctttgctgtgggacctcagacaaaaccctgcacctctttgagccttggctgccttggtgcagcagggtcatctgtagggccaccccacagctctttccttcccctcctctctccagggagccggggctgtgagaggatcatctggggcaggccctccacttccaagcaagcagatgggggtgggcacctgaggcccaataatatttggaccaagtgggaaacaagaacactcggaggggcgggaatcagaagagcctggaaaaagacctagcccaacttcccttgtgggaaactgaggcccagcttggggaaggccaggaccatgcagggagaaaaag

SEQ ID NO: 164 - DNA, 1924 bp, Homo sapiens
atggagaccgaaccgcctcacaacgagcccatccccgtcgagaatgatggcgaggcctgtggacccccagaggtctccagacccaactttcaggtcctcaacccggcattcagggaagctggagcccatggaagctacagcccacctcctgaggaagcaatgcccttcgaggctgaacagcccagcttgggaggcttctggcctacactggagcagcctggattccccagtggggtccatgcaggccttgccakgstysgsccagcactcatggagcccggagccttcagtggtgccagaccaggcctgggaggatacagccctccaccagaagaagctatgccctttgagtttgaccagcctgcccagagaggctgcagtcaacttctcttacaggtcccagaccttgctccaggaggcccaggtgctgcaggggtccccggagctcctcccgaggagccccaagccctcaggcctgcaaaggctggctccagaggaggctacagccctccccctgaggagactatgccatttgagcttgatggagaaggatttggggacgacagcccacccccggggctttcccgagttatcgcacaagtcgacggcagcagccagttcgcggcagtcgcggcctcgagtgcggtccgcctcactcccgccgcgaacgcgcctcccctctgggtcccaggcgccatcggcagcccatcccaagaggctgtcagacctccttctaacttcacgggcagcagcccctggatggagatctccggacccccgttcgagattggcagcgcccccgctggggtcgacgacactcccgtcaacatggacagccccccaatcgcgcttgacggcccgcccatcaaggtctccggagccccagataagagagagcgagcagagagacccccagttgaggaggaagcagcagagatggaaggagccgctgatgccgcggagggaggaaaagtaccctctccggggtacggatcccctgccgccggggcagcctcagcggataccgctgccagggcagcccctgcagccccagccgatcctgactccggggcaaccccagaagatcccgactccgggacagcaccagccgatcctgactccggggcattcgcagccgatcccgactccggggcagcccctgccgccccagccgatcccgactccggggcggcccctgacgccccagccgatcccgactccggggcggcccctgacgccccagccgatccagatgccggggcggcccctgaggctcccgccgcccctgcggctgctgagacccgggcagcccatgtcgccccagctgcgccagacgcaggggctcccactgccccagccgcttctgccacccgggcagcccaagtccgccgggcggcctctgcagcccctgcctccggggccagacgcaagatccatctcagaccccccagccccgagatccaggctgccgatccgcctactccgcggcctactcgcgcgtctgcctggcggggcaagtccgagagcagccgcggccgccgcgtgtactacgatgaaggggtggccagcagcgacgatgactccagcggagacgagtccgacgatgggacctccggatgcctccgctggtttcagcatcggcgaaatcgccgccgccgaaagccccagcgcaacttactccgcaactttctcgtgcaagccttcgggggctgcttcggtcgatctgagagtccccagcccaaagcctcgcgctctctcaaggtcaagaaggtacccctggcggagaagcgcagacagatgcgcaaagaagccctggagaagcgggcccagaagcgcgcagagaagaaacgcagtaagctcatcgacaaacaactccaggacgaaaagatgggctacatgtgtacgcaccgcctgctgcttctag

SEQ ID NO: 165 - DNA, 2436 bp, Homo sapiens; identical to SEQ ID NO: 161

SEQ ID NO: 166 - DNA, 2286 bp, Homo sapiens
ttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcactcagttacaactagatgaagagacaggtgaatttctcccaattcagccagcccagcacacccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccacgatgtacttttttataaatactgtatggacaaaaaatggcattttttataattaaattgtttagctctggcaaaaaaaaaaaattttttaagagctggtactaataaaggattattatgactgtt

SEQ ID NO: 167 - DNA, 2154 bp, Homo sapiens
gggggcagagggagcgagcgggcggccgcctagggtgcaagagccgggcgagcagagttgcgctgcgggcgtcctgggaagggagttccggagccaacagggggcttcgcctctggcccagcccttccggagccaacaggggacttcgcctctggcccagccctcccgctgatcccccagtcagcggtccgcaagccttgccgcatccacgaaactttgcccatactgcgggcgtacactttgcacttgaacttacaacacccgagcaaggacgcgactctcccgacgcggggagactattctgcccatttggggacacttccccgccgctgccaggacccggttctctggaaggctgtccttgaagctccttagacgctggagttttttcgggaagtgggaaagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaactttttatgcttaccatcttttttttttctttaacagatttgtatttaagaattgtttttaaaaaattttaagatttacacaatgtttctctgtaaatattgccattaaatgtaaataactttaataaaaacgtttatagcagttacacagaatttcaatcctagtatatagtacctagtattataggtactataaaccctaattttttttatttaagtacattttgctttttaaagttgattt

SEQ ID NO: 168 - DNA, 2154 bp, Homo sapiens; identical to SEQ ID NO: 167

SEQ ID NO: 169 - DNA, 2154 bp, Homo sapiens; identical to SEQ ID NO: 167

SEQ ID NO: 170 - DNA, 2395 bp, Homo sapiens
tcggagaccacattgcctcgtgtccaactatccattaccaagaagaaatctattcgtttgagcctgagacactctttgaggtaaaaaattagaatgaaagaacctttggatggtgaatgtggcaaagcagtggtaccacagcaggagcttctggacaaaattaaagaagaaccagacaatgctcaagagtatggatgtgtccaacagccaaaaactcaagaaagtaaattgaaaattggtggtgtgtcttcagttaatgagagacctattgcccagcagttgaacccaggctttcagctttcttttgcatcatctggcccaagtgtgttgcttccttcagttccagctgttgctattaaggttttttgttctggttgtaaaaaaatgctttataagggccaaactgcatatcataagacaggatctactcagctcttctgctccacacgatgcatcaccagacattcttcacctgcctgcctgccacctcctcccaagaaaacctgcacaaactgctcgaaagacattttaaatcctaaggatgtgatcacaactcgctttgagaattcctatcctagcaaagatttctgcagccaatcatgcttgtcatcttatgagctaaagaaaaaacctgttgttaccatatataccaaaagcatttcaactaagtgcagtatgtgtcagaagaatgctgatactcgatttgaagttaaatatcaaaatgtggtacatggtctttgtagtgatgcctgtttttcaaaatttcactctacaaacaacctcaccatgaactgttgtgagaactgtgggagctattgctatagtagctctggtccttgccaatcccagaaggtttttagttcaacaagtgtcacggcatacaagcagaattctgcccaaattcctccatatgccctggggaagtcattgaggccctcagctgaaatgattgagactacaaatgattcaggaaaaacagagcttttctgctctattaattgcttatctgcttacagagttaagactgttacttcttcaggtgtccaggtttcatgtcatagttgtaaaacctcagcaatccctcagtatcacctagccatgtcaaatggaactatatacagcttctgcagctccagttgtgtggttgctttccagaatgtatttagcaagccaaaaggaacaaactcttcggcggtgcccctgtctcagggccaagtggttgtaagcccgccctcctccaggtcagcagtgtcaataggaggaggtaacacctctgccgtttcccccagctccatccgtggctctgctgcagccagcctccaacctcttggtgaacaatcccagcaagttgctttaacccatacagttgttaaactcaagtgtcagcactgtaaccatctatttgccacaaaaccagaacttcttttttacaagggtaaaatgtttctgttttgtggcaagaattgctctgatgaatacaagaagaaaaataaagttgtggcaatgtgtgactactgtaaactgcagaaaattataaaggagactgtgcgattctcaggggttgataagccattctgtagtgaagtttgcaaattcctctctgcccgtgactttggagaacgatggggaaactactgtaagatgtgcagctactgttcacagacatccccaaatttggtagaaaatcgattggagggcaagttagaagagttttgttgtgaagattgtatgtccaaatttacagttctgttttatcagatggccaagtgtgatggttgtaaacgacagggtaaactaagcgagtccataaagtggcgaggcaacattaaacatttctgtaacctattttgtgtcttggagttttgtcatcagcaaattatgaatgactgtcttccacaaaataaagtaaatatttctaaagcaaaaactgctgtgacggagctcccttctgcaaggacagatacaacaccagttataaccagtgtgatgtcattggcaaaaatacctgctaccttatctacagggaacactaacagtgttttaaaaggtgcagttactaaagaggcagcaaagatcattcaagatgaaagtacacaggaagatgctatgaaatttccatcttcccaatcttcccagccttccaggcttttaaagaacaaaggcatatcatgcaaacccgtcacacagaccaaggccacttcttgcaaaccacatacacagcacaaagaatgtcagacagaatgccctgttcgtgcagtttgctgaggtgttcccgctgaagtatttggctaccagccagatcccctgaactaccaaatagctgtgggctttctggaactgctggctgggttgctgctggtcatgggcccaccgatgctgcaagagatcagtaact

SEQ ID NO: 171 - DNA, 2154 bp, Homo sapiens; identical to SEQ ID NO: 167

SEQ ID NO: 172 - DNA, 2154 bp, Homo sapiens; identical to SEQ ID NO: 167

SEQ ID NO: 173 - DNA, 2154 bp, Homo sapiens; identical to SEQ ID NO: 167

SEQ ID NO: 174 - DNA, 1696 bp, Homo sapiens
gcatcccggcatctgcacgtggttatgctgccggagtttgggccgccactgtaggaaaagtaacttcagctgcagccccaaagcgagtgagccgagccggagccatggagggccagagcgtggaggagctgctcgcaaaggcagagcaggacgaggcagagaagttgcaacgcatcacggtgcacaaggagctggagctgcagtttgacctgggcaacctgctggcgtcggaccggaaccccccgaccgggctgcggtgcgccggacccacgccggaggccgagctacaggccctggcgcgggacaacacgcaactgctcatcaaccagctgtggcagctgcccacggagcgcgtggaagaggcgatagtggcgcggctgccggagcccaccacacgcctgccgcgagagaagcctctgccccgaccgcggccacttacacgctggcagcagttcgcgcgcctcaagggcatccgtcccaagaagaagaccaacctggtgtgggacgaggtgagtggccagtggcggcggcgctggggctaccagcgcgcccgggacgacaccaaagaatggctgattgaggtgcccggcaatgccgaccccttggaggaccagttcgccaagcggattcaggccaagaaggaaagggtggccaagaacgagctgaaccggctgcgtaacctggcccgcgcgcacaagatgcagctgcccagcgcggccggcttgcaccctaccggacaccagagtaaggaggagctgggccgcgccatgcaagtggccaaggtctccaccgcctctgtggggcgctttcaggagcgcctccccaaggagaaggtgccccggggctccggcaagaaaaggaagtttcaaccccttttcggggactttgcagccgagaaaaagaaccagttggagctgcttcgtgtcatgaacagcaagaagcctcagctggatgtgactagggccaccaataagcagatgagggaggaggaccaggaggaggccgccaagaggaggaaaatgagccagaagggcaagagaaagggaggccggcaggggcctgggggcaagaggaaagggggcccgcccagccagggagggaagaggaaagggggcttgggaggcaagatgaattctgggccgcctggcttgggtggcaagagaaaaggaggacagcgcccaggaggaaagaggaggaagtaatagtttctaactgtcggacccgtctgtaaaccaaggactatgaatactaaatgttaagttctaggcaattatacggggactcagaaggacctggccgctgccttcattgagtttaaagggacaggattgcccttccgtcaagaaagtatgtaagtgttggactgcacaaattaatgtttttcccacaaccgagactttggagattaagaacttatttgaggatttaagaattagggaaataatttggtggaaaccgggaatgagttctattcttaaacagcctttttttttctttttaatgttggatatacggcgaggtagagttggccatatttcagagacttagattgacgtatatgtttctgcattatttttacaacaagtttgtgtatcagagcgggagttcgggggagggaaagaaaacaaacagtttcagaattgaataggcaagtgactgttttaaagattaagtaataaagatgtcttatctagtg

SEQ ID NO: 175 - DNA, 2154 bp, Homo sapiens; identical to SEQ ID NO: 167

SEQ ID NO: 176 - DNA, 2154 bp, Homo sapiens; identical to SEQ ID NO: 167

SEQ ID NO: 177 - DNA, 2154 bp, Homo sapiens; identical to SEQ ID NO: 167

SEQ ID NO: 178 - DNA, 3454 bp, Mus musculus; identical to SEQ ID NO: 151

SEQ ID NO: 179 - protein, 724 aa, Mus musculus
MSAEGYQYRALYDYKKEREEDIDLHLGDILTVNKGSLVALGFSDGQEARPEDIGWLNGYNETTGERGDFPGTYVEYIGRKRISPPTPKPRPPRPLPVAPGSSKTEADTEQQALPLPDLAEQFAPPDVAPPLLIKLLEAIEKKGLECSTLYRTQSSSNPAELRQLLDCDAASVDLEMIDVHVLADAFKRYLADLPNPVIPVAVYNEMMSLAQELQSPEDCIQLLKKLIRLPNIPHQCWLTLQYLLKHFFKLSQASSKNLLNARVLSEIFSPVLFRFPAASSDNTEHLIKAIEILISTEWNERQPAPALPPKPPKPTTVANNSMNNNMSLQDAEWYWGDISREEVNEKLRDTADGTFLVRDASTKMHGDYTLTPRKGGNNKLIKIFHRDGKYGFSDPLTFNSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNHDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 180 - DNA, 3372 bp, Homo sapiens
tacaaccaggctcaactgttgcatggtagcagatttgcaaacatgagtgctgaggggtaccagtacagagcgctgtatgattataaaaaggaaagagaagaagatattgacttgcacttgggtgacatattgactgtgaataaagggtccttagtagctcttggattcagtgatggacaggaagccaggcctgaagaaattggctggttaaatggctataatgaaaccacaggggaaaggggggactttccgggaacttacgtagaatatattggaaggaaaaaaatctcgcctcccacaccaaagccccggccacctcggcctcttcctgttgcaccaggttcttcgaaaactgaagcagatgttgaacaacaagctttgactctcccggatcttgcagagcagtttgcccctcctgacattgccccgcctcttcttatcaagctcgtggaagccattgaaaagaaaggtctggaatgttcaactctatacagaacacagagctccagcaacctggcagaattacgacagcttcttgattgtgatacaccctccgtggacttggaaatgatcgatgtgcacgttttggctgacgctttcaaacgctatctcctggacttaccaaatcctgtcattccagcagccgtttacagtgaaatgatttctttagctccagaagtacaaagctccgaagaatatattcagctattgaagaagcttattaggtcgcctagcatacctcatcagtattggcttacgcttcagtatttgttaaaacatttcttcaagctctctcaaacctccagcaaaaatctgttgaatgcaagagtactctctgaaattttcagccctatgcttttcagattctcagcagccagctctgataatactgaaaacctcataaaagttatagaaattttaatctcaactgaatggaatgaacgacagcctgcaccagcactgcctcctaaaccaccaaaacctactactgtagccaacaacggtatgaataacaatatgtccttacaaaatgctgaatggtactggggagatatctcgagggaagaagtgaatgaaaaacttcgagatacagcagacgggacctttttggtacgagatgcgtctactaaaatgcatggtgattatactcttacactaaggaaagggggaaataacaaattaatcaaaatatttcatcgagatgggaaatatggcttctctgacccattaaccttcagttctgtggttgaattaataaaccactaccggaatgaatctctagctcagtataatcccaaattggatgtgaaattactttatccagtatccaaataccaacaggatcaagttgtcaaagaagataatattgaagctgtagggaaaaaattacatgaatataacactcagtttcaagaaaaaagtcgagaatatgatagattatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaccagaccttatccagctgagaaagacgagagaccaatacttgatgtggttgactcaaaaaggtgttcggcaaaagaagttgaacgagtggttgggcaatgaaaacactgaagaccaatattcactggtggaagatgatgaagatttgccccatcatgatgagaagacatggaatgttggaagcagcaaccgaaacaaagctgaaaacctgttgcgagggaagcgagatggcacttttcttgtccgggagagcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaatttaaagccacaaccacatacaacacaaagagaaaaagaaatgcaaaaatctctgcgtgcagggacaaagaggcctttaaccatggtgcttgttaatgctttctgaagctttaccagctgaaagttgggactctggagagcggaggagagagaggcagaagaaccctggcctgagaaggtttggtccagcctggtttagcctggatgttgctgtgcacggtggacccagacacatcgcactgtggattatttcattttgtaacaaatgaacgatatgtagcagaaaggcacgtccactcacaagggacgctttgggagaatgtcagttcatgtatgttcagaagaaattctgtcatagaaagtgccagaaagtgtttaacttgtcaaaaaacaaaaacccagcaacagaaaaatggagtttggaaaacaggacttaaaatgacattcagtatataaaatatgtacataatattggatgactaactatcaaatagatggatttgtatcaataccaaatagcttctgttttgttttgctgaaggctaaattcacagcgctatgcaattcttaattttcattaagttgttatttcagttttaaatgtaccttcagaataagcttccccaccccagtttttgttgcttgaaaatattgttgtcccggatttttgttaatattcatttttgttatccttttttaaaaataaatgtacaggatgccagtaaaaaaaaaaatggcttcagaattaaaactatgaaatattttacagtttttcttgtacagagtacttgctgttagcccaaggttaaaaagttcataacagattttttttggactgttttgttgggcagtgcctgataagcttcaaagctgctttattcaataaaaaaaaaacccgaattcactgg

SEQ ID NO: 181 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQNAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 182 - DNA, 252 bp, Mus musculus; identical to SEQ ID NO: 56

SEQ ID NO: 183 - DNA, 2655 bp, Mus musculus; identical to SEQ ID NO: 139

SEQ ID NO: 184 - protein, 756 aa, Mus musculus
MEGSPTTATAVEGKVPSPERGDGSSTQPEAMDAKPAPAAQAVSTGSDAGAPTDSAMLTDSQSDAGEDGTAPGTPSDLQSDPEELEEAPAVRADPDGGAAPVAPATPAESESEGSRDPAAEPASEAVPATTAESASGAAPVTQVEPAAAAVSATLAEPAARAAPITPKEPTTRAVPSARAHPAAGAVPGAPAMSASARAAAARAAYAGPLVWGARSLSATPAARASLPARAAAAARAASAARAVAAGRSASAAPSRAHLRPPSPEIQVADPPTPRPPPRPTAWPDKYERGRSCCRYEASSGICEIESSSDESEEGATGCFQWLLRRNRRPGLPRSHTVGSNPVRNFFTRAFGSCFGLSECTRSRSLSPGKAKDPMEERRKQMRKEAIEMREQKRADKKRSKLIDKQLEEEKMDYMCTHRLLLLGAGESGKSTIVKQMRILHVNGFNGDSEKATKVQDIKNNLKEAIETIVAAMSNLVPPVELANPENQFRVDYILSVMNVPNFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 185 - DNA, 1924 bp, Homo sapiens; identical to SEQ ID NO: 164

SEQ ID NO: 186 - protein, 388 aa, Homo sapiens
MEISGPPFEIGSAPAGVDDTPVNMDSPPIALDGPPIKVSGAPDKRERAERPPVEEEAAEMEGAADAAEGGKVPSPGYGSPAAGAASADTAARAAPAAPADPDSGATPEDPDSGTAPADPDSGAFAADPDSGAAPAAPADPDSGAAPDAPADPDSGAAPDAPADPDAGAAPEAPAAPAAAETRAAHVAPAAPDAGAPTAPAASATRAAQVRRAASAAPASGARRKIHLRPPSPEIQAADPPTPRPTRASAWRGKSESSRGRRVYYDEGVASSDDDSSGDESDDGTSGCLRWFQHRRNRRRRKPQRNLLRNFLVQAFGGCFGRSESPQPKASRSLKVKKVPLAEKRRQMRKEALEKRAQKRAEKKRSKLIDKQLQDEKMGYMCTHRLLLL

SEQ ID NO: 187 - DNA, 1111 bp, Mus musculus
gagaggatcagtggaggcacctctcggagtcttagacttcagagtctgagacttagcgagaggagcctcgaggagactccttctctcttctttacccatccctttcttttacttacagcctcaagctgaggcgcggagctttagaaagttcgcagtggtttgaagtccttgcgcagtggggccactctctgcagagccagagggtgagtcggcttctcggtgagcacctaagagaatggatcgcaggtcccgggctcagcagtggcgccgagctcgccataattacaacgacctgtgcccgcccataggccgccgggctgccaccgctctcctctggctctcctgctccattgctctcctccgcgccctagcctcttccaacgcccgcgcccagcagcgtgctgcccatcgccggagcttccttaacgcccaccaccgctccgctgccgctgcagctgccgcacaggtactccctgagtcctctgaatctgagtctgatcacgagcacgaggaggttgagcctgagctggcccgccccgagtgcctagagtacgatcaggacgactacgagaccgagaccgattctgagaccgagcctgagtccgatatcgaatccgagaccgaaatcgagaccgagccagagaccgagccagaaaccgagccagagaccgagccagaggacgagcgcggcccccggggtgccaccttcaaccagtcactcactcagcgtctgcacgctctgaagttgcagagcgccgacgcctccccgagacgtgcgcagcccaccactcaggagcctgagagcgcaagcgagggggaggagccccagcgagggcccttagatcaggatcctcgggaccccgaggaggagccagaggagcgcaaggaggaaaacaggcagccccgccgctgcaagaccaggaggccagcccgccgtcgcgaccagtccccggagtcccctcccagaaaggggcccatccccatccggcgtcactaatgggtgactccgtccagattctccttgttttcatggataaaggtgctggagagtctggcaaaagcaccattgtgaagcagatgaggatcctgcatgttaatgggtttaacggag

SEQ ID NO: 188 - protein, 253 aa, Mus musculus
MDRRSRAQQWRRARHNYNDLCPPIGRRAATALLWLSCSIALLRALASSNARAQQRAAHRRSFLNAHHRSAAAAAAAQVLPESSESESDHEHEEVEPELARPECLEYDQDDYETETDSETEPESDIESETEIETEPETEPETEPETEPEDERGPRGATFNQSLTQRLHALKLQSADASPRRAQPTTQEPESASEGEEPQRGPLDQDPRDPEEEPEERKEENRQPRRCKTRRPARRRDQSPESPPRKGPIPIRRH

SEQ ID NO: 189 - DNA, 3677 bp, Homo sapiens
ctcgcctcagtctcctctgtcctctcccaggcaagaggaccggcggaggcacctctctcgagtcttaggctgcggaatctaagactcagcgagaggagcccgggaggagacagaactttccccttttttcccatcccttcttcttgctcagagaggcaagcaaggcgcggagctttagaaagttcttaagtggtcaggaaggtaggtgcttccctttttctcctcacaaggaggtgaggctgggacctccgggccagcttctcacctcatagggtgtacctttcccggctccagcagccaatgtgcttcggagccgctctctgcagagccagagggcaggccggcttctcggtgtgtgcctaagaggatggatcggaggtcccgggctcagcagtggcgccgagctcgccataattacaacgacctgtgcccgcccataggccgccgggcagccaccgcgctcctctggctctcctgctccatcgcgctcctccgcgcccttgccacctccaacgcccgtgcccagcagcgcgcggctgcccaacagcgccggagcttccttaacgcccaccaccgctccggcgcccaggtattccctgagtcccccgaatcggaatctgaccacgagcacgaggaggcagaccttgagctgtccctccccgagtgcctagagtacgaggaagagttcgactacgagaccgagagcgagaccgagtccgaaatcgagtccgagaccgacttcgagaccgagcctgagaccgcccccaccactgagcccgagaccgagcctgaagacgatcgcggcccggtggtgcccaagcactccaccttcggccagtccctcacccagcgtctgcacgctctcaagttgcgaagccccgacgcctccccaagtcgcgcgccgcccagcactcaggagccccagagccccagggaaggggaggagctcaagcccgaggacaaagatccaagggaccccgaagagtcgaaggagcccaaggaggagaagcagcggcgtcgctgcaagccaaagaagcccacccgccgtgacgcgtccccggagtccccttccaaaaagggacccatccccatccggcgtcactaatggaggacgccgtccagattctccttgttttcatggattcaggtgctggagaatctggtaaaagcaccattgtgaagcagatgaggatcctgcatgttaatgggtttaatggagagggcggcgaagaggacccgcaggctgcaaggagcaacagcgatggcagtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaaaaaaaaagagaggatcagtggaggcacctctcggagtcttagacttcagagtctgagacttagcgagaggagcctcgaggagactccttctctcttctttacccatccctttcttttacttacagcctcaagctgaggcgcggagctttagaaagttcgcagtggtttgaagtccttgcgcagtggggccactctctgcagagccagagggtgagtcggcttctcggtgagcacctaagagaatggatcgcaggtcccgggctcagcagtggcgccgagctcgccataattacaacgacctgtgcccgcccataggccgccgggctgccaccgctctcctctggctctcctgctccattgctctcctccgcgccctagcctcttccaacgcccgcgcccagcagcgtgctgcccatcgccggagcttccttaacgcccaccaccgctccgctgccgctgcagctgccgcacaggtactccctgagtcctctgaatctgagtctgatcacgagcacgaggaggttgagcctgagctggcccgccccgagtgcctagagtacgatcaggacgactacgagaccgagaccgattctgagaccgagcctgagtccgatatcgaatccgagaccgaaatcgagaccgagccagagaccgagccagaaaccgagccagagaccgagccagaggacgagcgcggcccccggggtgccaccttcaaccagtcactcactcagcgtctgcacgctctgaagttgcagagcgccgacgcctccccgagacgtgcgcagcccaccactcaggagcctgagagcgcaagcgagggggaggagccccagcgagggcccttagatcaggatcctcgggaccccgaggaggagccagaggagcgcaaggaggaaaacaggcagccccgccgctgcaagaccaggaggccagcccgccgtcgcgaccagtccccggagtcccctcccagaaaggggcccatccccatccggcgtcactaatgggtgactccgtccagattctccttgttttcatggataaaggtgctggagagtctggcaaaagcaccattgtgaagcagatgaggatcctgcatgttaatgggtttaacggag

SEQ ID NO: 190 - protein, 245 aa, Homo sapiens
MDRRSRAQQWRRARHNYNDLCPPIGRRAATALLWLSCSIALLRALATSNARAQQRAAAQQRRSFLNAHHRSGAQVFPESPESESDHEHEEADLELSLPECLEYEEEFDYETESETESEIESETDFETEPETAPTTEPETEPEDDRGPVVPKHSTFGQSLTQRLHALKLRSPDASPSRAPPSTQEPQSPREGEELKPEDKDPRDPEESKEPKEEKQRRRCKPKKPTRRDASPESPSKKGPIPIRRH

SEQ ID NO: 191 - DNA, 1389 bp, Mus musculus
ccccgcgccccgccgccgcatgggctgcctcggcaacagtaagaccgaggaccagcgcaacgaggagaaggcgcagcgcgaggccaacaaaaagatcgagaagcagctgcagaaggacaagcaggtctaccgggccacgcaccgcctgctgctgctgggtgctggagagtctggcaaaagcaccattgtgaagcagatgaggatcctgcatgttaatgggtttaacggagagggcggcgaagaggacccgcaggctgcaaggagcaacagcgatggtgagaaggccactaaagtgcaggacatcaaaaacaacctgaaggaggccattgaaaccattgtggccgccatgagcaacctggtgccccctgtggagctggccaaccctgagaaccagttcagagtggactacattctgagcgtgatgaacgtgcccgactttgacttcccacctgaattctatgagcatgccaaggctctgtgggaggatgagggagtgcgtgcctgctacgagcgctccaatgagtaccagctgattgactgtgcccagtacttcctggacaagattgatgtgatcaagcaggccgactacgtgccaagtgaccaggacctgcttcgctgccgtgtcctgacctctggaatctttgagaccaagttccaggtggacaaagtcaacttccacatgttcgatgtgggcggccagcgcgatgaacgccgcaagtggatccagtgcttcaatgatgtgactgccatcatcttcgtggtggccagcagcagctacaacatggtcattcgggaggacaaccagactaaccgcctgcaggaggctctgaacctcttcaagagcatctggaacaacagatggctgcgcaccatctctgtgattctcttcctcaacaagcaagacctgcttgctgagaaagtcctcgctggcaaatcgaagattgaggactactttccagagttcgctcgctacaccactcctgaggatgcgactcccgagccgggagaggacccacgcgtgacccgggccaagtacttcattcgggatgagtttctgagaatcagcactgctagtggagatgggcgccactactgctaccctcactttacctgcgccgtggacactgagaacatccgccgtgtcttcaacgactgccgtgacatcatccagcgcatgcatctcccccaatacgagctgctctaagaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagaagctt

SEQ ID NO: 192 - protein, 394 aa, Mus musculus
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLPQYELL

SEQ ID NO: 193 - DNA, 1610 bp, Homo sapiens
gcgggcgtgctgccgccgctgccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagc

SEQ ID NO: 194 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 195 - DNA, 331 bp, Mus musculus; identical to SEQ ID NO: 13
  misc_feature 7, 16, 18, 26, 41, 50, 61, 70, 86, 96, 124, 129, 299,306, 329: n = a, t, c, or g

SEQ ID NO: 196 - DNA, 7594 bp, Mus musculus; identical to SEQ ID NO: 130

SEQ ID NO: 197 - protein, 1210 aa, Mus musculus
MASQLQVFSPPSVSSSAFCSAKKLKIEPSGWDVSGQSSNDKYYTHSKTLPATQGQASSSHQVANFNLPAYDQGLLLPAPAVEHIVVTAADSSGSAATATFQSSQTLTHRSNVSLLEPYQKCGLKRKSEEVESNGSVQIIEEHPPLMLQNRTVVGAAATTTTVTTKSSSSSGEGDYQLVQHEILCSMTNSYEVLEFLGRGTFGQVAKCWKRSTKEIVAIKILKNHPSYARQGQIEVSILSRLSSENADEYNFVRSYECFQHKNHTCLVFEMLEQNLYDFLKQNKFSPLPLKYIRPILQQVATALMKLKSLGLIHADLKPENIMLVDPVRQPYRVKVIDFGSASHVSKAVCSTYLQSRYYRAPEIILGLPFCEAIDMWSLGCVIAELFLGWPLYPGASEYDQIRYISQTQGLPAEYLLSAGTKTTRFFNRDPNLGYPLWRLKTPEEHELETGIKSKEARKYIFNCLDDMAQVNMSTDLEGTDMLAEKADRREYIDLLKKMLTIDADKRITPLKTLNHQFVTMSHLLDFPHSSHVKSCFQNMEICKRRVHMYDTVSQIKSPFTTHVAPNTSTNLTMSFSNQLNTVHNQASVLASSSTAAAATLSLANSDVSLLNYQSALYPSSAAPVPGVAQQGVSLQPGTTQICTQTDPFQQTFIVCPPAFQTGLQATTKHSGFPVRMDNAVPIVPQAPAAQPLQIQSGVLTQGSCTPLMVATLHPQVATITPQYAVPFTLSCAAGRPALVEQTAAVLQAWPGGTQQILLPSAWQQLPGVALHNSVQPAAVIPEAMGSSQQLADWRNAHSHGNQYSTIMQQPSLLTNHVTLATAQPLNVGVAHVVRQQQSSSLPSKKNKQSAPVSSKSSLEVLPSQVYSLVGSSPLRTTSSYNSLVPVQDQHQPIIIPDTPSPPVSVITIRSDTDEEEDNKYKPNSSSLKARSNVISYVTVNDSPDSDSSLSSPHPTDTLSALRGNSGTLLEGPGRPAADGIGTRTIIVPPLKTQLGDCTVATQASGLLSSKTKPVASVSGQSSGCCITPTGYRAQRGGASAVQPLNLSQNQQSSSASTSQERSSNPAPRRQQAFVAPLSQAPYAFQHGSPLHSTGHPHLAPAPAHLPSQPHLYTYAAPTSAAALGSTSSIAHLFSPQGSSRHAAAYTTHPSTLVHQVPVSVGPSLLTSASVAPAQYQHQFATQSYIGSSRGSTIYTGYPLSPTKISQYSYL

SEQ ID NO: 198 - DNA, 5761 bp, Homo sapiens; identical to SEQ ID NO: 157

SEQ ID NO: 199 - protein, 490 aa, Homo sapiens
TPQYAVPFTLSCAAGRPALVEQTAAVLAWPGGTQQILLPSTWQQLPGVALHNSVQPTAMIPEAMGSGQQLADWRNAHSHGNQYSTIMQQPSLLTNHVTLATAQPLNVGVAHVVRQQQSSSLPSKKNKQSAPVSSKSSLDVLPSQVYSLVGSSPLRTTSSYNSLVPVQDQHQPIIIPDTPSPPVSVITIRSDTDEEEDNKYKPSSSGLKPRSNVISYVTVNDSPDSDSSLSSPYSTDTLSALRGNSGSVLEGPGRVVADGTGTRTIIVPPLKTQLGDCTVATQASGLLSNKTKPVASVSGQSSGCCITPTGYRAQRGGTSAAQPLNLSQNQQSSAAPTSQERSSNPAPRRQQAFVAPLSQAPYTFQHGSPLHSTGHPHLAPAPAHLPSQAHLYTYAAPTSAAALGSTSSIAHLFSPQGSSRHAAAYTTHPSTLVHQVPVSVGPSLLTSASVAPAQYQHQFATQSYIGSSRGSTIYTGYPLSPTKISQYSYL

SEQ ID NO: 200 - DNA, 4191 bp, Mus musculus
cagccgcggagtagccggcagccgctgacgcgccgcgggtccgccccagcctcgctcgtcctttcggtgcctctccttagccgcgggtgtccacgccggaccctgcacggcaggctgagttgcctgccagactcctgacccagatcgaccctgcgccaaggagccgcgcggcccggcgcacacggaagtgatcagctctgaatgggctttggaaggtaaagaagaaaaatccagtctgctttcagggacactggacaaccgaataaatgcagtatctaaatataaaagaggactgcaatgccatggcgttctgtgctaaaatgaggagcttcaagaagactgaggtgaagcaggtggtccctgagcctggagtggaggtgactttctatctgttggacagggagcccctccgcctgggcagcggagagtatacagccgaggagctgtgcatcagggccgcccaggagtgcagtatctctcctctctgtcacaacctcttcgccctgtacgatgagagcaccaagctctggtacgctccgaaccgaatcatcactgtggatgacaaaacgtctctccggctccactaccgcatgaggttctactttaccaactggcacggaaccaatgacaacgaacagtctgtatggcgacattctccaaagaagcagaaaaacggctatgagaagaaaagggttccagaagcaaccccactccttgatgccagttcactggagtatctgtttgcacagggacagtatgatttgatcaaatgcctggctcccattcgggaccccaagacggagcaagacggacatgatattgaaaatgagtgcctgggcatggcggtcctggccatctcccactatgccatgatgaagaagatgcagttgccggaacttcccaaagacatcagctacaagcgatatattccagaaacattgaataaatccatcagacagaggaaccttcttaccaggatgcgaataaataatgttttcaaggatttcttgaaggaatttaacaacaagaccatctgtgacagcagtgtgcatgacctgaaggtgaaatacctggctaccttggaaacttctacattgacaaaacattatggagctgaaatatttgagacttctatgctactgatttcatcagaaaatgaattgagtcgatgccattcgaatgacagtggcaatgttctctatgaggtcatggtgactggaaatctcgggatccagtggcggcagaaaccaaatgttgttcctgttgaaaaggaaaaaaataaactgaagcggaaaaaactggaatataataaacacaagaaggatgatgagagaaacaaactccgggaagagtggaacaatttttcctatttccctgaaatcacccacattgtaataaaggagtctgtggtcagcattaacaaacaggacaacaaaaacatggaactcaagctctcttctcgagaggaagccttgtcctttgtgtccctggtggatggctacttccggctcactgcagatgcccaccattacctctgtactgatgtggctcccccactgattgtccacaatatacagaacggctgccacggtccaatctgcacagaatatgccatcaataagctgcggcaggaagggagtgaagaggggatgtacgtgctgaggtggagctgcaccgactttgacaacattcttatgactgtcacctgctttgaaaagtctgaggtattgggtggccagaagcagttcaagaactttcagattgaggtacagaagggccgctacagcctgcatggctctatggaccactttcccagcctgcgagacctcatgaaccacctcaagaagcagatcctgcgcacggacaacataagctttgtgctgaaacgatgctgtcagcctaagcctcgagaaatctccaatctgctcgtagccactaagaaagcccaggagtggcagcctgtctactccatgagccagctgagctttgatcggatccttaagaaagatattatacaaggtgagcaccttggcagaggcacaagaacacatatctattctgggaccctgctggactacaaggatgaggaaggaattgctgaagagaagaagataaaagtgatcctcaaagtcctagaccccagccaccgggacatctctctggccttctttgaggctgctagcatgatgagacaggtttcccacaaacatatagtgtacctctacggcgtgtgtgtccgagatgtggaaaatatcatggtggaagagtttgtggagggggggccgttggatctcttcatgcaccggaaaagtgatgcgcttactaccccctggaagttcaaggttgccaaacagctggccagtgccctgagttacttggaagataaagacctggttcatggaaatgtgtgcactaaaaacctccttctggcccgtgagggcattgacagtgacattggcccgttcatcaagcttagtgaccctggcatcccagtctctgtgctgaccaggcaagagtgcatagagcgaatcccctggatcgctcctgagtgtgttgaagactccaagaacctgagtgtggctgctgacaagtggagctttggaaccacgctctgggaaatctgctacaacggagagattcctctcaaagacaagaccctcattgagaaagagaggttttatgaaagccgctgcaggcctgtgactccatcttgcaaggagctagctgacctcatgactcgctgcatgaactatgaccccaaccagagacccttcttccgagccatcatgagggacattaacaagctggaggagcagaatccagacattgtttcagaaaagcagccaacaacagaggtggaccccactcactttgaaaagcggttcctgaagaggattcgtgacttgggagagggtcactttgggaaggttgagctctgcagatatgatcctgagggagacaacacaggggagcaggtagctgtcaagtccctgaagcctgagagtggaggtaaccacatagctgatctgaagaaggagatagagatcttacggaacctctaccatgagaacattgtgaagtacaaaggaatctgcatggaagacggaggcaatggtatcaagctcatcatggagtttctgccttcgggaagcctaaaggagtatctgccaaagaataagaacaaaatcaacctcaaacagcagctaaaatatgccatccagatttgtaaggggatggactacttgggttctcggcaatacgttcaccgggacttagcagcaagaaatgtccttgttgagagtgagcatcaagtgaagatcggagactttggtttaaccaaagcaattgaaaccgataaggagtactacacagtcaaggacgaccgggacagcccagtgttctggtacgctccggaatgtttaatccagtgtaaattttatatcgcctctgatgtctggtcttttggagtgacactgcacgagctgctcacttactgtgactcagattttagtcccatggccttgttcctgaaaatgataggcccaactcatggccagatgacagtgacacggcttgtgaagactctgaaagaaggaaagcgtctgccatgtccacccaactgtcctgatgaggtttatcagcttatgagaaaatgctgggaattccaaccatctaaccggacaacttttcagaaccttattgaaggatttgaagcacttttaaaataagaagcatgaacaacatttaaattcccatttatcaaatccttctctcccaagccatttaaaaacgttttttaagtgaaaagtttgtattctgcctctaaagttcctcaacaaatactcgagttacacatatgcatatgtcacactgtcactcagtgtgtggatatgcctatgtcacactgtcactcagtgtgtggaactttctctttaaaggtgtaacatcttaaatttggtgatgaatagtgacaaccaaaagactagattgtgcctaagcactccttctggaacaaccgaatgatcagctgcatagcaaaggactgtgccgctggcatattgatctcagataaaaacttgtggacttggctgacactctcccttgccctgaaatctcaatgtctattcagtgatagtacaagcacgtagataccacttagtatactattgtttctattaaaaaaaaaaaaaa

SEQ ID NO: 201 - DNA, 3538 bp, Homo sapiens
tccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcaggtaaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtgggctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccgttcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacgaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaa

SEQ ID NO: 202 - protein, 1153 aa, Mus musculus
MQYLNIKEDCNAMAFCAKMRSFKKTEVKQVVPEPGVEVTFYLLDREPLRLGSGEYTAEELCIRAAQECSISPLCHNLFALYDESTKLWYAPNRIITVDDKTSLRLHYRMRFYFTNWHGTNDNEQSVWRHSPKKQKNGYEKKRVPEATPLLDASSLEYLFAQGQYDLIKCLAPIRDPKTEQDGHDIENECLGMAVLAISHYAMMKKMQLPELPKDISYKRYIPETLNKSIRQRNLLTRMRINNVFKDFLKEFNNKTICDSSVHDLKVKYLATLETSTLTKHYGAEIFETSMLLISSENELSRCHSNDSGNVLYEVMVTGNLGIQWRQKPNVVPVEKEKNKLKRKKLEYNKHKKDDERNKLREEWNNFSYFPEITHIVIKESVVSINKQDNKNMELKLSSREEALSFVSLVDGYFRLTADAHHYLCTDVAPPLIVHNIQNGCHGPICTEYAINKLRQEGSEEGMYVLRWSCTDFDNILMTVTCFEKSEVLGGQKQFKNFQIEVQKGRYSLHGSMDHFPSLRDLMNHLKKQILRTDNISFVLKRCCQPKPREISNLLVATKKAQEWQPVYSMSQLSFDRILKKDIIQGEHLGRGTRTHIYSGTLLDYKDEEGIAEEKKIKVILKVLDPSHRDISLAFFEAASMMRQVSHKHIVYLYGVCVRDVENIMVEEFVEGGPLDLFMHRKSDALTTPWKFKVAKQLASALSYLEDKDLVHGNVCTKNLLLAREGIDSDIGPFIKLSDPGIPVSVLTRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKQPTTEVDPTHFEKRFLKRIRDLGEGHFGKVELCRYDPEGDNTGEQVAVKSLKPESGGNHIADLKKEIEILRNLYHENIVKYKGICMEDGGNGIKLIMEFLPSGSLKEYLPKNKNKINLKQQLKYAIQICKGMDYLGSRQYVHRDLAARNVLVESEHQVKIGDFGLTKAIETDKEYYTVKDDRDSPVFWYAPECLIQCKFYIASDVWSFGVTLHELLTYCDSDFSPMALFLKMIGPTHGQMTVTRLVKTLKEGKRLPCPPNCPDEVYQLMRKCWEFQPSNRTTFQNLIEGFEALLK

SEQ ID NO: 203 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 204 - DNA, 975 bp, Mus musculus; identical to SEQ ID NO: 135
  misc_feature 799: n = a, t, c, or g

SEQ ID NO: 205 - DNA, 1354 bp, Mus musculus
gttggtcctcgctccagttctccccgcccaccctgcagaaagtgtcttctgattggcttcgaggccgcagggctcaggttacattcgcaagagttgcggagcgcgggagaccggacccaagaggagagaggctggttctgcaaggattctgcgctggtcggggagtgcccgacagcccctgagctgccacccagcatcgtacaaacccacccccgctctgcgccaggctccaccccagccaaggaccctcaacaccggcaatggactgctgcacggagagcgcctgctccaagccagacgacgatattcttgacatcccgctggatgatcccggagccaacgccgctgcagccaaaatccaggcgagtttccggggccacatggcgaggaagaagataaagagcggagagtgtggccggaagggaccgggccccgggggaccaggcggagctgggggcgcccggggaggcgcgggcggcggccccagcggagactaggccagagctgaacgttttagaagttccagaggagagtcggatgccgcgtccccttcgcagtgacaagacttccctactgtgtttgtgagcccctccttcccaccaaccagccagcttcaggagccccccccctccccccgccgcgtcccagagactccctctcccaggctggcttcgtcttgggcgtagcaagtccgtgccctttttagctcttcagtctaacgtggtctccttttgccttttctcccaccctcgtcccaaacccatactccaaaatgtccttttgcttcacgcccacctgtccacgcgcccagcatgcagctctgcctccgcagcctcggtgcgcttcgctgcgcgtactgcagagggcgcccaatgcgtcgcccaaatactctcaaaaaaagaaagaaaaaaagaaaaagaaagaaagaaaaaaaaagcaaccaccaagtcctttcgttctgtgggcaacgaaagggggcgcccgcgtctttccaccctagcctaacctcaacctcctaaacctggggctaggaaagaggggaggaggttttcatggttatctgataatttcccttgctcaaatggaaagtgaagtcctatcccatacctgcctgtcaccctcttttttcttgaaaacgcaccctgagagcagcccctcccgctcttctttgtttatgcaaaagcctcctgagcgcctggaggctccggcaggaggagacttccgcagccccgccccatgatagcctctcccccgttgggctcctcgggttgtggctggaaggcttttaatctctgcgtgtgcatgttaccatactgggttggaatgtgaataataaagaggaatgtcgaagtgt

SEQ ID NO: 206 - DNA, 1135 bp, Homo sapiens
ggcacgaggcgccagccttcgtccccgcagaggaccccccgacaccagcatggactgctgcaccgagaacgcctgctccaagccggacgacgacattctagacatcccgctggacgatcccggcgccaacgcggccgccgccaaaatccaggcgagttttcggggccacatggcgcggaagaagataaagagcggagagcgcggccggaagggcccgggccctggggggcctggcggagctggggtggcccggggaggcgcgggcggcggccccagcggagactaggccagaagaactgagcattttcaaagttcccgaggagagatggatgccgcgtccccttcgcagcgacgagacttccctgccgtgtttgtgaccccctcctgcccagcaacctgccagctacaggagccccctgcgtcccagagactccctcacccaggcaggctccgtcgcggagtcgctgagtccgtgcccttttagttagttctgcagtctagtatggtccccatttgcccttccactccaccccaccctaaaccatgcgctcccaatcttccttcttttgcttctcgcccacctcttcccgcacccagcatgcagctctgcctccgcagcctcagtgcgctttcctgcgcgcactgcggagggcgccctaagcgtcacccaagcacactcacttaaagaaaaaacgagttctttcgttctgtgcgcagctaaaaggggcgccctacatctccgtgccactcccgccccagcctagccccaagactttggatccggggcgagatgaagggaagagggttgttttggtttcggacgacccttgctctgaccggaagagaagtccctatcccacacctgcctgtcacgttccctcccctttccccagcgcactgttgagggcagcctctccagctctcttgtttatgcaaacgccgagcgcctgggaggctcggtaggaggagtcttccacggccccgccccgcccctgtcggtcccgccctcccccccgccgggctcctggggctgtggccgaaaggtttctgatctccgtgtgtgcatgtgactgtgctgggttggaatgtgaacaataaagaggaatgtccaagtgaaaaaaaaaaaaaaaaaaaa

SEQ ID NO: 207 - protein, 78 aa, Mus musculus
MDCCTESACSKPDDDILDIPLDDPGANAAAAKIQASFRGHMARKKIKSGECGRKGPGPGGPGGAGGARGGAGGGPSGD

SEQ ID NO: 208 - protein, 78 aa, Homo sapiens
MDCCTENACSKPDDDILDIPLDDPGANAAAAKIQASFRGHMARKKIKSGERGRKGPGPGGPGGAGVARGGAGGGPSGD

SEQ ID NO: 209 - DNA, 359 bp, Mus musculus; identical to SEQ ID NO: 92
  misc_feature 116, 257, 301: n = a, t, c, or g

SEQ ID NO: 210 - DNA, 2482 bp, Mus musculus
tgctccatgcccttgtcctcgctctggcccttgcctcttgccctagccttttctccgcctctaagttcttgtcccgtccctaggtccttgttccagggggtgggggcggggcggactaaggctggcctgccactccagcgagcaggctatctcctagttctcgctgctcggactagccattgccgccgcctcacctctgctgcaagtagcctcgccgtcggggagccctaccacacggtccgccctcagcatgatggacttggagttgccaccgccagactacagtcccagcaggacatggatttgattgacatcctttggaggcaagacatagatcttggagtaagtcgagaagtgtttgactttagtcagcgacagaaggactatgagctggaaaaacagaaaaaactcgaaaaggaaagacaagagcaactccagaaggaacaggagaaggccttttttgctcagtttcaactggatgaagaaacaggagaattcctcccaattcagccggcccagcacatccagacagacaccagtggatccgccagctactcccaggttgcccacattcccaaacaagatgccttgtactttgaagactgtatgcagcttttggcagagacattcccatttgtagatgaccatgagtcgcttgccctggatatccccagccacgctgaaagttcagtcttcactgcccctcatcaggcccagtccctcaatagctctctggaggcagccatgactgatttaagcagcatagagcaggacatggagcaagtttggcaggagctattttccattcccgaattacagtgtcttaataccgaaaacaagcagctggctgatactaccgctgttcccagcccagaagccacactgacagaaatggacagcaattaccatttttactcatcgatctcctcgctggaaaaagaagtgggcaactgtggtccacatttccttcatggttttgaggattctttcagcagcatcctctccactgatgatgccagccagctgacctccttagactcaaatcccaccttaaacacagattttggcgatgaattttattctgctttcatagcagagcccagtgacggtggcagcatgccttcctccgctgccatcagtcagtcactctctgaactcctggacgggactattgaaggctgtgacctgtcactgtgtaaagctttcaacccgaagcacgctgaaggcacaatggaattcaatgactctgactctggcatttcactgaacacgagtcccagccgagcgtccccagagcactccgtggagtcttccatttacggagacccaccgcctgggttcagtgactcggaaatggaggagctagatagtgcccctggaagtgtcaaacagaacggccctaaagcacagccagcacattctcctggagacacagtacagcctctgtcaccagctcaagggcacagtgctcctatgcgtgaatcccaatgtgaaaatacaacaaaaaaagaagttcccgtgagtcctggtcatcaaaaagccccattcacaaaagacaaacattcaagccgcttagaggctcatctcacacgagatgagcttagggcaaaagctctccatattccattccctgtcgaaaaaatcattaacctccctgttgatgacttcaatgaaatgatgtccaaggagcaattcaatgaagctcagctcgcattgatccgagatatacgcaggagaggtaagaataaagtcgccgcccagaactgtaggaaaaggaagctggagaacattgtcgagctggagcaagacttgggccacttaaaagacgagagagaaaaactactcagagaaaagggagaaaacgacagaaacctccatctactgaaaaggcggctcagcaccttgtatcttgaagtcttcagcatgttacgtgatgaggatggaaagccttactctcccagtgaatactctctgcagcaaaccagagatggcaatgtgttccttgttcccaaaagcaagaagccagatacaaagaaaaactaggttcgggaggatggagccttttctgagctagtgtttgttttgtactgctaaaacttcctactgtgatgtgaaatgcagaaacactttataagtaactatgcagaattatagccaaagctagtatagcaataatatgaaactttacaaagcattaaagtctcaatgttgaatcagtttcattttaactctcaagttaatttcttaggcaccatttgggagagtttctgtttaagtgtaaatactacagaacttatttatactgttctcacttgttacagtcatagacttatatgacatctggctaaaagcaaactattgaaaactaaccagaccactatacttttttatatactgtatgaacaggaaatgacatttttatattaaattgtttagctcataaaaattaaggagctagcactaataaaagaatatcatgact

SEQ ID NO: 211 - protein, 581 aa, Mus musculus
MDLIDILWRQDIDLGVSREVFDFSQRQKDYELEKQKKLEKERQEQLQKEQEKAFFAQFQLDEETGEFLPIQPAQHIQTDTSGSASYSQVAHIPKQDALYFEDCMQLLAETFPFVDDHESLALDIPSHAESSVFTAPHQAQSLNSSLEAAMTDLSSIEQDMEQVWQELFSIPELQCLNTENKQLADTTAVPSPEATLTEMDSNYHFYSSISSLEKEVGNCGPHFLHGFEDSFSSILSTDDASQLTSLDSNPTLNTDFGDEFYSAFIAEPSDGGSMPSSAAISQSLSELLDGTIEGCDLSLCKAFNPKHAEGTMEFNDSDSGISLNTSPSRASPEHSVESSIYGDPPPGFSDSEMEELDSAPGSVKQNGPKAQPAHSPGDTVQPLSPAQGHSAPMRESQCENTTKKEVPVSPGHQKAPFTKDKHSSRLEAHLTRDELRAKALHIPFPVEKIINLPVDDFNEMMSKEQFNEAQLALIRDIRRRGKNKVAAQNCRKRKLENIVELEQDLGHLKDEREKLLREKGENDRNLHLLKRRLSTLYLEVFSMLRDEDGKPYSPSEYSLQQTRDGNVFLVPKSKKPDTKKN

SEQ ID NO: 212 - DNA, 2304 bp, Homo sapiens
ttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcactcagttacaactagatgaagagacaggtgaatttctcccaattcagccagcccagcacacccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccacgatgtacttttttataaatactgtatggacaaaaaatggcattttttataattaaattgtttagctctggcaaaaaaaaaaaattttttaagagctggtactaataaaggattattatgactgttaaaaaaaaaaaaaaaaaa

SEQ ID NO: 213 - protein, 589 aa, Homo sapiens
MDLIDILWRQDIDLGVSREVFDFSQRRKEYELEKQKKLEKERQEQLQKEQEKAFFTQLQLDEETGEFLPIQPAQHTQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 214 - protein, 5 aa, Unknown
  cytokine receptor extracellular motif found in many species
  VARIANT 3: Xaa = any amino acid
WSXWS

SEQ ID NO: 215 - DNA, 19 bp, Mus musculus
gctgagtcatgatgagtca

(19) United States
(12) Patent Application Publication
(10) Pub. No.: US 2007/0059724 A1
(43) Pub. Date: Mar. 15, 2007

(54) Novel compositions and methods for lymphoma and leukemia

(76) Inventors: Finn Skou Pedersen, Aarhus (DK); Annette Balle Sorensen, Aarhus (DK); Javier Martin Hernandez, Aarhus (DK); Anne Ahlmann Nielsen, Aarhus (DK); Helle Moving, Aarhus (DK)

Correspondence Address: SAGRES DISCOVERY INC.;INTELLECTUAL PROPERTY - R440, P.O. BOX 8097, EMERYVILLE, CA, 94662-8097, US

(21) Appl. No.: 11/365,889

(22) Filed: Mar. 1, 2006

Related U.S. Application Data
(62) Division of application No. 09/963,131, filed on Sep. 24, 2001, now abandoned.
Application No. 09/963,131 is a continuation-in-part of application No. 09/668,644, filed on Sep. 22, 2000, now abandoned.
Application No. 09/963,131 is a continuation-in-part of application No. 09/905,390, filed on Jul. 13, 2001, now abandoned.
Application No. 09/963,131 is a continuation-in-part of application No. 09/905,491, filed on Jul. 13, 2001, now abandoned.

(51) Int. Cl. C12Q 1/68; G01N 33/574; A61K 48/00

(52) U.S. Cl. 435/6; 435/7.23; 514/44

(57) ABSTRACT

The present invention relates to novel sequences for use in diagnosis and treatment of lymphoma and leukemia. In addition, the present invention describes the use of novel compositions for use in screening methods.

This application is a continuing application of U.S. Ser. No. 09/668,644, filed Sep. 22, 2000; U.S. Ser. No. 09/905,390, filed Jul. 13, 2001; U.S. Ser. No. 09/905,491, filed Jul. 13, 2001; Methods for Diagnosis and Treatment of Diseases Associated with Altered Expression of Pik3r1, filed Sep. 24, 2001; Methods for Diagnosis and Treatment of Diseases Associated with Altered Expression of JAK1, filed Sep. 24, 2001; Methods for Diagnosis and Treatment of Diseases Associated with Altered Expression of Neurogranin, filed Sep. 24, 2001; Methods for Diagnosis and Treatment of Diseases Associated with Altered Expression of Nrf2, filed Sep. 24, 2001; all of which are expressly incorporated herein by reference.

### FIELD OF THE INVENTION

The present invention relates to novel sequences for use in diagnosis and treatment of lymphoma and leukemia, as well as the use of the novel compositions in screening Methods.

### BACKGROUND OF THE INVENTION

Lymphomas are a collection of cancers involving the lymphatic system and are generally categorized as Hodgkin's disease and Non-Hodgkin lymphoma. Hodgkin's lymphomas are of B lymphocyte origin. Non-Hodgkin lymphomas are a collection of over 30 different types of cancers including T and B lymphomas. Leukemia is a disease of the blood forming tissues and includes B and T cell lymphocytic leukemias. It is characterized by an abnormal and persistent increase in the number of leukocytes and the amount of bone marrow, with enlargement of the spleen and lymph nodes.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways, including promoter insertion, enhancer insertion, and/or truncation of a protooncogene or tumor suppressor gene. The analysis of sequences at or near the insertion sites led to the identification of a number of new protooncogenes.

With respect to lymphoma and leukemia, murine leukemia retrovirus (MuLV), such as SL3-3 or Akv, is a potent inducer of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide sequences involved in oncogenesis, particularly with respect to lymphomas.

In this regard, the present invention provides a mammalian Pik3r1 gene which is shown herein to be involved in lymphoma.

The phosphatidyl inositol 3′-kinases (PI3K, PI3 kinase) represent a ubiquitous family of heterodimeric lipid kinases that are found in association with the cytoplasmic domain of hormone and growth factor receptors and oncogene products. PI3Ks act as downstream effectors of these receptors, are recruited upon receptor stimulation and mediate the activation of second messenger signaling pathways through the production of phosphorylated derivatives of inositol (reviewed in Fry, Biochim. Biophys. Acta., 1226:237-268, 1994). There are multiple forms of PI3K having distinct mechanisms of regulation and different substrate specificities (reviewed in Carpenter et al., Curr. Opin. Biol. 8:153-158, 1996; Zvelebill et al., Phil. Trans. R. Soc. Lond. 351:217-223, 1996).

The PI3K heterodimers consist of a 110 kD (p110) catalytic subunit associated with an 85 kD (Pik3r1) regulatory subunit, and it is through the SH2 domains of the p85 regulatory subunit that the enzyme associates with membrane-bound receptors (Escobedo et al., Cell 65:75-82, 1991; Skolnik et al., Cell 65:83-90, 1991).

Pik3r1 was originally isolated from bovine brain and shown to exist in two forms, α and β. In these studies, p85 isoforms were shown to bind to and act as substrates for tyrosine-phosphorylated receptor kinases and the polyoma virus middle T antigen complex (Otsu et al., Cell 65:910104, 1991). Since then, the Pik3r1 subunit has been further characterized and shown to interact with a diverse group of proteins including receptor tyrosine kinases such as the erythropoietin receptor, the PDGR-β receptor and Tie2, an endothelieum-specific receptor involved in vascular development and tumor angigenesis (He et al., Blood 82:3530-3538, 1993; Kontos et al., MCB 18:4131-4140, 1998; Escobedo et al., Cell 65:75-82, 1991). Pik3r1 also interacts with focal adhesion kinase (FAK), a cytoplasmic tyrosine kinase that is involved in integrin signaling, an is though to be a substrate and effector of FAK. Pik3r1 also interacts with profilin, an actin-binding protein that facilitates actin polymerization (Bhagarvi et al., Biochem. Mol. Biol. Int. 46:241-248, 1998; Chen et al., PNAS 91:10148-10152, 1994) and the Pik3r1/profilin complex inhibits actin polymerization.

PI3K has been implicated in the regulation of many cellular activities, including but not limited to survival, proliferation, apoptosis, DNA synthesis, protein transport and neurite extension (reviewed in Fry, supra).

A truncated form of Pik3r1 including the first 571 amino acids of the native protein (as encoded by nucleotides 43-1755 in SEQ ID NO:3 and at Genbank accession number M61906) fused to an amino acid sequence conserved in the eph family of receptor tyrosine kinases causes constitutive activation of PI3K and contributes to cellular transformation of mammalian fibroblasts.

A dominant negative isoform of PI3K which inhibits downstream signaling to PKB (Akt) has been isolated (Burgering er al, Nature 376:599-602, 1995). In addition, a constitutively active form of PI3K has been isolated (Klippel et al., MCB 16:4117-4127, 1996; Mante et al., Curr. Biol. 7:63-70, 1996; Franke et al., Cell 81:727-736, 1995).

Many approaches to the inhibition of PI3K activity have focussed on the use of inhibitors. Several inhibitors of PI3K activity are known in the literature. These include wortmannin, a fungal metabolite (Ui et al., Trends Biochem. Sci., 20:303-307, 1995), demethoxyviridin, an antifungal agent (Woscholski et al., FEBS Lett. 342:109-114, 1994), quercetin and LY294002 (Vlahos et al., JBC 269:5241-5248, 1994). These inhibitors primarily target the p110 subunit of PI3k.

An additional approach taken to inhibit PI3K activity involves the inhibition of Pik3r1 expression, as through the use of antisense oligonucleotides directed to Pik3r1 nucleic acid sequence (for example, see U.S. Pat. No. 6,100,090 issued to Monia et al.).

As disclosed herein, alteration and/or dysregulation of Pik3r1 leads to lymphoma. Provided herein are novel compositions and methods for the diagnosis, treatment, and prophylaxis of lymphoma.

As demonstrated herein, GNAS genes are also implicated in lymphomas and leukemias. GNAS is a complex locus encoding multiple proteins, including an α subunit of a stimulatory G protein (Gₛα). G proteins transduce extracellular signals in signal transduction pathways. Each G protein is a heterotrimer, composed of an α, β and γ subunit. The β and γ subunits anchor the protein to the cytoplasmic side of the plasma membrane. Upon binding of a ligand, Gₛα dissociates from the complex, transducing signals from hormone receptors to effector molecules including adenylyl cyclase resulting in hormone-stimulated cAMP generation (Molecular Biology of the Cell, 3d edition, Alberts, B et al., Garland Publishing 1994).

Other proteins generated from the GNAS locus, through alternative splicing, include XLαs, a Gₛα isoform with an extended NH₂ terminal extension, and NESP55, a chromogranin-like neurosecretory protein (Weinstein L S et al., Am J Physiol Renal Physiol 2000, 278:F507-14). In mice, Nesp, the mouse homolog of NESP55, is located 15 kb upstream of Gnasxl, the mouse homolog of Xlαs, which is in turn, 30 kb upstream of Gnas (Wroe et al., Proc. Natl. Acad. Sci. 97:3342 (2000)). NESP55 is processed into smaller peptides, one of which acts as an inhibitor of the serotonergic 5-HT_{1B} receptor (Ischia et. al. J. Biol. Chem. 272:11657 (1997). The function of XLαs is not known, but it is also expressed primarily in the neuroendocrine system and may be involved in pseudohypoparathyroidsm type Ia (Hayward et al., Proc. Natl. Acad. Sci. 95:10038 (1998)). Xlαs and NESP55 have been found to be expressed in opposite parental alleles, as a result of imprinting (Wroe et al., Proc. Natl. Acad. Sci. 97:3342 (2000)).

GNAS also plays a role in diseases other than leukemias and lymphomas. Mutations in GNAS1, the human GNAS gene, result in Albright hereditary osteodystrophy (AHO), a disease characterized by short stature and obesity. Studies with the mouse homolog demonstrate that the obesity seen is a consequence of the reduced expression of GNAS. In contrast, other mutations have been shown to result in constitutive activation of Gₛα, resulting in endocrine tumors and McCune-Albright syndrome, a condition characterized by abnormalities in endocrine function (Aldred M A and Trembath, R C, Hum Mutat 2000, 16:183-9). The mechanism behind this disease as well as fibrous dysplasia, a progressive bone disease, is caused by increased cAMP levels which results in increase IL-6 levels, triggering abnormal osteoblast differentiation and increased osteoclastic activity (Stanton R P et al., J. Bone Miner. Res. 1999, 14:1104-14).

Accordingly, it is an object of the invention to provide methods for detection and screening of drug candidates for diseases involving GNAS, particularly with respect to lymphomas.

As demonstrated herein, a HIPK1 gene is also implicated in lymphomas and leukemias. HIPK1 is a member of a novel family of nuclear protein kinases that act as transcriptional co-repressors for NK class of homeoproteins (Kim Y H et al., J. Biol. Chem. 1998, 273:25875-25879). Homeoproteins are transcription factors that regulate homeobox genes, which are involved in various developmental processes, such as pattern formation and organogenesis (McGinnis, W. and Krumlauf, R., Cell 1992, 68:283-302).

Homeoproteins may play a role in human disease. Aberrant expression of the NKX2-5 homeodomain transcription factor has been found to be involved in a congenital heart disease (Schott, J.-J. et al., Science 1998, 281:108-111).

Accordingly, it is an object of the invention to provide methods for detection and screening of drug candidates for diseases involving HIPK1, particularly with respect to lymphomas.

Cytokines and Interferons regulate a wide range of cellular functions in the lympho-hematopoletic system. This regulation is mediated, in part, by the Jak-STAT pathway. In this pathway a Cytokine or Interferon initially binds to the extracellular portion of a membrane bound receptor. Binding of a Cytokine or Interferon activates members of the Janus family of Tyrosine Kinases (JAKs), including JAKI. Activated JAKs phosphorylate docking sites on the intracellular portion of the receptor which in turn activate transcription factors known as the signal transducers and activators of transcription (STATs). Once activated, STATs dimerize and translocate to the nucleus to bind target DNA sequences resulting in modulation of gene expression.

Given the integral role JAKs play in this signal transduction pathway it is not surprising that a number of studies have shown that JAK dysreguation leads to severe disease states. JAK mutations in Drosophila termed Tum-I, Tumorous lethal, for example, lead to leukemia in flies. Harrison et al., EMBO J. 14:1412-20 (1995); Luo et al., EMBO J. 14:1412-20 (1995); Luo et al., Mol. Cell. Biol. 17:1562-71 (1997). Additionally, constitutive activation of JAKs in mammalian cells has been shown to lead to malignant transformation in several settings. Migone et al., Science 269:79-81 (1995); Zhang et al., Proc. Natl. Acad. Sci. USA 93:9148-53 (1996); Danial et al., Science 269:1875-77 (1995); Meydan et al., Nature 379:645-48 (1996). Accordingly, understanding the various aspects of JAK function, its binding capabilities, catalytic aspects, etc., will give insight into a number of disease states not the least of which being either lymphoma or leukemia.

Neurogranin is a neuronal protein thought to play a role in dendritic spine formation and synaptic plasticity. The Neurogranin gene encodes a 78-amino acid protein that functions as a postsynaptic kinase substrate and has been shown to bind calmodulin in the absence of calcium. Martinez de Arrieta et al., Endocrinology 140(1):335-43 (1999). Though little is understood at the present time, dysregulation of Neurogranin gene expression has been implicated in disease states. Recent studies have shown Neurogranin expression is tightly regulated by thyroid hormone. Morte et al., FEBS Lett December 31; 464(3):179-83 (1999). This regulation may explain the role hypothyroidism has on mental states during development as well as in adult subjects. Additionally, a transactivator overexpressed in prostate cancer, EGR1, has been shown to induce Neurogranin which may explain the neuroendocrine differentiation that often accompanies prostate cancer progression. Svaren et al., J. Biol. Chem. December 8; 275(49):38524-31 (2000). Accordingly, understanding the various aspects of Neurogranin structure and function will likely lead to a clearer view of its role in hypothyroidism and prostate cancer, as well as other diseases such as lymphoma and leukemia.

Accordingly, it is an object of the invention to provide compositions involved in oncogenesis, particularly with respect to the role of Neurogranin in lymphomas.

Also, in this regard, the present invention provides a mammalian Nrf2 gene which is shown herein to be involved in lymphoma.

The Nrf2 gene encodes a DNA binding transcriptional regulatory protein (transcription factor) belonging to the “cap 'n collar” subfamily of the basic leucine zipper family of transcription factors (Chan et al., PNAS 93:13943-13948, 1996; Moi et-al., PNAS 91:9926-9930, 1994). The Nrf2 gene produces a 2.2 kb transcript which predicts a 66 kDa protein (Moi et al., PNAS 91:9926-9930, 1994). The Nrf2 protein binds to a DNAse hypersensitive site located in the β-globin locus control region (Mol et al., PNAS 91:9926-9930, 1994), as well as to the antioxidant response element (ARE) which is found in the regulatory regions of many detoxifying enzyme genes (Venugopal et al., Oncogene, 17:3145-3156, 1998).

Nrf2 gene function is not required for normal development, as evidenced by homozygous disruption of the Nrf2 loci in transgenic mice (Chan et al., PNAS 93:13943-13948, 1996). However, loss of Nrf2 gene function compromises the ability of haematopioetic cells to endure oxidative stress (Ishii et al., J. Biol. Chem., 275:16023-16029, 2000; Enomoto et al., Toxicol. Sci., 59:169-177, 2001) and sensitizes cells to the carcinogenic activity of oxidative agents (Ramos-Gomez et al., PNAS, 98:3410-3415, 2001).

Nrf2 proteins are capable of interacting with other transcription factors, including Jun proteins (Venugopal et al., Oncogene, 17:3145-3156, 1998) and Maf proteins (Marini et al., J. Biol. Chem., 272-16490-16497, 1997). Jun proteins appear to cooperate with Nrf2 to regulate the transcription of target genes (Venugopal et al., Oncogene, 17:3145-3156, 1998) while Maf proteins appear to antagonize the transcription promoting activity of Nrf2 protein (Nguyen et al., J. Biol. Chem., 275:15466-15473, 2000). In addition, the human cytomegalovirus protein IE-2 has also been found to interact with Nrf2 and to inhibit its transcription promoting activity (Huang et al., J. Biol. Chem., 275:12313-12320, 2000).

Despite being dispensable for the normal development of lymphoid cells and tissues, which includes the normal processes of B cell and T cell determination, differentiation, proliferation, and death, it is demonstrated herein that dysregulation of the Nrf2 gene leads to lymphoma.

### SUMMARY OF THE INVENTION

In accordance with the objects outlined above, the present invention provides methods for screening for compositions which modulate lymphomas. Also provided herein are methods of inhibiting proliferation of a cell, preferably a lymphoma cell. Methods of treatment of lymphomas, including diagnosis, are also provided herein.

In one aspect, a method of screening drug candidates comprises providing a cell that expresses a lymphoma associated (LA) gene or fragments thereof. Preferred embodiments of LA genes are genes which are differentially expressed in cancer cells, preferably lymphoma or leukemia cells, compared to other cells. Preferred embodiments of LA genes used in the methods herein include, but are not limited to the nucleic acids selected from Tables 1, 2 or 3. Additional preferred embodiments include, but are not limited to, the nucleic acids set forth in Tables 4, 6, 8, 9, 10, 11, 12, 13, 14, 15, 16, 17, 18, 19, 22, 23, 24, 27, 28 or 30. The method further includes adding a drug candidate to the cell and determining the effect of the drug candidate on the expression of the LA gene.

In one embodiment, the method of screening drug candidates includes comparing the level of expression in the absence of the drug candidate to the level of expression in the presence of the drug candidate.

Also provided herein is a method of screening for a bioactive agent capable of binding to a LA protein (LAP), the method comprising combining the LAP and a candidate bioactive agent, and determining the binding of the candidate agent to the LAP. In a preferred embodiment, a LA protein is selected from the amino acid sequences set forth in Tables 5, 7, 9, 10, 11, 12, 13, 14, 16, 17, 20, 21, 25, 26, 29 or 31.

Further provided herein is a method for screening for a bioactive agent capable of modulating the activity of a LAP. In one embodiment, the method comprises combining the LAP and a candidate bioactive agent, and determining the effect of the candidate agent on the bioactivity of the LAP.

Also provided is a method of evaluating the effect of a candidate lymphoma drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of an LA protein is provided. In one embodiment, the method comprises administering to a patient an inhibitor of an LA protein preferably encoded by a nucleic acid selected from the group consisting of the sequences outlined in Tables 1, 2 or 3. Additional preferred embodiments include, but are not limited to, the nucleic acids set forth in Tables 4, 6, 8, 9, 10, 11, 12, 13, 14, 15, 16, 17, 18, 19, 22, 23, 24, 27, 28 or 30. In a preferred embodiment, a LA protein is selected from the amino acid sequences set forth in Tables 5, 7, 9, 10, 11, 12, 13, 14, 16, 17, 20, 21, 25, 26, 29 or 31.

A method of neutralizing the effect of a LA protein, preferably selected from the group of sequences outlined in Tables, 1, 2 or 3, is also provided. Additional preferred embodiments include, but are not limited to, the nucleic acids set forth in Tables 4, 6, 8, 9, 10, 11, 12, 13, 14, 15, 16, 17, 18, 19, 22, 23, 24, 27, 28 or 30. In a preferred embodiment, a LA protein is selected from the amino acid sequences set forth in Tables 5, 7, 9, 10, 11, 12, 13, 14, 16, 17, 20, 21, 25, 26, 29 or 31. Preferably, the method comprises contacting an agent specific for said protein with said protein in an amount sufficient to effect neutralization.

Moreover, provided herein is a biochip comprising a nucleic acid segment which encodes a LA protein, preferably selected from the sequences outlined in Tables 1, 2 or 3. Additional preferred embodiments include, but are not limited to, the nucleic acids set forth in Tables 4, 6, 8, 9, 10, 11, 12, 13, 14, 15, 16, 17, 18, 19, 22, 23, 24, 27, 28 or 30. In a preferred embodiment, a LA protein is selected from the amino acid sequences set forth in Tables 5, 7, 9, 10, 11, 12, 13, 14, 16, 17, 20, 21, 25, 26, 29 or 31.

Also provided herein is a method for diagnosing or determining the propensity to lymphomas by sequencing at least on LA gene of an individual. In yet another aspect of the invention, a method is provided for determining LA gene copy number in an individual.

Novel sequences are also provided herein. Other aspects of the invention will become apparent to the skilled artisan by the following description of the invention.

In one aspect the present invention provides an LA protein known as Pik3r1 comprising the amino acid sequence set forth in SEQ ID NO:179 and at Genbank Accession number AAC52847, which is encoded by the Pik3r1 nucleic acid sequence set forth by nucleotides 575 to 2749 in SEQ ID NO:178 and at Genbank Accession Number U50413. In one aspect the present invention provides an LA nucleic acid referred to herein as Pik3r1 and comprising the nucleic acid sequence set forth in SEQ ID NO:178 and at Genbank Accession number U50413, which encodes an Pik3r1 protein.

In one aspect the present invention provides an LA protein known as Pik3r1 comprising the amino acid sequence set forth in SEQ ID NO:181 and at Genbank Accession number A38748. In one aspect the present invention provides an LA nucleic acid referred to herein as Pik3r1 and comprising the nucleic acid sequence set forth by nucleotides 43 to 2217 in SEQ ID NO:3 and at Genbank Accession number M61906, which encodes an Pik3r1 protein.

Also provided herein are Pik3r1 nucleic acids comprising a nucleic acid sequence having at least about 90% identity to the nucleic acid sequence set forth in SEQ ID NO:178 and at Genbank Accession number U50413, or complements thereof.

Also provided herein are Pik3r1 nucleic acids comprising a nucleic acid sequence having at least about 90% identity to the nucleic acid sequence set forth in SEQ ID NO:180 and at Genbank accession number M61906, or complements thereof.

Also provided herein are Pik3r1 nucleic acids which will hybridize under high stringency conditions to a nucleic acid comprising the nucleic acid sequence set forth in SEQ ID NO:178 and at Genbank accession number U50413, or complements thereof.

Also provided herein are Pik3r1 nucleic acids which will hybridize under high stringency conditions to a nucleic acid comprising the nucleic acid sequence set forth in SEQ ID NO:180 and at Genbank accession number M61906, or complements thereof.

Also provided herein are Pik3r1 proteins encoded by Pik3r1 nucleic acids as described herein.

Also provided herein are Pik3r1 proteins comprising an amino acid sequence having at least about 90% identity to the amino acid sequence set forth in SEQ ID NO:179 and at Genbank accession number AAC52847.

Also provided herein are Pik3r1 proteins comprising an amino acid sequence having at least about 90% identity to the amino acid sequence set forth in SEQ ID NO:181 and at Genbank accession number A38748.

Also provided herein are Pik3r1 genes encoding Pik3r1 proteins comprising an amino acid sequence having at least about 90% identity to the amino acid sequence set forth in SEQ ID NO:179 and at Genbank accession number MC52847.

Also provided herein are Pik3r1 genes encoding Pik3r1 proteins comprising an amino acid sequence having at least about 90% identity to the amino acid sequence set forth in SEQ ID NO:181 and at Genbank accession number A38748.

In one aspect, the present invention provides a method for screening for a candidate bioactive agent capable of modulating the activity of a Pik3r1 gene. In one embodiment, such a method comprises adding a candidate agent to a cell and determining the level of expression of a Pik3r1 gene in the presence and absence of the candidate agent. In a preferred embodiment, a Pik3r1 gene comprises the nucleic acid sequence set forth in SEQ ID NO:178 and at Genbank accession number U50413. In another preferred embodiment, a Pik3r1 gene comprises the nucleic acid sequence set forth in SEQ ID NO:180 and at Genbank accession number M61906.

Further provided herein is a method for screening for a candidate bioactive agent capable of modulating the activity of a Pik3r1 protein encoded by a Pik3r1 gene. In one embodiment, such a method comprises contacting a Pik3r1 protein or a cell comprising a Pik3r1 protein, and a candidate bioactive agent, and determining the effect on the activity of the Pik3r1 protein in the presence and absence of the candidate agent. In another embodiment, such a method comprises contacting a cell comprising a Pik3r1 protein, and a candidate bioactive agent, and determining the effect on the cell in the presence and absence of the candidate agent. In a preferred embodiment, a Pik3r1 protein comprises the amino acid sequence set forth in SEQ ID NO:179 and at Genbank accession number AAC52847, or a fragment thereof. In another preferred embodiment, a Pik3r1 protein comprises the amino acid sequence set forth in SEQ ID NO:181 and at Genbank accession number A38748, or a fragment thereof. In a preferred embodiment, a Pik3r1 protein comprises an amino acid sequence encoded by the nucleic acid sequence set forth in SEQ ID NO:178 and at Genbank accession number U50413, or a fragment thereof. In another preferred embodiment, a Pik3r1 protein comprises an amino acid sequence encoded by the nucleic acid sequence set forth in SEQ ID NO:180 and at Genbank accession number M61906, or a fragment thereof. In one embodiment, a Pik3r1 protein is a recombinant protein. In one embodiment, a Pik3r1 protein is isolated. In one embodiment, a Pik3r1 protein is cell-free, as in a cell lysate.

Also provided herein is a method for screening for a bioactive agent capable of binding to a Pik3r1 protein encoded by a Pik3r1 gene. In one embodiment, such a method comprises combining a Pik3r1 protein or a cell comprising a Pik3r1 protein, and a candidate bioactive agent, and determining the binding of the candidate agent to the Pik3r1 protein. In a preferred embodiment, a Pik3r1 protein comprises the amino acid sequence set forth in SEQ ID NO:179, or a fragment thereof. In another preferred embodiment, a Pik3r1 protein comprises the amino acid sequence set forth in SEQ ID NO:181, or a fragment thereof. In a preferred embodiment, a Pik3r1 protein comprises an amino acid sequence encoded by the nucleic acid sequence set forth in SEQ ID NO:178, or a fragment thereof. In another preferred embodiment, a Pik3r1 protein comprises an amino acid sequence encoded by the nucleic acid sequence set forth in SEQ ID NO:180, or a fragment thereof. In one embodiment, a Pik3r1 protein is a recombinant protein. In one embodiment, a Pik3r1 protein is isolated. In one embodiment, a Pik3r1 protein is cell-free, as in a cell lysate.

Also provided is a method for evaluating the effect of a candidate lymphoma drug, comprising administering the drug to a patient and removing a cell sample or a cell fraction sample from the patient. A gene expression profile for the sample is then determined, including determination of the expression of a Pik3r1 gene. In a preferred embodiment, a Pik3r1 gene comprises the nucleic acid sequence set forth in SEQ ID NO:178, or a fragment thereof. In another preferred embodiment, a Pik3r1 gene comprises the nucleic acid sequence set forth in SEQ ID NO:180, or a fragment thereof. Such a method may further comprise comparing the expression profile of the patient sample to an expression profile of a healthy individual sample.

In a further aspect, a method for inhibiting the activity of a Pik3r1 protein is provided. In one embodiment, the method comprises administering to a patient an inhibitor of a Pik3r1 protein. In a preferred embodiment, a Pik3r1 protein comprises the amino acid sequence set forth in SEQ ID NO:179 or a fragment thereof. In another preferred embodiment, a Pik3r1 protein comprises the amino acid sequence set forth in SEQ ID NO:181 or a fragment thereof. In a preferred embodiment, a Pik3r1 protein comprises an amino acid sequence encoded by the nucleic acid sequence set forth in SEQ ID NO:178 or a fragment thereof. In another preferred embodiment, a Pik3r1 protein comprises an amino acid sequence encoded by the nucleic acid sequence set forth in SEQ ID NO:180 or a fragment thereof.

Also provided herein is a method for neutralizing Pik3r1 protein activity with a bioactive agent. In a preferred embodiment, a Pik3r1 protein comprises the amino acid sequence set forth in SEQ ID NO:179 or a fragment thereof. In another preferred embodiment, a Pik3r1 protein comprises the amino acid sequence set forth in SEQ ID NO:181 or a fragment thereof. In a preferred embodiment, a Pik3r1 protein comprises an amino acid sequence encoded by the nucleic acid sequence set forth in SEQ ID NO:178, or a fragment thereof. In another preferred embodiment, a Pik3r1 protein comprises an amino acid sequence encoded by the nucleic acid sequence set forth in SEQ ID NO:180, or a fragment thereof. In one embodiment, such a method comprises contacting a Pik3r1 protein with an agent that specifically modulates Pik3r1 protein activity, in an amount sufficient to effect neutralization.

Moreover, provided herein is a biochip comprising a nucleic acid which encodes a Pik3r1 protein or a portion thereof. In a preferred embodiment, a Pik3r1 nucleic acid comprises the nucleic acid sequence set forth in SEQ ID NO:178, or complement thereof, or a fragment thereof or complement of a fragment thereof. In another preferred embodiment, a Pik3r1 nucleic acid comprises the nucleic acid sequence set forth in SEQ ID NO:180, or complement thereof, or a fragment thereof or complement of a fragment thereof.

Also provided herein is a method for diagnosing or determining a predisposition for lymphomas, comprising sequencing at least one Pik3r1 gene from an individual and determining the nucleic acid sequence of the Pik3r1 gene or a fragment thereof. In a preferred embodiment, a Pik3r1 gene comprises the nucleic acid sequence set forth in SEQ ID NO:178, or a fragment thereof. In another preferred embodiment, a Pik3r1 gene comprises the nucleic acid sequence set forth in SEQ ID NO:180, or a fragment thereof.

Similarly provided are methods for determining lymphoma subtype and determining a prognosis for an individual having lymphoma, which comprise sequencing at least one Pik3r1 gene from an individual and determining the nucleic acid sequence of the Pik3r1 gene or a fragment thereof. In a preferred embodiment, a Pik3r1 gene comprises the nucleic acid sequence set forth in SEQ ID NO:178, or a fragment thereof. In another preferred embodiment, a Pik3r1 gene comprises the nucleic acid sequence set forth in SEQ ID NO:180, or a fragment thereof.

In yet another aspect of the invention, a method is provided for determining the number of copies of a Pik3r1 gene in an individual. In a preferred embodiment, a Pik3r1 gene comprises the nucleic acid sequence set forth in SEQ ID NO:178, or complement thereof, or a fragment thereof or complement of a fragment thereof. In a preferred embodiment, a Pik3r1 gene comprises the nucleic acid sequence set forth in SEQ ID NO:180, or complement thereof, or a fragment thereof or complement of a fragment thereof.

In yet another aspect of the invention, a method is provided for determining the chromosomal location of a Pik3r1 gene. In a preferred embodiment, a Pik3r1 gene comprises the nucleic acid sequence set forth in SEQ ID NO:178, or a fragment thereof. In another preferred embodiment, a Pik3r1 gene comprises the nucleic acid sequence set forth in SEQ ID NO:180, or a fragment thereof. Such a method may be used to determine Pik3r1 gene rearrangements or translocations. Without being bound by theory, Pik3r1 gene rearrangement and translocation events appear to be important in the aetiology of lymphoma.

It is an object of this invention that the identification Pik3r1 genes and recognition of their involvement in lymphoma provide diagnostic agents to distinguish between lymphoma subtypes, and analytical agents for further analysis of mechanisms involved in dysregulated growth and/or survival and/or apoptosis in cells of the hematopoietic system. An additional object of the invention is to provide appropriate and potentially novel targets for therapeutic interventions, particularly with regard to lymphoma, which are identified through the use of the diagnostic and analytical agents provided herein.

Without being bound by theory, it is recognized herein that the involvement of Pik3r1 genes in the cellular dysregulation underlying lymphoma implicates genes having products which are regulated by the PI3K pathway, preferably by phosphorylation by protein kinase B (PKB; AKT) and/or protein kinase C (PKC), in the cellular dysregulation underlying lymphoma.

Moreover, it is recognized herein that dysregulated growth in the hematopoietic system has been attributed to the inhibition of apoptosis, for example as by the deregulated expression of Bcl-2. Without being bound by theory, the present disclosure provides a new molecular mechanism for lymphoma in which alterations in Pik3r1 lead to alterations in the activity of PKB and the phosphorylation of proteins involved in survival and cell death, such as the Bcl-2 family member “BAD” (see Datta et al., Cell 91:231-241, 1997; del Peso et al., Science 278:687-689, 1997).

Novel sequences are also provided herein. Other aspects of the invention will become apparent to the skilled artisan by the following description of the invention.

In one aspect, a method of screening drug candidates comprises providing a cell that expresses a GNAS gene or fragments thereof. The method further includes adding a drug candidate to the cell and determining the effect of the drug candidate on the expression of a GNAS gene.

In one embodiment, the method of screening drug candidates includes comparing the level of expression in the absence of the drug candidate to the level of expression in the presence of the drug candidate.

Also provided herein is a method of screening for a bioactive agent capable of binding to a protein encoded by a GNAS gene, e.g. Gₛα, the method comprising combining a Gnas protein and a candidate bioactive agent, and determining the binding of the candidate agent to the Gnas protein.

Further provided herein is a method for screening for a bioactive agent capable of modulating the activity of a protein encoded by a GNAS gene. In one embodiment, the method comprises combining a Gnas protein and a candidate bioactive agent, and determining the effect of the candidate agent on the bioactivity of a Gnas protein.

Also provided is a method of evaluating the effect of a candidate lymphoma drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of a protein encoded by a GNAS gene is provided. In one embodiment, the method comprises administering to a patient an inhibitor of a Gnas protein.

A method of neutralizing the effect of Gnas proteins is also provided. Preferably, the method comprises contacting an agent specific for said protein with said protein in an amount sufficient to effect neutralization.

Moreover, provided herein is a biochip comprising a nucleic acid segment which encodes a Gnas protein.

Also provided herein is a method for diagnosing or determining the propensity to diseases, including lymphomas, by sequencing at least one GNAS gene of an individual. In yet another aspect of the invention, a method is provided for determining GNAS gene copy number in an individual.

In one aspect, a method of screening drug candidates comprises providing a cell that expresses a HIPK1 gene or fragments thereof. The method further includes adding a drug candidate to the cell and determining the effect of the drug candidate on the expression of a HIPK1 gene.

In one embodiment, the method of screening drug candidates includes comparing the level of expression in the absence of the drug candidate to the level of expression in the presence of the drug candidate.

Also provided herein is a method of screening for a bioactive agent capable of binding to a protein encoded by a HIPK1 gene, the method comprising combining a HIPK1 protein and a candidate bioactive agent, and determining the binding of the candidate agent to a HIPK1 protein.

Further provided herein is a method for screening for a bioactive agent capable of modulating the activity of a protein encoded by a HIPK1 gene. In one embodiment, the method comprises combining a HIPK1 protein and a candidate bioactive agent, and determining the effect of the candidate agent on the bioactivity of a HIPK1 protein.

Also provided is a method of evaluating the effect of a candidate lymphoma drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of a protein encoded by a HIPK1 gene is provided. In one embodiment, the method comprises administering to a patient an inhibitor of a HIPK1 protein.

A method of neutralizing the effect of HIPK1 protein is also provided. Preferably, the method comprises contacting an agent specific for said protein with said protein in an amount sufficient to effect neutralization.

Moreover, provided herein is a biochip comprising a nucleic acid segment which encodes HIPK1 protein.

Also provided herein is a method for diagnosing or determining the propensity to diseases, including lymphomas, by sequencing at least one HIPK1 gene of an individual. In yet another aspect of the invention, a method is provided for determining HIPK1 gene copy number in an individual.

In one aspect, a method of screening drug candidates comprises providing a cell that expresses a JAKI gene or fragments thereof. Preferred embodiments of JAKI genes are genes which are differentially expressed in cancer cells, preferably lymphoma or leukemia cells, compared to other cells. The method further includes adding a drug candidate to the cell and determining the effect of the drug candidate on the expression of the JAKI gene.

In one embodiment, the method of screening drug candidates includes comparing the level of expression in the absence of the drug-candidate to the level of expression in the presence of the drug candidate.

Also provided herein is a method of screening for a bioactive agent capable of binding to a JAKI protein, the method comprising combining the JAKI protein and a candidate bioactive agent, and determining the binding of the candidate agent to the JAKI protein.

Further provided herein is a method for screening for a bioactive agent capable of modulating the activity of JAKI protein. In one embodiment, the method comprises combining the JAKI protein and a candidate bioactive agent, and determining the effect of the candidate agent on the bioactivity of the JAKI protein.

Also provided is a method of evaluating the effect of a candidate lymphoma drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of a JAKI protein is provided.

A method of neutralizing the effect of a JAKI protein, is also provided. Preferably, the method comprises contacting an agent specific for said protein with said protein in an amount sufficient to effect neutralization.

Moreover, provided herein is a biochip comprising a nucleic acid segment which encodes a JAKI protein.

Also provided herein is a method for diagnosing or determining the propensity to lymphomas by sequencing the JAKI gene of, an individual. In yet another aspect of the invention, a method is provided for determining JAKI gene copy number in an individual.

In one aspect, a method of screening drug candidates comprises providing a cell that expresses a Neurogranin gene or fragments thereof. Preferred embodiments of Neurogranin genes are genes which are differentially expressed in cancer cells, preferably lymphoma or leukemia cells, compared to other cells. The method further includes adding a drug candidate to the cell and determining the effect of the drug candidate on the expression of the Neurogranin gene.

In one embodiment, the method of screening drug candidates includes comparing the level of expression in the absence of the drug candidate to the level of expression in the presence of the drug candidate.

Also provided herein is a method of screening for a bioactive agent capable of binding to a Neurogranin protein, the method comprising combining the Neurogranin protein and a candidate bioactive agent, and determining the binding of the candidate agent to the Neurogranin protein.

Further provided herein is a method for screening for a bioactive agent capable of modulating the activity of Neurogranin protein. In one embodiment, the method comprises combining the Neurogranin protein and a candidate bioactive agent, and determining the effect of the candidate agent on the bioactivity of the Neurogranin protein.

Also provided is a method of evaluating the effect of a candidate lymphoma drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of a Neurogranin protein is provided. In one embodiment, the method comprises administering to a patient an inhibitor of a Neurogranin protein.

A method of neutralizing the effect of a Neurogranin protein, is also provided. Preferably, the method comprises contacting an agent specific for said protein with said protein in an amount sufficient to effect neutralization.

Moreover, provided herein is a biochip comprising a nucleic acid segment which encodes a Neurogranin protein.

Also provided herein is a method for diagnosing or determining the propensity to lymphomas by sequencing the Neurogranin gene of an individual. In yet another aspect of the invention, a method is provided for determining Neurogranin gene copy number in an individual.

In one aspect the present invention provides an LA protein known as Nrf2. In a preferred embodiment Nrf2 comprises the amino acid sequence set forth in SEQ ID NO:211 and at Genbank Accession number AAA68291, which is encoded by the Nrf2 nucleic acid sequence set forth by nucleotides 298 to 2043 in SEQ ID NO:210 and at Genbank Accession Number U20532. In one aspect the present invention provides an LA nucleic acid referred to herein as Nrf2. In a preferred embodiment the Nrf2 nucleic acid comprises the nucleic acid sequence set forth in SEQ ID NO:210 and at Genbank Accession number U20532, which encodes an Nrf2 protein.

In one aspect the present invention provides an LA protein known as Nrf2 comprising the amino acid sequence set forth in SEQ ID NO:213 and at Genbank Accession number NP-006155, which is encoded by the Nrf2 nucleic acid sequence set forth by nucleotides 40 to 1809 in SEQ ID NO:212 and at Genbank Accession Number NM_{—}006164. In one aspect the present invention provides an LA nucleic acid referred to herein as Nrf2 and comprising the nucleic acid sequence set forth in SEQ ID NO:212 and at Genbank Accession number NM_{—}006164, which encodes an Nrf2 protein.

Also provided herein are Nrf2 nucleic acids comprising a nucleic acid sequence having at least about 90% identity to the nucleic acid sequence set forth in SEQ ID NO:210 and at Genbank Accession number U20532, or complements thereof.

Also provided herein are Nrf2 nucleic acids comprising a nucleic acid sequence having at least about 90% identity to the nucleic acid sequence set forth in SEQ ID NO:212 and at Genbank accession number NM_{—}006164, or complements thereof.

Also provided herein are Nrf2 nucleic acids which will hybridize under high stringency conditions to a nucleic acid comprising the nucleic acid sequence set forth in SEQ ID NO:210 and at Genbank accession number U20532, or complements thereof.

Also provided herein are Nrf2 nucleic acids which will hybridize under high stringency conditions to a nucleic acid comprising the nucleic acid sequence set forth in SEQ ID NO:212 and at Genbank accession number NM_{—}006164, or complements thereof.

Also provided herein are Nrf2 proteins encoded by Nrf2 nucleic acids as described herein.

Also provided herein are Nrf2 proteins comprising an amino acid sequence having at least about 90% identity to the amino acid sequence set forth in SEQ ID NO:211 and at Genbank accession number AAA68291.

Also provided herein are Nrf2 proteins comprising an amino acid sequence having at least about 90% identity to the amino acid sequence set forth in SEQ ID NO:213 and at Genbank accession number NP_{—}006155.

Also provided herein are Nrf2 genes encoding Nrf2 proteins comprising an amino acid sequence having at least about 90% identity to the amino acid sequence set forth in SEQ ID NO:211 and at Genbank accession number AAA68291.

Also provided herein are Nrf2 genes encoding Nrf2 proteins comprising an amino acid sequence having at least about 90% identity to the amino acid sequence set forth in SEQ ID NO:213 and at Genbank accession number NP_{—}006155.

In one aspect, the present invention provides a method for screening for a candidate bioactive agent capable of modulating the activity of an Nrf2 gene. In one embodiment, such a method comprises adding a candidate agent to a cell and determining the level of expression of an Nrf2 gene in the presence and absence of the candidate agent. In a preferred embodiment, an Nrf2 gene comprises the nucleic acid sequence set forth in SEQ ID NO:210 and at Genbank accession number U20532. In another preferred embodiment, an Nrf2 gene comprises the nucleic acid sequence set forth in SEQ ID NO:212 and at Genbank accession number NM_{—}006164.

Further provided herein is a method for screening for a candidate bioactive agent capable of modulating the activity of an Nrf2 protein encoded by an Nrf2 gene. In one embodiment, such a method comprises contacting an Nrf2 protein or a cell comprising an Nrf2 protein, and a candidate bioactive agent, and determining the effect on the activity of the Nrf2 protein in the presence and absence of the candidate agent. In another embodiment, such a method comprises contacting a cell comprising an Nrf2 protein, and a candidate bioactive agent, and determining the effect on the cell in the presence and absence of the candidate agent. In a preferred embodiment, an Nrf2 protein comprises the amino acid sequence set forth in SEQ ID NO:211 and at Genbank accession number AAA68291, or a fragment thereof. In another preferred embodiment, an Nrf2 protein comprises the amino acid sequence set forth in SEQ ID NO:213 and at Genbank accession number NP_{—}006155, or a fragment thereof. In a preferred embodiment, an Nrf2 protein comprises an amino acid sequence encoded by the nucleic acid sequence set forth in SEQ ID NO:210 and at Genbank accession number U20532, or a fragment thereof. In another preferred embodiment, an Nrf2 protein comprises an amino acid sequence encoded by the nucleic acid sequence set forth in SEQ ID NO:212 and at Genbank accession number NM_{—}006164, or a fragment thereof. In one embodiment, an Nrf2 protein is a recombinant protein. Intone embodiment, an Nrf2 protein is isolated. In one embodiment, an Nrf2 protein is cell-free, as in a cell lysate.

Also provided herein is a method for screening for a bioactive agent capable of binding to an Nrf2 protein encoded by an Nrf2 gene. In one embodiment, such a method comprises combining an Nrf2 protein or a cell comprising an Nrf2 protein, and a candidate bioactive agent, and determining the binding of the candidate agent to the Nrf2 protein. In a preferred embodiment, an Nrf2 protein comprises the amino acid sequence set forth in SEQ ID NO:211, or a fragment thereof. In another preferred embodiment, an Nrf2 protein comprises the amino acid sequence set forth in SEQ ID NO:213, or a fragment thereof. In a preferred embodiment, an Nrf2 protein comprises an amino acid sequence encoded by the nucleic acid sequence set forth in SEQ ID NO:210, or a fragment thereof. In another preferred embodiment, an Nrf2 protein comprises an amino acid sequence encoded by the nucleic acid sequence set forth in SEQ ID NO:212, or a fragment thereof. In one embodiment, an Nrf2 protein is a recombinant protein. In one embodiment, an Nrf2 protein is isolated. In one embodiment, an Nrf2 protein is cell-free, as in a cell lysate.

Also provided is a method for evaluating the effect of a candidate lymphoma drug, comprising administering the drug to a patient and removing a cell sample or a cell fraction sample from the patient. A gene expression profile for the sample is then determined, including determination of the expression of an Nrf2 gene. In a preferred embodiment, an Nrf2 gene comprises the nucleic acid sequence set forth in SEQ ID NO:210, or a fragment thereof. In another preferred embodiment, an Nrf2 gene comprises the nucleic acid sequence set forth in SEQ ID NO:212, or a fragment thereof. Such a method may further comprise comparing the expression profile of the patient sample to an expression profile of a healthy individual sample.

In a further aspect, a method for inhibiting the activity of an Nrf2 protein is provided. In one embodiment, the method comprises administering to a patient an inhibitor of ah Nrf2 protein. In a preferred embodiment, an Nrf2 protein comprises the amino acid sequence set forth in SEQ ID NO:211 or a fragment thereof. In another preferred embodiment, an Nrf2 protein comprises the amino acid sequence set forth in SEQ ID NO:213 or a fragment thereof. In a preferred embodiment, an Nrf2 protein comprises an amino acid sequence encoded by the nucleic acid sequence set forth in SEQ ID NO:210 or a fragment thereof. In another preferred embodiment, an Nrf2 protein comprises an amino acid sequence encoded by the nucleic acid sequence set forth in SEQ ID NO:212 or a fragment thereof.

Also provided herein is a method for neutralizing Nrf2 protein activity with a bioactive agent. In a preferred embodiment, an Nrf2 protein comprises the amino acid sequence set forth in SEQ ID NO:211 or a fragment thereof. In another preferred embodiment, an Nrf2 protein comprises the amino acid sequence set forth in SEQ ID NO:213 or a fragment thereof. In a preferred embodiment, an Nrf2 protein comprises an amino acid sequence encoded by the nucleic acid sequence set forth in SEQ ID NO:210, or a fragment thereof. In another preferred embodiment, an Nrf2 protein comprises an amino acid sequence encoded by the nucleic acid sequence set forth in SEQ ID NO:212, or a fragment thereof. In one embodiment, such a method comprises contacting an Nrf2 protein with an agent that specifically modulates Nrf2 protein activity, in an amount sufficient to effect neutralization.

Moreover, provided herein is a biochip comprising a nucleic acid which encodes an Nrf2 protein or a portion thereof. In a preferred embodiment, an Nrf2 nucleic acid comprises the nucleic acid sequence set forth in SEQ ID NO:210, or complement thereof, or a fragment thereof or complement of a is fragment thereof. In another preferred embodiment, an Nrf2 nucleic acid comprises the nucleic acid sequence set forth in SEQ ID NO:212, or complement thereof, or a fragment thereof or complement of a fragment thereof.

Also provided herein is a method for diagnosing or determining a predisposition for lymphomas, comprising sequencing at least one Nrf2 gene from an individual and determining the nucleic acid sequence of the Nrf2 gene or a fragment thereof. In a preferred embodiment, an Nrf2 gene comprises the nucleic acid sequence set forth in SEQ ID NO:210, or a fragment thereof. In another preferred embodiment, an Nrf2 gene comprises the nucleic acid sequence set forth in SEQ ID NO:212, or a fragment thereof.

Similarly provided are methods for determining lymphoma subtype and determining a prognosis for an individual having lymphoma, which comprise sequencing at least one Nrf2 gene from an individual and determining the nucleic acid sequence of the Nrf2 gene or a fragment thereof. In a preferred embodiment, an Nrf2 gene comprises the nucleic acid sequence set forth in SEQ ID NO:210, or a fragment thereof. In another preferred embodiment, an Nrf2 gene comprises the nucleic acid sequence set forth in SEQ ID NO:212, or a fragment thereof.

In yet another aspect of the invention, a method is provided for determining the number of copies of an Nrf2 gene in an individual. In a preferred embodiment, an Nrf2 gene comprises the nucleic acid sequence set forth in SEQ ID NO:210, or complement thereof, or a fragment thereof or complement of a fragment thereof. In a preferred embodiment, an Nrf2 gene comprises the nucleic acid sequence set forth in SEQ ID NO:212, or complement thereof, or a fragment thereof or complement of a fragment thereof.

In yet another aspect of the invention, a method is provided for determining the chromosomal location of an Nrf2 gene. In a preferred embodiment, an Nrf2 gene comprises the nucleic acid sequence set forth in SEQ ID NO:210, or a fragment thereof. In another preferred embodiment, an Nrf2 gene comprises the nucleic acid sequence set forth in SEQ ID NO:212, or a fragment thereof. Such a method may be used to determine Nrf2 gene rearrangements or translocations. Without being bound by theory, Nrf2 gene rearrangement and translocation events appear to be important in the aetiology of lymphoma.

It is an object of this invention that the identification Nrf2 genes and recognition of their involvement in lymphoma provide diagnostic agents to distinguish between lymphoma subtypes, and analytical agents for further analysis of mechanisms involved in dysregulated growth and/or survival and/or apoptosis in cells of the hematopoietic system. An additional object of the invention is to provide appropriate and potentially novel targets for therapeutic interventions, particularly with regard to lymphoma, which are identified through the use of the diagnostic and analytical agents provided herein.

Without being bound by theory, it is recognized herein that the involvement of Nrf2 genes in the cellular dysregulation underlying lymphoma implicates genes having an Nrf2 DNA binding sequence in the cellular dysregulation underlying lymphoma. In a preferred embodiment, the Nrf2 DNA binding sequence is bound by an Nrf2 protein comprising the amino acid sequence set forth in SEQ ID NO:211 and at Genbank accession number AAA68291, or a fragment thereof. In another preferred embodiment, the Nrf2 DNA binding sequence is bound by an Nrf2 protein comprising the amino acid sequence set forth in SEQ ID NO:213 and at Genbank accession number NP_{—}006155, or a fragment thereof.

Novel sequences are also provided herein. Other aspects of the invention will become apparent to the skilled artisan by the following description of the invention.

### DETAILED DESCRIPTION OF THE INVENTION

The present invention is directed to a number of sequences associated with lymphoma. The use of oncogenic retroviruses, whose sequences insert into the genome of the host organism resulting in lymphoma, allows the identification of host sequences involved in lymphoma. These sequences may then be used in a number of different ways, including diagnosis, prognosis, screening for modulators (including both agonists and antagonists), antibody generation (for immunotherapy and imaging), etc.

Accordingly, the present invention provides nucleic acid and protein sequences that are associated with lymphoma, herein termed “lymphoma/leukemia associated” or “lymphoma/leukemia defining” or “LA” sequences.

In a preferred embodiment, the present invention sets forth LA nucleic acids referred to herein as Pik3r1 nucleic acids. In another preferred embodiment, the present invention sets forth LA proteins referred to herein as Pik3r1 proteins.

In addition, the present invention provides GNAS nucleic acid and protein sequences that are associated with lymphoma. Gnas protein sequences include those encoded by a GNAS nucleic acid. Known proteins encoded by GNAS include Gₛα, XLαₛ and NESP55.

In addition, the present invention provides HIPK1 nucleic acid and protein sequences that are associated with lymphoma.

In a preferred embodiment the LA sequence is JAKI.

In a preferred embodiment, the LA sequence is Neurogranin.

In a preferred embodiment, the present invention sets forth LA nucleic acids referred to herein as Nrf2 nucleic acids. In another preferred embodiment, the present invention sets forth LA proteins referred to herein as Nrf2 proteins.

“Association” in this context means that the nucleotide or protein sequences are either differentially expressed or altered in lymphoma as compared to normal lymphoid tissue. As outlined below, LA sequences include those that are up-regulated (i.e. expressed at a higher level) in lymphoma, as well as those that are down-regulated (i.e. expressed at a lower level), in lymphoma. LA sequences also include sequences which have been altered (i.e., truncated sequences or sequences with a point mutation) and show either the same expression profile or an altered profile. In a preferred embodiment, the LA sequences are from humans; however, as will be appreciated by those in the art, LA sequences from other organisms may be useful in animal models of disease and drug evaluation; thus, other LA sequences are provided, from vertebrates, including mammals, including rodents (rats, mice, hamsters, guinea pigs, etc.), primates, farm animals (including sheep, goats, pigs, cows, horses, etc). LA sequences from other organisms may be obtained using the techniques outlined below.

LA sequences can include both nucleic acid and amino acid sequences. In a preferred embodiment, the LA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an LA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the LA sequences are nucleic acids. As will be appreciated by those in the art and is more fully outlined below, LA sequences are useful in a variety of applications, including diagnostic applications, which will detect naturally occurring nucleic acids, as well as screening applications; for example, biochips comprising nucleic acid probes to the LA sequences can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995), non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium. Series 580, “Carbohydrate Modifications in Antisense Research”. Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand (“Watson”) also defines the sequence of the other strand (“Crick”); thus the sequences described herein also includes the complement of the sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An LA sequence can be initially identified by substantial nucleic acid and/or amino acid sequence homology to the LA sequences outlined herein. Such homology can be based upon the overall nucleic acid or amino acid sequence, and is generally determined as outlined below, using either homology programs or hybridization conditions.

The LA sequences of the invention were identified as described in the examples; basically, infection of mice with murine leukemia viruses (MuLV; including SL3-3, Akv and mutants thereof) resulted in lymphoma. The LA sequences outlined herein comprise the insertion sites for the virus. In general, the retrovirus can cause lymphoma in three basic ways: first of all, by inserting upstream of a normally silent host gene and activating it (e.g. promoter insertion); secondly, by truncating a host gene that leads to oncogenesis; or by enhancing the transcription of a neighboring gene. By neighboring gene is meant a gene within 100 kb to 500 kb or more, more preferably 50 kb to 100 kb, more preferably 1 kb to 50 kb, of the insertion site. For example, retrovirus enhancers, including SL3-3, are known to act on genes up to approximately 200 kilobases of the insertion site.

In a preferred embodiment, LA sequences are those that are up-regulated in lymphoma; that is, the expression of these genes is higher in lymphoma as compared to normal lymphoid tissue of the same differentiation stage. “Up-regulation”, as used herein means at least about 50%, more preferably at least about 100%, more preferably at least about 150%, more preferably, at least about 200%, with from 300 to at least 1000% being especially preferred.

In a preferred embodiment, LA sequences are those that are down-regulated in lymphoma; that is, the expression of these genes is lower in lymphoma as compared to normal lymphoid tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, LA sequences are those that are altered but show either the same expression profile or an altered profile as compared to normal lymphoid tissue of the same differentiation stage. “Altered LA sequences” as used herein refers to sequences which are truncated, contain insertions or contain point mutations.

In a preferred embodiment, Pik3r1 sequences are those that are altered but show either the same expression profile or an altered profile as compared to normal lymphoid tissue of the same differentiation stage. “Altered Pik3r1 sequences” as used herein refers to sequences which are truncated, contain insertions, deletions, fusions, or contain point mutations.

In one embodiment, the present invention provides an Pik3r1 gene comprising the nucleic acid sequence set forth in SEQ ID NO:178 and at Genbank Accession number U50413. In one embodiment, the present invention provides an Pik3r1 gene comprising the nucleic acid sequence set forth by nucleotides 575 to 2749 in SEQ ID NO:178 and at Genbank Accession number U50413.

In one embodiment, the present invention provides an Pik3r1 gene comprising the nucleic acid sequence set forth in SEQ ID NO:180 and at Genbank Accession number M61906. In one embodiment, the present invention provides an Pik3r1 gene comprising the nucleic acid sequence set forth by nucleotides 43 to 2217 in SEQ ID NO:180 and at Genbank Accession number M61906.

In one embodiment, the present invention provides a Pik3r1 gene comprising a nucleic acid sequence having at least about 90% identity to the nucleic acid sequence set forth in SEQ ID NO:178 and at Genbank Accession number U50413. In one embodiment, the present invention provides an Pik3r1 gene comprising a nucleic acid sequence having at least about 90% identity to the nucleic acid sequence set forth by nucleotides 575 to 2749 in SEQ ID NO:178 and at Genbank Accession number U50413.

In one embodiment, the present invention provides a Pik3r1 gene comprising a nucleic acid sequence having at least about 90% identity to the nucleic acid sequence set forth in SEQ ID NO:180 and at Genbank Accession number M61906. In one embodiment, the present invention provides an Pik3r1 gene comprising a nucleic acid sequence having at least about 90% identity to the nucleic acid sequence set forth by nucleotides 43 to 2217 in SEQ ID NO:180 and at Genbank Accession number M61906.

In one embodiment, the present invention provides an Pik3r1 gene comprising a nucleic acid that hybridizes under high stringency conditions to a nucleic acid comprising the nucleic acid sequence set forth in SEQ ID NO:178 and at Genbank Accession number U50413.

In one embodiment, the present invention provides an Pik3r1 gene comprising a nucleic acid that hybridizes under high stringency conditions to a nucleic acid comprising the nucleic acid sequence set forth in SEQ ID NO:180 and at Genbank Accession number M61906.

In one embodiment, the present invention provides an Pik3r1 gene encoding an SH2 domain-containing protein, comprising the nucleic acid sequence set forth by nucleotides 1568-1811, or 1571-1796, or 2444-2666, or 2444-2681 in SEQ ID NO:1 and at Genbank Accession number U50413. In one embodiment, the present invention provides an Pik3r1 gene encoding an SH2 domain-containing protein, comprising a nucleic acid which hybridizes under high stringency conditions to a nucleic acid comprising the nucleic acid sequence set forth by nucleotides 1568-1811, or 1571-1796, or 2444-2666, or 2444-2681 in SEQ ID NO:178 and at Genbank Accession number U50413. In one embodiment, the present invention provides an Pik3r1 gene encoding an SH2 domain-containing protein, comprising a nucleic acid sequence having at least about 90% identity to the nucleic acid sequence set forth by nucleotides 1568-1811, or 1571-1796, or 2444-2666, or 2444-2681 in SEQ ID NO:178 and at Genbank Accession number U50413.

In one embodiment, the present invention provides an Pik3r1 gene encoding an SH3 domain-containing protein, comprising the nucleic acid sequence set forth by nucleotides 4-75, or 7-77 in SEQ ID NO:178 and at Genbank accession number U50413. In one embodiment, the present invention provides an Pik3r1 gene encoding an SH3 domain-containing protein, comprising a nucleic acid which will hybridize under high stringency conditions to a nucleic acid comprising the nucleic acid sequence set forth by nucleotides 4-75, or 7-77 in SEQ ID NO:178 and at Genbank accession number U50413. In one embodiment, the present invention provides an Pik3r1 gene encoding an SH3 domain-containing protein, comprising a nucleic acid sequence having at least about 90% identity to the nucleic acid sequence set forth by nucleotides 4-75, or 7-77 in SEQ ID NO:178 and at Genbank accession number U50413.

In one embodiment, the present invention provides an Pik3r1 gene encoding a protein comprising a RhoGAP domain, comprising the nucleic acid sequence set forth by nucleotides 142-277, or 143-293 in SEQ ID NO:178 and at Genbank accession number U50413. In one embodiment, the present invention provides an Pik3r1 gene encoding a protein comprising a RhoGAP domain, comprising a nucleic acid which will hybridize under high stringency conditions to a nucleic acid comprising the nucleic acid sequence set forth by nucleotides 142-277, or 143-293 in SEQ ID NO:178 and at Genbank accession number U50413. In one embodiment, the present invention provides an Pik3r1 gene encoding a protein comprising a RhoGAP domain, comprising a nucleic acid sequence having at least about 90% identity to the nucleic acid sequence set forth by nucleotides 142-277, or 143-293 in SEQ ID NO:178 and at Genbank accession number U50413.

In one embodiment, the present invention provides an Pik3r1 gene encoding an SH2 domain-containing protein, comprising the nucleic acid sequence set forth by nucleotides 1037-1280, or 1913-2150, or 1040-1265, or 1913-3035 in SEQ ID NO:180 and at Genbank Accession number M61906. In one embodiment, the present invention provides an Pik3r1 gene encoding an SH2 domain-containing protein, comprising a nucleic acid which hybridizes under high stringency conditions to a nucleic acid comprising the nucleic acid sequence set forth by nucleotides 1037-1280, or 1913-2150, or 1040-1265, or 1913-3035 in SEQ ID NO:180 and at Genbank Accession number M61906. In one embodiment, the present invention provides an Pik3r1 gene encoding an SH2 domain-containing protein, comprising a nucleic acid sequence having at least about 90% identity to the nucleic acid sequence set forth by nucleotides 1037-1280, or 1913-2150, or 1040-1265, or 1913-3035 in SEQ ID NO:180 and at Genbank Accession number M61906.

In one embodiment, the present invention provides an Pik3r1 gene encoding ah SH3 domain-containing protein, comprising the nucleic acid sequence set forth by nucleotides 53-266 or 62-272 in SEQ ID NO:180 and at Genbank accession number M61906. In one embodiment, the present invention provides an Pik3r1 gene encoding an SH3 domain-containing protein, comprising a nucleic acid which will hybridize under high stringency conditions to a nucleic acid comprising the nucleic acid sequence set forth by nucleotides 53-266 or 62-272 in SEQ ID NO:180 and at Genbank accession number M61906. In one embodiment, the present invention provides an Pik3r1 gene encoding an SH3 domain-containing protein, comprising a nucleic acid sequence having at least about 90% identity to the nucleic acid sequence set forth by nucleotides 53-266 or 62-272 in SEQ ID NO:180 and at Genbank accession number M61906.

In one embodiment, the present invention provides an Pik3r1 gene encoding a protein comprising a RhoGAP domain, comprising the nucleic acid sequence set forth by nucleotides 428-929 or 428-872 in SEQ ID NO:180 and at Genbank accession number M61906. In one embodiment, the present invention provides an Pik3r1 gene encoding a protein comprising a RhoGAP domain, comprising a nucleic acid which will hybridize under high stringency conditions to a nucleic acid comprising the nucleic acid sequence set forth by nucleotides 428-929 or 428-872 in SEQ ID NO:180 and at Genbank accession number M61906. In one embodiment, the present invention provides an Pik3r1 gene encoding a protein comprising a RhoGAP domain, comprising a nucleic acid sequence having at least about 90% identity to the nucleic acid sequence set forth by nucleotides 428-929 or 428-872 in SEQ ID NO:180 and at Genbank accession number M61906.

In one embodiment, the present invention provides an Pik3r1 gene comprising a nucleic acid sequence that encodes an Pik3r1 protein comprising the amino acid sequence set forth in SEQ ID NO:179 and at Genbank Accession Number AAC52847.

In one embodiment, the present invention provides an Pik3r1 gene comprising a nucleic acid sequence that encodes an Pik3r1 protein comprising the amino acid sequence set forth in SEQ ID NO:181 and at Genbank Accession Number A38748.

In one embodiment, the present invention provides an Pik3r1 gene encoding an SH2 domain-containing Pik3r1 protein comprising the amino acid sequence set forth by amino acids 332-413, or 333-408, or 624-703, or 624-698, in SEQ ID NO:179 and at Genbank Accession Number AAC52847.

In one embodiment, the present invention provides an Pik3r1 gene encoding an SH2 domain-containing Pik3r1 protein comprising the amino acid sequence set forth by amino acids 332-413, or 333-408, or 624-703, or 624-698, in SEQ ID NO:181 and at Genbank Accession Number A38748.

In one embodiment, the present invention provides an Pik3r1 gene encoding an SH3 domain-containing Pik3r1 protein comprising the amino acid sequence set forth by amino acids 4-75 or 7-77 in SEQ ID NO:179 and at Genbank accession number AAC52847.

In one embodiment, the present invention provides an Pik3r1 gene encoding an SH3 domain-containing Pik3r1 protein comprising the amino acid sequence set forth by amino acids 4-75 or 7-77 in SEQ ID NO:181 and at Genbank accession number A38748.

In one embodiment, the present invention provides an Pik3r1 gene encoding RhoGAP domain-containing Pik3r1 protein comprising the amino acid sequence set forth by amino acids 142-277 or 143-293 in SEQ ID NO:179 and at Genbank accession number AAC52847.

In one embodiment, the present invention provides an Pik3r1 gene encoding RhoGAP domain-containing Pik3r1 protein comprising the amino acid sequence set forth by amino acids 129-296 or 129-277 in SEQ ID NO:179 and at Genbank accession number M61906.

In one embodiment, the present invention provides Pik3r1 proteins encoded by Pik3r1 nucleic acids as described herein.

In a preferred embodiment, the present invention sets forth LA nucleic acids referred to herein as Nrf2 nucleic acids. In another preferred embodiment, the present invention sets forth LA proteins referred to herein as Nrf2 proteins.

In one embodiment, the present invention provides an Nrf2 gene comprising the nucleic acid sequence set forth in SEQ ID NO:210 and at Genbank Accession number U20532. In one embodiment, the present invention provides an Nrf2 gene comprising the nucleic acid sequence set forth by nucleotides 298 to 2043 in SEQ ID NO:210 and at Genbank Accession number U20532.

In one embodiment, the present invention provides an Nrf2 gene comprising the nucleic acid sequence set forth in SEQ ID NO:212 and at Genbank Accession number NM_{—}006164. In one embodiment, the present invention provides an Nrf2 gene comprising the nucleic acid sequence set forth by nucleotides 40 to 1809 in SEQ ID NO:212 and at Genbank Accession number NM_{—}006164.

In one embodiment, the present invention provides a Nrf2 gene comprising a nucleic acid sequence having at least about 90% identity to the nucleic acid sequence set forth in SEQ ID NO:210 and at Genbank Accession number U20532. In one embodiment, the present invention provides an Nrf2 gene comprising a nucleic acid sequence having at least about 90% identity to the nucleic acid sequence set forth by nucleotides 298 to 2043 in SEQ ID NO:210 and at Genbank Accession number U20532.

In one embodiment, the present invention provides a Nrf2 gene comprising a nucleic acid sequence having at least about 90% identity to the nucleic acid sequence set forth in SEQ ID NO:212 and at Genbank Accession number NM_{—}006164. In one embodiment, the present invention provides an Nrf2 gene comprising a nucleic acid sequence having at least about 90% identity to the nucleic acid sequence set forth by nucleotides 40 to 1809 in SEQ ID NO:212 and at Genbank Accession number NM_{—}006164.

In one embodiment, the present invention provides an Nrf2 gene comprising a nucleic acid that hybridizes under high stringency conditions to a nucleic acid comprising the nucleic acid sequence set forth in SEQ ID NO:210 and at Genbank Accession number U20532.

In one embodiment, the present invention provides an Nrf2 gene comprising a nucleic acid that hybridizes under high stringency conditions to a nucleic acid comprising the nucleic acid sequence set forth in SEQ ID NO:212 and at Genbank Accession number NM_{—}006164.

In one embodiment, the present invention provides an Nrf2 gene comprising the nucleic acid sequence set forth by nucleotides 1716 to 1850 in SEQ ID NO:210 and at Genbank Accession number U20532. In one embodiment, the present invention provides an Nrf2 gene comprising a nucleic acid which hybridizes under high stringency conditions to a nucleic acid comprising the nucleic acid sequence set forth by nucleotides 1716 to 1850 in SEQ ID NO:210 and at Genbank Accession number U20532. In one embodiment, the present invention provides an Nrf2 gene comprising a nucleic acid sequence having at least about 90% identity to the nucleic acid sequence set forth by nucleotides 1716 to 1850 in SEQ ID NO:210 and at Genbank Accession number U20532.

In one embodiment, the present invention provides an Nrf2 gene comprising the nucleic acid sequence set forth by nucleotides 1482 to 1616, more preferably 1482 to 1550, in SEQ ID NO:212 and at Genbank Accession number NM_{—}006164. In one embodiment, the present invention provides an Nrf2 gene comprising a nucleic acid which hybridizes under high stringency conditions to a nucleic acid comprising the nucleic acid sequence set forth by nucleotides 1482 to 1616, more preferably 1482 to 1550, in SEQ ID NO:212 and at Genbank Accession number NM_{—}006164. In one embodiment, the present invention provides an Nrf2 gene comprising a nucleic-acid sequence having at least about 90% identity to the nucleic acid sequence set forth by nucleotides 1482 to 1616, more preferably 1482 to 1550, in SEQ ID NO:212 and at Genbank Accession number NM_{—}006164.

In one embodiment, the present invention provides an Nrf2 gene comprising a nucleic acid sequence that encodes an Nrf2 protein comprising the amino acid sequence set forth in SEQ ID NO:211 and at Genbank Accession Number AAA68291.

In one embodiment, the present invention provides an Nrf2 gene comprising a nucleic acid sequence that encodes an Nrf2 protein comprising the amino acid sequence set forth in SEQ ID NO:213 and at Genbank Accession Number NP_{—}006155.

In one embodiment, the present invention provides an Nrf2 gene comprising a nucleic acid sequence encoding an Nrf2 protein comprising the amino acid sequence set forth by amino acids 474 to 518 in SEQ ID NO:211 and at Genbank Accession Number AAA68291.

In one embodiment, the present invention provides an Nrf2 gene comprising a nucleic acid sequence encoding an Nrf2 protein comprising the amino acid sequence set forth by amino acids 482 to 526, more preferably 482 to 504, in SEQ ID NO:213 and at Genbank Accession Number NP_{—}006155.

In one embodiment, the present invention provides an Nrf2 gene comprising a nucleic acid sequence encoding an Nrf2 protein comprising the amino acid sequence set forth in SEQ ID NO:211 and at Genbank Accession Number AAA68291, except for lacking a fragment of the amino acid sequence set forth by amino acids 474 to 518 in SEQ ID NO:211 and at Genbank Accession Number AAA68291.

In one embodiment, the present invention provides an Nrf2 gene comprising a nucleic acid sequence encoding an Nrf2 protein comprising the amino acid sequence set forth in SEQ ID NO:213 and at Genbank Accession Number NP_{—}006155, except for lacking a fragment of the amino acid sequence set forth by amino acids 482 to 526, more preferably 482 to 504, in SEQ ID NO:213 and at Genbank Accession Number NP_{—}006155.

In one embodiment, the present invention provides Nrf2 proteins encoded by Nrf2 nucleic adds as described herein.

LA proteins of the present invention may be classified as secreted proteins, transmembrane proteins or intracellular proteins.

In a preferred embodiment the LA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

In its native form, Pik3r1 protein is an intracellular protein comprising SH2, Sh3, and RhoGAP domains. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, phosphatidyl inositol-conjugated lipid kinase activity, protein phosphatase activity, phosphatidyl inositol-conjugated lipid phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

Common protein motifs have also been identified among transcription factors and have been used to divide these factors into families. These motifs include the basic helix-loop-helix, basic leucine zipper, zinc finger and homeodomain motifs.

HIPK1 is known to contain several conserved domains, including a homeoprotein interaction domain, a protein kinase domain, a PEST domain, and a YH domain enriched in tyrosine and histidine residues (Kim et al., J. Biol. Chem. 273:25875 (1998). In the mouse HIPK1 amino acid sequence depicted in Table 16 as SEQ ID NO. 197, the homeoprotein interaction domain is from about amino 15, acid 190 to about amino acid 518, the protein kinase domain is from about amino acid 581 to about amino acid 848, the PEST domain is from about amino acid 890 to about amino acid 974, and the YH domain is from about amino acid 1067 to about amino acid 1210.

In a preferred embodiment, the LA sequences are transmembrane proteins or can be made to be transmembrane proteins through the use of recombinant DNA technology. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor, etc.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been-ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate is receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

LA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

It is further recognized that Nrf2 proteins can be made to be secreted proteins though recombinant methods. Secretion can be either constitutive or regulated. Secreted proteins have a signal peptide or signal sequence that targets the molecule to the secretory pathway.

In another preferred embodiment, the Nrf2 proteins are nuclear proteins, preferably transcription factors. Transcription factors are involved in numerous physiological events and act by regulating gene expression at the transcriptional level. Transcription factors often serve as nodal points of regulation controlling multiple genes. They are capable of effecting a multifarious change in gene expression and can integrate many convergent signals to effect such a change. Transcription factors are often regarded as “master regulators” of a particular cellular state or event. Accordingly, transcription factors have often been found to faithfully mark a particular cell state, a quality which makes them attractive for use as diagnostic markers. In addition, because of their important role as coordinators of patterns of gene expression associated with particular cell states, transcription factors are attractive therapeutic targets. Intervention at the level of transcriptional regulation allows one to effectively target multiple genes associated with a dysfunction which fall under the regulation of a “master regulator” or transcription factor.

In a preferred embodiment, the LA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. LA proteins that are secreted proteins are particularly preferred in the present invention as they serve as good targets for diagnostic markers, for example for blood tests.

An LA sequence is initially identified by substantial nucleic acid and/or amino acid sequence homology to the LA sequences outlined herein. Such homology can be based upon the overall nucleic acid or amino acid sequence, and is generally determined as outlined below, using either homology programs or hybridization conditions.

In one embodiment, an Pik3r1 sequence can be identified by substantial nucleic acid sequence identity or homology to the Pik3r1 nucleic acid sequence set forth in SEQ ID NO:178 and at Genbank Accession number U50413.

In another embodiment, an Pik3r1 sequence can be identified by substantial nucleic acid sequence identity or homolgy to the Pik3r1 nucleic acid sequence set forth in SEQ ID NO:180 and at Genbank Accession number M61906.

In one embodiment, an Pik3r1 sequence can be identified by substantial amino acid sequence identity or homology to the Pik3r1 amino acid sequence set forth in SEQ ID NO:17.9 and at Genbank Accession number AAC52847.

In another embodiment, an Pik3r1 sequence can be identified by substantial amino acid sequence identity or homology to the Pik3r1 amino acid sequence set forth in SEQ ID NO:181 and at Genbank Accession number A38478.

In one embodiment, an Nrf2 sequence can be identified by substantial nucleic acid sequence identity or homology to the Nrf2 nucleic acid sequence set forth in SEQ ID NO:210 and at Genbank Accession number U20532.

In another embodiment, an Nrf2 sequence can be identified by substantial nucleic acid sequence identity or homolgy to the Nrf2 nucleic acid sequence set forth in SEQ ID NO:210 and at Genbank Accession number NM_{—}006164.

It one embodiment, an Nrf2 sequence can be identified by substantial amino acid sequence identity or homology to the Nrf2 amino acid sequence set forth in SEQ ID NO:211 and at Genbank Accession number AAA68291.

In another embodiment, an Nrf2 sequence can be identified by substantial amino acid sequence identity or homology to the Nrf2 amino acid sequence set forth in SEQ ID NO:213 and at Genbank Accession number NP_{—}006155.

As used herein, a nucleic acid is a “LA nucleic acid” if the overall homology of the nucleic acid sequence to one of the nucleic acids of Tables 1, 2, 4, 6, 8, 9, 10, 11, 12, 13, 14, 15, 16, 17, 18, 19, 22, 23, 24, 27, 28 or 30 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences which are used to determine sequence identity or similarity are selected from those of the nucleic acids of Tables 1, 2, 4, 6, 8, 9, 10, 11, 12, 13, 14, 15, 16, 17, 18, 19, 22, 23, 24, 27, 28 or 30. In another embodiment, the sequences are naturally occurring allelic variants of the sequences of the nucleic acids of Table 1, 2, 3, 4, 6, 8, 9, 10, 11, 12, 13, 14, 15, 16, 17, 18, 19, 22, 23, 24, 27, 28 or 30. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Aitschul et al., Methods in Enzymology, 266:460A-480 (1996); http://blast.wustl]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic-acids of the SEQ ID NOS. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of the SEQ ID NOS, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the figures, or their complements, are considered LA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, the LA nucleic acid sequences of the invention are fragments of larger genes, i.e. they are nucleic acid segments. Alternatively, the LA nucleic acid sequences can serve as indicators of oncogene position, for example, the LA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the LA genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the LA nucleic acid is identified, it can be cloned and, if necessary, its constituent parts recombined to form the entire LA nucleic acid. Once isolated from its natural source, e.g., contained within a plasmid or other vector or excised therefrom as a linear nucleic acid segment, the recombinant LA nucleic acid can be further used as a probe to identify and isolate other LA nucleic acids, for example additional coding regions. It can also be used as a “precursor” nucleic acid to make modified or variant LA nucleic acids and proteins.

The LA nucleic acids of the present invention are used in several ways. In a first embodiment, nucleic acid probes to the LA nucleic acids are made and attached to biochips to be used in screening and diagnostic methods, as outlined below, or for administration, for example for gene therapy and/or antisense applications. Alternatively, the LA nucleic acids that include coding regions of LA proteins can be put into expression vectors for the expression of LA proteins, again either for screening purposes or for administration to a patient.

In a preferred embodiment, nucleic acid probes to LA nucleic acids (both the nucleic acid sequences outlined in the figures and/or the complements thereof) are made. The nucleic acid probes attached to the biochip are designed to be substantially complementary to the LA nucleic acids, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two Moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both Molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5 or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

In a preferred embodiment, LA nucleic acids encoding LA proteins are used to make a variety of expression vectors to express LA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the LA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient-restriction sites. If such sites do not exist, synthetic oligonucleotide adaptors or linkers are used in accordance with conventional practice. The transcriptional and translational regulatory nucleic acid will generally be appropriate to the host cell used to express the LA protein; for example, transcriptional and translational regulatory nucleic acid sequences from Bacillus are preferably used to express the LA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The LA proteins of the present invention are produced by culturing a host cell transformed with an expression vector containing nucleic acid encoding an LA protein, under the appropriate conditions to induce or cause expression of the LA protein. The conditions appropriate for LA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the LA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, LA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the LA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, LA proteins are produced in insect cells. Expression vectors for the transformation of insect cells, and in particular, baculovirus-based expression vectors, are well known in the art.

In a preferred embodiment, LA protein is produced in yeast cells. Yeast expression systems, are well known in the art, and include expression vectors for Saccharomyces cerevisiae, Candida albicans and C. maltosa, Hansenula polytmorpha, Kluyveromyces fragilis and K. lactis, Pichia guillerimondii and P. pastoris, Schizosaccharomyces pombe, and Yarrowia lipolytica.

The LA protein may also be made as a fusion protein, using techniques well known in the art. Thus, for example, for the creation of monoclonal antibodies. If the desired epitope is small, the LA protein may be fused to a carrier protein to form an immunogen. Alternatively, the LA protein may be made as a fusion protein to increase expression, or for other reasons. For example, when the LA protein is an LA peptide, the nucleic acid encoding the peptide may be linked to other nucleic acid for expression purposes.

In one embodiment, the LA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the LA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label May be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides LA protein sequences. An LA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the LA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as Sequence in FASTA format. The organism list is “none”. The “expect” is 10; the filter is default. The “descriptions” is 500, the “alignments” is 500, and the “alignment view” is pairwise. The “Query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of LA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

LA proteins of the present invention may be shorter or longer than the wild type amino acid sequences. Thus, in a preferred embodiment, included within the definition of LA proteins are portions or fragments of the wild type sequences herein. In addition, as outlined above, the LA nucleic acids of is the invention may be used to obtain additional coding regions, and thus additional protein sequence, using techniques known in the art.

In a preferred embodiment, the LA proteins are derivative or variant LA proteins as compared to the wild-type sequence. That is, as outlined more fully below, the derivative LA peptide will contain at least one amino acid substitution, deletion or insertion, with amino acid substitutions being particularly preferred. The amino acid substitution, insertion or deletion may occur at any residue within the LA peptide.

Also included in an embodiment of LA proteins of the present invention are amino acid sequence variants. These variants fall into one or more of three classes: substitutional, insertional or deletional variants. These variants ordinarily are prepared by site specific mutagenesis of nucleotides in the DNA encoding the LA protein, using cassette or PCR mutagenesis or other techniques well known in the art, to produce DNA encoding the variant, and thereafter expressing the DNA in recombinant cell culture as outlined above. However, variant LA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the LA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed LA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of LA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the LA protein are desired, substitutions are generally made in accordance with the following chart:

CHART I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one hot having a side chain, e.g. glycine.

The variants typically exhibit the same qualitative biological activity and will elicit the same immune response as the naturally-occurring analogue, although variants also are selected to modify the characteristics of the LA proteins as needed. Alternatively, the variant may be designed such that the biological activity of the LA protein is altered. For example, glycosylation sites may be altered or removed, dominant negative mutations created, etc.

Covalent modifications of LA polypeptides are included within the scope of this invention, for example for use in screening. One type of covalent modification includes reacting targeted amino acid residues of an LA polypeptide with an organic derivatizing agent that is capable of reacting with selected side chains or the N- or C-terminal residues of an LA polypeptide. Derivatization with bifunctional agents is useful, for instance, for crosslinking LA to a water-insoluble support matrix or surface for use in the method for purifying anti-LA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the LA polypeptide included within the scope of this invention comprises altering the native glycosylation pattern of the polypeptide. “Altering the native glycosylation pattern” is intended for purposes herein to mean deleting one or more carbohydrate moieties found in native sequence LA polypeptide, and/or adding one or more glycosylation sites that are not present in the native sequence LA polypeptide.

Addition of glycosylation sites to LA polypeptides may be accomplished by altering the amino acid sequence thereof. The alteration may be made, for example, by the addition of, or substitution by; one or more serine or threonine residues to the native sequence LA polypeptide (for O-linked glycosylation sites). The LA amino acid sequence may optionally be altered through changes at the DNA level, particularly by mutating the DNA encoding the LA polypeptide at preselected bases such that codons are generated that will translate into the desired amino acids.

Another means of increasing the number of carbohydrate moieties on the LA polypeptide is by chemical or enzymatic coupling of glycosides to the polypeptide. Such methods are described in the art, e.g., in WO 87/05330 published 11 Sep. 1987, and in Aplin and Wriston, L A Crit. Rev. Biochem., pp. 259-306 (1981).

Removal of carbohydrate moieties present on the LA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of LA comprises linking the LA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

LA polypeptides of the present invention may also be modified in a way to form chimeric molecules comprising an LA polypeptide fused to another, heterologous polypeptide or amino acid sequence. In one embodiment, such a chimeric molecule comprises a fusion of an LA polypeptide with a tag polypeptide which provides an epitope to which an anti-tag antibody can selectively bind. The epitope tag is generally placed at the amino- or carboxyl-terminus of the LA polypeptide, although internal fusions may also be tolerated in some instances. The presence of such epitope-tagged forms of an LA polypeptide can be detected using an antibody against the tag polypeptide. Also, provision of the epitope tag enables the LA polypeptide to be readily purified by affinity purification using an anti-tag antibody or another type of affinity matrix that binds to the epitope tag. In an alternative embodiment, the chimeric molecule may comprise a fusion of an LA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of LA protein in one embodiment are other LA proteins of the LA family, and LA proteins from other organisms, which are cloned and expressed as outlined below. Thus, probe or degenerate polymerase chain reaction (PCR) primer sequences may be used to find other related LA proteins from humans or other organisms. As will be appreciated by those in the art, particularly useful probe and/or PCR primer sequences include the unique areas of the LA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, LA proteins can be made that are longer than those encoded by the nucleic acids of the figures, for example, by the elucidation of additional sequences, the addition of epitope or purification tags, the addition of other fusion sequences, etc.

LA proteins may also be identified as being encoded by LA nucleic acids. Thus, LA proteins are encoded by nucleic acids that will hybridize to the sequences of the sequence listings, or their complements, as outlined herein.

In one embodiment, the present invention provides an LA protein referred to herein as Pik3r1 which comprises the amino acid sequence set forth in SEQ ID NO:179 and at Genbank accession number AAC52847, and which is encoded by the nucleic acid sequence set forth by nucleotides 575-2749 in SEQ ID NO:178 and at Genbank accession number U50413.

In one embodiment, the present invention provides an LA protein referred to herein as Pik3r1 which comprises the amino acid sequence set forth in SEQ ID NO:181 and at Genbank accession number A38748. In one embodiment, the present invention provides an LA protein referred to herein as Pik3r1 which is encoded by the nucleic acid sequence set forth by nucleotides 43-2217 in SEQ ID NO:180 and at Genbank accession number M61906.

In one embodiment, the present invention provides an Pik3r1 protein encoded by a nucleic acid which hybridizes under high stringency conditions to a nucleic acid comprising the nucleic acid sequence set forth in SEQ ID NO:178 and at Genbank accession number U50413.

In one embodiment, the present invention provides an Pik3r1 protein encoded by a nucleic acid which hybridizes under high stringency conditions to a nucleic acid comprising the nucleic acid sequence set forth in SEQ ID NO:180 and at Genbank accession number M61906.

In one embodiment, the present invention provides an Pik3r1 protein encoded by a nucleic acid which comprises a nucleic acid sequence having at least about 90% identity to the nucleic acid sequence set forth in SEQ ID NO:178 and at Genbank accession number U50413.

In one embodiment, the present invention provides an Pik3r1 protein encoded by a nucleic acid which comprises a nucleic acid sequence having at least about 90% identity to the nucleic acid sequence set forth in SEQ ID NO:180 and at Genbank accession number M61906.

In one embodiment, the present invention provides an Pik3r1 protein encoded by a nucleic acid which comprises a nucleic acid sequence having at least about 90% identity to the nucleic acid sequence set forth by nucleotides 575-2749 in SEQ ID NO:178 and at Genbank accession number U50413.

In one embodiment, the present invention provides an Pik3r1 protein encoded by a nucleic acid which comprises a nucleic acid sequence having at least about 90% identity to the nucleic acid sequence set forth by nucleotides 43-2217 in SEQ ID NO:180 and at Genbank accession number M61906.

In one embodiment, the present invention provides an Pik3r1 protein comprising an SH2 domain encoded by the nucleic acid sequence set forth by nucleotides 1568-1811, or 1571-1796, or 2444-2681, or 2444-2666 in SEQ ID NO:178 and at Genbank Accession Number U50413.

In one embodiment, the present invention provides an Pik3r1 protein comprising an SH2 domain encoded by the nucleic acid sequence set forth by nucleotides 1037-1280, or 1040-1265, or 1913-2150, or 1913-3035 in SEQ ID NO:180 and at Genbank Accession Number M61906.

In one embodiment, the present invention provides an Pik3r1 protein comprising an SH3 domain encoded by the nucleic acid sequence set forth by nucleotides 584-797 or 593-803 in SEQ ID NO:178 and at Genbank Accession Number U50413.

In one embodiment, the present invention provides an Pik3r1 protein comprising an SH3 domain encoded by the nucleic acid sequence set forth by nucleotides 53-266 or 62-272 in SEQ ID NO:180 and at Genbank Accession Number M61906.

In one embodiment, the present invention provides an Pik3r1 protein comprising a RhoGAP domain encoded by the nucleic acid sequence set forth by nucleotides 998-1403 or 1001-1451 in SEQ ID NO:178 and at Genbank Accession Number U50413.

In one embodiment, the present invention provides an Pik3r1 protein comprising a RhoGAP domain encoded by the nucleic acid sequence set forth by nucleotides 428-929 or 428-872 in SEQ ID NO:180 and at Genbank Accession Number M61906.

In one embodiment, the present invention provides an Pik3r1 protein comprising the amino acid sequence set forth in SEQ ID NO:179 and at Genbank Accession number AAC52847.

In one embodiment, the present invention provides an Pik3r1 protein comprising the amino acid sequence set forth in SEQ ID NO:181 and at Genbank Accession number A38748.

In one embodiment, the present invention provides an Pik3r1 protein comprising an amino acid sequence having at least about 90% identity to the amino acid sequence set forth in SEQ ID NO:179 and at Genbank Accession Number AAC52847.

In one embodiment, the present invention provides an Pik3r1 protein comprising an amino acid sequence having at least about 90% identity to the amino acid sequence set forth in SEQ ID NO:181 and at Genbank Accession Number A38748.

In one embodiment, the present invention provides an Pik3r1 protein comprising an SH2 domain comprising the amino acid sequence set forth by amino acids 332-413, or 333-408, or 624-703, or 624-698 in SEQ ID NO:179 and at Genbank Accession Number AAC52847.

In one embodiment, the present invention provides an Pik3r1 protein comprising an SH2 domain comprising the amino acid sequence set forth by amino acids 332-413, or 333-408, or 624-703, or 624-698 in SEQ ID NO:181 and at Genbank Accession Number A38748.

In one embodiment, the present invention provides an Pik3r1 protein comprising an SH3 domain comprising the amino acid sequence set forth by amino acids 4-75 or 7-77 in SEQ ID NO:179 and at Genbank Accession Number AAC52847.

In one embodiment, the present invention provides an Pik3r1 protein comprising an SH3 domain comprising the amino acid sequence set forth by amino acids 4-75 or 7-77 in SEQ ID NO:181 and at Genbank Accession Number A38748.

In one embodiment, the present invention provides an Pik3r1 protein comprising a RhoGAP domain comprising the amino acid sequence set forth by amino acids 142-277 or 143-293 in SEQ ID NO:179 and at Genbank Accession Number AAC52847.

In one embodiment, the present invention provides an Pik3r1 protein comprising a RhoGAP domain comprising the amino acid sequence set forth by amino acids 129-296 or 129-277 in SEQ ID NO:181 and at Genbank Accession Number A38748.

In a preferred embodiment, a Pik3r1 protein is a subunit of a PI3K enzyme. In a preferred embodiment, such a subunit modulates the activity of a PI3K catalytic subunit, preferably p110 as described herein. In a preferred embodiment, a Pik3r1 protein binds to phosphorylated tyrosine residues in receptor tyrosine kinases, as in the erythropoietin receptor, preferably by an SH2 domain, and tethers a PI3K catalytic subunit to the receptor. In a preferred embodiment, a Pik3r1 protein additionally binds to intracellular proteins involved in signal transduction through an SH3 domain.

In a preferred embodiment, a Pik3r1 protein modulates the production of phosphorylated phosphatidyl inositol lipids. In a preferred embodiment, such modulation in turn modulates the activity of serine/threonine protein kinases, preferably PKB or PKC. In a preferred embodiment, a Pik3r1 protein modulates the phosphorylation of proteins mediating cell death and/or survival.

In a preferred embodiment, the invention provides LA antibodies. In a preferred embodiment, when the LA protein is to be used to generate antibodies, for example for immunotherapy, the LA protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller LA protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can is be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1, 2, and 3 or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-1031. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by a nucleic acid of the Tables 1, 2, 4, 6, 8, 9, 10, 11, 12, 13, 14, 15, 16, 17, 18, 19, 22, 23, 24, 27, 28 or 30 or a fragment thereof, the other one is for any other antigen, and preferably for a cell-surface protein or receptor or receptor subunit, preferably one that is tumor specific.

In a preferred embodiment, the antibodies to LA are capable of reducing or eliminating the biological function of LA, as is described below. That is, the addition of anti-LA antibodies (either polyclonal or preferably monoclonal) to LA (or cells containing LA) may reduce or eliminate the LA activity. Generally, at least a 25% decrease in activity is preferred, with at least about 50% being particularly preferred and about a 95-100% decrease being especially preferred.

In a preferred embodiment the antibodies to the LA proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature. 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies [Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of lymphoma with an antibody raised against an LA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against LA proteins that are secreted proteins as described above. Without being bound by theory, antibodies used for treatment, bind and prevent the secreted protein from binding to its receptor, thereby inactivating the secreted LA protein.

In a preferred embodiment, subunits of kinase holoenzymes, which holoenzymes phosphorylate substrates, preferably lipid substrates, preferably phosphatidyl inositol-conjugated lipid substrates, are inhibited by antibodies raised against Pik3r1 proteins or portions thereof. In a preferred embodiment, such anti Pik3r1 antibodies modulate the activity of PI3 kinase. It is recognized herein that other means of holoenzyme inhibition, preferably PI3 kinase inhibition, are known to exist and include fungal toxins, preferably wortmannin, and synthetic inhibitors, preferably LY294002.

In one embodiment, an anti-Pik3r1 antibody binds to an SH3 domain of a Pik3r1 protein. In a preferred embodiment, such an SH3 domain comprises the amino acid sequence set forth by amino acids 4-75 or 7-77 in SEQ ID NO:179 and at Genbank accession number AAC52847. In another preferred embodiment, such an SH3 domain comprises the amino acid sequence set forth by amino acids 4-75 or 7-77 in SEQ ID NO:181 and at Genbank accession number A38748. In another preferred embodiment, such an SH3 domain comprises an amino acid sequence having at least about 90% identity to the amino acid sequence set forth by amino acids 4-75 or 7-77 in SEQ ID NO:179 and at Genbank accession number AAC52847. In another preferred embodiment, such an SH3 domain comprises an amino acid sequence having at least about 90% identity to the amino acid sequence set forth by amino acids 4-75 or 7-77 in SEQ ID NO:181 and at Genbank accession number A38748.

In a preferred embodiment, an antibody recognizing an SH3 domain in a Pik3r1 protein alters the activity of Pik3r1. In a preferred embodiment, such an alteration in activity is a decrease in activity. In a preferred embodiment, such an alteration in activity alters PI3K activity. In a preferred embodiment, such an alteration in activity decreases PI3K activity.

In a preferred embodiment, an antibody recognizing an SH3 domain in a Pik3r1 protein inhibits the ability of Pik3r1 to bind to a proline rich amino acid sequence, preferably in the context of the amino acid sequence of an intracellular protein, preferably an intracellular protein involved in intracellular signal transduction.

In one embodiment, an anti-Pik3r1 antibody binds to an SH2 domain of a Pik3r1 protein. In a preferred embodiment, such an SH2 domain comprises the amino acid sequence set forth by amino acids 332-413, or 333-408, or 624-703, or 624-698 in SEQ ID NO:179 and at Genbank accession number AAC52847. In another preferred embodiment, such an SH2 domain comprises the amino acid sequence set forth by amino acids 332-413, or 333-408, or 624-703, or 624-698 in SEQ ID NO:181 and at Genbank accession number A38748. In another preferred embodiment, such an SH2 domain comprises an amino acid sequence having at least about 90% identity to the amino acid sequence set forth by amino acids 332-413, or 333-408, or 624-703, or 624-698 in SEQ ID NO:179 and at Genbank accession number AAC52847. In another preferred embodiment, such an SH2 domain comprises an amino acid sequence having at least about 90% identity to the amino acid sequence set forth by amino acids 332-413, or 333-408, or 624-703, or 624-698 in SEQ ID NO:181 and at Genbank accession number A38748.

In a preferred embodiment, an antibody recognizing an SH2 domain in a Pik3r1 protein alters the activity of Pik3r1. In a preferred embodiment, such an alteration in activity is a decrease in activity. In a preferred embodiment, such an alteration in activity leads to a decrease in PI3K activity.

In a preferred embodiment, an antibody recognizing an SH2 domain in a Pik3r1 protein inhibits the ability of Pik3r1 to bind to phosphorylated tyrosine, preferably in the context of the amino acid sequence of a receptor tyrosine kinase.

In one embodiment, an anti-Pik3r1 antibody binds to a RhoGAP domain of a Pik3r1 protein. In a preferred embodiment, such a RhoGAP domain comprises the amino acid sequence set forth by amino acids 142-277 or 143-293 in SEQ ID NO:179 and at Genbank accession number MC52847. In another preferred embodiment, such a RhoGAP domain comprises the amino acid sequence set forth by amino acids 129-296 or 129-277 in SEQ ID NO:181 and at Genbank accession number A38748. In another preferred embodiment, such a RhoGAP domain comprises an amino acid sequence having at least about 90% identity to the amino acid sequence set forth by amino acids 142-277 or 143-293 in SEQ ID NO:179 and at Genbank accession number AAC52847. In another preferred embodiment, such a RhoGAP domain comprises an amino acid sequence having at least about 90% identity to the amino acid sequence set forth by amino acids 129-296 or 129-277 in SEQ ID NO:181 and at Genbank accession number A38748.

In a preferred embodiment, an antibody recognizing a RhoGAP domain in a Pik3r1 protein alters the activity of Pik3r1. In a preferred embodiment, such an alteration in activity is a decrease in activity. In a preferred embodiment, such an alteration in activity leads to a decrease in PI3K activity.

In another preferred embodiment, the LA protein to which antibodies are raised is a transmembrane protein. Without being bound by theory, antibodies used for treatment, bind the extracellular domain of the LA protein and prevent it from binding to other proteins, such as circulating ligands or cell-associated molecules. The antibody may cause down-regulation of the transmembrane LA protein. As will be appreciated by one of ordinary skill in the art, the antibody may be a competitive, non-competitive or uncompetitive inhibitor of protein binding to the extracellular domain of the LA protein. The antibody is also an antagonist of the LA protein. Further, the antibody prevents activation of the transmembrane LA protein. In one aspect, when the antibody prevents the binding of other molecules to the LA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin-D, cisplatin, methotrexate, and the like. In some instances the antibody belongs to a sub-type that activates serum complement when complexed with the transmembrane protein thereby mediating cytotoxicity. Thus, lymphoma may be treated by administering to a patient antibodies directed against the transmembrane LA protein.

In another preferred embodiment, the antibody is conjugated to a therapeutic moiety. In one aspect the therapeutic moiety is a small molecule that modulates the activity of the LA protein. In another aspect the therapeutic moiety modulates the activity of molecules associated with or in dose proximity to the LA protein. The therapeutic moiety may inhibit enzymatic activity such as protease or protein kinase activity associated with lymphoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against LA proteins, or binding of a radionuclide to a chelating agent that has been covalently attached to the antibody. Targeting the therapeutic moiety to transmembrane LA proteins not only serves to increase the local concentration of therapeutic moiety in the lymphoma, but also serves to reduce deleterious side effects that may be associated with the therapeutic moiety.

In another preferred embodiment, the LA protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the LA protein can be targeted within a cell, i.e., the nucleus, an antibody thereto contains a signal for that target localization, i.e., a nuclear localization signal.

The LA antibodies of the invention specifically bind to LA proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In a preferred embodiment, the LA protein is purified or isolated after expression. LA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the LA protein may be purified using a standard anti-LA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the LA protein. In some instances no purification will be necessary.

Once expressed and purified if necessary, the LA proteins and nucleic acids are useful in a number of applications.

In one aspect, the expression levels of genes are determined for different cellular states in the lymphoma phenotype; that is, the expression levels of genes in normal tissue and in lymphoma tissue (and in some cases, for varying severities of lymphoma that relate to prognosis, as outlined below) are evaluated to provide expression profiles. An expression profile of a particular cell state or point of development is essentially a “fingerprint” of the state; while two states May have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or lymphoma tissue.

“Differential expression,” or grammatical equivalents as used herein, refers to both qualitative as well as quantitative differences in the genes' temporal and/or cellular expression patterns within and 15′ among the cells. Thus, a differentially expressed gene can qualitatively have its expression altered, including an activation or inactivation, in, for example, normal versus lymphoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the LA protein and standard immunoassays (ELISAs, etc.) or other techniques, including mass spectroscopy assays, 2D gel electrophoresis assays, etc. Thus, the proteins corresponding to LA genes, i.e. those identified as being important in a lymphoma phenotype, can be evaluated in a lymphoma diagnostic test.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the LA nucleic acid probes may be attached to biochips as outlined herein for the detection and quantification of LA sequences in a particular cell. The assays are done as is known in the art. As will be appreciated by those in the art, any number of different LA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect LA sequences that are up-regulated or down-regulated in lymphoma as compared to normal lymphoid tissue. In instances where the LA sequence has been altered but shows the same expression profile or an altered expression profile, the protein will be detected as outlined herein.

In a preferred embodiment nucleic acids encoding the LA protein are detected. Although DNA or RNA encoding the LA protein may be detected, of particular interest are methods wherein the mRNA encoding a LA protein is detected. The presence of mRNA in a sample is an indication that the LA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a LA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The LA proteins, antibodies, nucleic acids, modified proteins and cells containing LA sequences are used in diagnostic assays. This can be done on an individual gene or corresponding polypeptide level, or as sets of assays.

As described and defined herein, LA proteins find use as markers of lymphoma. Detection of these proteins in putative lymphomic tissue or patients allows for a determination or diagnosis of lymphoma. Numerous methods known to those of ordinary skill in the art find use in detecting lymphoma. In one embodiment, antibodies are used to detect LA proteins. A preferred method separates proteins from a sample or patient by electrophoresis on a gel (typically a denaturing and reducing protein gel, but may be any other type of gel including isoelectric focusing gels and the like). Following separation of proteins, the LA protein is detected by immunoblotting with antibodies raised against the LA protein. Methods of immunoblotting are well known to those of ordinary skill in the art.

In another preferred method, antibodies to the LA protein find use in in situ imaging techniques. In this method cells are contacted with from one to many antibodies to the LA protein(s). Following washing to remove non-specific antibody binding, the presence of the antibody or antibodies is detected. In one embodiment the antibody is detected by incubating with a secondary antibody that contains a detectable label. In another method the primary antibody to the LA protein(s) contains a detectable label. In another preferred embodiment each one of multiple primary antibodies contains a distinct and detectable label. This method finds particular use in simultaneous screening for a plurality of LA proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing lymphoma from blood samples. As previously described, certain LA proteins are secreted/circulating molecules. Blood samples, therefore, are useful as samples to be probed or tested for the presence of secreted LA proteins. Antibodies can be used to detect the LA by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled LA nucleic acid probes to tissue arrays is done. For example, arrays of tissue samples, including LA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the LA proteins, antibodies, nucleic acids, modified proteins and cells containing LA sequences are used in prognosis assays. As above, gene expression profiles can be generated that correlate to lymphoma severity, in terms of long term prognosis. Again, this may be done on either a protein or gene level, with the use of genes being preferred. As above, the LA probes are attached to biochips for the detection and quantification of LA sequences in a tissue or patient. The assays proceed as outlined for diagnosis.

In a preferred embodiment, any of the LA sequences as described herein are used in drug screening assays. The LA proteins, antibodies, nucleic acids, modified proteins and cells containing LA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokamik, et al., Science 279, 84-8 (1998), Heid, et al., Genome Res., 6:986-994 (1996).

In a preferred embodiment, the LA proteins, antibodies, nucleic acids, modified proteins and cells containing the native or modified LA proteins are used in screening assays. That is, the present invention provides novel methods for screening for compositions which modulate the lymphoma phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokamik, supra.

Having identified the LA genes herein, a variety of assays to evaluate the effects of agents on gene expression may be executed. In a preferred embodiment, assays may be run on an individual gene or protein level. That is, having identified a particular gene as aberrantly regulated in lymphoma, candidate bioactive agents may be screened to modulate the gene's response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the LA sequence has been altered but shows the same expression profile or an altered expression profile, 300 the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the LA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the LA nucleic acid probes are attached to biochips as outlined herein for the detection and quantification of LA sequences in a particular cell. The assays are further described below.

Generally, in a preferred embodiment, a candidate bioactive agent is added to the cells prior to analysis. Moreover, screens are provided to identify a candidate bioactive agent which modulates lymphoma, modulates LA proteins, binds to a LA protein, or interferes between the binding of a LA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the lymphoma phenotype, binding to and/or modulating the bioactivity of an LA protein, or the expression of a LA sequence, including both nucleic acid sequences and protein sequences. In a particularly preferred embodiment, the candidate agent suppresses a LA phenotype, for example to a normal tissue fingerprint. Similarly, the candidate agent preferably suppresses a severe LA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an LA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more LA genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition screens can be done for novel genes that are induced in response to a candidate agent. After identifying a candidate agent based upon its ability to suppress a LA expression pattern leading to a normal expression pattern, or modulate a single LA gene expression profile so as to mimic the expression of the gene from normal tissue, a screen as described above can be performed to identify genes that are specifically modulated in response to the agent. Comparing expression profiles between normal tissue and agent treated LA tissue reveals genes that are not expressed in normal tissue or LA tissue, but are expressed in agent treated tissue. These agent specific sequences can be identified and used by any of the methods described herein for LA genes or proteins. In particular these sequences and the proteins they encode find use in marking or identifying agent treated cells. In addition, antibodies can be raised against the agent induced proteins and used to target novel therapeutics to the treated LA tissue sample.

Thus, in one embodiment, a candidate agent is administered to a population of LA cells, that thus has an associated LA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, LA tissue may be screened for agents that reduce or suppress the LA phenotype. A change in at least one gene of the expression profile indicates that the agent has an effect on LA activity. By defining such a signature for the LA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “LA proteins” or an “LAP”. The LAP may be a fragment, or alternatively, be the full length protein to the fragment encoded by the nucleic acids of the figures. Preferably, the LAP is a fragment. In another embodiment, the sequences are sequence variants as further described herein.

Preferably, the LAP is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the LA proteins are conjugated to an immunogenic agent as discussed herein. In one embodiment the LA protein is conjugated to BSA.

In a preferred embodiment, screening is done to alter the biological function of the expression product of the LA gene. Again, having identified the importance of a gene in a particular state, screening for agents that bind and/or modulate the biological activity of the gene product can be run as is more fully outlined below.

In a preferred embodiment, screens are designed to first find candidate agents that can bind to LA proteins, and then these agents may be used in assays that evaluate the ability of the candidate agent to modulate the LAP activity and the lymphoma phenotype. Thus, as will be appreciated by those in the art, there are a number of different assays which may be run; binding assays and activity assays.

In a preferred embodiment, binding assays are done. In general, purified or isolated gene product is used; that is, the gene products of one or more LA nucleic acids are made. In general, this is done as is known in the art. For example, antibodies are generated to the protein gene products, and standard immunoassays are run to determine the amount of protein present. Alternatively, cells comprising the LA proteins can be used in the assays.

Thus, in a preferred embodiment, the methods comprise combining a LA protein and a candidate bioactive agent, and determining the binding of the candidate agent to the LA protein. Preferred embodiments utilize the human or mouse LA protein, although other mammalian proteins may also be used, for example for the development of animal models of human disease. In some embodiments, as outlined herein, variant or derivative LA proteins may be used.

Generally, in a preferred embodiment of the methods herein, the LA protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microliter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the LA protein is bound to the support, and a candidate bioactive agent is added to the assay. Alternatively, the candidate agent is bound to the support and the LA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the LA protein may be done in a number of ways. In a preferred embodiment, the candidate bioactive agent is labeled, and binding determined directly. For example, this may be done by attaching all or a portion of the LA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. LA protein), such as an antibody, peptide, binding partner, ligand, etc. Under certain circumstances, there may be competitive binding as between the bioactive agent and the binding moiety, with the binding moiety displacing the bioactive agent.

In a preferred embodiment, the Nrf2 binding moiety is a nucleic acid comprising the Nrf2 binding sequence GCTGAGTCATGATGAGTCA. In another preferred embodiment, the Nrf2 binding moiety is a transcriptional cofactor involved in Nrf2-mediated gene regulation. In a preferred embodiment, the DNA binding domain of Nrf2 is used in binding assays. In one embodiment, the transcriptional activation domain of Nrf2 is used in binding assays.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the LA protein and thus is capable of binding to, and potentially modulating, the activity of the LA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the LA protein with a higher affinity. Thus, if the candidate bioactive agent is labeled, the presence of the label on the support, coupled with a lack of competitor binding, may indicate that the candidate agent is capable of binding to the LA protein.

In a preferred embodiment, the methods comprise differential screening to identity bioactive agents that are capable of modulating the activity of the LA proteins. In this embodiment, the methods comprise combining a LA protein and a competitor in a first sample. A second sample comprises a candidate bioactive agent, a LA protein and a competitor. The binding of the competitor is determined for both samples, and a change, or difference in binding between the two samples indicates the presence of an agent capable of binding to the LA protein and potentially modulating its activity. That is, if the binding of the competitor is different in the second sample relative to the first sample, the agent is capable of binding to the LA protein.

Alternatively, a preferred embodiment utilizes differential screening to identify drug candidates that bind to the native LA protein, but cannot bind to modified LA proteins. The structure of the LA protein may be modeled, and used in rational drug design to synthesize agents that interact with that site. Drug candidates that affect LA bioactivity are also identified by screening drugs for the ability to either enhance or reduce the activity of the protein.

In a preferred embodiment, transcription assays as known in the art, for example as disclosed in (Ausubel, supra) and Caterina et al., NAR 22:2383-2391, 1994, are used in screens to identify candidate bioactive agents that can affect Nrf2 protein activity, particularly transcription regulating activity. In a preferred embodiment, the transcription assays employ the Nrf2 DNA binding sequence GCTGAGTCATGATGAGTCA. In a preferred embodiment, an Nrf2 protein comprises the amino acid sequence st forth in SEQ. ID NO:211 and at Genbank accession number AAA68291, or a fragment thereof. In another preferred embodiment, an Nrf2 protein comprises the amino acid sequence set forth in SEQ ID NO:213 and at Genbank accession number NP_{—}006155, or a fragment thereof. In another preferred embodiment, an Nrf2 protein comprises the amino acid sequence set forth by amino acids 477 to 518 in SEQ ID NO:211 and at Genbank accession number AAA68291. In another preferred embodiment, an Nrf2 protein comprises the amino acid sequence set forth by amino acids 482 to 526, more preferably 482 to 504, in SEQ ID NO:213 and at Genbank accession number NP_{—}006155.

In one embodiment, the portion of Nrf2 protein used comprises the DNA binding domain, such as the basic domain of a basic leucine zipper domain-containing protein. In one embodiment, the portion of Nrf2 used comprises the transcriptional activation domain, such as the acidic domain of a basic leucine zipper domain-containing protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of LA proteins may also be done. In a preferred embodiment, methods for screening for a bioactive agent capable of modulating the activity of LA proteins comprise the steps of adding a candidate bioactive agent to a sample of LA proteins, as above, and determining an alteration in the biological activity of LA proteins. “Modulating the activity of an LA protein” includes an increase in activity, a decrease in activity, or a change in the type or kind of activity present. Thus, in this embodiment, the candidate agent should both bind to LA proteins (although this may not be necessary), and alter its biological or biochemical activity as defined herein. The methods include both in vitro screening methods, as are generally outlined above, and in vivo screening of cells for alterations in the presence, distribution, activity or amount of LA proteins.

Thus, in this embodiment, the methods comprise combining a LA sample and a candidate bioactive agent, and evaluating the effect on LA activity. By “LA activity” or grammatical equivalents herein is meant one of the LA protein's biological activities, including, but not limited to, its role in lymphoma, including cell division, preferably in lymphoid tissue, cell proliferation, tumor growth and transformation of cells. In one embodiment, LA activity includes activation of or by a protein encoded by a nucleic acid of the table. An inhibitor of LA activity is the inhibition of any one or more LA activities.

In a preferred embodiment, the activity of the LA protein is increased; in another preferred embodiment the activity of the LA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a LA protein. The methods comprise adding a candidate bioactive agent, as defined above, to a cell comprising LA proteins. Preferred cell types include almost any cell. The cells contain a recombinant nucleic acid that encodes a LA protein. In a preferred embodiment, a library of candidate agents are tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the LA protein.

In one embodiment, a method of inhibiting lymphoma cancer cell division is provided. The method comprises administration of a lymphoma cancer inhibitor.

In another embodiment, a method of inhibiting tumor growth is provided. The method comprises administration of a lymphoma cancer inhibitor.

In a further embodiment, methods of treating cells or individuals with cancer are provided. The method comprises administration of a lymphoma cancer inhibitor.

In one embodiment, a lymphoma cancer inhibitor is an antibody as discussed above. In another embodiment, the lymphoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for lymphoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various LA sequences are important in lymphoma. Accordingly, disorders based on mutant or variant LA genes may be determined. In one embodiment, the invention provides methods for identifying cells containing variant LA genes comprising determining all or part of the sequence of at least one endogenous LA genes in a cell. As will be appreciated by those in the art, this may be done using any number of sequencing techniques. In a preferred embodiment, the invention provides methods of identifying the LA genotype of an individual comprising determining all or part of the sequence of at least one LA gene of the individual. This is generally done in at least one tissue of the individual, and may include the evaluation of a number of tissues or different samples of the same tissue. The method may include comparing the sequence of the sequenced LA gene to a known LA gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the LA gene can then be compared to the sequence of a known LA gene to determine if any differences exist. This can be done using any number of known homology programs, such as Bestfit, etc. In a preferred embodiment, the presence of a difference in the sequence between the LA gene of the patient and the known LA gene is indicative of a disease state or a propensity for a disease state, as outlined herein.

It will be recognized that in some cases, particularly those concerning tumor suppresser genes, or recessive mutations generally, Nrf2 sequences characteristic of an Nrf2 phenotype will be found in normal lymphoid tissue. In these case it will be recognized that other Nrf2 gene alleles found in the tissue are likely involved in the maintenance of the normal lymphoid phenotype.

It will also be recognized that many transcription factors function as multimers, and as such, dominant negative effects in respect of the physiological processes they regulate are often encountered with altered alleles. That is, a single alternate allele (alternate in respect of the recognized wildtype allele) is often sufficient to alter transcription as normally regulated by wildtype protein, through protein-protein interactions and the dominant dysfunction of an alternate protein.

In a preferred embodiment, the LA genes are used as probes to determine the number of copies of the LA gene in the genome. For example, some cancers exhibit chromosomal deletions or insertions, resulting in an alteration in the copy number of a gene.

In another preferred embodiment LA genes are used as probes to determine the chromosomal location of the LA genes. Information such as chromosomal location finds use in providing a diagnosis or prognosis in particular when chromosomal abnormalities such as translocations, and the like are identified in LA gene loci.

Thus, in one embodiment, methods of modulating LA in cells or organisms are provided. In one embodiment, the methods comprise administering to a cell an anti-LA antibody that reduces or eliminates the biological activity of an endogenous LA protein. Alternatively, the methods comprise administering to a cell or organism a recombinant nucleic acid encoding a LA protein. As will be appreciated by those in the art, this may be accomplished in any number of ways. In a preferred embodiment, for example when the LA sequence is down-regulated in lymphoma, the activity of the LA gene is increased by increasing the amount of LA in the cell, for example by overexpressing the endogenous LA or by administering a gene encoding the LA sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the LA sequence is up-regulated in lymphoma, the activity of the endogenous LA gene is decreased, for example by the administration of a LA antisense nucleic acid.

In one embodiment, the LA proteins of the present invention may be used to generate polyclonal and monoclonal antibodies to LA proteins, which are useful as described herein. Similarly, the LA proteins can be coupled, using standard technology, to affinity chromatography columns. These columns may then be used to purify LA antibodies. In a preferred embodiment, the antibodies are generated to epitopes unique to a LA protein; that is, the antibodies show little or no cross-reactivity to other proteins. These antibodies find use in a number of applications. For example, the LA antibodies may be coupled to standard affinity chromatography columns and used to purify LA proteins. The antibodies may also be used as blocking polypeptides, as outlined above, since they will specifically bind to the LA protein.

In one embodiment, a therapeutically effective dose of a LA or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for LA degradation, systemic versus localized delivery, and rate of new protease synthesis, as well as the age, body weight, general health, sex, diet, time of administration, drug interaction and the severity of the condition May be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the LA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the LA proteins and modulators may be directly applied as a solution or spray.

The pharmaceutical compositions of the present invention comprise a LA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, LA proteins and modulators are administered as therapeutic agents, and can be formulated as outlined above. Similarly, LA genes (including both the full-length sequence, partial sequences, or regulatory sequences of the LA coding regions) can be administered in gene therapy applications, as is known in the art. These LA genes can include antisense applications, either as gene therapy (i.e. for incorporation into the genome) or as antisense compositions, as will be appreciated by those in the art.

In a preferred embodiment, LA genes are administered as DNA vaccines, either single genes or combinations of LA genes. Naked DNA vaccines are generally known in the art. Brower, Nature Biotechnology, 16:1304-1305 (1998).

In one embodiment, LA genes of the present invention are used as DNA vaccines. Methods for the use of genes as DNA vaccines are well known to one of ordinary skill in the art, and include placing a LA gene or portion of a LA gene under the control of a promoter for expression in a LA patient. The LA gene used for DNA vaccines can encode full-length LA proteins, but more preferably encodes portions of the LA proteins including peptides derived from the LA protein. In a preferred embodiment a patient is immunized with a DNA vaccine comprising a plurality of nucleotide sequences derived from a LA gene. Similarly, it is possible to immunize a patient with a plurality of LA genes or portions thereof as defined herein. Without being bound by theory, expression of the polypeptide encoded by the DNA vaccine, cytotoxic T-cells, helper T-cells and antibodies are induced which recognize and destroy or eliminate cells expressing LA proteins.

In a preferred embodiment, the DNA vaccines include a gene encoding an adjuvant molecule with the DNA vaccine. Such adjuvant molecules include cytokines that increase the immunogenic response to the LA polypeptide encoded by the DNA vaccine. Additional or alternative adjuvants are known to those of ordinary skill in the art and find use in the invention.

In another preferred embodiment LA genes find use in generating animal models of Lymphoma. As is appreciated by one of ordinary skill in the art, when the LA gene identified is repressed or diminished in LA tissue, gene therapy technology wherein antisense RNA directed to the LA gene will also diminish or repress expression of the gene. An animal generated as such serves as an animal model of LA that finds use in screening bioactive drug candidates. Similarly, gene knockout technology, for example as a result of homologous recombination with an appropriate gene targeting vector, will result in the absence of the LA protein. When desired, tissue-specific expression or knockout of the LA protein may be necessary.

It is also possible that the LA protein is overexpressed in lymphoma. As such, transgenic animals can be generated that overexpress the LA protein. Depending on the desired expression level, promoters of various strengths can be employed to express the transgene. Also, the number of copies of the integrated transgene can be determined and compared for a determination of the expression level of the transgene. Animals generated by such methods find use as animal models of LA and are additionally useful in screening for bioactive molecules to treat lymphoma.

LA nucleic acid sequences of the invention are depicted in Table 1. All of the nucleic acid sequences shown are from mouse.

TABLE 1

SEQ.
ID

| | | |
|---|---|---|
| TAG # | NO. | SEQUENCE |

| | | |
|---|---|---|
| S00001 | 1 | AGCAAGCAGGGAGCCAGCTGCGGGCCAAGGAGGAGGG |
| | | GNGACTTTCGGTAACCGCACAGCANCCGGCGGGACAG |
| | | CAGCGGAGTGTAGGGCAGCGC |
| S00002 | 2 | CCGGGNTTTAAAAAGCACGCG |
| S00003 | 3 | CTGGAGAGCATNTTCAGGGTGNACAGGGCNGGCCGNG |
| | | GGCNGGGTGGACAAAGGTCAGGANNCANTCGATNTAG |
| | | CCCANATGGTCCTTCAGTCACAGAGCCGGAACAGGCA |
| | | ATTCTCTANCCATAAACAGCCACTCAGGCAGCCCCAA |
| | | ACCACACGCATGCACATGTGAAGACTCTGATGAAGTA |
| | | CAGCTGCT |
| S00004 | 4 | GGAGCTGTGGTCGAGGCTGGTCCAGCATATCCCTGGA |
| | | GACTAGAACTGTGCAGTGGGAAATGCGGTAGACTCTG |
| | | AGTTCTGGAACTTGTTTGAATCTCTGTTTTGAATCTC |
| | | CGTTTCCTCATCTGTAAGAGGTTAGTAAGTTGTCTAA |
| | | GGAAAGGT |
| S00005 | 5 | AGATAAGAGCTAGGAGACACCCACAGCTGGAAAATCA |
| | | CCAAGTTTCTAAGACCAC |
| S00006 | 6 | AAAACATGGGATTAACTTTATAACCCAGGATCAAACT |
| | | GGCTTCGGTCCGCTCTTGCGGTCATCTTAGACTTGTG |
| | | TTTTTCCTTCCCTTAGGAACTTCCTCAGCATGCTTTT |
| | | TCTAAAAGCACTCCAGTGTATCTGCAC |
| S00007 | 7 | AGTGGAAGATGGGAATTCTTAGCCCAAGACCTGATCA |
| | | GGCTACACTTGCCCTCGTTCACCTCATCCATTTGCAT |
| | | GGAGGTGACTTTGGCTTCCTGACANTATCCCTCCTGC |
| | | AATTCAGTCCCCATAGAGAACTGCCAATTGCCAGTTT |
| | | AAGACCTTCTGTTCCTCCCTGCGGGGCATAAGTCCAT |
| | | GCGCTGAGCCCGGTCACGTGACNGACCTCCAACGCCT |
| | | CATCCTGCTGTCTCAGTCT |
| S00008 | 8 | CCCTGACAGTATGTNGTGTGGGTTGGGTAAANACNTA |
| | | NCGCTGTGGGTGTGGATTGGCTTAGANGTGCATCTGG |
| | | TATGTGCCTACAGGCTTTCTAACTGTNCCTACNCGTC |
| | | TATGTAC |
| S00009 | 9 | CACCCTTGTATCGGTCTCCGCCACCACCACCACTACC |
| | | AGCATCCCCCAAAGAAGAAAATCTCCTCCGAAATGCC |
| | | CCGAAGAGTGCTGCTGCTGGCTCTGAAGCCGTGTAGA |
| | | ATTTCGTAATGGAATGTGAACTGCTCGTCCGGATCTG |
| | | GGCTCACGTTCTATCTCTTAACCAGTAAGGAACGAGG |
| | | GAGGGCAAATCTGCTGAGCAAGGAAAAATAACTTTCC |
| | | TCCTCTTTTATAACCCATCACGGATGCACCGCGGACG |
| | | AGGGCAGCTAGCAAC |
| S00010 | 10 | TNATGGTGGCCCCNGACNAGGTCCCCTACCTGCTTGA |
| | | CCTACACTTGTTCCTGGGCCGCTCTGTCACCCTGGCC |
| | | CGTCCTTGTGAGGAGCCTTCAGGTGAGGCCAGGCTGG |
| | | ACTGGGCTTGGGTCCCCATGGACCATGGAGATCATGA |
| | | GCAGGCTGGGGTGCAGTGGTCTGACCACAGGAGATGT |
| | | CTGCTGGGTCTGACCGTACGGCCTGGGTGCTGGGNTA |
| | | CCCTTGGGCTATTGTNTGCCAGAGTGGGGGGTCTGGT |
| | | TGCATATAATACTCTAGCCTGTATCTGTT |
| S00011 | 11 | GGAGCAGTCATCATTTGGAAAACTGAGAGAAGATCTT |
| | | TAAAANGAGCCCAATCTGAGGTGTGGTGCACTTCTCT |
| | | TCTGCTGGGCACACCTTACCCGAACTCCGCGTGCTTG |
| | | CTGCTGTCTGGACCTTACTTGTCACCTCTACTTCCTG |
| | | CTGTGAGGACTGCCACCCAGTCTCAGCCACCACCACC |
| | | TCTGCCCCCACTGTGATGACACAGGAACTGCGC |
| S00012 | 12 | CTCGTTTCAGGGTTGCTTANAGGATTCTTAAAAACCA |
| | | GACAATTNAGCAATTCCATGTTTACCANGGGCAGTTG |
| | | GAAATCCAGTTTCTAAAATCACTGTCAACTCTCCNCA |
| | | CTTTCTATTGT |
| S00013 | 13 | CTCCGTNGGGAGCCANCNTGGACGGNGTGTGGGGACC |
| | | GGTNTCCCAGTCNTCTCCGCAAANCGGTCTCCNAGGT |
| | | GGTTTAACCGGNGTTTGGTGGNGGTCGGGTTTCTTAC |
| | | AGTTAGATGTCANCTCANCTAGTGTGACATCACCCAA |
| | | ACCAGTGTGATTTTTCCCCCAACATCCCAATCACATC |
| | | CCAGCGATTGGGCAGCGCAGGGAGACATTGACTACCT |
| | | GGGGGATGACTCTGAGGGTTTAGAATTCTCAGTTTTT |
| | | ACTTAAATTGTTTGCTGCCATGTCGATTTCAGGGCAG |
| | | CNAPGGGGNATTTAGATGCCTCCCTGTCCTTNGA |
| S00014 | 14 | ACTTCACCGANATGTAGGCAAGAATTCAGACGGATGG |
| | | G |
| S00015 | 15 | ATCTCATCTCATCTCATCTCATCTTCTTTCCTCTCCA |
| | | TACTTATGTTGCCTATTCAGGAATATTTTGGCTATTG |
| | | TACCTGTGGATATTCATTACAAAGGAGGCAGTGGCTC |
| | | AAATGAAGCCAAAGAGCCTGGCTCTGAAGGACTGATG |
| | | GCCAGGTGGCCAGACATAGGTATTCAAAANAAGATTT |
| | | GAGGCTTCTGTTTACCTCTTCGCTGATGGTGCCACTG |
| | | CTGAAGTAGTACTTCTTTACCCTGGCAGCATTGTCTC |
| | | AGTGACAGCTGTGTCTTGTCCACGGGGCCTCTGTGTC |
| | | CCATGCTCTTCACAA |
| S00016 | 16 | TCTTGGANGCTCNAAAGCTTGCGGGGNGTTGGTGTAT |
| | | CCATGGCAGGGACTTGAGTTGATTATTTTTACCCCGC |
| | | AAACAGGGTANTGCTGACCTCGAACTCTCAATCTTTT |
| | | CCCCAAGTGTCTGGATTACAAATGTTTGTCTACACAC |
| | | CCAAACAAATTTTAATGATNCAAGAATTNTCCCCGTG |
| | | GCC |
| S00017 | 17 | CCCAACACTGCCCATGCCTCCCCAAGCCGATTAAACT |
| | | CTTCTCTCGATTGCCTCTTTATACTTCTCTACTCTCG |
| | | GATAATCCCAGTCTTCAAGGCCCTAGAGAAGGAATGA |
| | | CTGTGCGTCCCTTTTAATTTTTACCCTAGAACTCCCC |
| | | TGATTTTTTAACTCAGTGACCAC |
| S00018 | 18 | AAAGTGCCAACCTCTGCAGNTGNTCTTCACTCCACCA |
| | | CACTNGGNCCTGACTGGCTACAGAGATGGAGTCTCAG |
| | | NCCAGCTCCCCGCCAG |
| S00019 | 19 | TTAGGACTGAAGGAGCTGAAGGGGTTTGCAACCCCAT |
| | | AGGAAGNATAACNATATCAACCAACCAG |
| S00020 | 20 | GAGCCACACTGGNAAGTCTGACAAGAGTCAGTGCTGT |
| | | CCATGCTGACTCCACCCTG |
| S00021 | 21 | CTATAATGATATACCAGATAAAGGTCAGAAAGGGTGG |
| | | TAGTCTCTTTATGGAGTATGTTTTTGGGGTTAAAAGT |
| | | TTTATTTTGATATTAGAAGAGCTTCAATTCAAAACTG |
| | | ACTTTTAAGGCTCAAACATAACAGAGATAGATAACCA |
| | | GTATCCTTGTAAATGATCAAATAATTTAATCTGTTCA |
| | | GAAATATATAAGAAGCCATGCTAAGAACTGATGCAGT |
| | | TAATTTCAAGATTAGCTTTATTTAGTCTTCTGTTGTA |
| | | TATTTTCAAGGTATAGTTTAGAGCAGATAACTAAAAA |
| | | CAGGTAGGTACTAGCCCTCAAACCAGTCACAGATCTC |
| | | CTGAATGTGGCATTTAG |
| S00022 | 22 | CTACTTGGATCTGATGATGNTGCCCAGGATACAAGAA |
| | | GAGACACAGTCAGCCAGTCCTAGACAGACAGACTTCC |
| | | TAGGAAGCCAGTGACTCTCAGCATGAAAGGCACCAAG |
| | | NACTGGGCAGCCAGGACTCAGGNCCCTCTGGCATTCT |
| | | GGCTACCTCCCTGTCCCCC |
| S00023 | 23 | TNAAAGATTGGGACACCCCCTCCGCGGCCCGCCCACC |
| | | GCCCTCCCGCCGGGAAACCAGGCCCGCGTCCTCTAGC |
| | | TCTCAGGCCGAGGGCAGAAGTCCATAGTAGCCCCGAT |
| | | CAATATTATCCCGAGCTTGCTCCCTGGAGGGAGGTTT |
| | | AAACCAGGGCCCCTGTCGCACTACCCCGATGGGCACA |
| | | GGCAGG |
| S00024 | 24 | CNTCTGACCAGCTCTAAATGGCTCTNATTACNTTTCA |
| | | ATGGAGCATAGAGTCAAATTTTGACAAGCACATAACT |
| | | TAATAGCTGATCTGCAGGCATACCACCAGACTGATTT |
| | | GTAACTGCCAGCGAATAAGCCCACGAGACGGTTATCC |
| | | AAAGTCTTCCAGTTCAAAGACCGAAGTTGTGAGGATG |
| | | AAGCCACTACAGCCACGTTGGAGCTAAGCGTCTGCTG |
| | | CATTCGAGGCTCTAGACACAATGCAGGGAACTGAGCC |
| | | ATCTCAAAGCATCACTC |
| S00025 | 25 | GTTTCAATTCAGCCCTGTAAAAAACTACACTTCCTCG |
| | | TGG |
| S00026 | 26 | TCTTACCAAAACCACAGCTCTAGGGTGATTCTCACAA |
| | | TATTAGGCCAGTGCTTCACTGATTGCATCAAAAGCTA |
| | | GGGGNCTCCAGTGGANAACATTCCAGCTGTGTTTTTT |
| | | GCCTGATGACACACACACATAGATAT |
| S00027 | 27 | AAAGGTGCTTCTTAGAGGTGCTAATTGGGAAGAGCCA |
| | | AGGTGAAGGCTGCAGGACACAAATGTATCTCTGTGAA |
| | | ATCTGCTATGGAAATCGTCTGGGACCTGTTGGTGGAA |
| | | ATCCTATTGGCCTTGAGCAAAAAGGCGAAA |
| S00028 | 28 | TTAAAAGAACCCTGGCTTCCCAAGTTCTGCCTCAGGC |
| | | AAAGGAGCCTGCTTACATTCCAAGCAGGACTTGTGCC |
| | | CTCCAGATAGGGAACCCCAGGAAGCCACCGCCCGTCC |
| | | CAGACCAATTCTTTCCCTCCCTTCAGCTCGGTAGGTC |
| | | TTTGCATCTAGGATCCCCGCCCCAGACCGCCTGTGAG |
| | | CAGAGCAAAGCGGTCCCAGCAGCTCTCAGATACTGCT |
| | | GTGGGTTCTGTGTCTGCGAGGAAGGCAGCACAGAAAC |
| | | TTTCAGTCCCCGGGTATTTTGTCAGTGTGGCTCTTTT |
| | | ATGTTACCGCATCCCACAGGGAGACACGGTTATGCCA |
| | | TTTTTATTATCTCTCTCCCCTGCTGGGAGCTTCTTC |
| S00029 | 29 | ACAGAAAGAAGTCTGGTCACAACTGGCTACAGCAAAC |
| | | GAGCCAGGTACCCCAGGGACGACTCNCCATTCCNGCC |
| | | AGAGATCTGATCTACGTACACCTGCGTCATGCTGAGA |
| | | CCCTCNAGCCTCACTAAAAGGGTCCCTGCCTAGTTCT |
| | | GTTTACNAATCTGCCTTATTCTGTTTTGTTCCCATGT |
| | | TAAAGATAGAGTNAATACCGTATT |
| S00030 | 30 | TGTGAGCAGAGGGTTAAAGACATGAAATCTGGGGCTG |
| | | CAGAGACAGCTCCATAGTTNGCAACACCTGCTGCTCT |
| | | CTAAGAGGACCCAGAGTTTGGCTCCCAGCACCCACAT |
| | | CAGGTNGNNNATGCACCTGAAACCACAGCTCTAGGGG |
| | | TCTCAACCTCCTGGGGCTCTGCAGCGCCAGCATATGC |
| | | ACTTGCAC |
| S00031 | 31 | GGTTGCGGTCACATTCGGCGTGTCCCCAGCCCGGGGG |
| | | ACGGGGCCCCGGGGAGGCCCCGCATCGCTGCANT |
| S00032 | 32 | CTTGCAAGAGTNATTTGTGTGCTCCTTCTACCANCTT |
| | | CTAAAGATNAGACGCTGGTTGTCAGCCTCTGTGGCCA |
| | | AGC |
| S00033 | 33 | GATNNCCCANTATTCACTCTGATAGTGAATATACCCA |
| | | AACATGACACCACCCTCCGGGACAAAGGAAGCACATG |
| | | CTGGCTTGCTGGGACCCCTTAAGTCTGGCCAGCTCTA |
| | | GGTANGGACTTCCTGTCCTCATNCACTGGGGAAAAGA |
| | | AGTGTTGGAGAAACGTGTCACCANTAGGTGTCGCCCG |
| | | ACAACGGTCTCGATCAACCAAACAAACCAATACAGAT |
| | | CNCTC |
| S00034 | 34 | ATTCCACAGGTAGAAATGTCCACATCTTACCTCATGT |
| | | GTTGCTATACTAAAATATTCATGCATTGAAAATACTG |
| | | TATGAAGCCGGCCAGTGGTGGCGCATGCCTTTAATCC |
| | | CAGCACTCGGGAGGCAGAGGCAGGCAGATTTCTCTGA |
| | | GTTTG |
| S00035 | 35 | CTATAATGATATACCAGATAAAGGTCAGAAAGGGTGG |
| | | TAGTCTCTTTATGGAGTATGTTTTTGGGGTTAAAAAG |
| | | TTTTATTTTGATATTAGAAGAGCTTCAATTCAAAACT |
| | | GACTTTTAAGGCTCAACATAACAGAGATAGATAACCA |
| | | GTATCCTTGTAAATGATCAAATAATTTAATCTGTTCA |
| | | GAAATATATAAGAAGCCATGCTAAGAACTGATGCAGT |
| | | TAATTTCAAGATTAAGCTTTATTTAGTCTTCTGTTGT |
| | | ATATTTTCAAGGTATAGTTTAGAGCAGATAACTAAAA |
| | | ACAGGTAGGTACTAGCCCTCAAACCAGTCAGAGATCT |
| | | CCTGAATGTGGCATTTAG |
| S00036 | 36 | GCTGAAAATGCTAGGCTTTGTNGAGCTATGAGCCCCG |
| | | GGAATCCTCCTGTCTCTCTCCAGCNGAAGGATTACAA |
| | | ATCTACTCCACCTTGAACATGGGTGCTGNAGGNGAAC |
| | | ACTTAANCTCACGGAAGNTCANCAGCATTTNACAAAC |
| | | CTGTCATGCCTTGNTTTGTTTTAAAGATTNATTTATT |
| | | CATAGGCATGATTGTTTTGCCTGCATGAATTTCT |
| S00037 | 37 | CTTTAACCGTCCTCTCCTAAAAAATATAAGAAATGAG |
| | | TAAATGGGTGACTGGAGGAACAAGAGAAATAATAGTG |
| | | TGTAANAGGGTGAGTCTCCGCTTTGGTCAGCACAACG |
| | | CACCTGCAGAGGCTTTCTTTCTCTTTTATACGTTTTA |
| | | ATAATGCTGCTTCCATCTCCCAGGGACGTTTGAGGCT |
| | | CAGCCTCACCAATGTTTCTCTCCTCTTGTTCTCCCCT |
| | | AGCCTACCCATCACCACTCACCCCTGCGGCAGCCACA |
| | | CAGGCCTTCCTCAGCTTCTGTTCCTGAACTTTGAATC |
| | | GAT |
| S00038 | 38 | GTCTCTCCTGCTTGCTGAAGTAGCTGTTTGTGTCNCC |
| | | TCCCCCANCCCACCCTCAAGCTCACACAGATCCTCCG |
| | | AACATATGAAGCAGAGGAGGGGCTTAGGCTGCGGAAC |
| | | TCCC |
| S00039 | 39 | GTCTGCTCTTCCTTCCCGACAGTATCTAATATAAAAG |
| | | AGGACTGCAATGCCATGGCGTTCTGTGCTAAAATGAG |
| | | GAGCTTCAAGAAGACTGAGGTGAAGCAGGTGGTCCCT |
| | | GAGCCTGGAGTGGAGGTGACTTTCTATCTGTTGGACA |
| | | GGG |
| S00040 | 40 | AAATGACAACGAGGAAGATGAA |
| S00041 | 41 | GGGTACGTGGGCGAGGGGCTCGCCCACTGGTGAGGTC |
| | | TCTGGACCTATCGATTCCCGGCTGATGCT |
| S00042 | 42 | CCATAAGCACACATATGTAAAAGGTTTGCACACCTCA |
| | | TAAGCTTCACTTTGTGAACGTGTACAGCGTTAGTATG |
| | | TGCAAAAAATATCATGTCGGAAGAGCAGTTTCTATTT |
| | | GTGCTACCCAAAAACGGGTTTGTATTTTGAGAGGGGA |
| | | GAATCACGCTGTTAGGCTTTATTTATATCCAAGTGTC |
| | | CTCAGCCTTCTGCAAAAAAGGCAAAAGCTTTGTGTGT |
| | | GCGTGTGTGTGTTTTAATGCAGAACAACGAAGGACTC |
| | | AGACACTTTCGACTCTACAGAACCTAAGCATACACGC |
| | | GGGCCTGTGTTACATCGCGGGCCTGTGT |
| S00043 | 43 | CCCNTCNANAAANAAGAACAAAAGCTTTCTCGCTCCT |
| | | ACATGGCAAAACACAAACCACTA |
| S00044 | 44 | ATAAAAACCCAAGGCATGCAAAGGTGAAAGAAACCAG |
| | | TCAATCACCAGACGACGGCC |
| S00045 | 45 | CCAGGCTGGAGGGCCTGCGGGGACCGGTGCGTGAAAG |
| | | GCACCTCG |
| S00046 | 46 | CCCCTGCCTCCGCCACCACCACCTCCTCCAACG |
| S00047 | 47 | ATATTATCACTACAGAACATGAGGATGTCGTTGATTG |
| | | CGGCAACCACTAGACCACCACTCACTGGATGAGGAGC |
| | | TCAGGAAGCTGGCCCCATTTCTCACTGGCAGCAGCAC |
| | | AGTAGAGCTGGCCCTAGTGGCAGGGGTGTAGGTGAGC |
| | | CAGCCCTGAGGGCATGAGTGTGGGAGAACTGTCCCTG |
| | | CCACAGGTATGCTGTAGGCTGGTAGCATGGGCACAGA |
| | | GATGATTCCCCCTCCACCGCTCCTTGTCATCTCTGTC |
| | | AGTGGGGAAGGCTGCCTGCTGGTCCTGAGCTTGGGAG |
| | | TGCTATCCATGATGCTGGGAGTGCTATCTGTGATGCA |
| | | CACGAGCTTCACCAGGTAGGAGAAC |
| S00048 | 48 | TTATCCCCGCGAGACAGTCGTGCATGCTCNAAGTCAG |
| | | CCTTATCGATGTGTTACCGTGTCTTTGGTGGGGGCCT |
| | | GGCAGCAGGGTGGGAGCAGCCCGCGCGCTCTGCGGCT |
| | | GGACTGAGCGGGTCTGTAAATTAACAAGCTGGACGAC |
| | | CAGTGGCACATCCAGGCTGGCTACAAGGGGTCTTCTC |
| | | GGGAGGGACCACAGGGCCTTTTTCCAACTCGGCCGAT |
| | | GGGAGTGCGCGAGGCACACTGATGCGAGCCTCCACTG |
| | | CTCGGGCCGAGGCCATCTCTCAGTGACAGGTTTGGGA |
| | | GGACTCGCCCACGTGCGGGAAACTTAAGCAGAGGCCT |
| | | CCATTCTACGATGAGTGGTGCCACCTGAGGGGTCGGC |
| | | TCTTGGCATCAGGCC |
| S00049 | 49 | GGTTCTTTGGAAGAGCAGTCAGTGCTCCCAATTGCTG |
| | | AGATATCTTTCCAGCCCCTATTTTTAAANATTTNAGA |
| | | CAGGCTTTCAAGGGCTAGCTTGAAACTCACTATGCAA |
| | | TAGAGAAGGACTTGAACTTCGTATCCNCCTGCCTCTA |
| | | CCTCCCAAGTGCTGGGATTACAGCCCCCACCCCCACC |
| | | CCCAATGCCAGTTTGTATACTGTAACAGTGGAACCCA |
| | | GGGCTCCAGCATGCTGATGCTGGTATGCATGGGCCAC |
| | | ATCGCC |
| S00050 | 50 | ACAGAAAGGAAACGCGATTCGTTCCACTTGGAATTTC |
| | | CTTGAAATCTCCGAATCTAATCCAGCGTTAACTCACC |
| | | GTGAGAAGAGCGCTTGTCTCATAGGAGGCTGNGTTAA |
| S00051 | 51 | AAATGTTTTTTGGTTTTTTAAATCGGGCAGGGTGCTG |
| | | CGCACCTTTAATCCCAGAAAGAGGAAAGCAGAGGCGC |
| | | GTGGCTCTCCAAGCAAGCCAGGCTAGTTTCCCATCCA |
| | | TCTGCGGGTTATCCAACCAGAGAGAATTTCTCTCACT |
| | | TTGGTTTCCGACATGCTTTAGGCATAACCTGGGGAAC |
| | | GAGGGTAGGAGGGAGCTCCAGGCTCTAAGGACAAAGG |
| | | AACCGCAGGTGCAGGAAGCTCAAGGAA |
| S00052 | 52 | GTTTCAATTCAGCCCTGTAAAAAACTACACTTCCTTC |
| | | GTGGCG |
| S00053 | 53 | TTCATAAATCTGAGGCCAGCGTACAGCTATAGAGTGA |
| | | GATCCTATCT |
| S00054 | 54 | AAGTTCTCTGAGACGTGTNGACTCNGGGCGTGGGCGT |
| | | GGGTGTTTGAGTGGATCTGTCAATCCGTTGTGTGATA |
| | | AACTGTCAACAATGAAGGGATATTTATTTAGCTTATA |
| | | GAAAGTCCTGAGCCANGAACTGAAGAGGGAGGCACGC |
| | | ACTCATGGCTAGGANGCAGCTGGCTCTGGCTGGCCTT |
| | | GTCCTCATCCTACTGGGGACT |
| S00055 | 55 | CCACTCCCCCCCTTTGGCCCTGGCGTTCCCCTGTACC |
| | | GGGGCACACAAAGTCTGCGTGTCCAATGGGCCTCTCT |
| | | TTCCAGTGATGGCCGACTAGGCCATCTTTTGATACAT |
| | | ATGCAGCTAGAGTCAAGAGCTCAGGGGTACTGGTTAG |
| | | TTCATAATGTTGTTCCACCTATAGGGTTGAAGATCCC |
| | | TTTANCTCCTTGGGTACTTTCTCTAGCTCCTCCATTG |
| | | GGAGCCCTGTGATCCATCCATTAGCTGACTGTGAGCA |
| | | TCCACTTCTGTGTTTGCT |
| S00056 | 56 | GACGGTGATGCAGTAGAAATAAAGGTCTCAGCAGTGC |
| | | ACTGCAGAAAATCAAGCAAGCCCCCTTAGGAGTTATT |
| | | CATGTTTGCCGCTTTCGTGCAAATAGGGGAGGGGGCT |
| | | TAAGGCTTACCGGAAGACCCCCCACCTAGCTCAGGTC |
| | | TTGTACTTCTGTCTTTCTGGGTAAAGGCAAAGGAGAT |
| | | TTGGGGTGTAGTTGATGGCCCATTTAGGGTGGTCTCG |
| | | CAGACTAGAAAACCTGAAATGCACTTAAC |
| S00057 | 57 | AGGGAATCCAGAGTTGTACACAGCGAGGTCTGAAC |
| S00058 | 58 | AGAAGAGTTTGGTAAACTCATAGAAGCCCTTGAAGTA |
| | | TTGTAGGTTTGGTTTGCCAGTTTAATCGTAATTGCTG |
| | | CTTTTCTACAGGTTTGCTGGTGTGAAATGACTGAGTA |
| | | CAAACTGGTGGTGGTTGGAGCAGGTGGTGTTGGGAAA |
| | | AGCGCCTTGACGATCCAGCTAATCCAGAACCACTTTG |
| | | TGGATGAATATGATCCCACCATAGAGGTG |
| S00059 | 59 | CCCCCCAAAAAAATANTTGTTGGAGCACCAGTTGATA |
| | | AATATTTGCCTCAAGAAATTTGCCCCGAGGACTTGGA |
| | | GCTGACAGAAGTCAAAGCGAAGTGTGTGATTTATGTT |
| | | CTCCTGACAAGATACTGGCTGTTCTACAGACACAAGG |
| | | TTTTGAGNCTCCACGGTCCACAGACA |
| S00060 | 60 | CTATGTTGATCTGGGATATTAATTACAATATNCAAAA |
| | | CAAAAGCTGGGTATATAGCCTAGTGGTAATGTACTGA |
| | | CTTAGCATGCCCGAAGGCAGGCTTGGTCCTTTATGGA |
| | | ACTTACAGCCTGTCGGTTTTATCAGATCAGCACATAC |
| | | AGCTGGTATCTGTGTCTGTGGAACTGGTAGGTTGAGA |
| | | CTCTTCCCCATGGGCC |
| S00061 | 61 | AAAAAAGTTCTAATTATCATGTGAGGAAGANAGTAAG |
| | | TTATGAGCAGCCTCCTGGAAGCATNGCAGCGCCTCGC |
| | | TCTCTGCTCCCCTCTCTCTCTGTCTGGGTGAG |
| S00062 | 62 | TTCTCTCCNCTAGACTTCTGGGGACTGGGAGACTGCA |
| | | GTATGGGTCGTGCAGGATTGGAGTGATATACTTAGCA |
| | | AGCCTCCAGCGTGCTTGGGTCTGCAGTGACCCTGTGC |
| | | ATTCCTACAGTGNTTGCCAGAACAATTTTTGAAGTGG |
| | | TTTGAGGCCTTGCCCTGCCCTCTCCAGAGCAAGGTTA |
| | | TAGAATCAGACAATATGGCAGACACCTGCCACGTGGA |
| | | TAAATTACAAGCCGGTAAGATTTGCAATGCTGCACTT |
| | | TGGGTTTTTTGTTTTGTTTAACTGTGTGGGATAGTTC |
| | | TGCACATGGTGCAGAGGCAAATAAGTCATTTCTTGTT |
| | | GGTTTTGTTTTGAGGCAAGGTTTCTCTGTAGTTCTTG |
| | | CTGTCCTGGAACTCAAAACAGAATCCACTCACCTCTG |
| | | CCTCCTGAGTGGTGGGGATTAAAANTGAAGAACCCTT |
| | | CATAAGGC |
| S00063 | 63 | CTGTTTNANATTAGAAGCTGAACTCCCAGCAACCACC |
| | | AAAATGCCAGGGGTGAAAGATGCATGACCATAATGGC |
| | | AGCAATGGGGATGCAGACACCTGAGAATCCCTGGCCA |
| | | ATCAGGATAGCAGAATCCATAAGCCTTAGACTCAATG |
| | | AGAGGCCTTCAGAAAATAAGGCACAGAACAAGAGAGG |
| | | AAGACACCCAGTGTCAACCTTGGATCTCAGCAGGTT |
| S00064 | 64 | TTTGANTCAGGATGTGCATAGCTTTGGCCTTAAATTT |
| | | ATGATCTCCCTTCCTCAGCCTGCCAAGTAACTAAGAT |
| | | TATAGCCCTCACCAGGCCCTAGGTATAAGNATTTGTT |
| | | TTTCTTTCTTTTTTTTTCNTTTTTTTGGGTTTGTTTT |
| | | GTTTTGGANACANTGTTTCTCTTTGTANCCCNGGCNN |
| | | TNTNTT |
| S00065 | 65 | ACCAAGAAGAGTAAGAGTCATGAGGGGCAATTAGAAC |
| | | ACTTGTGTTCAGCACTGGGTCGCCGAGGCTTAAACGA |
| | | CTGCAGTCAGCTAACTAGGGATGTCGTCAGTTGTCGC |
| | | ATCGGACGGCACTTCCNNNNNNNNCTAGTTTCATCAT |
| | | CATTGCAGCCGACACCCCGCCCACGCGCGGCGCCCCG |
| | | CGATGCAGACCTCGACTTACCAGGCTCCCCTAGATCT |
| | | GTGCAGCGCACAAGACGGAGCTGAAGAGGCCTGGGCC |
| | | CGGGCTCAGCATCGCTCCAGAACCGTCACCAGC |
| S00066 | 66 | TGTCCAGGGNATTCACTCAAAGCGCTCAGTNCAAGCT |
| | | NGTCCAANAATNCTGNATAAGCGNTCANTTCAAGNTT |
| | | NTCCAAAAATTCNGG |
| S00067 | 67 | GGACCTCAGCTTTCAGAGTCTGTTCTCTCCCATTCTG |
| | | TGGGTCCTGTGAACTCAAGTNAGCTCTCACAAGAGCA |
| | | ACAAGAGCCTTTACCCGCAGAGCCATCTCGACACCCC |
| | | ATCAGTCATTTTTTTNTTTTATTATTTGGAGAAACTT |
| | | AACCTGCTGGTCTTGGGGTGCCTTAGCCTCTGGAAAA |
| | | CTCCTACAACCTTCAAAACAACTGCAATAAGGAGTGG |
| | | AGGGATTCAAAAAGTCTCGGGGCGCTGGGTTGGGCTG |
| | | GAGGCNATGCATTGCGGCTGGTCAGTGGGTGGC |
| S00068 | 68 | GCANTTAGGAGGCAAAGGCNTGTNATCNTAAGATAAT |
| | | GAAGGTAAAGTTAGTTTTATAGAAGGAGTAGGTCATG |
| | | TTTGAAAGAGACGGNTANTTTGAGCGGTAGATAAAGT |
| | | AAGAAGAGAAAGATTTG |
| S00069 | 69 | TGTAGTTAATAACCTGGTAATCCCTGCTACCCCCAGG |
| | | GC |
| S00070 | 70 | GAGGAGAGGCTGTCCNCNTGGATGAGGTCGGATCATN |
| | | TGGGGTCGTAGACGTGTAGGTGGAGAGCACAAGTCTN |
| | | ATTCTNNGG |
| S00071 | 71 | TCTTGTNTTGTNTTNNGTTGATGATNTTGTTGAGTNN |
| | | GANNNNGGGGCCTGGNNTNNCGANNTNCTGTCTTTGA |
| | | TTNATTGGAGCGGGCGATTGAGANTTCGAGGCCGNNN |
| | | GAGTNNANTTNNNNNGAGGATTATNNGGGGANCTNGA |
| | | TGGTGGATATNNGGGTGGTG |
| S00072 | 72 | TNACTGAATGGGANCTGGGGCCAGAGGGCAGTTGGNC |
| | | TNTTGNAAAGTNCGGGTCTCAGCTCAGAGCCCTAATC |
| | | CCGAAACTGGCGCNACAGTCAGCCGGTGGAGCGAGAT |
| | | AAAGCGGGCAA |
| S00073 | 73 | TTTCTGGAAACTGAATNAAATNTTTTATTCACGTGAT |
| | | TNNGCNCTTCTGGATCTATTGATTTGAGTTGGTGATA |
| | | CTGTTGGATCACGGGATTAGGCCCAATGGGGACGCGG |
| | | CCGNCNGA |
| S00074 | 74 | TGATGCTAGGCNGGCTCTTTGCCAACTAGAGCCACAN |
| | | TCCTTNAGGNTNTTCTGTTNGGGTGCCTTGGGCTGTC |
| | | CTTGCCAACCAGGGAAATCTGGANTCCNCGGGAGGCC |
| | | AGCTGNGCTGGGGACAGCTCCAAGTCNGAGACCACNA |
| | | GCNGNGATGTNGCNCG |
| S00075 | 75 | GTNTCTTACTATAGGGGTTTTTTATTGGTAAAAACTT |
| | | CCTGACTTGACCAATACTTGAATCTACAGCAGTTTAA |
| | | TAGCACATCAGTGTCCCTGTGGTAGCATGGTCACCTG |
| | | TACCCCTGGTTCTAGGCTTGGGCTTGCAGATGAATCA |
| | | GCGTGTCTTCTGATTCTGCACATTCTCTGACGTGTCA |
| | | CCGGC |
| S00076 | 76 | AAATGTTTTATTTGTGTGATTTNGGTTGTTNTGGATG |
| | | TATTGATTTGNGTTGGTGATANTGTTGGGTNNGAANT |
| | | GGGGTGTGCNGNAGGGANGTT |
| S00077 | 77 | CAACNATTACCGTGCNNCAAAAATTTTTTNNATGCGG |
| | | GGGGNCCCCAAAAAAAAGGTNTTTAGTATGGCTGTTA |
| | | TTTNTTGGGATATTTAAGTTGGCTNTTTGGTTTGNGN |
| | | TATTGNAACTTTTTGGATNTGAGTATGThAGTGTGTC |
| | | TTGGGNTAAGTTTTGATGTGAATTTNTNTTATATGTG |
| | | TCTNACATGTGTAGNNGATNGAATAAATGGAGATTTG |
| | | TANGAGGAGACANTGCGATGANACNANTGGTAGNANA |
| | | AGNGTGGGTGTTTGATTTTGCATNTTGGGATGGACTG |
| | | ATTTTGAGTNAGATTNGGGAANGGTGAGTGGTGGTTT |
| | | AGATGCTGTGGAGATTTGGGGATGGTGCNTTCTTTGA |
| | | TGAGGATTTGGATTGGGTTAGNAAAANGATTGTTAGA |
| | | TTTAGANTTGTGTTCTNTTCNCNGGGTGGTGATNATT |
| | | GGAAAGTGTATTTTGGGGTNAAGATTTTTGGANTGAA |
| | | NTGTGGAAAAAAAAAT |
| S00078 | 78 | ANGTTTTTGTGAATTGATGGANATGNTTGANTTGGGT |
| | | GATTCCGNTTNTTCTGGATTTTTTGATTTGNGTTGGT |
| | | GATANTGTTGGGTNAG |
| S00079 | 79 | GCAAGGACATACATCGGGGACGCTTCAGACTTCCCAC |
| | | TCATACCTCACAGCTCAGGGACCCAAACAGGATCCTC |
| | | AGAAACACAAGTCTGGTACCCTGCCTAGAATCACTAC |
| | | GGGTGCTGTT |
| S00080 | 80 | TGGTGTACCATGGTGTGACTCTAGGGGGCCTGTACTG |
| | | TGTAACAGGGTCCTTCCCTCCACAGTGACCTGCTGTC |
| | | TGTATAGTCTGTCTGTTTCTTTGGGACATGACTGTGC |
| | | TGTGGAGAGCAAGATCGGCTGGGGCTCTGCCTCTGGC |
| | | CCAGCATGTGGCAGCTGTATGGCTGGGGACAGACACT |
| | | TTTGCATCCCTGTGTTTCTTTCACTCCAATAGGC |
| S00081 | 81 | CACTAGAGACCCCGTGTCCAGGTGACTCTGCCCAGGG |
| | | CTACAGAACCTGGAGCAGCCCGCCTGGGAAGGTGGCT |
| | | TTTCCTCCAGATGGCCATGGGCTTTACGTTAGCAACA |
| | | GGCTTTCTTGCAATTTCGCATTGCCATTTGTGGTGGC |
| | | ACCTCTTCAAAACAAAACTTCTAGGGCTGGAGAGATG |
| | | GCTCAGCTGTTTAACGGCGCTGGTGGTTCTAGCAACA |
| | | AGAATGGAGGTTCCNTTTCTGGCACCCANACTG |
| S00082 | 82 | ATGCTTTTCAAAAAACAACAAAATATCCAAGTGTTTA |
| | | TTGGCCTCACCTTCTGTTCTCTACTTTATTGGAAAGA |
| | | GATGTACTGTGGCACCATTGACAGATGCCTTTTCTGG |
| | | TGGCGGTTCTTGTGGTCTGACTCTGGACTCAGACTCT |
| | | TGCCTGTTTGCCATCTGTAATAGGGATGGGCCCTTCC |
| | | CCTCTTGCATTTTTTCAAACACNGTTCTCCAAGGTAT |
| | | GTTCTGTCATCTGGCAAATGGGCACCTGGGA |
| S00083 | 83 | ATGGGNTATTNTCGCGTCTAGNGNNTNTATTTNCACC |
| | | ACCCCANCTCCTATACNAATANTCTGCTGCAAACTGG |
| | | NTCCNCAGGGGCGAGGATTTGCCTCTTGTGAANCNAC |
| | | TGTGGNCNTGGAACTGTGTGGAGGTGTATGGGGTGTA |
| | | NACCGGCANANACTCNNCCGGAGGACNGGGTAGAGCG |
| | | CCCCCCCCGAATTCCTGGACAAGCTTTGACTGG |
| S00084 | 84 | TTNTCACNACGANTTGAGTATTNGTGAACTGTATTAT |
| | | CGGTNTTAAAAATATATTCCGTNTCAAAATTTNGTTT |
| | | NCTGAAGAANTGAGTCNTATTNTAANAAAATTTGATA |
| | | TCNAAGGGGGGACAAAAATATAAAATTCCNGGAAAAC |
| | | ANNTGACAAATACACAATAGACCGGGGNCCCCCGAAT |
| | | TCCTGGACANACTTGANTNGNACGC |
| S00085 | 85 | ACTATGCAGCCAGTTCAAGCTAGTTTTGAACTTGCTG |
| | | TTCGCTTGCCTTGCCTTGGACTTCCCAGTGTTCGGAT |
| | | GANAGCCCACGCG |
| S00086 | 86 | GCNANAANAGGAAAGAATCATTATTNGGTNGAGGTCT |
| | | CCCACCTTGTCAGACNCANGTCACCANCTTTGGTGAC |
| | | AAGTGCCTTTACCCTGAGCCATCTCACTGGCCCGGCC |
| | | TGTGCGTACTNGTGTGTGTCTGTGTGCGCACGCNTGT |
| | | GCACNCACAGTTCACTTTNAGCATGCTGTATGTCAGC |
| | | TATAGTCCTGAGCCCTTCGCAGGCAGGACTGTNGCTG |
| | | ACCTTTACATNTTCCG |
| S00087 | 87 | ACACATGCCTTCCCCGCGAGATGGAGTGGCTGTTTAT |
| | | CCCTAAGTGGCTCTCCAAGTATACGTGGCAGTGAGTT |
| | | GCTGAGCAATTTTAATAAAATTCCAGACATCGTTTTT |
| | | CCTGCATAGACCTCATCTGCGGTTGATCACCCTCTAT |
| | | CACTCCACACACTGAGCGGGGGCTCCTAGATAACTCA |
| | | TTCGTTCGTCCTTCCCCCTTTCTAAATTCTGTTTTCC |
| | | CCAGCCTTAGANANACCCTGGCCGCCCGGGACGTGCG |
| | | TGACGCGGTCCAGGGTACATGGCGTATTGTGTGGAGC |
| | | GANGCAGCTGTTCCACCTGCGGTGACTGATATACGCA |
| S00088 | 88 | CTTGGCAGCCATTGTGTTTGTTACNGCANANCANACT |
| | | GCTGCAGGCCTGCCTCCCCTCTGAAGCTGCTTGTGCT |
| | | GCTGATAAACTCTGCCCCTTAGTTGCTCACTGTTNCT |
| | | CATACTGTGTGCANCCTGAGCCAGCCCGGGATGACCA |
| | | TCCTTACNGCAGCG |
| S00089 | 89 | GCTACAGCTCGTCAATGCACACGTTCTTTATATAATA |
| | | CTACACAGATCTTGTAAACGAAGTCTGGACATCAAAG |
| | | CTTTTATGGGAACTGCTAAGTGGTCTAAGGACGC |
| S00090 | 90 | ATATAATAAATCTAGAACCAATGCACAGAGCAAAAGA |
| | | CTCATGTTTCTGGTTGGTTAATAAGCTAGATTATCGT |
| | | GTATATATAAAGTGTGTATGTATACGTTTGGGGATTG |
| | | TACAGTCAGCTTTTTAATTAGCTTAACACACACATAC |
| | | GAAGGCAAAAATGTAACGTTACTTTGATCAGCTTTTA |
| | | ATTAGCTTAACACACACATACGAAGGTGTAACGTTAC |
| | | TTTGATCTGATCAGGGCCGACTTTTTTTTTNAATTNC |
| | | ANANTTNTCAATCCCATTANTAAAAGGGNAAACCTNG |
| | | GNTTTTNCCNGGAAGNAAGGGNTTAACGGTTTCCTT |
| S00091 | 91 | TTAGNTNNNCTGGAACTTGNTATGTANATGANGCTTG |
| | | NCTCNAACTCTGATATNCACTTGTGTCTGCCTCCTGA |
| | | CTATGTTGAACCANACCANTCTNTNATTCAAANANAC |
| | | TGAGGTTGGACCATCCTTANTCACCTGGGTTGTTCTA |
| | | TTGTTCTATTAANTGTAACTACACTCATAAATTCGAA |
| | | GCAAANCAAACCGTACCANCTGTGCTACTTTGANGCA |
| | | CCTGANCATTCNACAANGGATCTTTTTAACCTCATGA |
| | | GGCCCAGTCCTGCTAATCCAGGTTGGCTCNATCCTGC |
| | | AATCCCCTGCTCACAACACCTGT |
| S00092 | 92 | GTCAAAATACTGAGAATTAGAGGCTATTGGATGCCAA |
| | | GTCATAGAGAGGACACATATATACCAATACTTCCAAG |
| | | GCTCAGGAAACATCATGGAAGAAGGGGTAGGAAGAAT |
| | | TTAANAACCAGAAGAAGGGGGGTGAGGTATGGAATGA |
| | | TGATTTCCAGTCATGACTTGGCTATTAACCAGAAGAA |
| | | GGGGGGTGAGGTATGGAATGATGATTTCCAGTCATGA |
| | | CTTGGCTATTGAGTTAACAACAGCTGGATCACCTGCA |
| | | CAAGATCTCCACAAGAGTGGGCCCATTAACACTCTAT |
| | | CATGGAAAAGAGGAGGGGNTATGAGGTACCACCCCAC |
| | | CCTGAAGATTTATACACAATTAATANTTGGTGAGGTA |
| | | GGGAGAGACATTTACTTTAGGGGTGCAAGTCCACTAG |
| | | TACAGTGCCTAC |
| S00093 | 93 | CCATCTCTCCAGCCCCCCTCTCTTTCTAATATGTAGG |
| | | TCCCAGGGACCAGGCTCTAGCTCTCAGACTTTGCTAT |
| | | CTTCGTGTTGGAATTGTTTTACATTTATAAGGACTTT |
| | | GAAGCCTCATGTCACCTGCACCACCCCTCTGAGTCTG |
| | | ACC |
| S00094 | 94 | CAGCTGCGTTGCGTCATCCAGCCAGAGCTCAGAACAA |
| | | ACTATGAACTACAAAGTTCTTCAGCACCAAATCTCAG |
| | | AGGCAGAAAACATTCTAGGCCTAGATTAGATTGTACA |
| | | GAGGCTAAGAGGCTTCTAATAGACCTAGGTTTCCAGA |
| | | GAGAGGTTGTAAGCCACAAAGACCACAATTACATCAG |
| | | GCGAATGAGTTACTTTTACATATCTGTAAAATGAGCA |
| | | GAGAAGAGTCTGGGGCTCCTCTGTTCCCCGTGGTTTC |
| | | CTTGCTGGCCCTGGTTTTCCTGTGAGATGTGCCTGAC |
| | | TCCCCGGATGCCTTCAACTGATGTTGGCTTAGGGGGC |
| | | TGAGCTTTTAAATGTCAGATCTTCTCATTTCCGCCTC |
| | | TGTCCAGG |
| S00095 | 95 | AGNGGTACGCGGTANAGCANANACTANCNTACCCTTT |
| | | GGGCGCCTGTGGTCTCCACACAGAGTGTGTGGGTGTA |
| | | NGAACANGCTGATGGGGACTGCCTCTCGGCAGCCTTC |
| | | ACGGGCACCTGTGAGTGGCAGTCTGAAGGGTGGTGGC |
| | | CGGACANACANCCTATANAGTGATATTCCAAAGCCTG |
| | | AACCATTGTNGCTCCCGGCTGATTCCTGGTCTCGCCT |
| | | GATAGTTTTAGATGCACCATCTTATTTGTTCTTCACA |
| | | NGCAGTTATGCTAGANTGGATGA |
| S00096 | 96 | AAACCTGTGAGCTCTGCTTTTGTGCTCTACCCACAGG |
| | | AGCAGCCAGCCTTAAAACTGGAGCG |
| S00097 | 97 | ACAGCACCTATGGCTGTCCTCTGACCTCCACACACAT |
| | | GTGACATATGTCCATGTATACATACATGCACACACAC |
| | | ACACACA |
| S00098 | 98 | GTCTTCCTGGNCCTCCTGAGTCCCATCACTTCTCCAA |
| | | CTCTAAATCGGCCTGGGNCAACATGCTCAGCCAGCAG |
| | | TTAAGTCCCGTGCCCTCCCACCTGGAGNAGGTGTANN |
| | | AAATAGNGGNAAGGCCCAGGCGGCCTCGANCCCGAAG |
| | | GCATGAAGCCCCCGGGNACCGAGCACACACTGTCCTT |
| | | CCCCGGGTGCCGCTCACCATCTGTTGTGACACGGGGG |
| | | CCGAGNCCTGAAAGNGCTTGGCAGCCCCGGTGAGCGC |
| | | GAANNANNCGCCAAGCAGAACCCGCAACACGCCTACC |
| | | CTGAACGACATAGCAGCGC |
| S00099 | 99 | GGTAAGGAANGGCTCTCTCTGGTTTCCTCCCATGACA |
| | | GGNTTCTGTGAGGGCCACGCGTCCTGTTTACAGAATG |
| | | GTTTCCAAGTCACCGG |
| S00100 | 100 | GTGTATACAACGCCTTGTTCTAAACAACAAACCAGTG |
| | | CAGGGCTGTGGCGAAGCTANGTGGCAGATGCTTGCTT |
| | | AGCCAGGGTGAGGCTGGGTGCCACCTAACACTGAAAA |
| | | CGGANGCAGTGCAGANCCTANTGCACGTGAATTATCT |
| | | TCTCGGAATCATTACTTCCCCTGTTCCGCTTGTGGTG |
| | | CGTCTATAT |
| S00101 | 101 | GTTTAATCNAGCTTCACTAATATCAATTCGGAAGCTT |
| | | TCTCTCTGCTCCATTTATTTAAAAGCAATATTTATGA |
| | | TTGAGCCTGGGCATCTTAGCCCTAGCTAAGANGTTTT |
| | | AGATGTGTATTTTAATGTANATTAAAAAAACC |
| S00102 | 102 | CAAGANAGGACACTGGCAGGCTGGGGANGTGACTCAT |
| | | TCTGTAAGGGCCTGTCGCACANNCAAAAAGACCTGAA |
| | | TTTGATTCCANAATTCACATAAAAGTCAAGCNTGGTG |
| | | GGGTTTGTGATCCNANCACTGGGGAANCAGAGATCGG |
| | | GGGTCTCTNGACCNGTTAATTANGCCAMNAATCTAT |
| S00103 | 103 | CACATATACACACATGCACACCTGTGTACACATATAT |
| | | ACACATGTGTATGCACACACATATAAGCACATGCATG |
| | | CATGCACACACATGCACATGTGTGTACACATACCCAC |
| | | ACNTGTATACACACACCCACACATGTGTGTACATACA |
| | | CATACACACNTGCGTATATAC |
| S00104 | 104 | CTGGGAAGTCCGGGTTTTCCCCACCCCCCAATTCATG |
| | | GCATATTCTCGCGTCTAGCGCCTTGATTTTCCCCACC |
| | | CCAGCTCCTAAACCAGAGTCTGCTGCAAACTGGCTCC |
| | | ACAGGGGCAAGAGGATTTGCCTCTTGTGAAAACCGAC |
| | | TGTGGCCCTGGAACTGTGTGGAGGTGTATGGGGTGTA |
| | | GACCGGCAGAGACTCCTCCCGGAGGAGCCGGGTAG |
| S00105 | 105 | GTGGAANACGCCTTTTACCCTAGCAGAGGCAGAAGCA |
| | | GAGGTAGACGGATCTCTGTAAACCTGAGGCC |
| S00106 | 106 | TTANAAAGTGTNTATGTANACGTCNGGGGATNGTNCA |
| | | NANTGCACNCCNTAATATTCANGANAAAGGAACTGGG |
| | | AAANTNATNTATNAATNNNAATCNCCTNTNAANTAGC |
| | | TTAA |
| S00107 | 107 | TTATNACTCCACANACTGAGCGGGGGCTCCNNGATAA |
| | | CTCATTCGTTCGTCCTTCNCCCTTTCNAATTCTGTTT |
| | | TCCCCAGCCTTAGAGAGACNCCTGGCCGCCCGGGACG |
| | | TGCGTGACGCGGTCCAGGGTACATGGCGTATTGTGTG |
| | | GAGCGAGGCAGCTGTTCCACCTGCGGTGACTGATATA |
| | | CGCAGGGCAAGAACACAGTTCAGCCG |
| S00108 | 108 | GGTACAGTCAAACCATTGGGTTTCCAGTTGTATAAAA |
| | | GCAAGCACATACAATTATGTANAGCACACAGGTNGTG |
| | | TGTGTGTGTGTGTGTGTGTGTGTGTGTGTGTGTGTGT |
| S00109 | 109 | GGTCCGCGTGGTCCATGTGTAATGTGTCAGATGTGGG |
| | | GCTATAGGTGTGACTCCAGTCTCAGAATTGGGGGCTA |
| | | TGCAGCTGCACCGG |
| S00110 | 110 | ANATCATCAGATGCATTCTGTGGAAAGGACCTGGAGC |
| | | ATGAATGNNNANCAGCCCCAGTCTGCAACACTACTGG |
| | | GCATNANGCTTCAACAAGGGAAACATAATGGNGGTTT |
| | | CCCCTCNAAAGCAATTATNGGATACTGGTCTCTTTTC |
| | | TAATCTCTTTACTTCCTANTT |
| S00111 | 111 | CTANACGTTCTGGAGAGCTCAAAAGGANATTATCACC |
| | | CACTANTAANCTANTAAGAAAATCCATGATGTGTCTA |
| | | CNCATNNGCACATGTAGCTTCNTGGCTGCGCNTCCTG |
| | | GAANTCTGCACAGTTCTCCCACACCACTCATANGTAC |
| | | ANCA |
| S00112 | 112 | CAAAAATNAAGAAACGTAAAAAACTAAGTGAGCTCTC |
| | | AGTCCTCTAAGAAAAAACNAACTTCTCAGTGCTGTTG |
| | | TGTCATCTGCTTTACACANAGGAAAACCGTGGCAGAG |
| | | CANAACGCANCACAGGCC |
| S00113 | 113 | CANTGANGNNGGCTCAAATGGTTAGTCCTGGTGTATG |
| | | TTGCAAAGGGCACTCATAGTTTACTCTGGCTTTGGGG |
| | | CTTTGGTTCCCCAGGAGGGAAACAGACCCATCCANTG |
| | | TGCCCCTCCACNAGGTCGGCTTTGTTTAAAAATACCT |
| | | GCNGCATTCCAGATCANCTGAGAACCNCTGAAAAAGA |
| | | CTTTTTTGTTCCCTTCCCCTTTCCAGGGTAGACGGCN |
| | | NAGTCAANCNTTNCNTCATTAACAANACTGCCACCGG |
| | | CTATNGCTTTGCCGAGCCCTACAACCTGTACAGC |
| S00114 | 114 | AGNACCNGTTCGCCAAGAGGACTCANGCCAAGAAAGA |
| | | ACGCGTGGCCAANAATGAGCTGAACCGTCTGCGGAAC |
| | | CTGGCTCGCGCGCACAATATGCANATGCCCANCTCNG |
| | | CCGGNCTGCACCCTACTGGACACCAGAGTAAGGAANA |
| | | GCTGGGCCGCGCCATGCAAGTGGCCAAGGTTTCCACC |
| | | GCTTCGGTGGGACGCTTCCAGGAGCGC |
| S00115 | 115 | TTCCCTTTCAGCTGCTTTCAGGCATGCCCACCCATCC |
| | | ANCACTCCCCCCAACCCCACCCCGTGAATACACAGAG |
| | | NGNGACAAACTCTGTGTGTGTGTGTGTGTGTGTGTGT |
| | | GTGTGTGTGNGAGAGAGAGAGAGAGAGAGAGANANAN |
| | | ANAGAGAGAGAGAGAGAGAGAGAGAGAGAGA |
| S00116 | 116 | AGTGTATGTATACNTTTGGGGATTGTACAGAANGCAC |
| | | AGCGTAGTANTCAGGAAAAAGGAAACTGGGAAANTAA |
| | | TGTATAAATTAAAATCAGCTTTTAANTAGCTTAACAC |
| | | ACACATACNAAGGCAAAAATGTAACGTTNCTTTGATC |
| | | TGATCAGGGCCGACTTTTTTTTTNANNTGNNNAATTN |
| | | CNATNCCNNNANTAAAAGGGGAAAGNTNGGNTTTNTC |
| | | NNGGGNGNAAGGGNTTAANGNTTTTNTTTNTT |
| S00117 | 117 | AATCCTTTCTGTACTGAGTGCCTGGGGAGGCAGAGAG |
| | | CAGAAGTCTCCAGCCCAGTGAATACTCTTCTCACCAC |
| | | TAGACCCCAGCTCCTGCCTCAGCCTCCCCAGCCTGGC |
| | | TATCAGAGCTTGCCCCACTCTATTTCCCAGGC |
| S00118 | 118 | AGTCAACATAACTGTACGACCAAANGCAAAATACACA |
| | | ATGCCTTCCCCGCGAGATGGAGTGGCTGTTTATCCCA |
| | | GTGGCTCTCCAAGTATACGTGGCAGTGAGTTGCTGAG |
| | | CAATTTTAATAAATTCCAGACATCGTTTTTCTGCATA |
| | | NACCTCATCTGCGGTTGATCACCCTCTATCACTCCAC |
| | | ACACTGAGCGGGGG |
| S00119 | 119 | TTATNTCTCCATGGCTCCAACTGGANGGAGANGNNGA |
| | | GGGACACTTANAATTCGNCNNNGCAACNTTGAATTTT |
| | | TCCAGAAAAGANTGCTTTCACGCCATGCAACATGGGA |
| | | NAAGGANATGGANGTGAAANTTTCCATGGACAGAAAG |
| | | TAANAACACTCANCNCTNANTTGAGGGCCTGAANTNT |
| | | GCNTCCATTATA |
| S00120 | 120 | TGNGCATACACACCTTAGCCGAAGGTGCCTGAAATCC |
| | | GCTCAGGGTAACCTAGGCGGAGCAGCCGTGTAGCACG |
| | | TGGGCTGCCACGCG |
| S00121 | 121 | CCCCCAATTCATGGCATATTCTCGNGTNTAGCGCCTT |
| | | GATTTTCCCCACCCCAGCTCCTAAACCAGANTCTGCT |
| | | GCAAACTGGCTCCACAGGGGCAAANAGGATTTGCCTC |
| | | TTGTGAAAACCGACTGTGGCCCTGGAACTGTGTGGAG |
| | | GTGTATGGGGTGTANACCGGCAGANACTCCTCCCGGA |
| | | GGAGCCGGGTAGAGCGCC |
| S00122 | 122 | CTGNTGCCAGCTTAAAGCTCAAAGCTTTTCCACTCCA |
| | | GTGCAAAGAGATGAGATTTGAATCAACAGAATTTGTT |
| | | GGACTTAAATGTCATTTTAATTTTTTAACTGATCTAG |
| | | AAAAGCACAAGGTGCACGTNTTTCTGGGGCAGCATGT |
| | | GTGTGTCAATATGCAAACCTGGGCTAATTAGACCACT |
| | | TCACTTCACTGAAACAGAAACCACTAGATTCCCTGTG |
| | | AATCCCTCTCTTCAGGAGGCCATGGGGGCAGGAGCAC |
| | | CCCTACTCTGGGGGGCACTGGACCCCC |
| S00123 | 123 | CTCCTATTCAGTCACACCCTGCTGCCCCATANATCTC |
| | | TACTTGAAAGAGGGGAGTTAACCAGCAAGCCTCAGGA |
| | | TAAGAGGACAGAAGTCACAAAAGCCACAGGAGGC |
| S00124 | 124 | TGGTGAAACTGGCCCAGGCTGGTCGGGAGGGCAAGGA |
| | | AGGAATACAGGACGATCTGCNCATCGTATTGCTTCCA |
| | | ACCTGAAAAAGGAGCAGTGTGGCAACAGGCTGCTTTT |
| | | TTACAGGCTGGGATGCATTTCGTCCCCCTACCTGCCT |
| | | CGACAGCCCTGCGCACTGCAGGAAGGAGACGAAAGCA |
| | | TTGACCACCCCGAACCGCCNAGGGAGGGCGGCTGGGA |
| | | GCGGACAAGACCGAAGACAGCACCCAGCTTCAGCCTT |
| | | TCTAAGCCCGGCGAGNTCAGGAACCCCACAGACAAGG |
| | | GCCGCAGCGACTCGTGNANCTGCCGCTGGGAGGCTGT |
| | | AG |
| S00125 | 125 | ATCTNNNCNNNCTNTGACCTGTTNNGCTCTACNTCTA |
| | | TTCTCCCAAAAACNAANNCCTAGACCAAGGTNTCTGT |
| | | TTCANCNTNNACTTTTAAGTGAAACCAAATTAAANCN |
| | | GGNGACACTGGNAGAGGGGAGTCACTGAC |
| S00126 | 126 | GTATGGAGAGTGCAATGCTTGGTGGCTTCCTGGGTGC |
| | | ACCCATGCCCAGCGC |
| S00127 | 127 | CTCAAACTCCCTCCTCTTGCTCTCCTCACCCACTTGC |
| | | GTTTATNTCGAAAGCTCTCTTACTCATCTTTCCCCTT |
| | | TTCTGTCCTTCGATGTCTCTGATTCTTTCTCCANCTC |
| | | TGTTCCCTCCTCTTTTCCCGGTGTCTCTGTCTCCGGC |
| | | T |

Contigs assembled from the mouse EST database by the NCBI having homology with all or parts of the LA nucleic acid sequences of the invention are depicted in Table 2.

TABLE 2

MOUSE

| | | |
|---|---|---|
| SAGRES | REF | SEQ |

| | | | |
|---|---|---|---|
| TAG # | # | ID # | SEQUENCE |

| | | | |
|---|---|---|---|
| S000004 | F1 | 128 | CGGCCAGGGACTCCCCTCCAGGCTCCTCAGAGAG |
| | | | CAACAGGCGAAGAGAACTAAACTGTTTTGCCCTC |
| | | | TTCAAGATCAATAACCCTCATATACCCCAGGGAT |
| | | | GAAGGATGCTAAGCCCAATCCTGCTGCCTTGTCA |
| | | | CCCCTCTCCCTGTTGTGGGACCCAGGAAAGGGCC |
| | | | TTGGAGCATCTTACCCCACAGGGGACTCTTAAGA |
| | | | TCACTGCCATCCCTTCTCTAAGACAAAACCTTCC |
| | | | CTAACTATCACACATTTAAGTGTGCCATTCCAGA |
| | | | GGGCTCTACAAGGTCATTTTACCTTTCCTTAGAC |
| | | | AACTTACTAACCTCTTACAGATGAGGCGGAGATT |
| | | | CAAACAGAGATTCAAACAAGTTCCAGAACTCAGA |
| | | | GTCTACCGCATTTCCCACTGCACAGTTCTAGTCT |
| | | | CCAGGGATATGCTG |
| S000010 | F2 | 129 | ACTAGAGGCAGTAAAGTTTATTACATTAAAACTC |
| | | | AATGCTGGGTCAGAGGCATCCACACGGCCCTGAT |
| | | | CTCTGAATCCTGAAGGTGTGGAACCAGAAGCCGC |
| | | | TGTGACTTGCAGGGTCAGGACTTGGGTCTGCCTG |
| | | | CTTTGCATAGCTAGACTCCTATGCATCCTTTCAG |
| | | | AGGTCACCCAATGTCCCAGTCAAAAGCAGCTGTT |
| | | | GCTCTGTGGCCATATGGCACTACTCCTCACAGAG |
| | | | CAGCGCCTGTGGAAGGATCTTCCAACAGCACATG |
| | | | GACATAGTCCCTGACGTCCACACCCGGGGCTACC |
| | | | AGGAAGCCCCAGGGCTGCGTCTGGCTCCTCACAT |
| | | | CCTTTTCCTCATCTTGCCCTTCCTGGAGGGAGCA |
| | | | CCCCGGCCAAAGGCGCCCTGGCGCCCGCTCCTGG |
| | | | GCTCGGCGTCGGTTGCTTGGGTCCTTGCTGGAGG |
| | | | CATTGATCTCAAAGATGGTTGTGCGCGTGCGATA |
| | | | GTTCTTGATGCTGTCCACCAGCCTCAGGCGTTGG |
| | | | AGCTCTCCCTCCTCAAAGCATGAGCTGAAGAGTG |
| | | | GGTGCAAGCCCAGCTCTGCCAGGTCCAGCTCCTT |
| | | | GGCTCTCTTGATGGACTCAGGCGAGGGCGCTGGC |
| | | | CGTGAGCGCACATACTGCTGCTGAGCGTTGT |
| S000013 | F3 | 130 | CCGCCACCAAACGCCGGTTAAACCACCTCGGAGA |
| | | | CTGCTGTGCGGAGAGGACTGGGAAACCGGTCCCC |
| | | | ACACACTGTCCACGCTGGCTCCCCACGGAGGCCC |
| | | | ACCCACACCCGCGGCCCGGGGCAAGATGCAGTGA |
| | | | TCTCAGCCCTCCCGCTCCTCCGCACTTCCGCCTC |
| | | | AGTATGGCCTCACAGCTGCAGGTGTTTTCGCCCC |
| | | | CATCAGTGTCGTCGAGTGCCTTCTGCAGTGCAAA |
| | | | GAAACTGAAATAGAGCCCTCTGGCTGGGATGTTT |
| | | | CAGGACAGAGCAGCAACGACAAATACTATACCCA |
| | | | CAGCAAAACCCTCCCAGCTACACAAGGGCAAGCC |
| | | | AGCTCCTCTCACCAGGTAGCAAATTTCAATCTTC |
| | | | CTGCTTACGACCAGGGCCTCCTTCTCCCAGCTCC |
| | | | TGCCGTGGAGCATATTGTGGTAACAGCTGCTGAT |
| | | | AGCTCAGGCAGCGCCGCTACAGCAACCTTCCAAA |
| | | | GCAGCCAGACCCTGACTCACAGGAGCAACGTTTC |
| | | | TTTGCTTGAGCCATATCAAAAATGTGGATTGAGA |
| | | | GAAGAGTGAGGAAGTGGAGAGCAACGGTAGCGTG |
| | | | CAGATCATAGAAGAACACCCCCCTCTCATGCTGC |
| | | | AGAACAGAACCGTGGTGGGTGCTGCTGCCACGAC |
| | | | CACCACTGTGACCACCAAGAGTAGCAGTTCCAGT |
| | | | GGAGAAGGGGATTACCAGCTGGTCCAGCATGAGA |
| | | | TCCTTTTGCTCTATGACCAACAGCTATGAAGTCC |
| | | | TGGAGTTCCTAGGCCGGGGGACATTTGGACAGGT |
| | | | GGCAAAGTGCTGGAAGCGGAGCACCAAGGAAAGT |
| | | | GGCCATTAAGATCTTGAAGAACCACCCCTCCTAT |
| | | | GCCAGACAAGGACAGATTGAAGTGAGCATCCTTT |
| | | | CCCGCCTAAGCAGTGAATGCTGATGAGTATAACT |
| | | | TTGTCCGTTCTTATGAGTGTCAGCACAAGAATCA |
| | | | TACCTGCCTTGTGAAAGAGATGTTGGAGCAGAAC |
| | | | TTGTACGATTTTCTAAAGCAGAACAAGTTTAGCC |
| | | | CACTGCCACTCAAGTACATAAGACCAATCTTGCA |
| | | | GCAGGTGGCCACAGCCCTGATGAAGCTGAAGAGT |
| | | | CTTGGTCTGATTCATGCTGACCTTAAACCTGAAA |
| | | | ACATAATGCTAGTCGATCCAGTCGCCAACCCTAC |
| | | | CGAGTGAAGGTCATTGACTTTGGTTCTGCTAGTC |
| | | | ATGTTTCCAAAGCCGTGTGTTCAACCTACCTGCA |
| | | | ATCACGCTACTACAGAGCTCCTGAAATTATCCTT |
| | | | GGATTACCATTCTGTGAAGCTATTGACATGTGGT |
| | | | CACTGGGCTGTGTAATAGCTGAGCTGTTCCTGGG |
| | | | ATGGCCTCTTTATCCTGGTGCTTCAGAATACGAT |
| | | | CAGATTCGCTATATTTCACAAACACAAGGCCTGC |
| | | | CAGCTGAGTATCTTCTCAGTGCCGGAACAAAAAC |
| | | | AACCAGGTTTTTTAACAGAGATCCTAATTTGGGG |
| | | | TACCCACTGTGGAGGCTTAAGACACCTGAAGAAC |
| | | | ATGAATTGGAAACTGGAATAAAGTCAAAAGAAGC |
| | | | TCGGAAGTACATTTTTAACTGTTTAGATGACATG |
| | | | GCTCAGGTAAATATGTCTACAGACTTAGAGGGGA |
| | | | CAGATATGTTAGCAGAGAAAGCAGATCGGAGAGA |
| | | | GTATATTGATCTTCTAAAGAAAATGCTGACGATT |
| | | | GATGCAGATAAGAGAATCACGCCTCTGAAGACTC |
| | | | TTAACCACCAATTTGTGACGATGAGTCACCTCCT |
| | | | GGACTTTCCTCACAGCAGCCACGTTAAGTCCTGT |
| | | | TTCCAGAACATGGAGATCTGCAAGCGGAGGGTTC |
| | | | ACATGTATGACACAGTGAGTCAGATCAAGAGTCC |
| | | | CTTCACTACACATGTCGCTCCAAATACAAGCACA |
| | | | AATCTAACCATGAGCTTCAGCAACCAGCTCAACA |
| | | | CAGTGCACAATCAGGCCAGTGTTCTAGCTTCCAG |
| | | | CTCTACTGCAGCAGCAGCTACCCTTTCTCTGGCT |
| | | | AATTCAGATGTCTCGCTGCTAAACTACCAATCGG |
| | | | CTTTGTACCCATCGTCGGCAGCGCCAGTTCCTGG |
| | | | AGTTGCCCAGCAGGGTGTTTCCTTACAACCTGGA |
| | | | ACCACCCAGATCTGCACTCAGACAGATCCATTCC |
| | | | AGCAAACATTTAATAGTATGCCCACCTGCTTTTC |
| | | | AGACTGGACTACAAGCAACAACAAAGCATTCTGG |
| | | | ATTCCCTGTGAGGATGGATAATGCTGTGCCAATT |
| | | | GTACCCCAGGCGCCTGCTGCTCAGCCGCTGCAGA |
| | | | TCCAGTCAGGAGTACTCACACAGGGAAGCTGTAC |
| | | | ACCACTAATGGTAGCAACTCTCCACCCTCAAGTA |
| | | | GCCACCATCACGCCGCAGTATGCGGTGCCCTTTA |
| | | | CCCTGAGCTGCGCAGCAGGCCGGCCGGCGCTGGT |
| | | | TGAACAGACTGCTGCTGTACTGCAAGCCTGGCCT |
| | | | GGAGGAACCCAACAAATTCTCCTGCCTTCAGCCT |
| | | | GGCAGCAGCTGCCCGGGGTAGCTCTGCACAACTC |
| | | | TGTCCAGCCTGCTGCAGTGATTCCAGAGGCCATG |
| | | | GGGAGCAGCCAACAGCTAGCTGACTGGAGGAATG |
| | | | CCCTCTCATTGGCAACCAGTACAGCACTATTATG |
| | | | CAGCAGCCATCTTTGCTGACCAACCATGTGACCT |
| | | | TGGCCACTGCTCAGCCTCTGAATGTGGTGTTGCC |
| | | | CATGTTGTCAGACAACAACAGTCTAGTTCCCTCC |
| | | | CTTCAAAGAAGAATAAGCAGTCTGCTCCAGTTTG |
| | | | ATCCAAATCCTCTCTGGAAGTCCTGCCTTCTCAA |
| | | | GTTTATTCTCTGGTTGGGAGTAGTCCTCTTCGTA |
| | | | CCACATCTTCTTCATAATTCCCTAGTTCCTGTCC |
| | | | AAGACCAGCATCAGCCAATCATCATTCCAGATAC |
| | | | CCCCAGCCCTCCTGTGAGTGTCATCACTATCCGT |
| | | | AGTGACACTGATGAAGAAGAGGACAACAAATACA |
| | | | AGCCCAATAGCTCGAGCCTGAAGGCGAGGTCTAA |
| | | | TGTCATCAGTTATGTCACTGTCAATGATTCTCCA |
| | | | GACTCTGACTCCTCCCTGAGCAGCCCACATCCCA |
| | | | CAGACACTCTGAGTGCTCTGCGGGGCAACAGTGG |
| | | | GACCCTTCTGGAGGGACCTGGCAGACCTGCAGCA |
| | | | GATGGCATTGGCACCCGTACTATCATTGTGCCTC |
| | | | CTTTGAAAACACAGCTTGGCGACTGCACTGTAGC |
| | | | AACACAGGCCTCAGGTCTCCTTAGCAGTAAGACC |
| | | | AAGCCAGTGGCCTCAGTGAGTGGGCAGTCATCTG |
| | | | GATGCTGTATCACTCCCACGGGGTACCGGGCTCA |
| | | | GCGAGGGGGAGCCAGCGCGGTGCAGCCACTCAAC |
| | | | CTTAGCCAGAACCAGCAGTCATCGTCAGCTTCAA |
| | | | CCTCGCAGGAAAGAAGCAGCAACCCTGCTCCCCG |
| | | | CAGACAGCAGGCATTTGTGGCCCCGCTCTCCCAA |
| | | | GCCCCCTACGCCTTCCAGCATGGCAGCCCACTGC |
| | | | ACTCGACGGGGCACCCACACTTGGCCCCAGCCCC |
| | | | TGCTCACCTGCCAAGCCAGCCTCACCTGTATACG |
| | | | TACGCTGCCCCCACTTCTGCTGCTGCATTGGGCT |
| | | | CCACCAGTTCCATTGCTCATCTGTTCTCCCCCCA |
| | | | GGGTTCCTCAAGGCATGCTGCAGCTTATACCACA |
| | | | CACCCTAGCACTCTGGTGCATCAGGTTCCTGTCA |
| | | | GTGTCGGGCCCAGCCTCCTCACTTCTGCCAGTGT |
| | | | GGCCCCTGCTCAGTACCAACACCAGTTTGCCACT |
| | | | CAGTCCTACATCGGGTCTTCCCGAGGCTCAACAA |
| | | | TTTACACTGGATACCCGCTGAGTCCTACCAAGAT |
| | | | CAGTCAGTATTCTTACTTGTAGTTGATGAGCACG |
| | | | AGGAGGGCTCCGTGGCTGCCTGCTAAGTAGCCCT |
| | | | GAGTTCTTAATGGGCTCTGGAGAGCACCTCCATT |
| | | | ATCTCCTCTTGAAAGTTCCTAGCCAGCAGCGCGT |
| | | | TCTGCGGGGCCCACTGAAGCAGAAGGCTTTTCCC |
| | | | TGGGAACAGCTCTCGGTGTTGACTGCATTGTTGC |
| | | | AGTCTCCCAAGTCTGCCCTGTTTTTTTAATTCTT |
| | | | TATTCTTGTGACAGCATTTTTGGACGTTGGAAGA |
| | | | GCTCAGAAGCCCATCTTCTGCAGTTACCAAGGAA |
| | | | GAAAGATCGTTCTGAAGTTACCCTCTGTCATACA |
| | | | TTTGGTCTCTTTGACTTGGTTTCTATAAATGTTT |
| | | | TTAAAATGAAGTAAAGCTCTTCTTTACGAGGGGA |
| | | | AATGCTGACTTGAAATCCTGTAGCAGATGAGAAA |
| | | | GAGTCATTACTTTTTGTTTGCTTAAAAAACTAAA |
| | | | ACACAAGACTTCCTTGTCTTTTATTTTGAAAGCA |
| | | | GCTTAGCAAGGGTGTGCTTATGGCGTATGGAACA |
| | | | GAATGATTTCATTTTCATGTCGTGCTGTCCTTAC |
| | | | TGGGCAGTTGTTAGAGTTTTAGTACAACGAGTCA |
| | | | CTGAAACCTGTGCAGCTGCTGCTGAGCTGCTCGC |
| | | | AGAGCAGCACTGAACAGGCAGCCAGCGCTGCTGG |
| | | | GAAGGAAGGTGAGGGTGAGGACTGTGCCCACCAG |
| | | | GATTCATTCTAAATGAAGACCATGAGTTCAAGTC |
| | | | CTCCTCCTCTCTCTAGTTTAACTTAAATTCTCCT |
| | | | TATAGAAAAGCCAGTGAGGTGGTAAGTGTATGGT |
| | | | GGTGGTTTGCATACAATAGTATGCAAAATCTCTC |
| | | | TCTAGAATGAGATACTGGCACTGATAAACATTGC |
| | | | CTAAGATTTCTATGAATTTCAATAATACACGTCT |
| | | | GTGTTTTCCTCATCTCTCCCTTCTGTTTCATGTG |
| | | | ACTTATTTGAGGGGAAAACTAAAGAAACTAAAAC |
| | | | CAGATAAGTTGTGTATAGCTTTTATACTTTAAGT |
| | | | AGCTTCCTTGTATGCCAACAGCAAAGAATGCTCT |
| | | | CTTACTAAGACTTATGTAATAAGTGCATGTAGGA |
| | | | ATTGCAGAAAATATTTTAAAAGTTTATTACTGAA |
| | | | TTTAAAAATATTTTAGAAGTTTTGTAATGGTGGT |
| | | | GTTTTAATATTTTGCATAATTAAATATGTACATA |
| | | | TTGATTAGAAGAAATATAACAATTTTTCCTCTAA |
| | | | CCCTGTTATTTGTAATCAAATGTTAGTGATTACA |
| | | | CTTGAATTGTGTATTTAGTGTGTATCTGATCCTC |
| | | | CAGTGTTACCCCGGAGATGGATTATGTCTCCATT |
| | | | GTATTTAAACCAAAATGAACTGATACTTGTTGGA |
| | | | ATGTATGTGAACTAATTGCAATTCTATTAGAGCA |
| | | | TATTACTGTAGTGCTGAGAGAGCAGGGGCATTGC |
| | | | CTGCAGAGAGGAGACCTTGGGATTTGTTTTGCAC |
| | | | AGGTGTGTCTGGTGAGGAGTTGTTCAGTGTGTGT |
| | | | CTTTTCCTTCCTCCTCTCCTCTCTCCCCTTATTG |
| | | | TAGTGCCTTATATGATAATGTAGTGGTAATAGAG |
| | | | TTTACAGTGAGCTTGCCTTAGGATGACCAGCAAG |
| | | | CCCCAGTGACCCCAAGCTGTTCGCTGGGATTTAA |
| | | | CAGAGCAGGTTGAGTAGCTGTGTTGTGTAAATGC |
| | | | GTTCGTGTTCTCAGTCTCCCTACCGACAGTGACA |
| | | | AGTCAAAGCCGCAGCTTTCCTCCTTAACTGCCAC |
| | | | CTCTGTCCCGTTCCATTTTGGATCTTCAGCTCAG |
| | | | TTCTCACAGAAGCATTCCCTAACGTGGCTCTCTC |
| | | | ACTGTGCCTTGCTACCTGGCTTCTGTGAGAGAGC |
| | | | AGGAAGCAGGCGAGAAGAGTGACGCCAGTGCTAA |
| | | | TATGCATATTTGAAGGTTTGTGCATTACTTAGGG |
| | | | TGGGATTCCTTTTTCTCTCCTCCATGTGATATGA |
| | | | TAGTCCTTTCTGCATAGCTGTCGTTTCCTGGTAA |
| | | | ACTTTGCTTGGTTTTTTTTTTTTTTGTTTGTTGT |
| | | | TTTTTTTTTAAAGCATGTAACAGATGTGTTTATA |
| | | | CCAAAGAGCCTGTTGTATTGCTTAATATGTCCCA |
| | | | TACTACCGAGAAGGGTTTTGTAGAACTACTGGTG |
| | | | ACAAGAAGCTCACAGAAAGGTTTCTTAATTAGTG |
| | | | ACGAATATGAAAAGAAAGCAAACCTCTTGAATCT |
| | | | GAACAATTCCTGAGGTTTCTTTGGGACAACATGT |
| | | | TGTTCTTGGGGCCCTGCACACTGTAAAAGTCCTA |
| | | | GTATTCAACCCCTCCATGGATTTGGGTCAAGTGA |
| | | | AGGTACTAGGGGTGGGGACATTCTTGCCCATGAG |
| | | | GGATTTGTGGGGAGAAGGTAACCCTAAGCTACAG |
| | | | AGTGGTCCACCTGAATTATATCAGAAGTGGTAAT |
| | | | TCTAGGATTGGTTCTGTGTAGGTGGTGTCAGGAG |
| | | | GTGCAGGATGGAGATGGGAGATTTCATGGAACCC |
| | | | GTTCAGGAAGCTCTGAACCAGGTGGAACACCGAG |
| | | | GGGCTGTCAACGAACTTGGAGTTTCTTCATCATG |
| | | | GGGAGGAAGAGTTTCCAGGGCAGGGCAGGTAGTC |
| | | | AGTTTAGCCTGCCGGCAACGTGGTGTGTGTTGTC |
| | | | TTTTCTTTAATCATTATATTAAGCTGTGCGTTCA |
| | | | GCAGTCTGTTGGTTGAGATAACCACGCATCATTG |
| | | | TGTAGTTTGTCACTAGTGTTATACCGTTTATGTC |
| | | | ATTCTGTGTGTGATCTTTGTGTTTCCTTTCCCCC |
| | | | AAGCATTCTGGGTTTTTCCTATTTAAATACAGTT |
| | | | CTAGTCTAGGCAAACATTTTTTTTAACCTTTTCT |
| | | | CTATAAGGGACAAGATTTATTGTTTTTATAGGAA |
| | | | TGAGATGCAGGGAAAAAACAAACCAACCCTGTCC |
| | | | CCACTCCTCACCTCCCTAATCCAATAAGCAGTTA |
| | | | TTGAAGATGGGAGTCTTAAATTTATGGGAAAGAG |
| | | | GATGCCTAGGAGTTTGCATCGTTACCTGAGACAT |
| | | | CTGGCTAGCAGTGTGACTTACAGACTTTGAGGTT |
| | | | GTCACTCTGCAAACTGACATTTCAGATTTTCCTA |
| | | | GATAACCCATCTGTGTCTGCTGAATGTGTATGCG |
| | | | CCAGACATAGTTTTACATTCATTCTGGCCTGGGG |
| | | | CTTAACATTGACTGCTTGCCCTGATGGCATGGAG |
| | | | GAGAGCCCTACGAACATAGCGCTGACTAGGTCAG |
| | | | CATTGCCTGACCTTGGAACAGCTTAAGGCTTTCC |
| | | | TTCTCTTAGAACGTGCATTTCCAGTTTCTCCCTC |
| | | | CCAGGTGAGAGAGGAACTGGAAGGGTTGCATAGG |
| | | | CACACACCAGGACACTTAGTCACTCCAGAGTCCC |
| | | | CAGTTGCAACTAGGAGGTGGTTACCCTGTTAACC |
| | | | CCAGGAAGAAGAACCCCATTTCAAACAGTTCCGG |
| | | | CCATTGAGAGCCTGCTTTTGTGGTTGCTCATCCG |
| | | | TCATCATCCGCTAGAGGGGCTTAGCCAGGCCAGC |
| | | | ACAGTACTGGCTGTCCTATTCTGCATTAGTATGC |
| | | | AGGAATTTACTAGTTGAGATGGTTTGTTTTAGGA |
| | | | TAGGAGATGAAATTGCCTTTCGGTGACAGGAATG |
| | | | GCCAAGCCTGCTTTGTGTTTTTTTTTAAATGATG |
| | | | GATGGTGCAGCATGTTTCCAAGTTTCCATGGTTG |
| | | | TTTGTTGCTAAAATTTATATAATGTGTGGTTTCA |
| | | | ATTCAATTCAGCTTGAAAAATAATTTCACTATAT |
| | | | GTAGCAGTACATTATATGTACATTATATGTAATG |
| | | | TTAGTAAAAAGCTTTGAATCCTTGATATTGCAAT |
| | | | GGAATCCTAATTTATTAAATGTATTTGATATGCT |
| | | | AAAAAA |
| S000015 | F4 | 131 | CCGGTCACATGCTTTCTTTGTGATGACCATCGTG |
| | | | ATGGGTTCCGTAGAGGTGGGAGCAGCAGCTAAGT |
| | | | CAAGAGCATTTGTGAGTATGACTCTAGCAGCTGG |
| | | | ACACACAGAGAAATGTGCATCCCAGCTATAACTA |
| | | | ATCAAGAAAGGCCTGGCTGTGGAATTCACAGGGG |
| | | | TCCTTACTGGATTCACAGGCTTTGATATACCTTG |
| | | | AAGAAGTGACACTTTTTTCCCCCCTTGGCTCTCA |
| | | | GCCTTTCTCCAGGCTAATTCATATTTACTTAGAT |
| | | | GGCTCTAGATATTCTCTCACTAACCTGAACCTTT |
| | | | GGCATCAACACAGGCTTAAAGGACATACTTAGGG |
| | | | TCTCTAGTGTCAATTGAATGGCAGCATCCTGACT |
| | | | TTGGTCTTCAAAGCAAAGATGACACTGAAGTCTG |
| | | | CCCCTTCCAAACAAGGGCTACCCTGCCTGCTTCC |
| | | | AGAAGCAAAGCACGCCTTACCATCTGCTTAGGAC |
| | | | TTCACAGTTCATAAAGTTCTTTCCATCCCGTCTG |
| | | | CTTTCTTTTTATTGCACAAGTGTTTACTTTTTAT |
| | | | TGCTCAGTATTTACTGAGATACCGCAGATGCCAC |
| | | | TGTGCAGGGCGCCTGCGGTCCTTGAGGAAGAGCT |
| | | | GTTGTTCCCATGCCTAGGCAATTCAGAAGGCCAT |
| | | | GGCTGGAATCTGGGGGCAATTGCATAGCCTGAAA |
| | | | TCAGGCTGCTAGCTGTAGTGGCTTTCCCAAGAGA |
| | | | ACACGGGGCTTCTGTTTCTGGACCTGTCTGATGA |
| | | | GGACACCCTTTCCTGTCTCCTGCCTTCTTCTCCA |
| | | | GCAGGGTTCCCCCTCCTTTCCTATTCCCCCACGT |
| | | | CTTCTCATCCCCTTCCCGTCTCCACTTACCCCCT |
| | | | CCTACCAGCTCATTTCTTCTGAAGATGAGCCGGA |
| | | | TTCTTTCTACAGTACTTTTGTGGGATGTGAATCT |
| | | | GACTATGCAGAGCTGGGCCTGGGATTTGTGTAAC |
| | | | TTCCCTTGAGAGCATAGCCTTAGCTCTTATTCTG |
| | | | TTATTCATTATTTGTAATGAATGCAGGATGCTCC |
| | | | AGTGCCCTCCTTGTCCTCAACTCTTCTGTGTCTA |
| | | | TAGTCAGGTGCTATAGCAGGTTGAGGTTCTAGCT |
| | | | ATATATAAGCTACTATCTCTATCATTAAAATATT |
| | | | TCAGGTTGTTGGTGGCACATGCCTTTAATCTCAG |
| | | | CATTTAGGAGGCAGAGGAAAAAGGATCTCTTGAG |
| | | | TTTGAGACTAGCCTGGCTGGTCTACAGAGTGAGT |
| | | | TTCAGGACAGCTACAGCCACACAGAAAAACCTTG |
| | | | TCTTGGGGGTTGGGGTGGGGAATCTAGATATATT |
| | | | AGTCAGGATTGTCTTGAACGATAGAGCCAATGTG |
| | | | CAATGAAAGATAGACATGTATCTCAATATCTGTG |
| | | | TCTATATGGAGAAGGATTTATTTTTCATAAGGCA |
| | | | TTGACAGAGATTATCATGGAGCTTGTGAAGTTCT |
| | | | GATGGTCTGCTGTGTATACCTGGAAACTAGAGAA |
| | | | GCTGGCTGTGTGCATAGACAGAATTATGAAAGAG |
| | | | TGTCTCAGCGCAAGTGCCCAGGCAGAGAAAGAAT |
| | | | GAACTTGCTTCTCCTGCTTCCTTATTCAGCTTTC |
| | | | TAGGCATCCTTGAGTTCTGATCCTCAGTGGGCTG |
| | | | GATGATGTTCACCCATACTGATGTAAGCTACTCA |
| | | | CCACACTCACTCACTTTCCCTCCCTTCTCTGGAA |
| | | | ACACCATCATCAATCCTCCTTAGAATGTCCTTAA |
| | | | CTGGTTCCCTTTGTAGCTCTTGGCCCAGCCAAAT |
| | | | TGACACACTGAGTAGACACAATGTATCTAACCAT |
| | | | CAATTGAGACACTGGGGAGACACAATGTATTCAA |
| | | | TTGTCTGAATCAGCTGGCTGACATCCACCTCAGG |
| | | | CCACAAGCTGAACGCACTTAGACTGCTGAGGGCA |
| | | | CAAAAGCACTCCCTTCCAATCCAATCCAAGTTTT |
| | | | GCAACAAGGTAGACCAAATCGAGTCATCATAAGT |
| | | | ATGTCCTTATCTGGCTATGCCCTGCTTTGATGTT |
| | | | TACCCAATACAGAACCCCCACTGATTGATGATAT |
| | | | TTGCTTCCTCATCACTACAACTTGGCCTGTAATG |
| | | | AGCACTGCTGTTTTACAGCATCAGGCTGCTAGGA |
| | | | CTATGTATAGAGAGAGAGCTTTGGCTTTGCTCTG |
| | | | GTCTTATACCTTGTGACCCATTGAACACCTCACT |
| | | | TTCAAGACCTGATGGGATTCATCTAGGACTCTGG |
| | | | TCCTTCCTTCAGATGTGTGTATGTTGTATCAGTC |
| | | | CCTCAGTCCCTTCTCCTGAATCCTGCTAGGAGAC |
| | | | CTCACAGCACAGTATTCTATCTGCTAAAGGAGTT |
| | | | TGCTTTCCTTCAATGATGCTGTAGTGATGCTGCT |
| | | | GGAGGAGTAGCTGGTTCTAGTAATGTTGGTGTTG |
| | | | AGGAAGATAATAATAATACTGGGGACATTGCTTT |
| | | | TGAATTAGGGGACTAGCTCAAGTATATTATTTTT |
| | | | CATATCTCATCTCATCTCATCTCATCTCATCTCA |
| | | | TCTCATCTCATCTCATCTCATCTCATCTTCTTTC |
| | | | CTCTCCATACTTATGTTGCCTATTCAGGAATATT |
| | | | TTGGCTATTGTACCTGTGGATATTCATTACAAAG |
| | | | GAGGCAGTGGCTCAAATGAAGCCAAAGAGCCTGG |
| | | | CTCTGAAGGACTGATGCCAGGTGGCCAGACATAG |
| | | | GTATTCAAAAGAAGATTTGAGGCTCTGTTACCTC |
| | | | TTCGCTGATGGTGCCACTGCTGAAGTAGTACTTC |
| | | | TTTACCCTGGCAGCATTGTCTCAGTGACAGCTGT |
| | | | GTCTTGTCCACGGGGCCTCTGTGTCCCATGCTCT |
| | | | TCACAAGTTCATCTCCATCCTCTCAATGCTGCAG |
| | | | AAGGCCCTGGGCTCCTCAGTTCTGCACCTACTAC |
| | | | TTTGCTTCTTCCCATTCCGAGGTGGTGTATTTGC |
| | | | CTCAGTTGCTGCTCCTCCTATCCCACCATTCCCT |
| | | | TTCTTACTCTCTCTCAGGTTTCTTGTCTTGTCCT |
| | | | TTCTCACCATTCTAAGATAGCCCTGTGACGCTTC |
| | | | CCTTGATGAGCCCTAATGAGACTCTGTAGCACCA |
| | | | ATCTCTCCTTTCCTGTAGTCACACGAGCTGGAAT |
| | | | CCAGATTCCACTTTGTCATTTGGAGACTCAGAGT |
| | | | ATTGCCACACACACCCCTCAGCGCCACCCCCCCC |
| | | | CCCATTAACTCCCTGCAGCCCCCACTTTCTCCAC |
| | | | GGCACCTACTCCCCCTTGCAGCTTGTGCCGGGAA |
| | | | GCCCTGTTTCCTAGCTGCAGCCTATTATGTTCCA |
| | | | GTCGACAGGCCGGGGGGGGGGGGTGTCACCGACA |
| | | | GCCCCAGAGCCTGCTGCACATGGTGTTAAGTAAG |
| | | | GCTTGGGTTTTCCATGACATTGGTCGGTCCCCAG |
| | | | GGTGGGCAGGGTTCATGTGTCTGCAGGAGTATGT |
| | | | GAGGGCATAGACTGGAAATAGCCTTGTCAAAATA |
| | | | GACCAAGGGCAAATGCTGAGAGGGGAAATGAGGC |
| | | | TGACCTGGGGCGGCGTAGGGCAGGTGCTTCTCCA |
| | | | GGGGCTTTCCTCTGTGAGGGGCCCTGTAGCTAAA |
| | | | GGCTGCCTGAAATACTTCCTGTGACCCTCTAGAC |
| | | | CTACATGAGGCCCCCATCACAAGAGCTTCCTGTT |
| | | | CCCTCTTCACTCCAATACTTACAGAGCAAGAAGG |
| | | | GTTTACTCAGTTCTTCTTTCTTTCTTGTCCCGTC |
| | | | AGCTCGTGTCTTAGTGCATTTGGCCTGCTCTAAG |
| | | | GAAGTGGGACTCTAGGCTGTGTGGCTGTGGAACA |
| | | | ACAGGGGTTGATTTCCTGGTTCTGGAGGCTAGGC |
| | | | ATCCCCGACTGTGTGCCACCGACGTCATTAGCGC |
| | | | GCGGCAAGGGCCTGCTTTTTGACTCATGGTCCCC |
| | | | TGTCTTCCAGGTCTAACCTGGGGGATGAGGTAAG |
| | | | GCGCTTGCTGGCATGTCTTTTCTAAGGATGCTTA |
| | | | TTGTAGTTCCTGGGTTCTGTTCGCATGACATTTC |
| | | | TCATGACCTTGGAGGTTAGGGATTCAACATAGGA |
| | | | ATTTTGAGGGCATAAACAGCCCATAATAGCCTCC |
| | | | TTGAAATATCTCTTGAGTGCACTCTCCTTCCTCA |
| | | | TCAGGCATGTCAACAAAATTTCATGTCACTGTAA |
| | | | AGCAGAAATAATTGTACTTTCTATAGTTCATATT |
| | | | GTGACTTGGGCTTCTTCTTCAATATGCTCAAACT |
| | | | GATGACCAGTTGCATGCCAAACTCACTTTTGCCG |
| | | | GTGTGGTAAAGTTTGTCTCCTAGGCTTCTTACTT |
| | | | AGCTTCAGCCTTTCTGTATTCCATGAAGTGAGGA |
| | | | GATTCATTGGTGGTGTGTGTCAATTAGTTTTTTT |
| | | | GCTGCTGTGATAAAACACCATGAGAAACTTGTAG |
| | | | CCATCATCCAGAGAAGTCAGGGTAGGAACCTGGA |
| | | | GGTAGGAACTGATGCAGAGGCCATCGAGGAGTGC |
| | | | TGCTTACTCCTCCTGGATCACACAGCCTGCTTTC |
| | | | TCAACAGTAGGTAGGACCAACAGCCTAGGTGGCA |
| | | | CCACCCACAGTGAGCTGGGCCTTCCACATCAATC |
| | | | ATCAATCAAGAAAAATAGCACAAAACCCTTTCCC |
| | | | GAAGGCCAATCTGCTGGAGGCATTTTCTCAGTTG |
| | | | AGATTCCCTCTTCCCAAATGACTGCATAAAACTT |
| | | | GTGTCATGTTGACATGAAACTAGCCAGCACAGGG |
| | | | TGTCTGTTAGTTTTTCGGGGCTACTAAACAATCT |
| | | | GAAACACGCTAGATTGCTCAAATCCTCTGGGATG |
| | | | CATTCCGGTAGCTGTGGAGGCAGCAAAGCTGATA |
| | | | TGGTGATGCCCCTACAATCCAGGGGATCCATGGG |
| | | | AAGAGCCTGCCCTTTTTCCATGGGCTTTTAATGA |
| | | | CTACTGGACGCTCTAGGCATTTCTCAGCTTGACG |
| | | | GACGCTTCTCTAGCTGTTCTCCCATGGCTTACTT |
| | | | ATAGGCTTATATATTTATATATAGGCTCCCATGG |
| | | | CCTATGCCTATAACTCTTCTTATATGGATCAGCT |
| | | | TCCATGTACGTATGTATCTCAAATACTATACTGT |
| | | | GATAGTGTCTGTAGAACCCAGGTCCAAGTCACAT |
| | | | CTTATTTGCAAGTACTGCAGGATACAATAGGGTA |
| | | | TGAGAATGAAATGTTAACTCGGGATGAGATACAC |
| | | | AGGTCATCCCAGCTCTTGGGAAGCAGGAGAGGGA |
| | | | TGATCAGAGGTTCAGGACTACCTTCAATTACATT |
| | | | GTGAGTTTAAGGCTAGCCTGGGCTGCCAGAGACT |
| | | | TTGCCTCAACAACTCTACCTTTACGAGAGAAAAG |
| | | | AAAAAACAAGTTCTATGGCTTCTCTCTCTCTCTA |
| | | | AGTAGTATCTTTGGTTTTATATTTGCAATGATGT |
| | | | GGACAATCATATTGTCTTAGTGTTCTATGAAGAG |
| | | | ATGTCATGAACAAGGTATTCTTAAGTTTCAGACG |
| | | | TTAGCCCATGATTATGGTGACACAAAAAACAACA |
| | | | ACAACAACAACAAAAACGGACAAGGTTCTGGAGA |
| | | | AGGAACTGAGAGTCTTATATTCTGATCTGCACGC |
| | | | AGCAGAAGAGGGAGATACTGGGTCTGTCTTGGGC |
| | | | TTTTGAAACCTCAAAGCCCACCTCCAATGAAACA |
| | | | CCCCTACAATAAGACCACATCTGCTAATCTAAAT |
| | | | CCCCAAGTAGTGGTATTCCCTGAGGACTAAGCAT |
| | | | TTGAATATGAGCCTACAGGGGCCATTTTCATTCA |
| | | | AAGAATGCATGCATATGTATAAAGAAAAGCAAAT |
| | | | ACCTGCATAGATTTGGCACCTGTCAGAGAAGAGG |
| | | | TAAATTCAAAGCAGAAAAAGCAACCTAGGCTCTG |
| | | | GTCTGGTTTATGGAGACACTCTGTTTTGGCCTCC |
| | | | GCTCATTGCAATGACAAATTATTATCCTTGGCTT |
| | | | CAGGGTAAAATTTTCTCAGAGTTACGGATACCGA |
| | | | GAAGTTCAAGGACAAAGTATTAACAGTTCATTTT |
| | | | CTGGTGATGGTGTCTGCTTCGGTCATGGATGTCT |
| | | | GTCTTCTTTTGTCATCACAGTGGGGTCAAGGGTT |
| | | | CAGTGTGAGAGCATCTAATGAAACTCATTCTCCT |
| | | | TTAACAAAGAAATAAATATTTATGTTCCATGTGT |
| | | | GCATGTGTGTGTGTATGGGAGTATATATGGGGTC |
| | | | AGAACACAACTTGTAGGACTTGGATTTTTCCAAC |
| | | | TACCATGTAGATTCCTGGAAACTCAGGTCTTCAG |
| | | | GCTAGATAGACCACAAGCTCCATTTCCAAAACCG |
| | | | TCTCACCAGCCCCATCCAATGTCTCTTCTTATGG |
| | | | GAAACTTATGAGTTCAGATCTCTGCCAATGCATG |
| | | | AGGTATTATGTGTTCTTCCTAACTTCTATCAATA |
| | | | CCTCTTCTCCAATATAGTCTCATGGAAATGGTGG |
| | | | ACTAGAGCTGATAGGATGCGCAAGCACACGCACG |
| | | | CACGTGTGAGCACACACACACACACACACACACA |
| | | | CACACACACACCCTCACTTATTAGAATGACTTAT |
| | | | AGGTTGTGGTCCTGTCTTATGACAGAAGTCCAAG |
| | | | AACCCAATAGTTAGGTTACTTAGATACTCTCACA |
| | | | CTGCCCTCATGCTCACTGGCAAGTTCATCCGTCC |
| | | | TGGAGCTGAGGCATCCTTCACTGATATTAAAGCC |
| | | | TACCTCCTTCAGGATTCCAACATACATTGAATAG |
| | | | TTCAGTAGACCAGCTTGATCCCTTAGTTGGTCTT |
| | | | CGGTTGTAATCCTGAAGAAGTTAAAAA |
| S000023 | F5 | 132 | CAGAGTTGCTCTAGCCTGGCTGCCCAAGCCAAGC |
| | | | CGTTAGAAGCAGGAGCCCCTGGCCAGTGCCTGGT |
| | | | CACGGAGCTGAGCTGTGTTTAGATGTGTTGGCTG |
| | | | CTGGGTGGTGAAGGAAGACCCGTCTCCAGAAAAG |
| | | | CAATTTAGGCAAAAGGGATTCCGTTTGATGGCAG |
| | | | AGTCCCAGTGCTAGAAAGGTAGCGAAGGTGGACA |
| | | | GCTTACAGTCTCAACTCATTTCGTCGTAAATGTC |
| | | | CTCGTAACGACATTGATTCTTCTACCTGGATAAC |
| | | | CTTTTGTTTGTTTGTTTGTTTGTTTTTGTTTTGT |
| | | | TTTTCCCCTGTAACCATTTTTTTTTCTGACAAGA |
| | | | AAACATTTTAATTTTCTAAGCAAGAAGCATTTTT |
| | | | CAAATACCATGTCTGTGACCCAAAGTAAAAATGG |
| | | | ATGATAATTCATGTAAATGTGTGCAACATAGCAA |
| | | | CCTGAACCTGCACGCGATTCGGGCTCTGTAGGTT |
| | | | GTGAACCATGGCTATGTGGATACAGGCTCAGCAG |
| | | | CTCCAGGGCGATGCCCTTCACCAGATGCAGGCCT |
| | | | TGTACGGCCAGCATTTCCCCATCGAGGTGCGACA |
| | | | TTATTTATCACAGTGGATCGAAAGCCAAGCCTGG |
| | | | GACTCAATAGATCTTGATAATCCACAGGAGAACA |
| | | | TTAAGGCCACCCAGCTCCTGGAGGGCCTGGTGCA |
| | | | GGAGCTGCAGAAGAAGGCGGAGCACCAGGTGGGG |
| | | | GAAGATGGGTTTTTGCTGAAGATCAAGCTGGGGC |
| | | | ACTATGCCACACAGCTCCAGAGCACGTACGACCG |
| | | | CTGCCCCATGGAGCTGGAGCGCTGTATCCGGCAC |
| | | | ATTCTGTACAACGAACAGAGGCTGGTTCGCGAAG |
| | | | CCAACAACGGCAGCTCTCCAGCTGGAAGTCTTGC |
| | | | TGACGCCATGTCCCAGAAGCACCTTCAGATCAAC |
| | | | CAAACGTTTGAGGAGCTGCGCCTGATCACACAGG |
| | | | ACACGGAGAACGAGCTGAAGAAGCTGCAGCAGAC |
| | | | CCAAGAGTACTTCATCATCCAGTACCAGGAGAGC |
| | | | CTGCGGATCCAAGCTCAGTTTGCCCAGCTGGGAC |
| | | | AGCTGAACCCCCAGGAGCGCATGAGCAGGGAGAC |
| | | | GGCCCTCCAGCAGAAGCAAGTGTCCCTGGAGACC |
| | | | TGGCTGCAGCGAGAGGCACAGACACTGCAGCAGT |
| | | | ACCGAGTGGAGCTGGCTGAGAAGCACCAGAAGAC |
| | | | CCTGCAGCTGCTGCGGAAGCAGCAGACCATCATC |
| | | | CTGGACGACGAGCTGATCCAGTGGAAGCGGAGAC |
| | | | AGCAGCTGGCCGGGAACGGGGGTCCCCCCGAGGG |
| | | | CAGCCTGGACGTGCTGCAGTCCTGGTGTGAGAAG |
| | | | CTGGCCGAGATCATCTGGCAGAACCGGCAGCAGA |
| | | | TCCGCAGGGCTGAGCACTTGTGCCAGCAGCTGCC |
| | | | CATCCCAGGCCCCGTGGAGGAGATGCTGGCTGAG |
| | | | GTCAACGCCACCATCACGGACATCATCTCAGCCC |
| | | | TGGTCACCAGCACGTTCATCATCGAGAAGCAGCC |
| | | | TCCTCAGGTCCTGAAGACCCAGACCAAGTTTGCA |
| | | | GCCACCGTGCGCCTGCTGGTGGGGGGGAAGCTGA |
| | | | ATGTGCACATGAACCCCCCGCAGGTGAAGGCGAC |
| | | | CATCATCAGCGAGCAGCAGGCCAAGTCCCTGCTC |
| | | | AAGAATGAGAACACCCGCAATGATTACAGCGGCG |
| | | | AGATCCTGAACAACTGTTGCGTCATGGAGTACCA |
| | | | CCAGGCCACTGGCACACTCAGCGCCCACTTCAGA |
| | | | AACATGTCCCTGAAACGAATCAAGAGGTCTGACC |
| | | | GCCGTGGGGCAGGGTCAGTAACGGAAGAGAAGTT |
| | | | CACGATCCTGTTTGACTCACAGTTCAGCGTCGGT |
| | | | GGAAACGAGCTGGTCTTTCAAGTCAAGACCTTGT |
| | | | CGCTCCCGGTGGTGGTGATTGTTCACGGCAGCCA |
| | | | GGACAACAATGCCACAGCCACTGTCCTCTGGGAC |
| | | | AACGCCTTTGCAGAGCCTGGCAGGGTGCCATTTG |
| | | | CCGTGCCTGACAAGGTGCTGTGGCCGCAGCTGTG |
| | | | TGAAGCGCTCAACATGAAATTCAAGGCTGAAGTA |
| | | | CAGAGCAACCGGGGCTTGACCAAGGAGAACCTCG |
| | | | TGTTCCTGGCACAGAAACTGTTCAACATCAGCAG |
| | | | CAACCACCTCGAGGACTACAACAGCATGTCCGTG |
| | | | TCCTGGTCCCAGTTCAACCGGGAGAATTTGCCAG |
| | | | GACGGAATTACACTTTCTGGCAGTGGTTTGGCGT |
| | | | GATGGAAGTATTGAAAAAACATCTCAAGCCTCAC |
| | | | TGGAATGATGGGGCTATCCTGGGTTTCGTGAACA |
| | | | AGCAACAGGCCCACGACCTGCTCATCAACAAGCC |
| | | | AGACGGGACCTTCCTGCTGCGCTTCAGCGACTCG |
| | | | GAAATCGGGGGGCATCACCATTGCTTGGAAGTTT |
| | | | GACTCTCAGGAGAGAATGTTTTGGAATCTGATGC |
| | | | CTTTTACCACTAGAGACTTCTATCCGGTCCCTCG |
| | | | CTGACCGCCTGGGGGACCTGAATTACCTCATATA |
| | | | TGTGTTTCCTGATCGGCCAAAGGATGAAGTATAT |
| | | | TCTAAGTACTACACACCGGTCCCCTGTGAGCCCG |
| | | | CAACTGCGAAAGCTGACGGATACGTGAAGCCACA |
| | | | GATCAAGCAGGTGGTCCCCGAGTTTGCAAATGCA |
| | | | TCCACAGATGCTGGGAGTGGCGCCACCTACATGG |
| | | | ATCAGGCTCCTTCCCCAGTCGTGTGCCCTCAGGC |
| | | | TCACTACAACATGTACCCACCCAACCCGGACTCC |
| | | | GTCCGTCCTTGATACCGATGGGGACTTCGATCTG |
| | | | GAAGACACGATGGACGTGGCGCGGCGGGTCGAAG |
| | | | AGCTCTTAGGCCGGCCCATGGACAGTCAGTGGAT |
| | | | CCCTCACGCACAGTCATGACCAGACCTCACCACC |
| | | | TGCAGCTTCATCGCCCTCGTGGAGGAACTTCCTG |
| | | | TGGATGTTTTAATTCCATGAATCGCTTCTCTTTG |
| | | | GAAACAATACTCG |
| S000028 | F6 | 133 | CTGCCTTACAGCACTGTTCTCGGCAGCTTACAGG |
| | | | AAACCTTCCTTTCCTGATTCCCACCTTACCACAA |
| | | | GACCCAGGGCTGTGGGGTGAGGTGTGCTACCGAA |
| | | | CTGAACGCCAGCAATGATGTTCCAGAAAACATTT |
| | | | TAATATCTTCCCTTGGTTCCACTGCTGCTAAGCT |
| | | | GGGGACGGGGCTGGAATAGCCGCTCCGGTGGAGG |
| | | | AGGCTTCCCAGCAGGGGAGAGAGATAATTAAAAT |
| | | | GGCATTACCGTGTCTCCCTGTGGGATGCGGTGAC |
| | | | ATTAAAGAGCCACACTGACAAAATACCCGGGACT |
| | | | GGAAGGTTCTGTGCTGCCTTCCTCGCAGACACAG |
| | | | CAGCCACAGCAGTATCTGAGGCTGCTGGGACCGC |
| | | | TTGCTCTGCTCACAGGCGGTCTGGGGCGGGGATC |
| | | | CTAGATGCGAAGACCTACCGAGCTGAAGGGAGGG |
| | | | AAAGAATCGGTCTGGGACGGGCGGGGCTATCCCG |
| | | | GGGTTCCCTATCTGGAGGGCACAAGTCCTGCTGT |
| | | | GGATGTTAGCACGCTCCTTTTGGCTTGAGGAGAA |
| | | | CTTGGGAAGGCCGGCTCCATGAGGGTGGCTTCCC |
| | | | CTTTGTTGTGCCGGAGGTGGGGTTCCAACCCGGG |
| | | | AGGGTGGTAACGGCTAAGGGAGGCGGCTAAACAA |
| | | | CCGGAAGGCCAAATATTTGGATTGGCCG |
| S000031 | F7 | 134 | GTAAAGATCCTAAAGGTGGTTGACCCAACTCCAG |
| | | | AGCAACTTCAGGCCTTCAGGAACGAGGTGGCTGT |
| | | | TTTGCGCAAAACACGGCATGTTAACATCCTGCTG |
| | | | TTCATGGGGTACATGACAAAGGACAACCTGGCGA |
| | | | TTGTGACTCAGTGGTGTGAAGGCAGCAGTCTCTA |
| | | | CAAACACCTGCATGTCCAGGAGACCAAATTCCAG |
| | | | ATGTTCCAGCTAATTGACATTGCCCGACAGACAG |
| | | | CTCAGGGAATGGACTATTTGCATGCAAAGAACAT |
| | | | CATCCACAGAGACATGAAATCCAACAATATATTT |
| | | | CTCCATGAAGGCCTCACGGTGAAAATTGGAGATT |
| | | | TTGGTTTGGCAACAGTGAAGTCACGCTGGAGTTT |
| | | | GGTCCTCAGCAGGTTGAACAGCCCACTGCTCTGT |
| | | | GCTGTGGATGGCCCCAGAAGTAATCCGGATGCAG |
| | | | GATGACAACCCGTTCAGCTTCCAGTCCGACGTGT |
| | | | ACTCGTACGGCATCGTGCTGTACGAGCTGATGGC |
| | | | TGGGGAGCTTCCCTACGCCCACATCAACAACCGA |
| | | | GACCAGATCATCTTCATGGTAGGCCGTGGGTATG |
| | | | CATCCCCTGATCTCAGCAGGCTCTACAAGAACTG |
| | | | CCCCAAGGCAATGAAGAGGTTGGTGGCTGACTGT |
| | | | GTGAAGAAAGTCACAGAAGAGAGACCTTTGTTTC |
| | | | GCCAGATCCTGTCTTCCATCGAGCTGCTTCAGCA |
| | | | CTCTCTGCCGAAAATCCACAGGAACGCCTCTGAG |
| | | | CTTTCCCTGCATCGGGCAGCTCACACTGAGGGAC |
| | | | ATCATGCTTGCACGCTGACTACATTCCCAAGGCT |
| | | | ACCGTCTCCTAACTGATGATGTAGCCTGTCTTAG |
| | | | GCCACATGGGACCAAAAGAAGTCAGCAGGACCAA |
| | | | TTTT |
| S000039 | F8 | 135 | ACAAGACTTTGAAAAGCGGTTCCTGAAGAGGATT |
| | | | CGTGACTTGGGAGGGTCACTTTGGGAAGGTTGAG |
| | | | CTCTGCAGATATGATCCTGAGGGAGACAACACAG |
| | | | GGGAGCAGGTAGCTGTCAAGTCCCTGAAGCCTGA |
| | | | GAGTGGAGGTAACCACATAGCTGATCTGAAGAAG |
| | | | GAGATAGAGATCTTACGGAACCTCTACCATGAGA |
| | | | ACATTGTGAAGTACAAAGGAATCTGCATGGAAGA |
| | | | CGGAGGCAATGGTATCAAGCTCATCATGGAGTTT |
| | | | CTGCCTTCGGGAAGCCTAAAGGAGTATCTGCCAA |
| | | | AGAATAAGAACAAAATCAACCTCAAACAGCAGCT |
| | | | AAAAATATGCCATCCAGAATTGTAAGGGGATGGA |
| | | | CTACTTGGGTTCTCGGCAATAAGTTCACCGGGAC |
| | | | TTAGCAGCCAGAATGTCCTTGTTGAGAGTGAGCA |
| | | | TCCAGTTGAGATTGGAGACCTTGGGTTAACCCAA |
| | | | GCCATTTGAAACGATTAGGAGTACTACACAGTTC |
| | | | AGGACCACCGGGAAAAGCCAGTGTTCCGGTACGC |
| | | | TCCGGAATGTTTAATCCAGTGTTAATTTTAAAAC |
| | | | GCCTCCGATGTCCGGTCCTTTGGAGTGACACTGC |
| | | | ACGAGCTGCTCAATTACTGTGACTCCGAATTTAG |
| | | | TCCCATGGCCTTGGTCCCGAAAAGGTAAGCCCAA |
| | | | CTCCAGGCCAGAAGACAATTGAAGGCCTGTGGAT |
| | | | CACTGAAAGAAGGAAAGCCCTGGCATGTCCACCC |
| | | | AATGTCCTGATGAAGTTAACAGCCTATGGGAAAA |
| | | | TTCCTGGAATTCGANCTACTAACCGAACAATTTT |
| | | | CGGAACCTATGGAAGAGTTTAAGCCCCTTTAAAT |
| | | | AGAAGCCTGGCACACTTTAATCCCCATTTCAAAT |
| | | | CTTTCTCCAAGCCTTTAAAAAGGTTTAAAGGAAA |
| | | | GTTGAATCGGGCCTAAGTCCCAAAAAACCGCGGT |
| | | | ACAATTGCAATTCACGGGTCC |
| S000040 | F9 | 136 | TGGACTGGGTGCGGCCGGCTGCAAGACTCTAGTC |
| | | | GTCGGCCCACGTGGCTGGGGCGGGGACTGCCGTG |
| | | | GCGCCTAGTGATTACGTAGCGGGTGGGGCCCGAA |
| | | | GTGCCGCTCCCTGGCGGGGCTGTTCATGGCGGTT |
| | | | TCGGGGTCTCCAACAGCTCAGGTTGAAGTCCAAA |
| | | | AGCCTCCCGAGGCGGGCTGCGGAGTTTGAGGTTT |
| | | | TTGCTGGTGTGAAATGACTGAGTACAAACTGGTG |
| | | | GTGGTTGGAGCAGGTGGTGTTGGGAAAAGCGCCC |
| | | | TGACGATCCAGCTAATCCAGAACCACTTTGTGGA |
| | | | TGAATATGATCCCACCATAGAGGATTCTTACCGA |
| | | | AAGCAAGTGGTGATTGATGGTGAGACCTGCCTGC |
| | | | TGGACATACTGGACACAGCTGGACAAGAGGAGTA |
| | | | CAGTGCCATGAGAGACCAGTACATGAGGACAGGC |
| | | | GAAGGGTTCCTCTGTGTATTTGCCATCAATAATA |
| | | | GCAAATCATTTGCAGATATTAACCTCTACAGGGA |
| | | | GCAAATTAAGCGTGTGAAAGATTCTGATGATGTC |
| | | | CCCATGGTGCTGGTAGGCAACAAGTGTGACTTGC |
| | | | CAACAAGGACAGTTGACACAAAGCAAGCCCACGA |
| | | | ACTGGCCAAGAGTTACGGAATTCCATTCATTGAG |
| | | | ACCTCAGCCAAGACCCGACAGGGTGTGGAGGATG |
| | | | CCTTTTACACACTGGTAAGGGAGATACGCCAGTA |
| | | | CCGATTGAAAAAGCTCAACAGCAGTGACGATGGC |
| | | | ACTCAAGGTTGTATGGGGTCGCCCTGTGTGCTGA |
| | | | TGTGTAAGACACTTTGAAAGTTCTGTCATCAGAA |
| | | | AAGAGCCACTTTGAAGCTGCACTGATGCCCTGGT |
| | | | TCTGACATCCCTGGAGGAGACCTGTTCCTGCTGC |
| | | | TCTCTGCATCTCAGAGAAGCTCCTGCTTCCTGCT |
| | | | TCCCCGACTCAGTTACTGAGCACAGCCATCTAAC |
| | | | CTGAGACCTCTTCAGAATAACTACCTCCTCACTC |
| | | | GGCTGTCTGACCAGAGAAATGGACCTGTCTCTCC |
| | | | CGGTCGTTCTCTGCCCTGGGTTCCCCTAGAAACA |
| | | | GACACAGCCTCCAGCTGGCTTTGTCCTCTGAAAA |
| | | | GCAGTTTACATTGATGCAGAGAACCAAACTAGAC |
| | | | ATGCCATTCTGTTGACAACAGTTTCTTATACTCT |
| | | | AAGGTAACAACTGCTGGTGATTTTCCCCTGCCCC |
| | | | CAACTGTTGAACTTGGCCTTGTTGGTTTGGGGGG |
| | | | AAAATGTCATAAATTACTTTCTTCCCAAAATATA |
| | | | ATTAGTGTTGCTGATTGATTTGTAATGTGATCAG |
| | | | CTATATTCCATAAACTGGCATCTGCTCTGTATTC |
| | | | ATAAATGCAAACACGAATACTCTCAACTGCATGC |
| | | | AATTAAATCCAACATTCACAACAAAGTGCCTTTT |
| | | | TCCTAAAAGTGCTCTGTAGGCTCCATTACAGTTT |
| | | | GTAATTGGAATAGATGTGTCAAGAACCATTGTAT |
| | | | AGGAAAGTGACTCTGAGCCATCTACCTTTGAGGG |
| | | | AAAGGTGTATGTACCTGATGGCAGATGCTTTGTG |
| | | | TATGCACATGAAGATAGTTTCCCTGTCTGGGATT |
| | | | CTCCCAGGAGAAAGATGGAACTGAAACAATTACA |
| | | | AGTAATTTCATTTAATTCTAGCTAATCTTTTTTT |
| | | | TTTTTTTTTTTTTGGTAGACTATCACCTATAAAT |
| | | | ATTTGGAATATCTTCTAGCTTACTGATAATCTAA |
| | | | TAATTAATGAGCTTCCATTATAATGAATTGGTTC |
| | | | ATACCAGGAAGCCCTCCATTTATAGTATAGATAC |
| | | | TGTAAAAATTGGCATGTTGTTACTTTATAGCTGT |
| | | | GATTAATGATTCCTCAGACCTTGCTGAGATATAG |
| | | | TTATTAGCAGACAGGTTATATCTTTGCTGCATAG |
| | | | TTTGTTCATGGAATATATATCTATCTGTATGTGG |
| | | | AGAGAACGTGGCCCTCAGTTCCCTTCTCAGCATC |
| | | | CCTCATCTCTCAGCCTAGAGAAGTTCGAGCATCC |
| | | | TAGAGGGGCTTGAACAGTTATCTCGGTTAAACCA |
| | | | TGGTGCTAATGGACCGGGTCATGGTTTCAAAACT |
| | | | TGAACAAGCCAGTTAGCATCACAGAGAAACAGTC |
| | | | CATCCATATTTGCTCCCTGCCTATTATTCCTGCT |
| | | | TACAGACTTTTGCCTGATGCCTGCTGTTAGTGCT |
| | | | ACAAGGATAAAGCTTGTGTGGTTCACCAGGACTG |
| | | | GAAGTACCTGGTGAGCTCTGGGGTAAGCCTAGAT |
| | | | ATCTTTACATTTTCAGACCCTTATTCTTAGCCAC |
| | | | GTGGAAACTGAAGCCAGAGTCCATACCTCCATCT |
| | | | CCTTCCCCCCCCAAAAAAATTAGATTAATGTTCT |
| | | | TTATATAGCTTTTTTAAAGTATTTAAAACATGTC |
| | | | TATAAGTTAGGCTGCCAACTAACAAAAGCTGATG |
| | | | TGTTTGTTCAAATAAAGAGGTATCCTTCGCTACT |
| | | | CGAGAGAAGAATGTAAAATGCCATTGATTGTTGT |
| | | | CACTTGGAGGCTTGATGTTGCCCTGATAATTCAT |
| | | | TAGTGGGTTTTGTTTGTCACATGATACCTAAGAT |
| | | | GTAACTCAGCTCAGTAATTCTAATGAAAACATAA |
| | | | ATTGGATACCTTATTGAAAAAAGCAAACCTAATT |
| | | | CCAAAATGGCCATTTTCTCTTCTGATCTTGTAAT |
| | | | ACCTAAAATTCTCGAGGTCCTTGGGATTCTTTTG |
| | | | TTTATAACAGGATCTTGCTGTGTAGTCCTAGCTG |
| | | | GCCTCAAACTCACAATACTCTTCCTGGATCAATC |
| | | | TCCCAAGTGCTGGGATTACAGGCACATTCCACCA |
| | | | CACACACCTGACTGAGCTCGTTCCTAATGAGTTT |
| | | | TCATTAAGCAAACCCCATCACCTTGAAACTAATC |
| | | | AGAAGGGGGAACAAACATTTGCTATGCTCCTGAG |
| | | | TGCTAACACTGGGCTCATTCACATGGGGTTTGCA |
| | | | TTCCTAGGCAAACTAAAGCTGCCTTTTACAACAA |
| | | | GGCTCAGTCATCTTCCTGAAGCTGCTGAGACCAG |
| | | | CACTTGGTCTTGTTTTGTTTTAATATGTCTATAT |
| | | | GACTGGTGGTGGATCCGTCGACCTGCA |
| S000046 | F1 | 137 | TTATTATCAATGTGACTCCTCGGGGGAGTCAATG |
| | | | ATGGTGTTGGGGAGGAGGATGATGATGAATTATC |
| | | | AATGTGACTCCTCGGGGGAGTCAATGATGGTGTT |
| | | | GGGGAGGAGGATGATGATGAGACGCCTCTAAACT |
| | | | TGGAACAAGTTTAGGACTTTGAAAGAGAAGAGAA |
| | | | AAAAAAAATACAACCAACAAGACCGAAGAACAAT |
| | | | TATAACTATCCAGTGTTGATTATTTTTATAAACA |
| | | | ATACGAAAAAGTTGTCGGATTTTTTTTTTTAATG |
| | | | ATTACTTTTTGGGGGGAGGGAATTTTGTTACAGT |
| | | | TTGATGATGGAAAATGCAAAAACCGAGCCAGGTG |
| | | | CATAATCTTGTAATTTGTGGCTAACCCTGGAACA |
| | | | GGACTGACTTCTATTTAAATACTCTTTTGGGGGA |
| | | | ACACTCATGTGAGACACTAAGTTCTTGCAGAAGA |
| | | | TTTTTGTCTCTCTTTTTAAAGTCTCTTTCCTTGG |
| | | | AATATTGTGAGCATATTTGTGGCCATTGAAGGTT |
| | | | TGTGTGATTTTGCTAAAATGCATCACCAACAGCG |
| | | | AATGGCTGCCTTAGGGACGGACAAAGAGCTGAGT |
| | | | GATTTACTGGATTTCAGTGCGATGTTTTCGCCTC |
| | | | CTGTAAGCAGTGGGAAAAATGGACCAACTTCTTT |
| | | | GGCGAGTGGACATTTCACTGGCTCAATGTAGAAG |
| | | | ACAGAAGTAGCTCAGGGTCCTGGGGAACTGGAGG |
| | | | CCATCCAAGCCCGTCCAGGAACTATGGAGATGGG |
| | | | ACTCCCTATGACCACATGACTAGCAGGGATCTTG |
| | | | GGTCACATGACAATCTCTCTCCACCTTTTGTCAA |
| | | | CCAGAATACAAAGTAAAACAGAAAGGGGCTCATA |
| | | | CTCATCTTATGGGAGAGAAAACGTTCAGGGTTGC |
| | | | CACCAGCAGAGTCTCCTCGGAGGGGACATGGATA |
| | | | TGGGCAATCCAGGAACCCTTTCGCCCACCAAACC |
| | | | TGGCTCCCAGTACTATCAGTATTCAAGCAATAAT |
| | | | GCCCGCCGGAGGCCTCTTCACAGTAGTGCCATGG |
| | | | AGGTACAGACAAAGAAAGTCCGAAAAGTTCCTCC |
| | | | GGGTTTGCCGTCTTCAGTCTACGCTCCTTCAGCC |
| | | | AGCACTGCCGACTACAACAGGGACTCGCCAGGCT |
| | | | ATCCTTCCTCCAAGCCAGCAGCCAGCACTTTCCC |
| | | | TAGCTCCTTCTTCATGCAAGATGGCCATCACAGC |
| | | | AGCGACCCTTGGAGCTCCTCCAGCGGGATGAATC |
| | | | AGCCCGGCTACGGAGGGATGCTGGGCAATTCTTC |
| | | | TCATATCCCACAGTCCAGCAGCTACTGTAGCCTG |
| | | | CATCCACACGAACGTTTGAGCTATCCATCCCACT |
| | | | CCTCGGCAGACATCAACTCCAGTCTTCCTCCGAT |
| | | | GTCCCACGTTCCATCGTAGTGGCACAAACCATTA |
| | | | CAGCACCTCTTCCTGCACACCCCCTGCCAACGGA |
| | | | ACAGACAGTATAATGGCAAACAGAGGAACTGGGG |
| | | | CAGCAGGCAGCTCGCAGACTGGAGACGCTCTGGG |
| | | | GAAAGCCCTAGCTTCGATCTATTCTCCTGACCAC |
| | | | ACGAACAACAGCTTTTCCTCCAATCCTTCAACTC |
| | | | CTGTGGGCTCCCCTCCTTCACTCTCAGCAGGCAC |
| | | | AGCTGTTTGGTCTAGAAATGGAGGACAGGCCTCG |
| | | | TCATCTCCCAATTATGAAGGACCCTTGCACTCAC |
| | | | TGCAAAGCCGAATCGAAGACCGTTTGGAAAGACT |
| | | | GGACGATGCGATTCATGTTCTCCGGAACCACGCA |
| | | | GTGGGCCCGTCCAGCTGTGCCTGGTGGCCATGGG |
| | | | GACATGCATGGGATCATGGGACCCTCCACAACGG |
| | | | AGCGATGGGTAGCCTGGGCTCAGGGTACGGAACT |
| | | | AGTCTTCTCTCAGCCAACAGACACTCGCTCATGG |
| | | | TTGGGGCCCACCGTGAAGATGGCGTGGCTCTGAG |
| | | | AGGCAGCCATTCTCTCCTGCCAAACCAGGTTCCG |
| | | | GTCCCACAACTTCCGGTCCAGTCTGCAATTCCCC |
| | | | TGACTTGACCCACCCCAAGACCCTTACAGAGGAT |
| | | | GCCACCAGGCCTCCAGGGCCAGAGCGTGTCTTCT |
| | | | GGTAGCTCTGAGATCAATCCGATGACGAGGGCGA |
| | | | TGAGAACTGCAAGACACAAAATCTTCTGAGGACA |
| | | | AGAAATTAGATGACGACAAGAAGGATATCAAATC |
| | | | AATTACTAGGTCAAGATCTAGCAATAACGATGAT |
| | | | GAGGACCTGACCCCAGAGCAGAAGGCTGAGCGCG |
| | | | AGAAGGAACGGAGGATGGCCAATAATGCCCGTGA |
| | | | GCGCCTGAGGGTCCGAGATATCAACGAGGCTTTC |
| | | | AAGGAGCTGGCCGTATGGTGCAGCTCCACCTGAA |
| | | | GAGCGACAAGCCCCAGACCAAGCTCCTGATTCTC |
| | | | CACCAGGCCGTGGCTGTCATCCTCAGCCTGGAGC |
| | | | AGCAAGTTCGAGAAAGGAATCTGAACCCGAAAGC |
| | | | TGCCTGTCTGAAAAGAAGGGAGGAAGAGAAGGTG |
| | | | TCCTCAGAGCCTCCCCCACTCTCCTTGGCTGGCC |
| | | | CACACCCTGGGATGGGAGACGCAGCGAATCACAT |
| | | | GGGACAGATGTGAAAAGGTCCAAGTTGCTACCTT |
| | | | GCTTCATTAAACAAGAGACCACTTCCTTAACAGC |
| | | | TGTATTACCCTAAACCCACATACACTGCTCCTTA |
| | | | ACCCCGTTTTTTTTTGTAATATAAGACAAGTCTG |
| | | | AGTAGTTATGAATCGCAGACGCAAGAGGTTTCAG |
| | | | CATTCCCAATTATCAAAAAACAGAAAAACAAACA |
| | | | AAAAAATGAATGAAAGAAAGAAAGAAAGAAAAAA |
| | | | ATGCAACTTGAGGGACGACTTCTTTACATATCAC |
| | | | TCTGAATGTGCGACGGTATGTACAGGCTGAGACA |
| | | | CAGCCCAGAGACTGAATGGCAATCCTCCACACTG |
| | | | TGGAGCAATGCATTTGTGCCTAAACTTCTTTTGG |
| | | | AAAAAAAAAATATAATTAATTTGTAAGTCTGAAA |
| | | | AAAATATTTAATTTAAAAAAAATTGTAAACTTCA |
| | | | ATAATGAAAAAGTGTACTTCTGAAGAAAACGACA |
| | | | TGAACGTTTTGTTGGTATTCACGTCAGCTAGTGT |
| | | | TTCTAATTACCGGATATTGAATAGGGGAAGCCCG |
| | | | GCTGCCCTCGTAACAAAACCAGCAAACGTCCTGA |
| | | | TGGCAACGAAGTGATGACATTAGCCATTCCTTAG |
| | | | GGTAGGAGGGACAGATGGATGTTATAGACCTATG |
| | | | ACAAATATATATATAAATATATATATAAATATAT |
| | | | ATTAAAAATTTAGTGACTATGGTAAGCTTGTGAT |
| | | | GTCAGCTTTTCTCCTGTAAAAATAGTACTGATAA |
| | | | CTTTTTAAAAGAAAGATTTTACTGTAAATATGGA |
| | | | TTTTTTTTTTGTCTGATTTTTGTCCCTTCCCCCG |
| | | | GTTTGTTATCGTAACCTGTAGTGCCAACTCTGCT |
| | | | TCCGGAGGGGCAGTGCAGGACGAAATGCTGACCC |
| | | | TGAAGTTGCTTCTCATTCACAAATAGTAAAAAGT |
| | | | TGTTTCTCCAGTCTTTTGGGAACACAGGACTTAA |
| | | | AAGTCACATCATGTGTAGGAATTACATGCAGCAT |
| | | | TGCCCGGGCGAGGAAAAAAGCGTTTGTCTGGCTT |
| | | | GTGGCGCTGCCCTTGTTACCCTCCCCTGGGATTT |
| | | | TCAGAGGTACACGGTTAGAATGCTACAATGTTAC |
| | | | CACTGTGCCTTCCAATGTTTATATCATCGGAAAC |
| | | | ATAACATAATCAAAGTGGCTGTGATTTAACAAAA |
| | | | AAAACGATTCAAGTGTTACCTACCTGTGTAGCCG |
| | | | AAGTAGTGTGCAGTGACCGAGACGTTTCAGAATA |
| | | | CATGGTCAGATTTTTTTTGGAAAAAATACAAAAA |
| | | | TTA |
| S000050 | F1 | 138 | CTGTCCATTTCATCAAGTCCTGAAATATCGAAAT |
| | | | GGATTTAGAGAAAAATTACCCGACTCCTCGGACC |
| | | | ATCAGGACAGGACATGGAGGAGTGAATCAGCTTG |
| | | | GGGGGGTTTTTGTGAATGGACGGCCACTCCCAGA |
| | | | TGTAGTCCGCCAAAGGATAGTGGAACTTGCCCAT |
| | | | CAAGGTGTCAGGCCCTGCGACATCTCCAGGCAGC |
| | | | TTCGGGTCAGCCATGGTTGTGTCAGCAAAATTCT |
| | | | TGGCAGGTATTATGAGACAGGAAGGATCAAGCCG |
| | | | GGGGTGATTGGAGGTCCAAACCAAAGGTTGCCAC |
| | | | TCCCAAAGTGGTGGAAAAAATCGCTGAGTACAAA |
| | | | CGCCAAAACCCTACCATGTTTGCCTGGGAGATCA |
| | | | GGGACCGGCTGTTGGCAGAGCGAGTCTGTGACAA |
| | | | TGACACTGTGCCCAGCGTCAGCTCCATCAACAGG |
| | | | ATCATTCGGACAAAAGTACAGCAGCCCCCCAATC |
| | | | AGCCGGTCCCAGCTTCCAGTCACAGCATAGTGTC |
| | | | TACAGGCTCCGTGACGCAGGTGTCATCGGTGAGC |
| | | | ACCGACTCCGCGGGCTCCTCATACTCCATCAGTG |
| | | | GCATCCTGGGCATCACGTCCCCCAGTGCCGACAC |
| | | | CAACAAACGCAAGAGGGATGAAGGTATTCAGGAG |
| | | | TCTCCAGTGCCGAATGGCCACTCACTTCCGGGCC |
| | | | GGGACTTCCTCCGGAAGCAGATGCGGGGAGACCT |
| | | | GTTCACACAGCAGCAGCTGGAGGTGCTGGACCGC |
| | | | GTGTTTGAGAGACAGCACTACTCTGACATCTTCA |
| | | | CCACCACGGAACCCATCAAGCCAGAACAGACCAC |
| | | | AGAGTATTCAGCCATGGCTTCACTGGCTGGAGGC |
| | | | CTGGATGACATGAAAGCCAACTTGACGAGCCCCA |
| | | | CCCCCGCTGACATCGGGAGCAGCGTTCCAGGCCC |
| | | | ACAGTCCTACCCTATTGTCACAGGCCGAGACTTG |
| | | | GCGAGCACAACCCTCCCGGGGTACCCTCCACACG |
| | | | TCCCCCCCGCTGGACAGGGCAGCTACTCTGCACC |
| | | | GACGCTGACAGGGATGGTGCCTGGGAGTGAATTT |
| | | | TCTGGAAGTCCCTACAGCCACCCTCAGTATTCTT |
| | | | CCTACAATGATTCTTGGAGGTTCCCCAACCCGGG |
| | | | CTGCTTGGCTCCCCATACTATTACAGCCCTGCAG |
| | | | CCCGAGGAGCGGCCCCACCGGCCGCAGCCACTGC |
| | | | GTACGACCGCCACTGA |
| S000056 | F12 | 139 | GTTGAGCGCGAAGGAGCCGAGATGGAAGGAAGCC |
| | | | CTACCACCGCCACTGCGGTGGAAGGAAAAGTCCC |
| | | | CTCTCCGGAGAGAGGGGACGGATCTTCCACCCAG |
| | | | CCTGAAGCAATGGATGCCAAGCCAGCCCCTGCTG |
| | | | CCCAAGCCGTCTCTACCGGATCTGATGCTGGAGC |
| | | | TCCTACGGATTCCGCGATGCTCACAGATAGCCAG |
| | | | AGCGATGCCGGAGAAGACGGGACAGCCCCAGGAA |
| | | | CGCCTTCAGATCTCCAGTCGGATCCTGAAGAACT |
| | | | CGAAGAAGCCCCAGCTGTCCGCGCCGATCCTGAC |
| | | | GGAGGGGCAGCCCCAGTCGCCCCAGCCACACTCC |
| | | | TGCCGAGTCCGAGTCTGAAGGCAGCAGAGATCCA |
| | | | GCCGCCGAGCCAGCCTCCGAGGCAGTCCCTGCCA |
| | | | CCACGGCCGAGTCTGCCTCCGGGGCAGCCCCTGT |
| | | | CACCCAGGTGGAGCCCGCAGCCGCGGCAGTCTCT |
| | | | GCCACCCTGGCGGAGCCTGCCGCCCGGGCAGCCC |
| | | | CTATCACCCCCAAGGAGCCCACTACCCGGGCAGT |
| | | | CCCCTCTGCTAGAGCCCATCCGGCCGCTGGAGCA |
| | | | GTCCCTGGCGCCCCAGCAATGTCAGCCTCTGCTA |
| | | | GGGCAGCTGCCGCTAGGGCAGCCTATGCAGGTCC |
| | | | ACTGGTCTGGGGAGCCAGGTCACTCTCAGCTACT |
| | | | CCCGCCGCTCGGGCATCCCTTCCTGCCCGCGCAG |
| | | | CAGCTGCCGCCCGGGCAGCCTCTGCTGCCCGCGC |
| | | | GCAGTCGCTGCTGGCCGGTCAGCCTCTGCCGCGC |
| | | | CCAGCAGGGCCCATCTTAGACCCCCCAGCCCCGA |
| | | | GATCCAGGTTGCTGACCCGCCTACTCCGCGGCCT |
| | | | CCTCCGCGGCCGACTGCCTGGCCTGACAAGTACG |
| | | | AGCGGGGCCGAAGCTGCTGCAGGTACGAGGCATC |
| | | | GTCTGGCATCTGCGAGATCGAGTCCTCCAGTGAT |
| | | | GAGTCGGAAGAAGGGGCCACCGGCTGCTTCCAGT |
| | | | GGCTTCTGCGGCGAAACCGCCGCCCTGGCCTGCC |
| | | | CCGGAGCCACACGGTCGGGAGCAACCCAGTCCGC |
| | | | AACTTCTTCACCCGAGCCTTCGGAAGCTGCTTCG |
| | | | GTCTATCCGAGTGTACCCGATCACGATCCCTCAG |
| | | | CCCCGGGAAGGCCAAGGATCCTATGGAGGAGAGG |
| | | | CGCAAACAGATGCGCAAGAAGCCATTGAGATGCG |
| | | | AGAGCAGAAGCGCGCAGATAAGAAACGGAGCAAG |
| | | | CTCATCGACAAGCAACTGGAGGAGGAGAAGATGG |
| | | | ACTACATGTGTACACACCGCCTGCTGCTTCTAGG |
| | | | TGCTGGAGAGTCTGGCAAAAGCACCATTGTGAAG |
| | | | CAGATGAGGATCCTGCATGTTAATGGGTTTAACG |
| | | | GAGATAGTGAGAAGGCCACTAAAGTGCAGGACAT |
| | | | CAAAAACAACCTGAAGGAGGCTTGAAACCATTGT |
| | | | GGCCGCCATGAGCAACCTGGTGCCCCCTGTGGAG |
| | | | CTGGCCAACCCTGAGAACCAGTTCAGAGTGGACT |
| | | | ACATTCTGAGCGTGATGAACGTGCCGAACTTTGA |
| | | | CTTCCCACCTGAATTCTATGAGCATGCAAGGCTC |
| | | | TGTGGGAGGATGAGGGAGTGCGTGCCTGCTACGA |
| | | | GCGCTCCAATGAGTACCAGCTGATTGACTGTGCC |
| | | | CAGTACTTCCTGGACAAGATTGATGTGATCAAGC |
| | | | AGGCCGACTACGTGCCAAGTGACCAGGACCTGCT |
| | | | TCGCTGCCGTGTCCTGACCTCTGGAATCTTTGAG |
| | | | ACCAAGTTCCAGGTGGACAAAGTCAACTTCCACA |
| | | | TGTTCGATGTGGGCGGCCAGCGCGATGAGCGCCG |
| | | | CAAGTGGATCCAGTGCTTCAATGATGTGACTGCC |
| | | | ATCTTCGTGGTGGCCAGCAGCAGCTACAACATGG |
| | | | TCATTCGGGAGGACAACCAGACTAACCGCCTGCA |
| | | | GGAGGCTCTGAACCTCTTCAAGAGCATCTGGAAC |
| | | | AACAGATGGCTGCGCACCATCTCTGTGATTCTCT |
| | | | TCCTCAACAAGCAAGACCTGCTTGCTGAGAAAGT |
| | | | CCTCGCTGGCAAATCGAAGATTGAGGACTACTTT |
| | | | CCAGAGTTCGCTCGCTACACCACTCCTGAGGATG |
| | | | CGACTCCCGAGCCGGGAGAGGACCCACGCGTGAC |
| | | | CCGGGCCAAGTACTTCATTCGGGATGAGTTTCTG |
| | | | AGAATCAGCACTGCTAGTGGAGATGGGCGCCACT |
| | | | ACTGCTACCCTCACTTTACCTGCGCCGTGGACAC |
| | | | TGAGAACATCCGCCGTGTCTTCAACGACTGCCGT |
| | | | GACATCATCCAGCGCATGCATCTCCGCCAATACG |
| | | | AGCTGCTCTAAGAAGGGAACACCCAAATTTAATT |
| | | | CAGCCTTAAGCACAATTAATTAAGAGTGAAACGT |
| | | | AATTGTACAAGCAGTTGGTCACCCACCATAGGGC |
| | | | ATGATCAACACCGCAACCTTTCCTTTTTCCCCCA |
| | | | GTGATTCTGAAAAACCCCTCTTCCCTTCAGCTTG |
| | | | CTTAGATGTTCCAAATTTAGTAAGCTTAAGGCGG |
| | | | CCTACAGAAGAAAAAGAAAAAAAAGGCCACAAAA |
| | | | GTTCCCTCTCACTTTCAGTAAATAAAATAAAAGC |
| | | | AGCAACAGAAATAAAGAAATAAATGAAATTCAAA |
| | | | ATGAAATAAATATTGTGTTGTGCAGCATTAAAAA |
| | | | ATCAATAAAAATCAAAAATGAGCAAAAAAAAAAA |
| S000058 | F13 | 140 | TGGACTGGGTGCGGCCGGCTGCAAGACTCTAGTC |
| | | | GTCGGCCCACGTGGCTGGGGCGGGGACTGCCGTG |
| | | | GCGCCTAGTGATTACGTAGCGGGTGGGGCCCGAA |
| | | | GTGCCGCTCCCTGGCGGGGCTGTTCATGGCGGTT |
| | | | TCGGGGTCTCCAACAGCTCAGGTTGAAGTCCAAA |
| | | | AGCCTCCCGAGGCGGGCTGCGGAGTTTGAGGTTT |
| | | | TTGCTGGTGTGAAATGACTGAGTACAAACTGGTG |
| | | | GTGGTTGGAGCAGGTGGTGTTGGGAAAAGCGCCC |
| | | | TGACGATCCAGCTAATCCAGAACCACTTTGTGGA |
| | | | TGAATATGATCCCACCATAGAGGATTCTTACCGA |
| | | | AAGCAAGTGGTGATTGATGGTGAGACCTGCCTGC |
| | | | TGGACATACTGGACACAGCTGGACAAGAGGAGTA |
| | | | CAGTGCCATGAGAGACCAGTACATGAGGACAGGC |
| | | | GAAGGGTTCCTCTGTGTATTTGCCATCAATAATA |
| | | | GCAAATCATTTGCAGATATTAACCTCTACAGGGA |
| | | | GCAAATTAAGCGTGTGAAAGATTCTGATGATGTC |
| | | | CCCATGGTGCTGGTAGGCAACAAGTGTGACTTGC |
| | | | CAACAAGGACAGTTGACACAAAGCAAGCCCACGA |
| | | | ACTGGCCAAGAGTTACGGAATTCCATTCATTGAG |
| | | | ACCTCAGCCAAGACCCGACAGGGTGTGGAGGATG |
| | | | CCTTTTACACACTGGTAAGGGAGATACGCCAGTA |
| | | | CCGATTGAAAAAGCTCAACAGCAGTGACGATGGC |
| | | | ACTCAAGGTTGTATGGGGTCGCCCTGTGTGCTGA |
| | | | TGTGTAAGACACTTTGAAAGTTCTGTCATCAGAA |
| | | | AAGAGCCACTTTGAAGCTGCACTGATGCCCTGGT |
| | | | TCTGACATCCCTGGAGGAGACCTGTTCCTGCTGC |
| | | | TCTCTGCATCTCAGAGAAGCTCCTGCTTCCTGCT |
| | | | TCCCCGACTCAGTTACTGAGCACAGCCATCTAAC |
| | | | CTGAGACCTCTTCAGAATAACTACCTCCTCACTC |
| | | | GGCTGTCTGACCAGAGAAATGGACCTGTCTCTCC |
| | | | CGGTCGTCTCTGCCCTGGGTTCCCCTAGAAACAG |
| | | | ACACAGCCTCCAGCTGGCTTTGTCTCCTCTGAAA |
| | | | GCAGTTTACATTGATGCAGAGAACCAAACTAGAC |
| | | | ATGCCATTCTGTTGACAACAGTTTCTTATACTCT |
| | | | AAGGTAACAACTGCTGGTGATTTTCCCCTGCCCC |
| | | | CAACTGTTGAACTTGGCCTTGTTGGTTTGGGGGG |
| | | | AAAATGTCATAAATTACTTTCTTCCCAAAATATA |
| | | | ATTAGTGTTGCTGATTGATTTGTAATGTGATCAG |
| | | | CTATATTCCATAAACTGGCATCTGCTCTGTATTC |
| | | | ATAAATGCAAACACGAATACTCTCAACTGCATGC |
| | | | AATTAAATCCAACATTCACAACAAAGTGCCTTTT |
| | | | TCCTAAAAGTGCTCTGTAGGCTCCATTACAGTTT |
| | | | GTAATTGGAATAGATGTGTCAAGAACCATTGTAT |
| | | | AGGAAGTGACTCTGAGCCATCTACCTTTGAGGGA |
| | | | AAGGTGTATGTACCTGATGGCAGATGCTTTGTGT |
| | | | ATGCACATGAAGATAGTTTCCCTGTCTGGGATTC |
| | | | TCCCAGGAGAAAGATGGAACTGAAACAATTACAA |
| | | | GTAATTTCATTTAATTGTAGCTAATCTTTTTTTT |
| | | | TTTTTTTTTTTTGGTAGACTATCACCTATAAATA |
| | | | TTTGGAATATCTTCTAGCTTACTGATAATCTAAT |
| | | | AATTAATGAGCTTCCATTATAATGAATTGGTTCA |
| | | | TACCAGGAAGCCCTCCATTTATAGTATAGATACT |
| | | | GTAAAAATTGGCATGTTGTTACTTTATAGCTGTG |
| | | | ATTAATGATTCCTCAGACCTTGCTGAGATATAGT |
| | | | TATTAGCAGACAGGTTATATCTTTGCTGCATAGT |
| | | | TTCTTCATGGAATATATATCTATCTGTATGTGGA |
| | | | GAGAACGTGGCCCTCAGTTCCCTTCTCAGCATCC |
| | | | CTCATCTCTCAGCCTAGAGAAGTTCGAGCATCCT |
| | | | AGAGGGGCTTGAACAGTTATCTCGGTTAAACCAT |
| | | | GGTGCTAATGGACCGGGTCATGGTTTCAAAACTT |
| | | | GAACAAGCCAGTTAGCATCACAGAGAAACAGTCC |
| | | | ATCCATATTTGCTCCCTGCCTATTATTCCTGCTT |
| | | | ACAGACTTGCCTGATGCCTGCTGTTAGTGCTACA |
| | | | AGGATAAAGCTTGTGTGGTTCTCACCAGGACTGG |
| | | | AAGTACCTGGTGAGCTCTGGGGTAAGCCTAGATA |
| | | | TCTTTACATTCAGACCCTTATTCTTAGCCACGTG |
| | | | GAAACTGAAGCCAGAGTCCATACCTCCATCTCCT |
| | | | TCCCCCCCCAAAAAAATTAGATTAATGTTCTTTA |
| | | | TATAGCTTTTTTAAAGTATTTAAAACATGTCTAT |
| | | | AAGTTAGGCTGCCAACTAACAAAAGCTGATGTGT |
| | | | TTGTTCAAATAAAGAGGTATCCTTCGCTACTCGA |
| | | | GAGAAGAATGTAAAATGCCATTGATTGTTGTCAC |
| | | | TTGGAGGCTTGATGTTTGCCCTGATAATTCATTA |
| | | | GTGGGTTTTGTTTGTCACATGATACCTAAGATGT |
| | | | AACTCAGCTCAGTAATTCTAATGAAAACATAAAT |
| | | | TGGATACCTTAATTGAAAAAAGCAAACCTAATTC |
| | | | CAAAATGGCCATTTTCTCTTCTGATCTTGTAATA |
| | | | CCTAAAATTCTGAGGTCCTTGGGATTCTTTTGTT |
| | | | TATAACAGGATCTTGCTGTGTAGTCCTAGCTGGC |
| | | | CTCAAACTCACAATACTCTTCCTGGATCAATCTC |
| | | | CCAAGTGCTGGGATTACAGGCACATTCCACCACA |
| | | | CACTAATCAGAAGGGGGAACAAACATTTGCTATG |
| | | | CTCCTGAGTGCTAACTGGGCTCATTCACATGGGG |
| | | | TTTGCATTCCTAGGCAAACTAAACTGCTGCCTTT |
| | | | TACAACAAGGCTCAGTCATCTTCCTGAAGCTGCT |
| | | | GAGACCAGCACTTGGTCTTGTTTTGTTTTAATAT |
| | | | GTCTATATGACTGGTGGTGGATCCGTCGACCTGC |
| | | | A |
| S000065 | F14 | 141 | GCTGGTGCCTTCGCCGTGGCCTGCTGGTGACGGT |
| | | | CCGGAGCGATGCTGAGCCCGGGCCCAGCCTCTCA |
| | | | GCTCCGCCTTGTGCGCTGCACAGATCTAGGGGAG |
| | | | CCTGACGGGACGTTGACAACGTGGAATAGGAGCA |
| | | | GTATCATCCCACCATGAGGTTGGGGATTTAAGAG |
| | | | TGGAAGATGCCAACAGCTGTGTCCTCCCATGAGG |
| | | | GTGTCCCCTTTCAAGTTCTCAGAACGGATGCAGG |
| | | | ACTGCAGATCTGTGCTGGCAACAGCAGAGGCTAT |
| | | | ATTCCCAGAGGAGTCTCCAGCCGGCCTGAAAGCA |
| | | | AATATCTATCCTAAGTGACATGTCTGCCAATTTG |
| | | | GTTCTGGGTGGGCACATTTGGTAATCCTGGTCTG |
| | | | TACCACAGNGATCTTCTACGCCGTTTTAAAACAT |
| | | | AAACATTGGGTTTATTAAACCAGGAAAGAACAAA |
| | | | CAAAACAAAGAAACAACGGGGGGGGCGGGTCTAA |
| | | | GAATATCCG |
| S000072 | F15 | 142 | TGCTCCATGCCCTTGTCCTCGCTCTGGCCCTTGC |
| | | | CTCTTGCCCTAGCCTTTTCTCCGCCTCTAAGTTC |
| | | | TTGTCCCGTCCCTAGGTCCTTGTTCCAGGGGGTG |
| | | | GGGGCGGGGCGGACTAAGGCTGGCCTGCCACTCC |
| | | | AGCGAGCAGGCTATCTCCTAGTTCTCGCTGCTCG |
| | | | GACTAGCCATTGCCGCCGCCTCACCTCTGCTGCA |
| | | | AGTAGCCTCGCCGTCGGGGAGCCCTACCACACGG |
| | | | TCCGCCCTCAGCATGATGGACTTGGAGTTGCCAC |
| | | | CGCCAGACTACAGTCCCAGCAGGACATGGATTTG |
| | | | ATTGACATCCTTTGGAGGCAAGACATAGATCTTG |
| | | | GAGTAAGTCGAAGTGTTTGACTTTAGTCAGCGAC |
| | | | AGAAGGACTATGAGCTGGAAAAACAGAAAAAACT |
| | | | CGAAAAGGAAAGACAAGAGCAACTCCAGAAGGAA |
| | | | CAGGAGAAGGCCTTTTTTGCTCAGTTTCAACTGG |
| | | | ATGAAGAAACAGGAGAATCCTCCCAATTCAGCCG |
| | | | GCCCAGCACATCCAGACAGACACCAGTGGATCCG |
| | | | CCAGCTACTCCCAGGTTGCCCACATTCCCAAACA |
| | | | AGATGCCTTGTACTTTGAAGACTGTATGCAGCTA |
| | | | TTTTGGCAGAGACATTCCCATTTGTAGATGACCA |
| | | | TGAGTCGCTTGCCCTGGATATCCCCAGCCACGCT |
| | | | GAAAGTTCAGTCTTCACTGCCCCTCATCAGGCCC |
| | | | AGTCCCTCAATAGCTCTCTGGAGGCAGCCATGAC |
| | | | TGATTTAAGCAGCATAGAGCAGGACATGGAGCAA |
| | | | GTTTGGCAGGAGCTATTTTCCATTCCCGAATTAC |
| | | | AGTGTCTTAATACCGAAAACAAGCAGCTGGCTGA |
| | | | TACTACCGCTGTTCCCAGCCCAGAAGCCACACTG |
| | | | ACAGAAATGGACAGCAATTACCATTTTTACTCAT |
| | | | CGATCTCCTCGCTGGAAAAAGAAGTGGGCAACTG |
| | | | TGGTCCACATTTCCTTCATGGTTTTGAGGATTCT |
| | | | TTCAGCAGCATCCTCTCCACTGATGATGCCAGCC |
| | | | AGCTGACCTCCTAGACTCAAATCCCACCTTAAAC |
| | | | ACAGATTTTGGCGATGAATTTTATTCTGCTTTCA |
| | | | TAGCAGAGCCCAGTGACGGTGGCAGCATGCCTTC |
| | | | CTCCGCTGCCATCAGTCAGTCACTCTCTGAACTC |
| | | | CTGGACGGGACTATTGAAGGCTGTGACCTGTCAC |
| | | | TGTGTAAAGCTTTCAACCCGAAGCACGCTGAAGG |
| | | | CACAATGGAATTCAATGACTCTGACTCTGGCATT |
| | | | TCACTGAACACGAGTCCCAGCCGAGCGTCCCCAG |
| | | | AGCACTGCGTGGAGTCTTCCATTTACGGAGACCC |
| | | | ACCGCCTGGGTTCAGTGACTCGGAAATGGAGGAG |
| | | | CTAGATAGTGCCCCTGGAAGTGTCAAACAGAACG |
| | | | GCCCTAAAGCACAGCCAGCACATTCTCCTGGAGA |
| | | | CACAGTACAGCCTCTGTCACCAGCTCAAGGGCAC |
| | | | AGTGCTCCTATGCGTGAATCCCAATGTGAAAATA |
| | | | CAACAAAAAAAGAAGTTCCCGTGAGTCCTGGTCA |
| | | | TCAAAAAGCCCCATTCACAAAAGACAAACATTCA |
| | | | AGCCGCTTAGAGGCTCATCTCACACGAGATGAGC |
| | | | TTAGGGCAAAAGCTCTCCATATTCCATTCCCTGT |
| | | | CGAAAAAATCATTAACCTCCCTGTTGATGACTTC |
| | | | AATGAAATGATGTCCAAGGAGCAATTCAATGAAG |
| | | | CTCAGCTCGCATTGATCCGAGATATACGCAGGAG |
| | | | AGGTAAGAATAAAGTCGCCGCCCAGAACTGTAGG |
| | | | AAAAGGAAGCTGGAGAACATTGTCGAGCTGGAGC |
| | | | AAGACTTGGGCCACTTAAAAGACGAGAGAGAAAA |
| | | | ACTACTCAGAGAAAAGGGAGAAAACGACAGAAAC |
| | | | CTCCATCTACTGAAAAGGCGGCTCAGCACCTTGT |
| | | | ATCTTGAAGTCTTCAGCATGTTACGTGATGAGGA |
| | | | TGGAAAGCCTTACTCTCCCAGTGAATACTCTCTG |
| | | | CAGCAAACCAGAGATGGCAATGTGTTCCTTGTTC |
| | | | CCAAAAGCAAGAAGCCAGATACAAAGAAAAACTA |
| | | | GGTTCGGGAGGATGGAGCCTTTTCTGAGCTAGTG |
| | | | TTTGTTTTGTACTGCTAAAACTTCCTACTGTGAT |
| | | | GTGAAATGCAGAAACACTTTATAAGTAACTATGC |
| | | | AGAATTATAGCCAAAGCTAGTATAGCAATAATAT |
| | | | GAAACTTTACAAAGCATTAAAGTCTCAATGTTGA |
| | | | ATCAGTTTCATTTTAACTCTCAAGTTAATTTCTT |
| | | | AGGCACCATTTGGGAGAGTTTCTGTTTAAGTGTA |
| | | | AATACTACAGACTTATTTATACTGTTCTCACTTG |
| | | | TTACAGTCATAGACTTATATGACATCTGGCTAAA |
| | | | AGCAAACTATTGAAAACTAACCAGACCACTATAC |
| | | | TTTTTTATATACTGTATGAACAGGAAATGACATT |
| | | | TTTATATTAAATTGTTTAGCTCATAAAAATTAAA |
| | | | AGGAGCTAGCACTAATAAAAGAATATCATGACT |
| S000083 | F18 | 143 | TATATTCCGGGGGTCTGCGCGGCCGAGGACCCTT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAGAAGGGAG |
| | | | GGGAGGGATCCTGAGTCGCAGTATAAAAGAAGCT |
| | | | TTTCGGGCGTTTTTTTCTGACTCGCTGTAGTAAT |
| | | | TCCAGCGAGAGACAGAGGGAGTGAGCGGACGGTT |
| | | | GGAAGAGCCGTGTGTGCAGAGCCGCGCTCCGGGG |
| | | | CGACCTAAGAAGGCAGCTCTGGAGTGAGAGGGGC |
| | | | TTTGCCTCCGAGCCTGCCGCCCACTCTCCCCAAC |
| | | | CCTGCGACTGACCCAACATCAGCGGCCGCAACCC |
| | | | TCGCCGCCGCTGGGAAACTTTGCCCATTGCAGCG |
| | | | GGCAGACACTTCTCACTGGAACTTACAATCTGCG |
| | | | AGCCAGGACAGGACTCCCCAGGCTCCGGGGAGGG |
| | | | AATTTTTGTCTATTTGGGGACAGTGTTCTCTGCC |
| | | | TCTGCCCGCGATCAGCTCTCCTGAAAAGAGCTCC |
| | | | TCGAGCTGTTTGAAGGCTGGATTTCCTTTGGGCG |
| | | | TTGGAAACCCCGCAGACAGCCACGACGATGCCCC |
| | | | TCAACGTGAACTTCACCAACAGGAACTATGACCT |
| | | | CGACTACGACTCCGTACAGCCCTATTTCATCTGC |
| | | | GACGAGGAAGAGAATTTCTATCACCAGCAACAGC |
| | | | AGAGCGAGCTGCAGCCGCCCGCGCCCAGTGAGGA |
| | | | TATCTGGAAGAAATTCGAGCTGCTTCCCACCCCG |
| | | | CCCCTGTCCCCGAGCCGCCGCTCCGGGCTCTGCT |
| | | | CTCCATCCTATGTTGCGGTCGCTACGTCCTTCTC |
| | | | CCCAAGGGAAGACGATGACGGCGGCGGTGGGAAC |
| | | | TTCTCCACCGCCGATCAGCTGGAGATGATGACCG |
| | | | AGTTACTTGGAGGAGACATGGTGAACCAGAGCTT |
| | | | CATCTGCGATCCTGACGACGAGACCTTCATCAAG |
| | | | AACATCATCATCCAGGACTGTATGTGGAGCGGTT |
| | | | TCTCAGCCGCTGCCAAGCTGGTCTCGGAGAAGCT |
| | | | GGCCTCCTACCAGGCTGCGCGCAAAGACAGCACC |
| | | | AGCCTGAGCCCCGCCCGCGGGCACAGCGTCTGCT |
| | | | CCACCTCGAGCCTGTACCTGCAGGACCTCACCGC |
| | | | CGCCGCGTCCGAGTGCATTGACCCCTCAGTGGTC |
| | | | TTTCCCTACCCGCTCAACGACAGCAGCTCGCCCA |
| | | | AATCCTGTACCTCGTCCGATTCCACGGCCTTCTC |
| | | | TCCTTCCTCGGACTCGCTGCTGTCCTCCGAGTCC |
| | | | TCCCCACGGGCCAGCCCTGAGCCCCTAGTGCTGC |
| | | | ATGAGGAGACACCGCCCACCACCAGCAGCGACTC |
| | | | TGAAGAAGAGCAAGAAGATGAGGAAGAAATTGAT |
| | | | GTGGTGTCTGTGGAGAAGAGGCAAACCCCTGCCA |
| | | | AGAGGTCGGAGTCGGGCTCATCTCCATCCCGAGG |
| | | | CCACAGCAAACCTCCGCACAGCCCACTGGTCCTC |
| | | | AAGAGGTGCCACGTCTCCACTCACCAGCACAACT |
| | | | ACGCCGCACCCCCCTCCACAAGGAAGGACTATCC |
| | | | AGCTGCCAAGAGGGCCAAGTTGGACAGTGGCAGG |
| | | | GTCCTGAAGCAGATCAGCAACAACCGCAAGTGCT |
| | | | CCAGCCCCAGGTCCTCAGACACGGAGGAAAACGA |
| | | | CAAGAGGCGACACACAACGTCTTGGAACGTCAGA |
| | | | GGAGGAACGAGCTGAAGCGCAGCTTTTTTGCCCT |
| | | | GCGTGACCAGATCCCTGAATTGGAAAACAACGAA |
| | | | AAGGCCCCCAAGGTAGTGATCCTCAAAAAAGCCA |
| | | | CCGCCTACATCCTGTCCATTCAAGCAGACGAGCA |
| | | | CAAGCTCACCTCTGAAAAGGACTTATTGAGGAAA |
| | | | CGACGAGAACAGTTGAAACACAAACTCGAACAGC |
| | | | TTCGAAACTCTGGTGCATAAACTGACCTAACTCG |
| | | | AGGAGGAGCTGGAATCTCTCGTGAGAGTAAGGAG |
| | | | AACGGTTCCTTCTGACAGAACTGATGCGCTGGAA |
| | | | TTAAAATGCATGCTCAAAGCCTAACCTCACAACC |
| | | | TTGGCTGGGGCTTTGGGACTGTAAGCTTCAGCCA |
| | | | TAATTTTAACTGCCTCAAACTTAAATAGTATAAA |
| | | | AGAACTTTTTTTATGCTTCCCATCTTTTTTCTTT |
| | | | TTCCTTTTAACAGATTTGTATTTAATTGTTTTTT |
| | | | TAAAAAAATCTTAAAATCTATCCAATTTTCCCAT |
| | | | GTAAATAGGGCCTTGAAATGTAAATAACTTTAAT |
| | | | AAAACGTTTATAACAGTTACAAAAGATTTTAAGA |
| | | | CATGTACCATAATTTTTTTT |
| S000087 | F17 | 144 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGCCAGGACAGGACTCCCCAGGCTCCGGGGAGG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGCTACGTCCTTCT |
| | | | CCCCAAGGGAAGACGATGACGGCGGCGGTGGCAA |
| | | | CAACTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGCCGCTGCCAAGCTGGTCTCGGAGAAGC |
| | | | TGGCCTCCTACCAGGCTGCGCGCAAAGACAGCAC |
| | | | CAGCCTGAGCCCCGCCCGCGGGCACAGCGTCTGC |
| | | | TCCACCTCCAGCCTGTACCTGCAGGACCTCACCG |
| | | | CCGCCGCGTCCGAGTGCATTGACCCCTCAGTGGT |
| | | | CTTTCCCTACCCGCTCAACGACAGCAGCTCGCCC |
| | | | AAATCCTGTACCTCGTCCGATTCCACGGCCTTCT |
| | | | CTCCTTCCTCGGACTCGCTGCTGTCCTCCGAGTC |
| | | | CTCCCCACGGGCCAGCCCTCAGCCCCTAGTGCTG |
| | | | CATGAGGAGACACCGCCCACCACCAGCAGCGACT |
| | | | CTGAAGAAGAGCAAGAAGATGAGGAAGAAATTGA |
| | | | TGTGGTGTCTGTGGAGAAGAGGCAAACCCCTGCC |
| | | | AAGAGGTCGGAGTCGGGCTCATCTCCATCCCGAG |
| | | | GCCACAGCAAACCTCCGCACAGCCCACTGGTCCT |
| | | | CAAGAGGTGCCACGTCTCCACTCACCAGCACAAC |
| | | | TACGCCGCACCCCCCTCCACAAGGAAGGACTATC |
| | | | CAGCTGCCAAGAGGGCCAAGTTGGACAGTGGCAG |
| | | | GGTCCTGAAGCAGATCAGCAACAACCGCAAGTGC |
| | | | TCCAGCCCCAGGTCCTCAGACACGGAGGAAAACG |
| | | | ACAAGAGGCGGACACACAACGTCTTGGAACGTCA |
| | | | GAGGAGGAACGAGCTGAAGCGCAGCTTTTTTGCC |
| | | | CCTGCGTGACCAGATCCCTGAATTGGAAAACAAC |
| | | | GAAAAGGCCCCCAAGGTAGTGATCCTCAAAAAAG |
| | | | CCACCGCCTACATCCTGTCCATTCAAGCAGACGA |
| | | | GCACAAGCTCACCTCTGAAAACTTATTGAGGAAA |
| | | | CGACGAGAACAGTTGAAACACAAACTCGAACAGC |
| | | | TTCGAAACTCTGGTGCATAAACTGACCTAACTCG |
| | | | AGGAGGAGCTGGAATCTCTCGTGAGAGTAAGGAA |
| | | | CGGTTCCTTCTGACAGAACTGATGCGCTGGAATT |
| | | | AAAATGCATGCTCAAAGCCTAACCTCACAACCTT |
| | | | GGCTGGGGCTTTGGGACTGTAAGCTTCAGCCATA |
| | | | ATTTTAACTGCCTCAAATTAAATAGTATAAAAGA |
| | | | ACTTTTTTTATGCTTCCCATCTTTTTTCTTTTTC |
| | | | CTTTTAACAGATTTGTATTTAATTGTTTTTTTAA |
| | | | AAAAATCTTAAAATCTATCCAATTTTCCCATGTA |
| | | | AATAGGGCCTTGAAATGTAAATAACTTTAATAAA |
| | | | ACGTTTATAACAGTTACAAAAGATTTTAAGACAT |
| | | | GTACCATAATTTTTTTT |
| S000090 | F18 | 145 | TATATTCCGGGGGTCTGCGCGGCCGAGGACCCCT |
| | | | GGGTGCGCTGCTCTCAGCTGCCGGGTCCGACTCG |
| | | | ACCTCACTCAGCTCCCCTCCTGCCTCCTGAAGGG |
| | | | CCAGCTTCGCCGACGCTTGGCGGGAAAAAGAAGG |
| | | | GAAGGGGAGGGATCCTGAGTCGCAGTATAAAAGA |
| | | | AGCATTTTCGGGCGTTTTTTTCTGACTCGCTGTA |
| | | | GTAATTCCAGCGAGAGACAGAGGGAGTGAGCGGA |
| | | | CGGTTGGAAGAGCCGTGTGTGCAGAGCCGCGCTC |
| | | | CGGGGCGACCTAAGAAGGCAGCTCTGGAGTGAGA |
| | | | GGGGCTTTGCCTCCGAGCCTGCCGCCCACTCTCC |
| | | | CCAACCCTGCGACTGACCCAACATCAGCGGCCGC |
| | | | AACCCTCGCCGCCGCTGGGAAACTTTGCCCATTG |
| | | | CAGCGGGCAGACACTTCTCACTGGAACTTACAAT |
| | | | CTGCGAGCCAGGACAGGACTCCCCAGGCTCCGGG |
| | | | GAGGGAATTTTTGTCTATTTTGGGGACAGTGTTC |
| | | | TCTGCCTCTGCCCGCGATCAGCTCTCCTGAAAGA |
| | | | GCTCCTCGAGCTGTTTGAAGGCTGGATTTCCTTT |
| | | | GGGCGTTGGAAACCCCGCAGACAGCCACGACGAT |
| | | | GCCCCTCAACGTGAACTTCACCAACAGGAACTAT |
| | | | GACCTCGACTACGACTCCGTACAGCCCTATTTGA |
| | | | TCTGCGACGAGGAAGAGAATTTCTATCACCAGCA |
| | | | ACAGCAGAGCGAGCTGCAGCCGCCCGCGCCCAGT |
| | | | GAGGATATCTGGAAGAAATTCGAGCTGCTTCCCA |
| | | | CCCCGCCCCTGTCCCCGAGCCGCCGCTCCGGGCT |
| | | | CTGCTCTCCATCCTATGTTGCGGTCGCTACGTCC |
| | | | TTCTCCCCAAGGGAAGACGATGACGGCGGCGGTG |
| | | | GCAACTTCTCCACCGCCGATCAGCTGGAGATGAT |
| | | | GACCGAGTTACTTGGAGGAGACATGGTGAACCAG |
| | | | AGCTTCATCTGCGATCCTGACGACGAGACCTTCA |
| | | | TCAAGAACATCATCATCCAGGACTGTATGTGGAG |
| | | | CGGTTTCTCAGCCGCTGCCAAGCTGGTCTCGGAG |
| | | | AAGCTGGCCTCCTACCAGGCTGCGCGCAAAGACA |
| | | | GCACCAGCCTGAGCCCCGCCCGCGGGCACAGCGT |
| | | | CTGCTCCACCTCCAGCCTGTACCTGCAGGACCTC |
| | | | ACCGCCGCCGCGTCCGAGTGCATTGACCCCTCAG |
| | | | TGGTCTTTCCCTACCCGCTCAACGACAGCAGCTC |
| | | | GGCCAAATCCTGTACCTCGTCCGATTCCACGGCC |
| | | | TTCTCTCCTTCCTCGGACTCGCTGCTGTCCTCCG |
| | | | AGTCCTCCCCACGGGCCAGCCCTGAGCCCCTAGT |
| | | | GCTGCATGAGGAGACAGCGCCCACCACCAGCAGC |
| | | | GACTCTGAAGAAGAGCAAGAAGATGAGGAAGAAA |
| | | | TTGATGTGGTGTCTGTGGAGAAGAGGCAAACCCC |
| | | | TGCCAAGAGGTCGGAGTCGGGCTCATCTCCATCC |
| | | | CGAGGCCACAGCAAACCTCCGCACAGCCCACTGG |
| | | | TCCTCAAGAGGTGCCACGTCTCCACTCACCAGCA |
| | | | CAACTACGCCGCACCCCCCTCCACAAGGAAGGAC |
| | | | TATCCAGCTGCCAAGAGGGCCAAGTTGGACAGTG |
| | | | GCAGGGTCCTGAAGCAGATCAGCAACAACCGCAA |
| | | | GTGCTCCAGCCCCAGGTCCTCAGACACGGAGGAA |
| | | | AACGACAAGAGGCGGACACACAACGTCTTGGAAC |
| | | | GTCAGAGGAGGAACGAGCTGAAGCGCAGCTTTTT |
| | | | TGCCCTGCGTGACCAGATCCCTGAATTGGAAAAC |
| | | | AACGAAAAGGCCCCCAAGGTAGTGATCCTCAAAA |
| | | | AAGCCACCGCCTACATCCTGTCCATTCAAGCAGA |
| | | | CGAGCACAAGCTCACCTCTGAAAAGGACTTATTG |
| | | | AGGAAACGACGAGAACAGTTGAAACACAAACTCG |
| | | | AACAGCTTCGAAACTCTGGTGCATAAACTGACCT |
| | | | AACTCGAGGAGGAGCTGGAATCTCTCGTGAGAGT |
| | | | AAGGAGAACGGTTCCTTCTGACAGAACTGATGCG |
| | | | CTGGAATTAAAATGCATGCTCAAAGCCTAACCTC |
| | | | ACAACCTTGGCTGGGGCTTTGGGACTGTAAGCTT |
| | | | CAGCCATAATTTTAACTGCCTCAAACTTAAATAG |
| | | | TATAAAAGAACTTTTTTTATGCTTCCCATCTTTT |
| | | | TTCTTTTTCCTTTTAACAGATTTGTATTTAATTG |
| | | | TTTTTTTAAAAAAATCTTAAAATCTATCCAATTT |
| | | | TCCCATGTAAATAGGGCCTTGAAATGTAAATAAC |
| | | | TTTAATAAAACGTTTATAACAGTTACAAAAGATT |
| | | | TTAAGACATGTACCATAATTTTTTTT |
| S000092 | F19 | 146 | TTTTTTTTTTTGCTTTTTTTTTTCTTTCTTTCTT |
| | | | TTTCTTTTTTTCTTTCTTTTTTTGAGAGTATTTG |
| | | | GGCGACGCATTGGGCGCCCTCTGCAGTACGCGCA |
| | | | GCGAAGCGCACCGAGGCTGCGGAGGCAGAGCTGC |
| | | | ATGCTGGGCGCGTGGACAGGTGGGCGTGAAGCAA |
| | | | AAGGACATTTTTGGGAGTATGGGGTTTGGGACGA |
| | | | GGGTGGGGAGAAAAGGCAAAAGGAGACCACGTTA |
| | | | GACTGAAGAGCTAAAAAGGGCACGGACTTGGCTA |
| | | | CGCCAAGACGAAGCCAGCCTGGGAGAGGGAGTCT |
| | | | CTGGGACCGGCGGGGGGAGGGGGGGGGCTCCTGA |
| | | | AGCTGGCTGGTTGGTGGGAAGGAGGGGCTCACAA |
| | | | ACACAGTAGGGAAGTCTTGTCACTGCGAAGGGGA |
| | | | CGCGGCATCCGACTCTCCTCTGGAACTTCTAAAA |
| | | | CGTTCAGCTCTGGCCTAGTCTCCGCTGGGGCCGN |
| | | | CGCCCGCGCCTCCCCGGGCGCCCCCAG |
| S000098 | F20 | 147 | GCCTTTAAAAACGTTTATTTTTATGTGCATAAGT |
| | | | GCTTTGCATACTATGAGCATGTCTGGTGCTCCAA |
| | | | AAGGCCAGGAGAGGGTGCCAGATCCTCTGAAACC |
| | | | AGATGTAGAGGGTTATGAGCCGCCATGAGGATGC |
| | | | TGGGAACTGAACCCAGGCCCTTTGCACAAGCAGC |
| | | | AAGTGCTCCTAGCGCTTCAGCCACTTCTTCATCC |
| | | | TCAGCATGATGAACAGAGTAAAAGCCATGAACAT |
| | | | TGATGAAATAAAAACATGAGTCATGTTAAAGAAC |
| | | | TCTGGATCTTAACGGTGGACAATAGGCTATACTG |
| | | | TCTCATTTCATTTAAAAAAATATGCATCTTTATA |
| | | | TAATCATAGAAAAAGATGGCGAGGCACAGTCACA |
| | | | CCAAAACATTGAGAAGATTACTCATGGGGCATTA |
| | | | GAATTTGGAGTGGTTTTAGCTTCTTTCCCACTTA |
| | | | CTTCCTGTTTTCATGTCACATGAAAAGTATTAAT |
| | | | GCTGCCCTCAAAACAGAGCAACATAGTTTATTAG |
| | | | GGGAGACTGAGGCCTAGACAAGACAGCTCTTTTA |
| | | | CACTGAATGACTGTGGACCTGACAAAGTGGTAGA |
| | | | TGGTGTGCTGTGACTGTTCCTGCCGTGGTAGCTA |
| | | | CATGGTCTGAAGACAATTGCCGTGTGCAGGAGGA |
| | | | ATCTTCTTGCTCGGGCATCTGACCGCT |
| S000104 | F21 | 148 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTCGACCCAACATCAGCGGCCGCAAC |
| | | | CCTCGCCGCCGCTGGGAAACTTTGCCCATTGCAG |
| | | | CGGGCAGACACTTCTCACTGGAACTTACAATCTG |
| | | | CGAGCCAGGACAGGACTCCCCAGGCTCCGGGGGA |
| | | | GGGAATTTTTGTCTATTTGGGGACAGTGTTCTCT |
| | | | GCCTCTGCCCGCGATCAGCTCTCCTGAAAAGAGC |
| | | | TCCTCGAGCTGTTTGAAGGCTGGATTTCCTTTGG |
| | | | GCGTTGGAAACCCCGCAGACAGCCACGACGATGC |
| | | | CCCTCAACGTGAACTTCACCAACAGGAACTATGA |
| | | | CCTCGACTACGACTTCCGTACAGCCCTATTTCAT |
| | | | CTGCGACGAGGAAGAGAATTTCTATCACCAGCAA |
| | | | CAGCAGAGCGAGCTGCAGCCGCCCGCGCCCAGTG |
| | | | AGGATATCTGGAAGAAATTCGAGCTGCTTCCCAC |
| | | | CCCGCCCCTGTCCCCGAGCCGCCGCTCCGGGCTC |
| | | | TGCTCTCCATCCTATGTTGCGGTCGCTACGTCCT |
| | | | TCTCCCCAAGGGAAGACGATGACGGCGGCGGTGG |
| | | | CAACTTCTCCACCGCCGATCAGCTGGAGATGATG |
| | | | ACCGAGTTACTTGGAGGAGACATGGTGAACCAGA |
| | | | GCTTCATCTGCGATCCTGACGACGAGACCTTCAT |
| | | | CAAGAACATCATCATCCAGGACTGTATGTGGAGC |
| | | | GGTTTCTCAGCCGCTGCCAAGCTGGTCTCGGAGA |
| | | | AGCTGGCCTCCTACCAGGCTGCGCGCAAAGACAG |
| | | | CACCAGCCTGAGCCCCGCCCGCGGGCACAGCGTC |
| | | | TGCTCCACCTCCAGCCTGTACCTGCAGGACCTCA |
| | | | CCGCCGCCGCGTCCGAGTGCATTGACCCCTCAGT |
| | | | GGTCTTTCCCTACCCGCTCAACGACAGCAGCTCG |
| | | | CCCATCCTGTACCTCGTCCGATTCCACGGCCTTC |
| | | | TCTCCTTCCTCGGACTCGCTGCTGTCCTCCGAGT |
| | | | CCTCCCCACGGGCCAGCCCTGAGCCCCTAGTGCT |
| | | | GCATGAGGAGACACCGCCCACCACCAGCAGCGAC |
| | | | TCTGAAGAAGAGCAAGAAGATGAGGAAGAAATTG |
| | | | ATGTGGTGTCTGTGGAGAAGAGGCAAACCCCTGC |
| | | | CAAGAGGTCGGAGTCGGGCTCATCTCCATCCCGA |
| | | | GGCCACAGCAAACCTCCGCACAGCCCACTGGTCC |
| | | | TCAAGAGGTGCCACGTCTCCACTCACCAGCACAA |
| | | | CTACGCCGCACCCCCCTCCACAAGGAAGGACTAT |
| | | | CCAGCTGCCAAGAGGGCCAAGTTGGACAGTGGCA |
| | | | GGGTCCTGAAGCAGATCAGCAACAACCGCAAGTG |
| | | | CTCCAGCCCCAGGTCCTCAGACACGGAGGAAAAC |
| | | | GACAAGAGGCGGACACACAACGTCTTGGAACGTC |
| | | | AGAGGAGGAACGAGCTGAAGCGCAGCTTTTTTGC |
| | | | CCTGCGTGACCAGATCCCTGAATTGGAAAACAAC |
| | | | GAAAAGGCCCCCAAGGTAGTGATCCTCAAAAAAG |
| | | | CCACCGCCTACATCCTGTCCATTCAAGCAGACGA |
| | | | GCACAAGCTCACCTCTGAAAAGGACTTATTGAGG |
| | | | AAACGACGAGAACAGTTGAAACACAAACTCGAAC |
| | | | AGCTTCGAAACTCTGGTGCATAAACTGACCTAAC |
| | | | TCGAGGAGGAGCTGGAATCTCTCGTGAGAGTAAG |
| | | | GAGAACGGTTCCTTCTGACAGAACTGATGCGCTG |
| | | | GAATTAAAATGCATGCTCAAAGCCTAACCTCACA |
| | | | ACCTTGGCTGGGGCTTTGGGACTGTAAGCTTCAG |
| | | | CCATAATTTTAACTGCCTCAAACTTAAATAGTAT |
| | | | AAAAGAACTTTTTTTATGCTTCCCATCTTTTTTC |
| | | | TTTTTCCTTTTAACAGATTTGTATTTAATTGTTT |
| | | | TTTTAAAAAAATCTTAAAATCTATCCAATTTTCC |
| | | | CATGTAAATAGGGCCTTGAAATGTAAATAACTTT |
| | | | AATAAAACGTTTATAACAGTTACAAAAGATTTTA |
| | | | AGACATGTACCATAATTTTTTTT |
| S000106 | F22 | 149 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGCCAGGACAGGACTCCCCAGGCTCCGGGGAGG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGTTACGTCCTTCT |
| | | | CCCCAAGGGAAGACGATGACGGCGGCGGTGGCAA |
| | | | CTTCTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGCCGCTGCCAAGCTGGTCTCGGAGAAGC |
| | | | TGGCCTCCTACCAGGCTGCGCGCAAAGACAGCAC |
| | | | CAGCCTGAGCCCCGCCCGCGGGCACAGCGTCTGC |
| | | | TCCACCTCCAGCCTGTACCTGCAGGACCTCACCG |
| | | | CCGCCGCGTCCGAGTGCATTGACCCCTCAGTGGT |
| | | | CTTTCCCTACCCGCTCAACGACAGCAGCTCGCCC |
| | | | AAATCCTGTACCTCGTCCGATTCCACGGCCTTCT |
| | | | CTCCTTCCTCGGAGTCGGTGCTGTCCTCCGAGTC |
| | | | CTCCCCACGGGCCAGCCCTGAGCCCCTAGTGCTG |
| | | | CATGAGGAGACACCGCCCACCACCAGCAGCGACT |
| | | | CTGAAGAAGAGCAAGAAGATGAGGAAGAAATTGA |
| | | | TGTGGTGTCTGTGGAGAAGAGGCAAACCCCTGCC |
| | | | AAGAGGTCGGAGTCGGGCTCATCTCCATCCCGAG |
| | | | GCCACAGCAAACCTCCGCACAGCCCACTGGTCCT |
| | | | CAAGAGGTGCCACGTCTCCACTCACCAGCACAAC |
| | | | TACGCCGCACCCCCCTCCACAAGGAAGGACTATC |
| | | | CAGCTGCCAAGAGGGCCAAGTTGGACAGTGGCAG |
| | | | GGTCCTGAAGCAGATCAGCAACAACCGCAAGTGC |
| | | | TCCAGCCCCAGGTCCTCAGACACGGAGGAAAACG |
| | | | ACAAGAGGCGGACACACAACGTCTTGGAACGTCA |
| | | | GAGGAGGAACGAGCTGAAGCGCAGCTTTTTTGCC |
| | | | CTGCGTGACCAGATCCCTGAATTGGAAAACAACG |
| | | | AAAAGGCCCCCAAGGTAGTGATCCTCAAAAAAGC |
| | | | CACCGCCTACATCCTGTCCATTCAAGCAGACGAG |
| | | | CACAAGCTCACCTCTGAAAAGGACTTATTGAGGA |
| | | | AACGACGAGAACAGTTGAAACACAAACTCGAACA |
| | | | GCTTCGAAACTCTGGTGCATAAACTGACCTAACT |
| | | | CGAGGAGGAGCTGGAATCTCTCGTGAGAGTAAGG |
| | | | AGAACGGTTCCTTCTGACAGAACTGATGCGCTGG |
| | | | AATTTAAAAGCATGCTCAAAGCCTAACCTCACAA |
| | | | CCTTGGCTGGGGCTTTGGGACTGTAAGCTTCAGC |
| | | | CATAATTTTAACTGCCTCAAACTTAAATAGTATA |
| | | | AAAGAACTTTTTTTATGCTTCCCATCTTTTTTCT |
| | | | TTTTCCTTTTAACAGATTTGTATTTAATTGTTTT |
| | | | TTTAAAAAAATCTTAAAATCTATCCAATTTTCCC |
| | | | ATGTAAATAGGGCCTTGAAATGTAAATAACTTTA |
| | | | ATAAAACGTTTATAACAGTTACAAAAGATTTTAA |
| | | | GACATGTACCATAATTTTTTTT |
| S000107 | F3 | 150 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGCCAGGACAGGACTCCCCAGGCTCCGGGGAGG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGCTACGTCCTTCT |
| | | | CCCCAAGGGAAGACGATGACGGCGGCGGTGGCAA |
| | | | CTTCTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGCCGCTGCCAAGCTGGTCTCGGAGAAGC |
| | | | TGGGCCTCCTACCAGGCTGCGCGCAAAGACAGCA |
| | | | CCAGCCTGAGCCCCGCCCGCGGGCACAGCGTCTG |
| | | | CTCCACCTCCAGCCTGTACCTGCAGGACCTCACC |
| | | | GCCGCCGCGTCCGAGTGCATTGACCCCTCAGTGG |
| | | | TCTTTCCCTACCCGCTCAACGACAGCAGCTCGCC |
| | | | CAAATCCTGTACCTCGTCCGATTCCACGGCCTTC |
| | | | TCTCCTTCCTCGGACTCGCTGCTGTCCTCCGAGT |
| | | | CCTCCCCACGGGCCAGCCCTGAGCAACTAGTGCT |
| | | | GCATGAGGAGACACCGCCCACCACCAGCAGCGAC |
| | | | TCTGAAGAAGAGCAAGAAGATGAGGAAGAAATTG |
| | | | ATGTGGTGTCTGTGGAGAAGAGGCAAACCCCTGC |
| | | | CAAGAGGTCGGAGTCGGGCTCATCTCCATCCCGA |
| | | | GGCCACAGCAAACCTCCGCACAGCCCACTGGTCC |
| | | | TCAAGAGGTGCCACGTCTCCACTCACCAGCACAA |
| | | | CTACGCCGCACCCCCCACAAGGAAGGACTATCCA |
| | | | GCTGCCAAGAGGGCCAAGTTGGACAGTGGCAGGG |
| | | | TCCTGAAGCAGATCAGCAACAACCGCAAGTGCTC |
| | | | CAGCCCCAGGTCCTCAGACACGGAGGAAAACGAC |
| | | | AAGAGGCGGACACACAACGTCTTGGAACGTCAGA |
| | | | GGAGGAACGAGCTGAAGCGCAGCTTTTTTGCCCT |
| | | | GCGTGACCAGATCCCTGAATTGGAAAACAACGAA |
| | | | AAGGCCCCCAAGGTAGTGATCCTCAAAAAAGCCA |
| | | | CCGCCTACATCCTGTCCATTCAAGCAGACGAGCA |
| | | | CAAGCTCACCTCTGAAAAGGACTTATTGAGGAAA |
| | | | CGACGAGAACAGTTGAAACACAAACTCGAACAGC |
| | | | TTCGAAACTCTGGTGCATAAACTGACCTAACTCG |
| | | | AGGAGGAGCTGGAATCTCTCGTGAGAGTAAGGAG |
| | | | AACGGTTCCTTCTGACAGAACTGATGCGCTGGAA |
| | | | TTAAAATGCATGCTCAAAGCCTAACCTCACAACC |
| | | | TTGGCTGGGGCTTTGGGACTGTAAGCTTCAGCCA |
| | | | TAATTTTAACTGCCTCAAACTTAAATAGTATAAA |
| | | | AGAACTTTTTTTATGCTTCCCATCTTTTTTCTTT |
| | | | TTCCTTTTAACAGATTTGTATTTAATTGTTTTTT |
| | | | TAAAAAAATCTTAAAATCTATCCAATTTTCCCAT |
| | | | GTAAATAGGGCCTTGAAATGTAAATAACTTTAAT |
| | | | AAAACGTTTATAACAGTTACAAAAGATTTTAAGA |
| | | | CATGTACCATAATTTTTTTT |
| S000113 | F24 | 151 | GGCACGAGCCGAGTTGGAGGAAGCAGCGGCAGCG |
| | | | GCAGCGGCAGCGGTAGCGGTGAGGACGGCTGTGC |
| | | | AGCCAAGGAACCGGGACAAGCGCGCGACGGCAGG |
| | | | TCGCAGCTGGATCGCAGGAGCCTGGGAGCTGGGA |
| | | | GCTTCAGAGGCCGCTGAAGCCCAGGCTGGGCAGA |
| | | | GGAAGGAAGCGAGCCGACCCGGAGGTGAAGCTGA |
| | | | GAGTGGAGCGTGGCAGTAAAATCAGACGACAGAT |
| | | | GGACAGTGTGACAGGAACGTCAGAGAGGATTGGG |
| | | | CCTCGCTGCGAGAGTCAGCCTGGAGTCAAGGTGT |
| | | | TGACAAGTTGCTGAGAAGGACACGTGGGAGGACG |
| | | | GTGGCGCGCGGAGGGAGAGCCCTGTCTTCAGTCA |
| | | | CCCCGTTGATGGAGGACAGATGGACAGCAGCCGG |
| | | | ACGGCCAGTCACCTCTCTTAAACCTTTGGATAGT |
| | | | GGTCCTTTGTGCTCTGCTGGACACCTGTTGGGGA |
| | | | TTTTAGCCCATTCTCTGAACTCACTTTCTCTTAA |
| | | | AACGTAAACTCGGACGGCAGTGTGCGAGCCAGCT |
| | | | CCTCTGTGGCAGGGCACTAGAGCTGCAGACATGA |
| | | | GTGCAGAGGGCTACCAGTACAGAGCACTGTACGA |
| | | | CTACAAGAAGGAGCGAGAGGAAGACATTGACCTA |
| | | | CACCTGGGGGACATACTGACTGTGAATAAAGGCT |
| | | | CCTTAGTGGCACTTGGATTCAGTGATGGCCAGGA |
| | | | AGCCCGGCCTGAAGATATTGGCTGGTTAAATGGC |
| | | | TACAATGAAACCACTGGGGAGAGGGGAGACTTTC |
| | | | CAGGAACTTACGTTGAATACATTGGAAGGAAAAG |
| | | | AATTTCACCCCCTACTCCCAAGCCTCGGCCCCCT |
| | | | CGACCGCTTCCTGTTGCTCCGGGTTCTTCAAAAA |
| | | | CTGAAGCTGACACGGAGCAGCAAGCGTTGCCCCT |
| | | | TCCTGACCTGGCCGAGCAGTTTGCCCCTCCTGAT |
| | | | GTTGCCCCGCCTCTCCTTATAAAGCTCCTGGAAG |
| | | | CCATTGAGAAGAAAGGACTGGAATGTTCGACTCT |
| | | | ATACAGAACACAAAGCTCCAGCAACCCTGCAGAA |
| | | | TTACGACAGCTTCTTGATTGTGATGCCGCGTCAG |
| | | | TGGACTTGGAGATGATCGACGTACACGTCTTAGC |
| | | | AGATGCTTTCAAACGCTATCTCGCCGACTTACCA |
| | | | AATGCTGTCATTCCTGTAGCTGTTTACAATGAGA |
| | | | TGATGTCTTTAGCCCAAGAACTACAGAGCCCTGA |
| | | | AGACTGCATCCAGCTGTTGAAGAAGCTCATTAGA |
| | | | TTGCCTAATATACCTCATCAGTGTTGGCTTACGC |
| | | | TTCAGTATTTGCTCAAGCATTTTTTCAAGCTCTC |
| | | | TCAAGCCTCCAGCAAAAACCTTTTGAATGCAAGA |
| | | | GTCCTCTCTGAGATTTTCAGCCCCGTGCTTTTCA |
| | | | GATTTCCAGCCGCCAGCTCTGATAATACTGAACA |
| | | | CCTCATAAAAGCGATAGAGATTTTAATCTCAACG |
| | | | GAATGGAATGAGAGACAGCCAGCACCAGCACTGC |
| | | | CCCCCAAACCACCCAAGCCCACTACTGTAGCCAA |
| | | | CAACAGCATGAACAACAATATGTCCTTGCAGGAT |
| | | | GCTGAATGGTACTGGGGAGACATCTCAAGGGAAG |
| | | | AAGTGAATGAAAAACTCCGAGACACTGCTGATGG |
| | | | GACCTTTTTGGTACGAGACGCATCTACTAAAATG |
| | | | CACGGCGATTACACTCTTATACCTAGGAAAGGAG |
| | | | GAAATAACAAATTAATCAAAATCTTTCACCGTGA |
| | | | TGGAAAATATGGCTTCTCTGATCCATTAACCTTC |
| | | | AACTCTGTGGTTGAGTTAATAAACCACTACCGGA |
| | | | ATGAGTCTTTAGCTCAGTACAACCCCAAGCTGGA |
| | | | TGTGAAGTTGCTCTACCCAGTGTCCAAATACCAG |
| | | | CAGGATCAAGTTGTCAAAGAAGATAATATTGAAG |
| | | | CTGTAGGGAAAAAATTACATGAATATAATACTCA |
| | | | ATTTCAAGAAAAAAGTCGGGAATATGATAGATTA |
| | | | TATGAGGAGTACACCCGTACTTCCCAGGAAATCC |
| | | | AAATGAAAAGAACGGCTATCGAAGCATTTAATGA |
| | | | AACCATAAAAATATTTGAAGAACAATGCCAAACC |
| | | | CAGGAGCGGTACAGCAAAGAATACATAGAGAAGT |
| | | | TTAAACGCGAAGGCAACGAGAAAGAAATTCAAAG |
| | | | GATTATGCATAACCATGATAAGCTGAAGTCGCGT |
| | | | ATCAGTGAGATCATTGACAGTAGGAGGAGGTTGG |
| | | | AAGAAGACTTGAAGAAGCAGGCAGCTGAGTACCG |
| | | | AGAGATCGACAAACGCATGAACAGTATTAAGCCG |
| | | | GACCTCATCCAGTTGAGAAAGACAAGAGACCAAT |
| | | | ACTTGATGTGGCTGACGCAGAAAGGTGTGCGGCA |
| | | | GAAGAAGCTGAACGAGTGGCTGGGGAATGAAAAT |
| | | | ACCGAAGATCAATACTCCCTGGTAGAAGATGATG |
| | | | AGGATTTGCCCCACCATGACGAGAAGACGTGGAA |
| | | | TGTCGGGAGCAGCAACCGAAACAAAGCGGAGAAC |
| | | | CTATTGCGAGGGAAGCGAGACGGCACTTTCCTTG |
| | | | TCCGGGAGAGCAGTAAGCAGGGCTGCTATGCCTG |
| | | | CTCCGTAGTGGTAGACGGCGAAGTCAAGCATTGC |
| | | | GTCATTAACAAGACTGCCACCGGCTATGGCTTTG |
| | | | CCGAGCCCTACAACCTGTACAGCTCCCTGAAGGA |
| | | | GCTGGTGCTACATTATCAACACACCTCCCTCGTG |
| | | | CAGCACAATGACTCCCTCAATGTCACACTAGCAT |
| | | | ACCCAGTATATGCACAACAGAGGCGATGAAGCGC |
| | | | TGCCCTCGGATCCAGTTCCTCACCTTCAAGCCAC |
| | | | CCAAGGCCTCTGAGAAGCAAAGGGCTCCTCTCCA |
| | | | GCCCGACCTGTGAACTGAGCTGCAGAAATGAAGC |
| | | | CGGCTGTCTGCACATGGGACTAGAGCTTTCTTGG |
| | | | ACAAAAAGAAGTCGGGGAAGACACGCAGCCTCGG |
| | | | ACTGTTGGATGACCAGACGTTTCTAACCTTATCC |
| | | | TCTTTCTTTCTTTCTTTCTTTCTTTCTTTCTTTC |
| | | | TTTCTTTCTTTCTTTCTTTCTTTCTTTCTTTCTA |
| | | | ATTTAAAGCCACAACACACAACCAACACACAGAG |
| | | | AGAAAGAAATGCAAAAATCTCTCCGTGCAGGGAC |
| | | | AAAGAGGCCTTTAACCATGGTGCTTGTTAACGCT |
| | | | TTCTGAAGCTTTACCAGCTACAAGTTGGGACTTT |
| | | | GGAGACCAGAAGGTAGACAGGGCCGAAGAGCCTG |
| | | | CGCCTGGGGCCGCTTGGTCCAGCCTGGTGTAGCC |
| | | | TGGGTGTCGCTGGGTGTGGTGAACCCAGACACAT |
| | | | CACACTGTGGATTATTTCCTTTTTAAAAGAGCGA |
| | | | ATGATATGTATCAGAGAGCCGCGTCTGCTCACGC |
| | | | AGGACACTTTGAGAGAACATTGATGCAGTCTGTT |
| | | | CGGAGGAAAAATGAAACACCAGAAAACGTTTTTG |
| | | | TTTAAACTTATCAAGTCAGCAACCAACAACCCAC |
| | | | CAACAGAAAAAAAAAAAAAA |
| S000114 | F25 | 152 | GTTGCCGGTTTAGGGTGCTGCTGTAGTGGCGATA |
| | | | CGTCCCGCCGCTGTCCCGAAGTGAGGGATCCGAG |
| | | | CCGCAGCGAGTGCCATGGAGGGCCAGCGCGTGGA |
| | | | GGAGCTGCTGGCCAAGGCAGAGCAGGAGGAGGCG |
| | | | GAGAAGCTGCAGCGCATCACGGTGCACAAGGAGC |
| | | | TGGAGCTGGAGTTCGACCTGGGCAACCTGCTGGC |
| | | | TTCGGACCGCAACCCCCCGACCGTGCTGCGCCAG |
| | | | GCCGGGCCGTCGCCGGAGGCCGAGCTGCGGGCCC |
| | | | TGGCGCGGGACAACACGCAGCTGCTCATCAACCA |
| | | | GCTGTGGCGGCTGCCGACCGAGCGCGTGGAGGAG |
| | | | GCGGTGGTCGCGCGCTTGCCGGAGCCCGCCACTC |
| | | | GCCTGCCCCGCGAGAAGCCGCTGCCCCGACCACG |
| | | | GCCGCTCACCCGCTGGCAGCAGTTCGCGCGCCTT |
| | | | AAGGGAATCCGTCCCAAGAAGAAGACCAACCTCG |
| | | | TGTGGGACGAGGCTAGTGGCCAGTGGCGGCGCCG |
| | | | TTGGGGCTACAAGCGCGCCCGGGATGACACTAAA |
| | | | GAATGGCTGATCGAGGTGCCTGGGAGCGCCGACC |
| | | | CCATGGAAGACCAGTTCGCCAAGAGGACTCAGGC |
| | | | CAAGAAAGAACGCGTGGCCAAGAATGAGCTGAAC |
| | | | CGTCTGCGGAACCTGGCTCGCGCGCACAAGATGC |
| | | | AGATGCCCAGCTCAGCCGGCCTGCACCCTACTGG |
| | | | ACACCAGAGTAAGGAAGAGCTGGGCCGCGCCATG |
| | | | CAAGTGGCCAAGGTTTCCACCGCTTCGGTGGGAC |
| | | | GCTTCCAGGAGCGCCTTCCCAAGGAGAAAGCTCC |
| | | | CCGGGGCTCCGGCAAGAAGAGGAAGTTTCAGCCC |
| | | | CTCTTTGGGGACTTCGCAGCCGAGAAAAAGAACC |
| | | | AGTTGGAGCTACTTCGAGTCATGAACAGCAAGAA |
| | | | ACCTCGGCTGGACGTGACGAGGGCCACCAACAAG |
| | | | CAGATGAGGGAAGAGGACCAGGAGGAGGCTGCCA |
| | | | AGAGGAGGAAAATGAGCCAGAAAGGCAAGAGGAA |
| | | | AGGGGGCCGGCAAGGACCTTCGGGCAAGAGAAGG |
| | | | GGCGGCCCGCCGGGTCAGGGAGAAAAGAGGAAAG |
| | | | GAGGCTTGGGAAGCAAAAAGCATTCCTGGCCTTC |
| | | | TGCTTTAGCTGGCAAGAAGAAGGAGTGCCGCCCC |
| | | | AAGGTGGGAAGAGGAGGAAGTAGCGTTCTCCCCT |
| | | | CGGGCACCAGTTCTGAAAAGCTGGGACTGTACTA |
| | | | AAAGTTAACTTGGGCGGTATAGGTGGCCGCTGCC |
| | | | CTCAGTGACATTTGACATTAAAAGGACGGGTTTG |
| | | | CCTTCCCTCGAGTCAGTGCTGGACGAGTTAATAG |
| | | | AGACACTGACTGGAAATTGGTGTATTTTGAGAAT |
| | | | TATAGAAATGATATAGCCAGAACCAGGAATAAGT |
| | | | TAAGGCCTGCCTTTTTATCTTGACTTTGGATACT |
| | | | GCGTTACAGTAGATTGGTTTCAACATTTTTGCAT |
| | | | TATTTTTATAACAAAGCTTGTGTATTTATCAAAG |
| | | | CGGGGAGGGCGGGGAAAAATTATATCTACCTGTG |
| | | | ATTTGCAAGTATTGTAAATGGATGCAGGTACCTG |
| | | | GTGTTGCTTTTAACTTTTACTGTCGGTAGAGGTT |
| | | | GCATGTGAAGCCAGTAACCTGGGCACCAATATGG |
| | | | AGTGTGCTTGAGAAAAACAAAGTAGTTACAGTGG |
| | | | TTCTAAAAAAGACCCCTTGTTTTAGGAAAACTTT |
| | | | GGCCCTAACTATAATATTAAAAGTATAGTGCTTT |
| | | | TTGGTGTTGGTTCAGGTGGTGCATTTGGCCAATG |
| | | | GATTGCTTTAAGTCCAGAAATAGTTGTCATTTTG |
| | | | TTTGTAACCGGTGGCTTTTGTTTAATTGGCTTGG |
| | | | GTTTTAGATATTGTCAAAATATCTGGGATTCACT |
| | | | ATGGAACCAAGGCTGCCCTGGAACTCAGGGCCAA |
| | | | GTGCTGAGATTATAATCGAGCAGCAGATTTCATG |
| | | | TTTATTTCTGTCCTAGATGTTTTTCCCTGTTTCA |
| | | | TTGTCTTATTTTGTTCTTAATAAACTTATCTTTG |
| | | | CATAAAAAAAAAAAAAAGGCCACA |
| S000116 | F26 | 153 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGCCAGGACAGGACTCCCCAGGCTCCGGGGAGG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGCTACGTCCTTCT |
| | | | CCCCAAGGGAAGACGATGACGGCGGCGGTGGCAA |
| | | | CTTCTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGGCGCTGCCAAGCTGGTCTCGGAGAAGC |
| | | | TGGCCTCCTACCAGGCTGCGCGCAAAGACAGCAC |
| | | | CAGCCTGAGCCCCGCCCGCGGGCACAGCGTCTGC |
| | | | TCCACCTCCAGCCTGTACCTGCAGGACCTCACCG |
| | | | CCGCCGCGTCCGAGTGCATTGACCCCTCAGTGGT |
| | | | CTTTCCCTACCCGCTCAACGACAGCAGCTCGCCC |
| | | | AAATCCTGTACCTCGTCCGATTCCACGGCCTTCT |
| | | | CTCCTTCCTCGGACTCGCTGCTGTCCTCCGAGTC |
| | | | CTCCCCACGGGCCAGCCCTGAGCCCCTAGTGCTG |
| | | | CATGAGGAGACACCGCCCACCACCAGCAGCGACT |
| | | | CTGAAGAAGAGCAAGAAGATGAGGAAGAAATTGA |
| | | | TGTGGTGTCTGTGGAGAAGAGGCAAACCCCTGCC |
| | | | AAGAGGTCGGAGTCGGGCTCATCTCCATCCGGAG |
| | | | GCCACAGCAAACCTCCGCACAGCCCACTGGTCCT |
| | | | CAAGAGGTGCCACGTCTCCACTCACCAGCACAAC |
| | | | TACGCCGCACCCCCCTCCACAAGGAAGGACTATC |
| | | | CAGCTGCCAAGAGGGCCAAGTTGGACAGTGGCAG |
| | | | GGTCCTGAAGCAGATCAGCAACAACCGCAAGTGC |
| | | | TCCAGCCCCAGGTCCTCAGACACGGAGGAAAACG |
| | | | ACAAGAGGCGGACACACAACGTCTTGGAACGTCA |
| | | | GAGGAGGAACGAGCTGAAGCGCAGCTTTTTTGCC |
| | | | CTGCGTGACCAGATCCCTGAATTGGAAAACAACG |
| | | | AAAAGGCCCCCAAGGTAGTGATCCTCAAAAAAGC |
| | | | CACCGCCTACATCCTGTCCATTCAAGCAGACGAG |
| | | | CACAAGCTCACCTCTGAAAAGGACTTATTGAGGA |
| | | | AACGACGAGAACAGTTGAAACACAAACTCGAACA |
| | | | GCTTCGAAACTCTGGTGCATAAACTGACCTAACT |
| | | | CGAGGAGGAGCTGGAATCTCTCGTGAGAGTAAGG |
| | | | AGAACGGTTCCTTCTGACAGAACTGATGCGCTGG |
| | | | AATTAAAATGCATGCTCAAAGCCTAACCTCACAA |
| | | | CCTTGGCTGGGGCTTTGGGACTGTAAGCTTCAGC |
| | | | CATAATTTTAACTGCCTCAAACTTAAATAGTATA |
| | | | AAAGAACTTTTTTTATGCTTCCCATCTTTTTTCT |
| | | | TTTTCCTTTTTAACAGATTTGTATTTAATTGTTT |
| | | | TTTTAAAAAAATCTTAAAATCTATCCAATTTTCC |
| | | | CATGTAAATAGGGCCTTGAAATGTAAATAACTTT |
| | | | AATAAAACGTTTATAACAGTTACAAAAGATTTTA |
| | | | AGACATGTACCATAATTTTTTTT |
| S000118 | F27 | 154 | TATATTCCGGGGGTCTGCGCGGCCGAGGACCCCT |
| | | | GGGTGCGCTGCTCTCAGCTGCCGGGTCCGACTCG |
| | | | CCTCACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGGGCAGGACAGGACTCCCCAGGCTCCGGGGAG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGCTACGTCCTTCT |
| | | | CCCCAAGGGAAGACGATGACGGCGGCGGTGGCAA |
| | | | CTTCTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGCCGCTGCCAAGCTGGTCTCGGAGAAGC |
| | | | TGGCCTCCTACCAGGCTGCGCGCAAAGACAGCAC |
| | | | CAGCCTGAGCCCCGCCCGCGGGCACAGCGTCTGC |
| | | | TCCACCTCCAGCCTGTACCTGCAGGACCTCACCG |
| | | | CCGCCGCGTCCGAGTGCATTGACCCCTCAGTGGT |
| | | | CTTTCCCTACCCGCTCAACGACAGCAGCTCGCCC |
| | | | AAATCCTGTACCTCGTCCGATTCCACGGCCTTCT |
| | | | CTCCTTCCTCGGACTCGCTGCTGTCCTCCGAGTC |
| | | | CTCCCCACGGGCCAGCCCTGAGCCCCTAGTGCTG |
| | | | CATGAGGAGACACCGCCCACCACCAGCAGCGACT |
| | | | CTGAAGAAGAGCAAGAAGATGAGGAAGAAATTGA |
| | | | TGTGGTGTCTGTGGAGAAGAGGCAAACCCCTGCC |
| | | | AAGAGGTCGGAGTCGGGCTCATCTCCATCCCGAG |
| | | | GCCACAGCAAACCTCCGCACAGCCCACTGGTCCT |
| | | | CAAGAGGTGCCACGTCTCCACTCACCAGCACAAC |
| | | | TACGCCGCACCCCCCTCCACAAGGAAGGACTATC |
| | | | CAGCTGCCAAGAGGGCCAAGTTGGACAGTGGCAG |
| | | | GGTCCTGAAGCAGATCAGCAACAACCGCAAGTGC |
| | | | TCCAGCCCCAGGTCCTCAGACACGGAGGAAAACG |
| | | | ACAAGAGGCGGACACACAACGTCTTGGAACGTCA |
| | | | GAGGAGGAACGAGCTGAAGCGCAGCTTTTTTGCC |
| | | | CTGCGTGACCAGATCCCTGAATTGGAAAACAACG |
| | | | AAAAGGCCCCCAAGGTAGTGATCCTCAAAAAAGC |
| | | | CACCGCCTACATCCTGTCCATTCAAGCAGACGAG |
| | | | CACAAGCTCACCTCTGAAAAGGACTTATTGAGGA |
| | | | AACGACGAGAACAGTTGAAACACAAACTCGAACA |
| | | | GCTTCGAAACTCTGGTGCATAAACTGACCTAACT |
| | | | CGAGGAGGAGCTGGAATCTCTCGTGAGAGTAAGG |
| | | | AGAACGGTTCCTTCTGACAGAACTGATGCGCTGG |
| | | | AATTAAAATGCATGCTCAAAGCCTAACCACACAA |
| | | | CCTTGGCTGGGGCTTTGGGACTGTAAGCTTCAGC |
| | | | CATAATTTTAACTGCCTCAAACTTAAATAGTATA |
| | | | AAAGAACTTTTTTTATGCTTCCCATCTTTTTTCT |
| | | | TTTTCCTTTTAACAGATTTGTATTTAATTGTTTT |
| | | | TTTAAAAAAATCTTAAAATCTATCCAATTTTCCC |
| | | | ATGTAAATAGGGCCTTGAAATGTAAATAACTTTA |
| | | | ATAAAACGTTTATAACAGTTACAAAAGATTTTAA |
| | | | GACATGTACCATAATTTTTTTT |
| S000121 | F28 | 155 | TATATTCCGGGGGTCTGCGCGGCCGAGGACCCCT |
| | | | GGGTGCGCTGCTCTCAGCTGCCGGGTCCGACTCG |
| | | | CCTGACTCAGCTCCCCTCCTGCCTCCTGAAGGGC |
| | | | AGCTTCGCCGACGCTTGGCGGGAAAAAGAAGGGA |
| | | | GGGGAGGGATCCTGAGTCGCAGTATAAAAGAAGC |
| | | | TTTTCGGGCGTTTTTTTCTGACTCGCTGTAGTAA |
| | | | TTCCAGCGAGAGACAGAGGGAGTGAGCGGACGGT |
| | | | TGGAAGAGCCGTGTGTGCAGAGCCGCGCTCCGGG |
| | | | GCGACCTAAGAAGGCAGCTCTGGAGTGAGAGGGG |
| | | | CTTTGCCTCCGAGCCTGCCGCCCACTCTCCCCAA |
| | | | CCCTGCGACTGACCCAACATCAGCGGCCGCAACC |
| | | | CTCGCCGCCGCTGGGAAACTTTGCCCATTGCAGC |
| | | | GGGCAGACACTTCTCACTGGAACTTACAATCTGC |
| | | | GAGCCAGGACAGGACTCCCCAGGCTCCGGGGAGG |
| | | | GAATTTTTGTCTATTTGGGGACAGTGTTCTCTGC |
| | | | CTCTGCCCGCGATCAGCTCTCCTGAAAAGAGCTC |
| | | | CTCGAGCTGTTTGAAGGCTGGATTTCCTTTGGGC |
| | | | GTTGGAAACCCCGCAGACAGCCACGACGATGCCC |
| | | | CTCAACGTGAACTTCACCAACAGGAACTATGACC |
| | | | TCGACTACGACTCCGTACAGCCCTATTTCATCTG |
| | | | CGACGAGGAAGAGAATTTCTATCACCAGCAACAG |
| | | | CAGAGCGAGCTGCAGCCGCCCGCGCCCAGTGAGG |
| | | | ATATCTGGAAGAAATTCGAGCTGCTTCCCACCCC |
| | | | GCCCCTGTCCCCGAGCCGCCGCTCCGGGCTCTGC |
| | | | TCTCCATCCTATGTTGCGGTCGCTACGTCCTTCT |
| | | | CCCCAAGGGAAGACGATGACGGCGGCGGTGGCAA |
| | | | CTTCTCCACCGCCGATCAGCTGGAGATGATGACC |
| | | | GAGTTACTTGGAGGAGACATGGTGAACCAGAGCT |
| | | | TCATCTGCGATCCTGACGACGAGACCTTCATCAA |
| | | | GAACATCATCATCCAGGACTGTATGTGGAGCGGT |
| | | | TTCTCAGCCGCTGCCAAGCTGGTCTCGGAGAAGC |
| | | | TGGCCTCCTACCAGGCTGCGCGCAAAGACAGCAC |
| | | | CAGCCTGAGCCCCGCCCGCGGGCACAGCGTCTGC |
| | | | TCCACCTCCAGCCTGTACCTGCAGGACCTCACCG |
| | | | CCGCCGCGTCCGAGTGCATTGACCCCTCAGTGGT |
| | | | CTTTCCCTACCCGCTCAACGACAGCAGCTCGCCC |
| | | | AAATCCTGTACCTCGTCCGATTCCACGGCCTTCT |
| | | | CTCCTTCCTCGGACTCGCTGCTGTCCTCCGAGTC |
| | | | CTCCCCACGGGCCAGCCCTGAGCCCCTAGTGCTG |
| | | | CATGAGGAGACACCGCCCACCACCAGCAGCGACT |
| | | | CTGAAGAAGAGCAAGAAGATGAGGAAGAAATTGA |
| | | | TGTGGTGTCTGTGGAGAAGAGGCAAACCCCTGCC |
| | | | AAGAGGTCGGAGTCGGGCTCATCTCCATCCCGAG |
| | | | GCCACAGCAAACCTCCGCACAGCCCACTGGTCCT |
| | | | CAAGAGGTGCCACGTCTCCACTCACCAGCACAAC |
| | | | TACGCCGCACCCCCCTCCACAAGGAAGGACTATC |
| | | | CAGCTGCCAAGAGGGCCAAGTTGGACAGTGGCAG |
| | | | GGTCCTGAAGCAGATCAGCAACAACCGCAAGTGC |
| | | | TCCAGCCCCAGGTCCTCAGACACGGAGGAAAACG |
| | | | ACAAGAGGCGGACACACAACGTCTTGGAACGTCA |
| | | | GAGGAGGAACGAGCTGAAGCGCAGCTTTTTTGCC |
| | | | CTGCGTGACCAGATCCCTGAATTGGAAAACAACG |
| | | | AAAAGGCCCCCAAGGTAGTGATCCTCAAAAAAGC |
| | | | CACCGCCTACATCCTGTCCATTCAAGCAGACGAG |
| | | | CACAAGCTCACCTCTGAAAAGGACTTATTGAGGA |
| | | | AACGACGAGAACAGTTGAAACACAAACTCGAACA |
| | | | GCTTCGAAACTCTGGTGCATAAACTGACCTAACT |
| | | | CGAGGAGGAGCTGGAATCTCTCGTGAGAGTAAGG |
| | | | AGAACGGTTCCTTCTGACAGAACTGATGCGCTGG |
| | | | AATTAAAATGCATGCTCAAAGCCTAACCTCACAA |
| | | | CCTTGGCTGGGGCTTTGGGACTGTAAGCTTCAGC |
| | | | CATAATTTTAACTGCCTCAAACTTAAATAGTATA |
| | | | AAAGAACTTTTTTTATGCTTCCCATCTTTTTTCT |
| | | | TTTTCCTTTTAACAGATTTGTATTTAATTGTTTT |
| | | | TTAAAAAAATCTTAAAATCTATCCAATTTTCCCA |
| | | | TGTAAATAGGGCCTTGAAATGTAAATAACTTTAA |
| | | | TAAAACGTTTATAACAGTTACAAAAGATTTTAAG |
| | | | ACATGTACCATAATTTTTTTT |

Contigs assembled from the human EST database by the NCBI having homology with all or parts of the LA nucleic acid sequences of the invention are depicted in Table 3.

TABLE 3

HUMAN

| | | |
|---|---|---|
| SAGRES | REF | SEQ |

| | | | |
|---|---|---|---|
| TAG # | # | ID # | SEQUENCE |

| | | | |
|---|---|---|---|
| S000010 | F29 | 156 | GTGTGGCTGGACCTCGTGTCGCGAGCTGCCATTGCC |
| | | | CAGTGGATGGAAGAAGAAAGGGCTCCGCGCAAGCGC |
| | | | CGATGGCGCGGCCTCCCAGTGCCCTGCGGCAGCGAC |
| | | | TCGGAGGACGCGCGAGTTTGCAGATCCATGTGCTGG |
| | | | ACAGATGACTGCCCTGGGCCCGGAAGCTGGGACCTG |
| | | | GAAGACCCCTGCCCACCTTCCCCACCTCGGAATGCA |
| | | | CCTCGCGATGTGGAGCCCGGACACCCGGGCAGATGG |
| | | | CTGCGTGCCCAGAACAAGCAAGACAGAAGAACGTCT |
| | | | GGCAGGCTTCCAGTCCATGGGCCCTGAGCTACCCGG |
| | | | TGTTCAAAGGCATCATGACACGAAGGGGTACAAGGT |
| | | | GCCAACACCCATCCAGAGGAAGACCATCCCGGTGAT |
| | | | CTTGGATGGCAAGGACGTGGTGGCCATGGCCCGGAC |
| | | | GGGCAGTGGCAAGACATGCTGCTTCCTCCTCCTCCC |
| | | | AATGTCCGAGCGGCAAGACCCACAGTTGCCCAGACC |
| | | | CGGGGCCCTGTGCCCTCATCCTCTTCGCCGACCCGA |
| | | | GAGCTGGCCCTTGCAGACCCTGAAGTTCACTACGGA |
| | | | GCTAGGCCAGTCCCTTGGCCTCAAGACTGCCCTGAT |
| | | | CCTGGGTGGCGCCCGGATGCCCACCCGCCTCGCAGC |
| | | | CCTTGCACCGCAAATCCCGACATACTTTTGGCAGGC |
| | | | CCGGACCGGTTGGGGCCTGTGGGCTGTGGCAATTGA |
| | | | GCCTGCAGCTCCCAGTTTTGCGCTCCGTGGTGGTCC |
| | | | GCGCACCCTGCCGCGCTCTTCGCCCCGCGTTCTCGC |
| | | | TCATCCCCTTCCGTGGCGCTTTCCGCCGGCCTCCCC |
| | | | GCGGGGGCCCCACCACCGGCGGGCGCTCCCTGCGCC |
| | | | GGCCTCCCCACCCTGTCGTGCTCGGCGATTGTGCCC |
| | | | GGCTGTGCCTCCGGGGGGCGGTGGTCACCCCGGCTG |
| | | | CGGGCGACTACACCCCTCGCGCCTCAGTGCCCCTCT |
| | | | TCCCCCGGGCGGGAGGACCCACGCCGCGTCGCC |
| S000013 | F30 | 157 | CACACCGCAGTATGCGGTGCCCTTTACTCTGAGCTG |
| | | | CGCAGCCGGCCGGCCGGCGCTGGTTGAACAGACTGC |
| | | | CGCTGTACTGGCGTGGCCTGGAGGGACTCAGCAAAT |
| | | | TCTCCGCCTTCAACTTGGCAACAGTTGCCTGGGGTA |
| | | | GCTCTACACAACTCTGTCCAGCCCACAGCAATGATT |
| | | | CCAGAGGCCATGGGGAGTGGACAGCAGCTAGCTGAC |
| | | | TGGAGGAATGCCCACTCTCATGGCAACCAGTACAGC |
| | | | ACTATCATGCAGCAGCCATCCTTGCTGACTAACCAT |
| | | | GTGACATTGGCCACTGCTCAGCCTCTGAATGTTGGT |
| | | | GTTGCCCATGTTGTCAGACAACAACAATCCAGTTCC |
| | | | CTCCCTTCGAAGAATAAGCAGTCAGCTCCAGTCTCT |
| | | | TCCAAGTCCTCTCTAGATGTTCTGCCTTCCCAAGTC |
| | | | TATTCTCTGGTTGGGAGCAGTCCCCTCCGCACCACA |
| | | | TCTTCTTATATCCTTGGTCCCTGTCCAAGATCAGCA |
| | | | TCAGCCCATCATCATTCCAGATACTCCCAGCCCTCC |
| | | | TGTGAGTGTCATCACTATCCGAAGTGACACTGATGA |
| | | | GGAAGAGGACAACAAATACAAGCCCAGTAGCTCTGG |
| | | | ACTGAAGCCAAGGTCTAATGTCATCAGTTATGTCAC |
| | | | TGTCAATGATTCTCCAGACTCTGACTCTTCTTTGAG |
| | | | CAGCCCTTATTCCACTGATACCCTGAGTGCTCTCCG |
| | | | AGGCAATAGTGGATCCGTTTTGGAGGGGCCTGGCAG |
| | | | AGTTGTGGCAGATGGCACTGGCACCCGCACTATCAT |
| | | | TGTGCCTCCACTGAAAACTCAGCTTGGTGACTGCAC |
| | | | TGTAGCAACCCAGGCCTCAGGTCTCCTGAGCAATAA |
| | | | GACTAAGCCAGTCGCTTCAGTGAGTGGGCAGTCATC |
| | | | TGGATGCTGTATCACCCCCACAGGGTATCGAGCTCA |
| | | | ACGCGGGGGGACCAGTGCAGCACAACCACTCAATCT |
| | | | TAGCCAGAACCAGCAGTCATCGGCGGCTCCAACCTC |
| | | | ACAGGAGAGAAGCAGCAACCCAGCCCCCCGCAGGCA |
| | | | GCAGGCGTTTGTGGCCCCTCTCTCCCAAGCCCCCTA |
| | | | CACCTTCCAGCATGGCAGCCCGCTACACTCGACAGG |
| | | | GCACCCACACCTTGCCCCGGCCCCTGCTCACCTGCC |
| | | | AAGCCAGGCTCATCTGTATACGTATGCTGCCCCGAC |
| | | | TTCTGCTGCTGCACTGGGCTCAACCAGCTCCATTGC |
| | | | TCATCTTTTCTCCCCACAGGGTTCCTCAAGGCATGC |
| | | | TGCAGCCTATACCACTCACCCTAGCACTTTGGTGCA |
| | | | CCAGGTCCCTGTCAGTGTTGGGCCCAGCCTGCTCAC |
| | | | TTCTGCCAGCGTGGCCCCTGCTCAGTACCAACACCA |
| | | | GTTTGCCACCCAATCCTACATTGGGTCTTCCCGAGG |
| | | | CTCAACAATTTACACTGGATACCCGCTGAGTCCTAC |
| | | | CAAGATCAGCCAGTATTCCTACTTATAGTTGGTGAG |
| | | | CATGAGGGAGGAGGAATCATGGCTACCTTCTCCTGG |
| | | | CCCTGCGTTCTTAATATTGGGCTATGGAGAGATCCT |
| | | | CCTTTACCCTCTTGAAATTTCTTAGCCAGCAACTTG |
| | | | TTCTGCAGGGGCCCACTGAAGCAGAAGGTTTTTCTC |
| | | | TGGGGGAACCTGTCTCAGTGTTGACTGCATTGTTGT |
| | | | AGTCTTCCCAAAGTTTGCCCTATTTTTAAATTCATT |
| | | | ATTTTTGTGACAGTAATTTTGGTACTTGGAAGAGTT |
| | | | CAGATGCCCATCTTCTGCAGTTACCAAGGAAGAGAG |
| | | | ATTGTTCTGAAGTTACCCTCTGAAAAATATTTTGTC |
| | | | TCTCTGACTTGATTTCTATAAATGCTTTTAAAAACA |
| | | | AGTGAAGCCCCTCTTTATTTCATTTTGTGTTATTGT |
| | | | GATTGCTGGTCAGGAAAAATGCTGATAGAAGGAGTT |
| | | | GAAATCTGATGACAAAAAAAGAAAAATTACTTTTTG |
| | | | TTTGTTTATAAACTCAGACTTGCCTATTTTATTTTA |
| | | | AAAGCGGCTTACACAATCTCCCTTTTGTTTATTGGA |
| | | | CATTTAAAACTTACAGAGTTTCAGTTTTGTTTTAAT |
| | | | GTCATATTATACTTAATGGGCAATTGTTATTTTTGC |
| | | | AAAACTGGTTACGTATTACTCTGTGTTACTATTGAG |
| | | | ATTCTCTCAATTGCTCCTGTGTTTGTTATAAAGTAG |
| | | | TGTTTAAAAGGCAGCTCACCATTTGCTGGTAACTTA |
| | | | ATGTGAGAGAATCCATATCTGCGTGAAAACACCAAG |
| | | | TATTCTTTTTAAATGAAGCACCATGAATTCTTTTTT |
| | | | AAATTATTTTTTAAAAGTCTTTCTCTCTCTGATTCA |
| | | | GCTTAAATTTTTTTATCGAAAAAGCCATTAAGGTGG |
| | | | TTATTATTACATGGTGGTGGTGGTTTTATTATATGC |
| | | | AAAATCTCTGTCTATTATGAGATACTGGCATTGATG |
| | | | AGCTTTGCCTAAAGATTAGTATGAATTTTCAGTAAT |
| | | | ACACCTCTGTTTTGCTCATCTCTCCCTTCTGTTTTA |
| | | | TGTGATTTGTTTGGGGAGAAAGCTAAAAAAACCTGA |
| | | | AACCAGATAAGAACATTTCTTGTGTATAGCTTTTAT |
| | | | ACTTCAAAGTAGCTTCCTTTGTATGCCAGCAGCAAA |
| | | | TTGAATGCTCTCTTATTAAGACTTATATAATAAGTG |
| | | | CATGTAGGAATTGCAAAAAATATTTTAAAAATTTAT |
| | | | TACTGAATTTAAAAATATTTTAGAAGTTTTGTAATG |
| | | | GTGGTGTTTTAATATTTTACATAATTAAATATGTAC |
| | | | ATATTGATTAGAAAAATATAACAAGCAATTTTTCCT |
| | | | GCTAACCCAAAATGTTATTTGTAATCAAATGTGTAG |
| | | | TGATTACACTTGAATTGTGTACTTAGTGTGTATGTG |
| | | | ATCCTCCAGTGTTATCCCGGAGATGGAATTGATGTC |
| | | | TCCATTGTATTTAAACCAATGAACTGATACTTGTTG |
| | | | GAATGTATGTGAACTAATTGCAATTATATTAGAGCA |
| | | | TATTACTGTAGTGCTGAATGAGCAGGGGCATTGCCT |
| | | | GCAAGGAGAGGAGACCCTTGGAATTGTTTTGCACAG |
| | | | GTGTGTCTGGTGAGGAGTTTTTCAGTGTGTGTCTCT |
| | | | TCCTCCCTTTCTTCCTCCTTCCCTTATTGTAGTGCC |
| | | | TTATATGATAATGTAGTGGTTAATAGAGTTTACAGT |
| | | | GAGCTTGCCTTAGGATGGACCAGCAAGCCCCCGTGG |
| | | | ACCCTAAGTTGTTCACCGGGATTTATCAGAACAGGA |
| | | | TTAGTAGCTGTATTGTGTAATGCATTGTTCTCAGTT |
| | | | TCCCTGCCAACATTGAAAATAAAAACAGCAGCTTTT |
| | | | CTCCTTTACCACCACCTCTACCCCTTTCCATTTTGG |
| | | | ATTCTCGGCTGAGTTCTCACAGAAGCATTTTCCCCA |
| | | | TGTGGCTCTCTCACTGTGCGTTGCTACCTTGCTTCT |
| | | | GTGAGAATTCAGGAAGCAGGTGAGAGGAGTCAAGCC |
| | | | AATATTAAATATGCATTCTTTTAGTATGTGCAATCA |
| | | | CTTTTAGAATGAATTTTTTTTTCCTTTTCCCATGTG |
| | | | GCAGTCCTTCCTGCACATAGTTGACATTCCTAGTAA |
| | | | AATATTTGCTTGTTGAAAAAAACATGTTAACAGATG |
| | | | TGTTTATACCAAAGAGCCTGTTGTATTGCTTACCAT |
| | | | GTCCCCATACTATGAGGAGAAGTTTTGTGGTGCCGC |
| | | | TGGTGACAAGGAACTCACAGAAGGTTTCTTAGCTGG |
| | | | TGAAGAATATAGAGAAGGAACCAAAGCCTGTTGAGT |
| | | | CATTTGAGGCTTTTGAGGTTTCTTTTTTAACAGCTT |
| | | | GTATAGTCTTGGGGCCCTTCAAGCTGTGAAATTGTC |
| | | | CTTGTACTCTCAGCTCCTGCATGGATCTGGGTCAAG |
| | | | TAGAAGGTACTGGGGATGGGGACATTCCTGCCCATA |
| | | | AAGGATTTGGGGAAAGAAGATTAATCCTAAAATACA |
| | | | GGTGTGTTCCATCCGAATTGAAAATGATATATTTGA |
| | | | GATATAATTTTAGGACTGGTTCTGTGTAGATAGAGA |
| | | | TGGTGTCAAGGAGGTGCAGGATGGAGATGGGAGATT |
| | | | TCATGGAGCCTGGTCAGCCAGCTCTGTACCAGGTTG |
| | | | AACACCGAGGAGCTGTCAAAGTATTTGGAGTTTCTT |
| | | | CATTGTAAGGAGTAAGGGCTTCCAAGATGGGGCAGG |
| | | | TAGTCCGTACAGCCTACCAGGAACATGTTGTGTTTT |
| | | | CTTTATTTTTTAAAATCATTATATTGAGTTGTGTTT |
| | | | TCAGCACTATATTGGTCAAGATAGCCAAGCAGTTTG |
| | | | TATAATTTCTGTCACTAGTGTCATACAGTTTTCTGG |
| | | | TCAACATGTGTGATCTTTGTGTCTCCTTTTTGCCAA |
| | | | GCACATTCTGATTTTCTTGTTGGAACACAGGTCTAG |
| | | | TTTCTAAAGGACAAATTTTTTGTTCCTTGTCTTTTT |
| | | | TCTGTAAGGGACAAGATTTGTTGTTTTTGTAAGAAA |
| | | | TGAGATGCAGGAAAGAAAACCAAATCCCATTCCTGC |
| | | | ACCCCAGTCCAATAAGCAGATACCACTTAAGATAGG |
| | | | AGTCTAAACTCCACAGAAAAGGATAATACCAAGAGC |
| | | | TTGTATTGTTACCTTAGTCACTTGCCTAGCAGTGTG |
| | | | TGGCTTTAAAAACTAGAGATTTTTCAGTCTTAGTCT |
| | | | GCAAACTGGCATTTCCGATTTTCCAGCATAAAAATC |
| | | | CACCTGTGTCTGCTGAATGTGTATGTATGTGCTCAC |
| | | | TGTGGCTTTAGATTCTGTCCCTGGGGTTAGCCCTGT |
| | | | TGGCCCTGACAGGAAGGGAGGAAGCCTGGTGAATTT |
| | | | AGTGAGCAGCTGGCCTGGGTCACAGTGACCTGACCT |
| | | | CAAACCAGCTTAAGGCTTTAAGTCCTCTCTCAGAAC |
| | | | TTGGCATTTCCAACTTCTTCCTTTCCGGGTGAGAGA |
| | | | GAAGAAGCGGAGAAGGGTTCAGTGTAGCCACTCTGG |
| | | | GCTCATAGGGACACTTGGTCACTCCAGAGTTTTTAA |
| | | | TAGCTCCCAGGAGGTGATATTATTTTCAGTGCTCAG |
| | | | CTGAAATACCAACCCCAGGAATAAGAACTCCATTTC |
| | | | AAACAGTTCTGGCCATTCTGAGCCTGCTTTTGTGAT |
| | | | TGCTCATCCATTGTCCTCCACTAGAGGGGCTAAGCT |
| | | | TGACTGCCCTTAGCCAGGCAAGCACAGTAATGTGTG |
| | | | TGTTTTGTTCAGCATTATTATGCAAAAATTCACTAG |
| | | | TTGAGATGGTTTGTTTTAGGATAGGAAATGAAATTG |
| | | | CCTCTCAGTGACAGGAGTGGCCCGAGCCTGCTTCCT |
| | | | ATTTTGATTTTTTTTTTTTTTAACTGATAGATGGTG |
| | | | CAGCATGTCTACATGGTTGTTTGTTGCTAAACTTTA |
| | | | TATAATGTGTGGTTTCAATTCAGCTTGAAAATAATC |
| | | | TCACTACATGTAGCAGTACATTATATGTACATTATA |
| | | | TGTAATGTTAGTATTTCTGCTTGAATCCTTGATATT |
| | | | GCAATGGAATTCCTACTTTATTAAATGTATTTGATA |
| | | | TGCTAGTTATTGTGTGCGATTTAAACTTTTTTTGCT |
| | | | TTCTCCCTTTTTTTGGTTGTGCGCTTTCTTTTACAA |
| | | | CAAGCCTCTAGAAACAGATAGTTTCTGAGAATTACT |
| | | | GAGCTATGTTTGTAATGCAGATGTACTTAGGGAGTA |
| | | | TGTAAAATAATCATTTTAACAAAAGAAATAGATATT |
| | | | TAAAATTTAATACTAACTATGGGAAAAGGGTCCATT |
| | | | GTGTAAAACATAGTTTATCTTTGGATTCAATGTTTT |
| | | | GTCTTTGGTTTTACAAAGTAGCTTGTATTTTCAGTA |
| | | | TTTTCTACATAATATGGTAAAATGTAGAGCAATTGC |
| | | | AATGCATCAATAAAATGGGTAAATTTTCTG |
| S000023 | F31 | 158 | GGAGCCGTCACCCCGGGCGGGGACCCAGCGCAGGCA |
| | | | ACTCCGCGCGGCGCCCGGCCGAGGGAGGGAGCGAGC |
| | | | GGGCGGGCGGGCAAGCCAGACAGCTGGGCCGGAGCA |
| | | | GCCGCCGGCGCCCGAGGGGCCGAGCGAGATGTAAAC |
| | | | CATGGCTGTGTGGATACAAGCTCAGCAGCTCCAAGG |
| | | | AGAAGCCCTCATCAGATGCAAGCGTTATATGGCCAG |
| | | | CATTTTCCCATTGAGGTGCGGCATTATTTATCCCAG |
| | | | TGGATTGAAAGCCAAGCATGGGACTCAGTAGATCTT |
| | | | GATAATCCACAGGAGAACATTAAGGCCACCCAGCTC |
| | | | CTGGAGGGCCTGGTGCAGGAGCTGCAGAAGAAGGCA |
| | | | GAGCACCAGGTGGGGGAAGATGGGTTTTTACTGAAG |
| | | | ATCAAGCTGGGGCACTATGCCACACAGCTCCAGAAC |
| | | | ACGTATGACCGCTGCCCCATGGAGCTGGTCCGCTGC |
| | | | ATCCGCCATATATTGTACAATGAACAGAGGTTGGTC |
| | | | CGAGAAGCCAACAATGGTAGCTCTCCAGCTGGAAGC |
| | | | CTTGCTGATGCCATGTCCCAGAAACACCTCCAGATC |
| | | | AACCAGACGTTTGAGGAGCTGCGACTGGTCACGCAG |
| | | | GACACAGAGAATGAGTTAAAAAAGCTGCAGCAGACT |
| | | | CAGGAGTACTTCATCATCCAGTACCAGGAGAGCCTG |
| | | | AGGATCCAAGCTCAGTTTGGCCCGCTGGCCCAGCTG |
| | | | AGCCCCCAGGAGCGTCTGAGCCGGGAGACGGCCCTC |
| | | | CAGCAGAAGCAGGTGTCTCTGGAGGCCTGGTTGCAG |
| | | | CGTGAGGCACAGACACTGCAGCAGTACCGCGTGGAG |
| | | | CTGCCCGAGAAGCACCAGAAGACCCTGCAGCTGCTG |
| | | | CGGAAGCAGCAGACCATCATCCTGGATGACGAGCTG |
| | | | ATCCAGTGGAAGCGGCGGCAGCAGCTGGCCGGGAAC |
| | | | GGCGGGCCCCCCGAGGGCAGCCTGGACGTGCTACAG |
| | | | TCCTGGTGTGAGAAGTTGGCGGAGATCATCTGGCAG |
| | | | AACCGGCAGCAGATCCGCAGGGCTGAGCACCTCTGC |
| | | | CAGCAGCTGCCCATCCCCGGCCCAGTGGAGGAGATG |
| | | | CTGGCCGAGGTCAACGCCACCATCACGGACATTATC |
| | | | TCAGCCCTGGTGACCAGCACGTTCATCATTGAGAAG |
| | | | CAGCCTCCTCAGGTCCTGAAGACCCAGACCAAGTTT |
| | | | GCAGCCACTGTGCGGCTGCTGGTGGGCGGGAAGCTG |
| | | | AACGTGCACATGAACCCCCCCCAGGTGAAGGCCACC |
| | | | ATCATCAGTGAGCAGCAGGCCAAGTCTCTGCTCAAG |
| | | | AACGAGAACACCCGCAATGATTACAGTGGCGAGATC |
| | | | TTGAACAACTGCTGCGTCATGGAGTACCACCAAGCC |
| | | | ACAGGCACCCTTAGTGCCCACTTCAGGAATATGTCC |
| | | | CTGAAACGAATTAAGAGGTCAGACCGTCGTGGGGCA |
| | | | GAGTCGGTGACAGAAGAAAAATTTACAATCCTGTTT |
| | | | GAATCCCAGTTCAGTGTTGGTGGAAATGAGCTGGTT |
| | | | TTTCAAGTCAAGACCCTGTCCCTGCCAGTGGTGGTG |
| | | | ATCGTTCATGGCAGCCAGGACAACAATGCGACGGCC |
| | | | ACTGTTCTCTGGGACAATGCTTTTGCAGAGCCTGGC |
| | | | AGGGTGCCATTTGCCGTGCCTGACAAAGTGCTGTGG |
| | | | CCACAGCTGTGTGAGGCGCTCAACATGAAATTCAAG |
| | | | GCCGAAGTGCAGAGCAACCGGGGCCTGACCAAGGAG |
| | | | AACCTCGTGTTCCTGGCGCAGAAACTGTTCAACAAC |
| | | | AGCAGCAGCCACCTGGAGGACTACAGTGGCCTGTCT |
| | | | GTGTCCTGGTCCCAGTTCAACAGGGAGAATTTACCA |
| | | | GGACGGAATTACACTTTCTGGCAATGGTTTGACGGT |
| | | | GTGATGGAAGTGTTAAAAAAACATCTCAAGCCTCAT |
| | | | TGGAATGATGGGGCCATTTTGGGGTTTGTAAACAAG |
| | | | CAACAGGCCCATGACCTACTGATTAACAAGCCAGAT |
| | | | GGGACCTTCCTCCTGAGATTCAGTGACTCAGAAATT |
| | | | GGCGGCATCACCATTGCTTGGAAGTTTGATTCTCAG |
| | | | GAAAGAATGTTTTGGAATCTGATGCCTTTTACCACC |
| | | | AGAGACTTCTCCATCAGGTCCCTAGCCGACCGCTTG |
| | | | GGAGACTTGAATTACCTTATCTACGTGTTTCCTGAT |
| | | | CGGCGAAAAGATGAAGTATACTCCAAATACTACACA |
| | | | CCAGTTCCCTGCGAGTCTGCTACTGCTAAAGCTGTT |
| | | | GATGGATACGTGAAGCCACAGATCAAGCAAGTGGTC |
| | | | CCTGAGTTTGTGAACGCATCTGCAGATGCCGGGGGC |
| | | | GGCAGCGCCACGTACATGGACCAGGCCCCCTCCCCA |
| | | | GCTGTGTGTCCCCAGGCTCACTATAACATGTACCCA |
| | | | CAGAACCCTGACTCAGTCCTTGACACCGATGGGGAC |
| | | | TTCGATCTGGAGGACACAATGGACGTAGCGCGGCGT |
| | | | GTGGAGGAGCTCCTGGGCCGGCCAATGGACAGTCAG |
| | | | TGGATCCCGCACGCACAATCGTGACCCCGCGACCTC |
| | | | TCCATCTTCAGCTTCTTCATCTTCACCAGAGGAATC |
| | | | ACTCTTGTGGATGTTTTAATTCCATGAATCGCTTCT |
| | | | CTTTTGAAACAATACTCATAATGTGAAGTGTTAATA |
| | | | CTAGTTGTGACCTTAGTGTTTCTGTGCATGGTGGCA |
| | | | CCAGCGAAGGGAGTGCGAGTATGTGTTTGTGTGTGT |
| | | | GTGTGTGTGTGTGTGTGTGTGCGTTGGTGCACGTTA |
| | | | TGGTGTTTCTCCCTCTCACTGTCTGAGAGTTTAGTT |
| | | | GTAGCAGA |
| S000031 | F32 | 159 | CCGAATGTGACCGCCTCCCGCTCCCTCACCCGCCGC |
| | | | GGGGAGGAGGAGCGGGCGAGAAGCTGCCGCCGAACG |
| | | | ACAGGACGTTGGGGCGGCCTGGCTCCCTCAGGTTTA |
| | | | AGAATTGTTTAAGCTGCATCAATGGAGCACATACAG |
| | | | GGAGCTTGGAAGACGATCAGCAATGGTTTTGGATTC |
| | | | AAAGATGCCGTGTTTGATGGCTCCAGCTGCATCTCT |
| | | | CCTACAATAGTTCAGCAGTTTGGCTATCAGCGCCGG |
| | | | GCATCAGATGATGGCAAACTCACAGATCCTTCTAAG |
| | | | ACAAGCAACACTATCCGTGTTTTCTTGCCGAACAAG |
| | | | CAAAGAACAGTGGTCAATGTGCGAAATGGAATGAGC |
| | | | TTGCATGACTGCCTTATGAAAGCACTCAAGGTGAGG |
| | | | GGCCTGCAACCAGAGTGCTGTGCAGTGTTCAGACTT |
| | | | CTCCACGAACACAAAGGTAAAAAAGCACGCTTAGAT |
| | | | TGGAATACTGATGCTGCGTCTTTGATTGGAGAAGAA |
| | | | CTTCAAGTAGATTTCCTGGATCATGTTCCCCTCACA |
| | | | ACACACAACTTTGCTCGGAAGACGTTCCTGAAGCTT |
| | | | GCCTTCTGTGACATCTGTCAGAAATTCCTGCTCAAT |
| | | | GGATTTCGATGTCAGACTTGTGGCTACAAATTTCAT |
| | | | GAGCACTGTAGCACCAAAGTACCTACTATGTGTGTG |
| | | | GACTGGAGTAACATCAGACAACTCTTATTGTTTCCA |
| | | | AATTCCACTATTGGTGATAGTGGAGTCCCAGCACTA |
| | | | CCTTCTTTGACTATGCGTCGTATGCGAGAGTCTGTT |
| | | | TCCAGGATGCCTGTTAGTTCTCAGCACAGATATTCT |
| | | | ACACCTCACGCCTTCACCTTTAACACCTCCAGTCCC |
| | | | TCATCTGAAGGTTCCCTCTCCCAGAGGCAGAGGTCG |
| | | | ACATCCACACCTAATGTCCACATGGTCAGCACCACG |
| | | | CTGCCTGTGGACAGCAGGATGATTGAGGATGCAATT |
| | | | CGAAGTCACAGCGAATCAGCCTCACCTTCAGCCCTG |
| | | | TCCAGTAGCCCCAACAATCTGAGCCCAACAGGCTGG |
| | | | TCACAGCCGAAAACCCCCGTGCCAGCACAAAGAGAG |
| | | | CGGGCACCAGTATCTGGGACCCAGGAGAAAAACAAA |
| | | | ATTAGGCCTCGTGGACAGAGAGATTCAAGCTATTAT |
| | | | TGGGAAATAGAAGCCAGTGAAGTGATGCTGTCCACT |
| | | | CGGATTGGGTCAGGCTCTTTTGGAACTGTTTATAAG |
| | | | GGTAAATGGCACGGAGATGTTGCAGTAAAGATCCTA |
| | | | AAGGTTGTCGACCCAACCCCAGAGCAATTCCAGGCC |
| | | | TTCAGGAATGAGGTGGCTGTTCTGCGCAAAACACGG |
| | | | CATGTGAACATTCTGCTTTTCATGGGGTACATGACA |
| | | | AAGGACAACCTGGCAATTGTGACCCAGTGGTGCGAG |
| | | | GGCAGCAGCCTCTACAAACACCTGCATGTCCAGGAG |
| | | | ACCAAGTTTCAGATGTTCCAGCTAATTGACATTGCC |
| | | | CGGCAGACGGCTCAGGGAATGGACTATTTGCATGCA |
| | | | AAGAACATCATCCATAGAGACATGAAATCCAACAAT |
| | | | ATATTTCTCCATGAAGGCTTAACAGTGAAAATTGGA |
| | | | GATTTTGGTTTGGCAACAGTAAAGTCACGCTGGAGT |
| | | | GGTTCTCAGCAGGTTGAACAACCTACTGGCTCTGTC |
| | | | CTCTGGATGGCCCCAGAGGTGATCCGAATGCAGGAT |
| | | | AACAACCCATTCAGTTTCCAGTCGGATGTCTACTCC |
| | | | TATGGCATCGTATTGTATGAACTGATGACGGGGGAG |
| | | | CTTCCTTATTCTCACATCAACAACCGAGATCAGATC |
| | | | ATCTTCATGGTGGGCCGAGGATATGCCTCCCCAGAT |
| | | | CTTAGTAAGCTATATAAGAACTGCCCCAAAGCAATG |
| | | | AAGAGGCTGGTAGCTGACTGTGTGAAGAAAGTAAAG |
| | | | GAAGAGAGGCCTCTTTTTCCCCAGATCCTGTCTTCC |
| | | | ATTGAGCTGCTCCAACACTCTCTACCGAAGATCAAC |
| | | | CGGAGCGCTTCCGAGCCATCCTTGCATCGGGCAGCC |
| | | | CACACTGAGGATATCAATGCTTGCACGCTGACCACG |
| | | | TCCCCGAGGCTGCCTGTCTTCTAGTTGACTTTGCAC |
| | | | CTGTCTTCAGGCTGCCAGGGGAGGAGGAGAAGCCAG |
| | | | CAGGCACCACTTTTCTGCTCCCTTTCTCCAGAGGCA |
| | | | GAACACATGTTTTCAGQAGAAGCTCTGCTAAGGACC |
| | | | TTCTAGACTGCTCACAGGGCCTTAACTTCATGTTGC |
| | | | CTTCTTTTCTATCCCTTTGGGCCCTGGGAGAAGGAA |
| | | | GCCATTTGCAGTGCTGGTGTGTCCTGGTCCCTCCCC |
| | | | ACATTCCCCATGCTCAAGGCCCAGCCTTCTGTAGAT |
| | | | GCGCAAGTGGATGTTGATGGTAGTACAAAAAGCAGG |
| | | | GGCCCAGCCCCAGCTGTTGGCTACATGAGTATTTAG |
| | | | AGGAAGTAAGGTAGCAGGCAGTCCAGCCCTGATGTG |
| | | | GAGACACATGGGATTTTGGAAATCAGCTTCTGGAGG |
| | | | AATGCATGTCACAGGCGGGACTTTCTTCAGAGAGTG |
| | | | GTGCAGCGCCAGACATTTTGCACATAAGGCACCAAA |
| | | | CAGCCCAGGACTGCCGAGACTCTGGCCGCCCGAAGG |
| | | | AGCCTGCTTTGGTACTATGGAACTTTTCTTAGGGGA |
| | | | CACGTCCTCCTTTCACAGCTTCTAAGGTGTCCAGTG |
| | | | CATTGGGATGGTTTTCCAGGCAAGGCACTCGGCCAA |
| | | | TCCGCATCTCAGCCCTCTCAGGAGCAGTCTTCCATC |
| | | | ATGCTGAATTTTGTCTTCCAGGAGCTGCCCCTATGG |
| | | | GGCGGGCCGCAGGGCCAGCCTGTTTCTCTAACAAAC |
| | | | AAACAAACAAACAGCCTTGTTTCTCTAGTCACATCA |
| | | | TGTGTATACAAGGAAGCCAGGAATACAGGTTTTCTT |
| | | | GATGATTTGGGTTTTAATTTTGTTTTTATTGCACCT |
| | | | GACAAAATACAGTTATCTGATGGTCCCTCAATTATG |
| | | | TTATTTTAATAAAATAAAATTAAATTT |
| S000039 | F33 | 160 | TCCAGTTTGCTTCTTGGAGAACACTGGACAGCTGAA |
| | | | TAAATGCAGTATCTAAATATAAAAGAGGACTGCAAT |
| | | | GCCATGGCTTTCTGTGCTAAAATGAGGAGCTCCAAG |
| | | | AAGACTGAGGTGAACCTGGAGGCCCCTGAGCCAGGG |
| | | | GTGGAAGTGATCTTCTATCTGTCGGACAGGGAGCCC |
| | | | CTCCGGCTGGGCAGTGGAGAGTACACAGCAGAGGAA |
| | | | CTGTGCATCAGGGCTGCACAGGCATGCCGTATCTCT |
| | | | CCTCTTTGTCACAACCTCTTTGCCCTGTATGACGAG |
| | | | AACACCAAGCTCTGGTATGCTCCAAATCGCACCATC |
| | | | ACCGTTGATGACAAGATGTCCCTCCGGCTCCACTAC |
| | | | CGGATGAGGTTCTATTTCACCAATTGGCATGGAACC |
| | | | AACGACAATGAGCAGTCAGTGTGGCGTCATTCTCCA |
| | | | AAGAAGCAGAAAAATGGCTACGAGAAAAAAAAGATT |
| | | | CCAGATGCAACCCCTCTCCTTGATGCCAGCTCACTG |
| | | | GAGTATCTGTTTGCTCAGGGACAGTATGATTTGGTG |
| | | | AAATGCCTGGCTCCTATTCGAGACCCCAAGACCGAG |
| | | | CAGGATGGACATGATATTGAGAACGAGTGTCTAGGG |
| | | | ATGGCTGTCCTGGCCATCTCACACTATGCCATGATG |
| | | | AAGAAGATGCAGTTGCCAGAACTGCCCAAGGACATC |
| | | | AGCTACAAGCGATATATTCCAGAAACATTGAATAAG |
| | | | TCCATCAGACAGAGGAACCTTCTCACCAGGATGCGG |
| | | | ATAAATAATGTTTTCAAGGATTTCCTAAAGGAATTT |
| | | | AACAACAAGACCATTTGTGACAGCAGCGTGTCCACG |
| | | | CATGACCTGAAGGTGAAATACTTGGCTACCTTGGAA |
| | | | ACTTTGACAAAACATTACGGTGCTGAAATATTTGAG |
| | | | ACTTCCATGTTACTGATTTCATCAGAAAATGAGATG |
| | | | AATTGGTTTCATTCGAATGACGGTGGAAACGTTCTC |
| | | | TACTACGAAGTGATGGTGACTGGGAATCTTGGAATC |
| | | | CAGTGGAGGCATAAACCAAATGTTGTTTCTGTTGAA |
| | | | AAGGAAAAAAATAAACTGAAGCGGAAAAAACTGGAA |
| | | | AATAAAGACAAGAAGGATGAGGAGAAAAACAAGATC |
| | | | CGGGAAGAGTGGAACAATTTTTCATTCTTCCCTGAA |
| | | | ATCACTCACATTGTAATAAAGGAGTCTGTGGTCAGC |
| | | | ATTAACAAGCAGGACAACAAGAAAATGGAACTGAAG |
| | | | CTCTCTTCCCACGAGGAGGCCTTGTCCTTTGTGTCC |
| | | | CTGGTAGATGGCTACTTCCGGCTCACAGCAGATGCC |
| | | | CATCATTACCTCTGCACCGACGTGGCCCCCCCGTTG |
| | | | ATCGTCCACAACATACAGAATGGCTGTCATGGTCCA |
| | | | ATCTGTACAGAATACGCCATCAATAAATTGCGGCAA |
| | | | GAAGGAAGCGAGGAGGGGATGTACGTGCTGAGGTGG |
| | | | AGCTGCACCGACTTTGACAACATCCTCATGACCGTC |
| | | | ACCTGCTTTGAGAAGTCTGAGCAGGTGCAGGGTGCC |
| | | | CAGAAGCAGTTCAAGAACTTTCAGATCGAGGTGCAG |
| | | | AAGGGCCGCTACAGTCTGCACGGTTCGGACCGCAGC |
| | | | TTCCCCAGCTTGGGAGACCTCATGAGCCACCTCAAG |
| | | | AAGCAGATCCTGCGCACGGATAACATCAGCTTCATG |
| | | | CTAAAACGCTGCTGCCAGCCCAAGCCCCGAGAAATC |
| | | | TCCAACCTGCTGGTGGCTACTAAGAAAGCCCAGGAG |
| | | | TGGCAGCCCGTCTACCCCATGAGCCAGCTGAGTTTC |
| | | | GATCGGATCCTCAAGAAGGATCTGGTGCAGGGCGAG |
| | | | CACCTTGGGAGAGGCACGAGAACACACATCTATTCT |
| | | | GGGACCCTGATGGATTACAAGGATGACGAAGGAACT |
| | | | TCTGAAGAGAAGAAGATAAAAGTGATCCTCAAAGTC |
| | | | TTAGACCCCAGCCACAGGGATATTTCCCTGGCCTTC |
| | | | TTCGAGGCAGCCAGCATGATGAGACAGGTCTCCCAC |
| | | | AAACACATCGTGTACCTCTATGGCGTCTGTGTCCGC |
| | | | GACGTGGAGAATATCATGGTGGAAGAGTTTGTGGAA |
| | | | GGGGGTCCTCTGGATCTCTTCATGCACCGGAAAAGT |
| | | | GATGTCCTTACCACACCATGGAAATTCAAAGTTGCC |
| | | | AAACAGCTGGCCAGTGCCCTGAGCTACTTGGAGGAT |
| | | | AAAGACCTGGTCCATGGAAATGTGTGTACTAAAAAC |
| | | | CTCCTCCTGGCCCGTGAGGGAATCGACAGTGAGTGT |
| | | | GGCCCATTCATCAAGCTCAGTGACCCCGGCATCCCC |
| | | | ATTACGGTGCTGTCTAGGCAAGAATGCATTGAACGA |
| | | | ATCCCATGGATTGCTCCTGAGTGTGTTGAGGACTCC |
| | | | AAGAACCTGAGTGTGGCTGCTGACAAGTGGAGCTTT |
| | | | GGAACCACGCTCTGGGAAATCTGCTACAATGGCGAG |
| | | | ATCCCCTTGAAAGACAAGACGCTGATTGAGAAAGAG |
| | | | AGATTCTATGAAAGCCGGTGCAGGCCAGTGACACCA |
| | | | TCATGTAAGGAGCTGGCTGACCTCATGACCCGCTGA |
| | | | TGAACTATGACCCCAATCAGAGGCCTTTCTTGCGAG |
| | | | CCATCATGAGAGACATTAATAAGCTTGAAGAGCAGA |
| | | | ATCCAGATATTGTTTCCAGAAAAAAAAACCAGCCAA |
| | | | CTGAAGTGGACCCCACACATTTTGAGAAGCGCTTCC |
| | | | TAAAGAGGATCCGTGACTTGGGAGAGGGCCACTTTG |
| | | | GGAAGGTTGAGCTCTGCAGGTATGACCCCGAAGACA |
| | | | ATACAGGGGAGCAGGTGGCTGTTAAATCTCTGAAGC |
| | | | CTGAGAGTGGAGGTAACCACATAGCTGATCTGAAAA |
| | | | AGGAAATCGAGATCTTAAGGAACCTCTATCATGAGA |
| | | | ACATTGTGAAGTACAAAGGAATCTGCACAGAAGACG |
| | | | GAGGAAATGGTATTAAGCTCATCATGGAATTTCTGC |
| | | | CTTCGGGAAGCCTTAAGGAATATCTTCCAAAGAATA |
| | | | AGAACAAAATAAACCTCAAACAGCAGCTAAAATATG |
| | | | CCGTTCAGATTTGTAAGGGGATGGACTATTTGGGTT |
| | | | CTCGGCAATACGTTCACCGGGACTTGGCAGCAAGAA |
| | | | ATGTCCTTGTTGAGAGTGAACACCAAGTGAAAATTG |
| | | | GAGACTTCGGTTTAACCAAAGCAATTGAAACCGATA |
| | | | AGGAGTATTACACCGTCAAGGATGACCGGGACAGCC |
| | | | CTGTGTTTTGGTATGCTCCAGAATGTTTAATGCAAT |
| | | | CTAAATTTTATATTGCCTCTGACGTCTGGTCTTTTG |
| | | | GAGTCACTCTGCATGAGCTGCTGACTTACTGTGATT |
| | | | CAGATTCTAGTCCCATGGCTTTGTTCCTGAAAATGA |
| | | | TAGGCGCAACCCATGGCCAGATGACAGTCACAAGAC |
| | | | TTGTGAATACGTTAAAAGAAGGAAAACGCCTGCCGT |
| | | | GCCCACCTAACTGTCCAGATGAGGTTTATCAGCTTA |
| | | | TGAGAAAATGCTGGGAATTCCAACCATCCAATCGGA |
| | | | CAAGCTTTCAGAACCTTATTGAAGGATTTGAAGCAC |
| | | | TTTTAAAATAAGAAGCATGAATAACATTTAAATTCC |
| | | | ACAGATTATCAA |
| S000040 | F34 | 161 | CTGCAGCTTCTAGGACCCGGTTTCTTTTACTGATTT |
| | | | AAAAACAAAACAAAAAAAAATAAAAAAGTTGTGCCT |
| | | | GAAATGAATCTTGTTTTTTTTTTATAAGTAGCCGCC |
| | | | TGGTTACTGTGTCCTGTAAAATACAGACATTGACCC |
| | | | TTGGTGTAGCTTCTGTTCAACTTTATATCACGGGAA |
| | | | TGGATGGGTCTGATTTCTTGGCCCTCTTCTTGAATT |
| | | | GGCCATATACAGGGTCCCTGGCCAGTGGACTGAAGG |
| | | | CTTTGTCTAAGATGACAAGGGTCAGCTCAGGGGATG |
| | | | TGGGGGAGGGCGGTTTTATCTTCCCCCTTGTCGTTT |
| | | | GAGGTTTTGATCTCTGGGTAAAGAGGCCGTTTATCT |
| | | | TTGTAAACACGAAACATTTTTGCTTTCTCCAGTTTT |
| | | | CTGTTAATGGCGAAAGAATGGAAGCGAATAAAGTTT |
| | | | TACTGATTTTTGAGACACTAGCACCTAGCGCTTTCA |
| | | | TTATTGAAACGTCCCGTGTGGGAGGGGCGGGTCTGG |
| | | | GTGCGGCTGCCGCATGACTCGTGGTTCGGAGGCCCA |
| | | | CGTGGCCGGGGCGGGGACTCAGGCGCCTGGCAGCCG |
| | | | ACTGATTACGTAGCGGGCGGGGCCGGAAGTGCCGCT |
| | | | CCTTGGTGGGGGCTGTTCATGGCGGTTCCGGGGTCT |
| | | | CCAACATTTTTCCCGGTCTGTGGTCCTAAATCTGTC |
| | | | CAAAGCAGAGGCAGTGGAGCTTGAGGTTCTTGCTGG |
| | | | TGTGAAATGACTGAGTACAAACTGGTGGTGGTTGGA |
| | | | GCAGGTGGTGTTGGGAAAAGCGCACTGACAATCCAG |
| | | | CTAATCCAGAACCACTTTGTAGATGAATATGATCCC |
| | | | ACCATAGAGGATTCTTACAGAAAACAAGTGGTTATA |
| | | | GATGGTGAAACCTGTTTGTTGGACATACTGGATACA |
| | | | GCTGGACAAGAAGAGTACAGTGCCATGAGAGACCAA |
| | | | TACATGAGGACAGGCGAAGGCTTCCTCTGTGTATTT |
| | | | GCCATCAATAATAGCAAGTCATTTGCGGATATTAAC |
| | | | CTCTACAGGGAGCAGATTAAGCGAGTAAAAGACTCG |
| | | | GATGATGTACCTATGGTGCTAGTGGGAAACAAGTGT |
| | | | GATTTGCCAACAAGGACAGTTGATACAAAACAAGCC |
| | | | CACGAACTGGCCAAGAGTTACGGGATTCCATTCATT |
| | | | GAAACCTCAGCCAAGACCAGACAGGGTGTTGAAGAT |
| | | | GCTTTTTACACACTGGTAAGAGAAATACGCCAGTAC |
| | | | CGAATGAAAAAACTCAACAGCAGTGATGATGGGACT |
| | | | CAGGGTTGTATGGGATTGCCATGTGTGGTGATGTAA |
| | | | CAAGATACTTTTAAAGTTTTGTCAGAAAAGAGCCAC |
| | | | TTTCAAGCTGCACTGACACCCTGGTCCTGACTTCCT |
| | | | GGAGGAGAAGTATTCCTGTTGCTGTCTTCAGTCTCA |
| | | | CAGAGAAGCTCCTGCTACTTCCCCAGCTCTCAGTAG |
| | | | TTTAGTACAATAATCTCTATTTGAGAAGTTCTCAGA |
| | | | ATAACTACCTCCTCACTTGGCTGTCTGACCAGAGAA |
| | | | TGCACCTCTTGTTACTCCCTGTTATTTTTCTGCCCT |
| | | | GGGTTCTTCCACAGCACAAACACACCTCAACACACC |
| | | | TCTGCCACCCCAGGTTTTTCATGTGAAAAGCAGTTC |
| | | | ATGTCTGAAACAGAGAACCAAACCGCAAACGTGAAA |
| | | | TTCTATTGAAAACAGTGTCTTGAGCTCTAAAGTAGC |
| | | | AACTGCTGGTGATTTTTTTTTTCTTTTTACTGTTGA |
| | | | ACTTAGAACTATGCCTAATTTTTGGAGATGTCATAA |
| | | | TTACTGTTTTGCCAAGAATATAGTTATTATTATTGC |
| | | | TGTTTGGTTTGTTTATAATGTTATCGGCTCTATTCT |
| | | | CTAAACTGGCATCTGCTCTAGATTCATAAATACAAA |
| | | | AATGAATACTGAATTTTGAGTCTATCCTAGTCTTCA |
| | | | CAACTTTGACGTAATTAAATCCAACTTTTCACAGTG |
| | | | AAGTGCCTTTTTCCTAGAAGTGGTTTGTAGACTCCT |
| | | | TTATAATATTTCAGTGGAATAGATGTCTCAAAAATC |
| | | | CTTATGCATGAAATGAATGTCTGAGATACGTCTGTG |
| | | | ACTTATCTACCATTGAAGGAAAGCTATATCTATTTG |
| | | | AGAGCAGATGCCATTTTGTACATGTATGAAATTGGT |
| | | | TTTCCAGAGGCCTGTTTTGGGGCTTTCCCAGGAGAA |
| | | | AGATGAAACTGAAAGCATATGAATAATTTCACTTAA |
| | | | TAATTTTTACCTAATCTCCACTTTTTTCATAGGTTA |
| | | | CTACCTATACAATGTATGTAATTTGTTTCCCCTAGC |
| | | | TTACTGATAAACCTAATATTCAATGAACTTCCATTT |
| | | | GTATTCAAATTTGTGTCATACCAGAAAGCTCTACAT |
| | | | TTGCAGATGTTCAAATATTGTAAAACTTTGGTGCAT |
| | | | TGTTATTTAATAGCTGTGATCAGTGATTTTCAAACC |
| | | | TCAAATATAGTATATTAACAAATT |
| S000046 | F35 | 162 | CGGGGGGATCTTGGCTGTGTGTCTGCGGATCTGTAG |
| | | | TGGCGGCGGCGGCGGCGGCGGCGGGGAGGCAGCAGG |
| | | | CGCGGGAGCGGGCGCAGGAGCAGGCGGCGGCGGTGG |
| | | | CGGCGGCGGTTAGACATGAACGCCGCCTCGGCGCCG |
| | | | GCGGTGCACGGAGAGCCCCTTCTCGCGCGCGGGCGG |
| | | | TTTGTGTGATTTTGCTAAAATGCATCACCAACAGCG |
| | | | AATGGCTGCCTTAGGGACGGACAAAGAGCTGAGTGA |
| | | | TTTACTGGATTTCAGTGCGATGTTTTCACCTCCTGT |
| | | | GAGCAGTGGGAAAAATGGACCAACTTCTTTGGCAAG |
| | | | TGGACATTTTACTGGCTCAAATGTAGAAGACAGAAG |
| | | | TAGCTCAGGGTCCTGGGGGAATGGAGGACATCCAAG |
| | | | CCCGTCCAGGAACTATGGAGATGGGACTCCCTATGA |
| | | | CCACATGACCAGCAGGGACCTTGGGTCACATGACAA |
| | | | TCTCTCTCCACCTTTTGTCAATTCCAGAATACAAAG |
| | | | TAAAACAGAAAGGGGCTCATACTCATCTTATGGGAG |
| | | | AGAATCAAACTTACAGGGTTGCCACCAGCAGAGTCT |
| | | | CCTTGGAGGTGACATGGATATGGGCAACCCAGGAAC |
| | | | CCTTTCGCCCACCAAACCTGGTTCCCAGTACTATCA |
| | | | GTATTCTAGCAATAATCCCCGAAGGAGGCCTCTTCA |
| | | | CAGTAGTGCCATGGAGGTACAGACAAAGAAAGTTCG |
| | | | AAAAGTTCCTCCAGGTTTGCCATCTTCAGTCTATGC |
| | | | TCCATCAGCAAGCACTGCCGACTACAATAGGGACTC |
| | | | GCCAGGCTATCCTTCCTGCAAACCAGCAACCAGCAC |
| | | | TTTCCCTAGCTCCTTCTTCATGCAAGATGGCCATCA |
| | | | CAGCAGTGACCCTTGGAGCTCCTCCAGTGGGATGAA |
| | | | TCAGCCTGGCTATGCAGGAATGTTGGGCAACTCTTC |
| | | | TCATATTCCACAGTCCAGCAGCTACTGTAGCCTGCA |
| | | | TCCACATGAACGTTTGAGCTATCCATCACACTCCTC |
| | | | AGCAGACATCAATTCCAGTCTTCCTCCGATGTCCAC |
| | | | TTTCCATCGTAGTGGTACAAACCATTACAGCACCTC |
| | | | TTCCTGTACGCCTCCTGCCAACGGGACAGACAGTAT |
| | | | AATGGCAAATAGAGGAAGCGGGGCAGCCGGCAGCTC |
| | | | CCAGACTGGAGATGCTCTGGGGAAAGCACTTGCTTC |
| | | | GATCTATTCTCCAGATCACACTAACAACAGCTTTTC |
| | | | ATCAAACCCTTCAACTCCTGTTGGCTCTCCTCCATC |
| | | | TCTCTCAGCAGGCACAGCTGTTTGGTCTAGAAATGG |
| | | | AGGACAGGCCTCATCGTCTCCTAATTATGAAGGACC |
| | | | CTTACACTCTTTGCAAAGCCGAATTGAAGATCGTTT |
| | | | AGAAAGACTGGATGATGCTATTCATGTTCTCCGGAA |
| | | | CCATGCAGTGGGCCCATCCACAGCTATGCCTGGTGG |
| | | | TCATGGGGACATGCATGGAATCATTGGACCTTCTCA |
| | | | TAATGGAGCCATGGGTGGTCTGGGCTCAGGGTATGG |
| | | | AACCGGCCTTCTTTCAGCCAACAGACATTCACTCAT |
| | | | GGTGGGGACGCATCGTGAAGATGGCGTGGCCCTGAG |
| | | | AGGCAGCCATTCTCTTCTGCCAAACCAGGTTCCGGT |
| | | | TCCACAGCTTCCTGTCCAGTCTGCGACTTCCCCTGA |
| | | | CCTGAACCCACCCCAGGACCCTTACAGAGGCATGCC |
| | | | ACCAGGACTACAGGGGCAGAGTGTCTCCTCTGGCAG |
| | | | CTCTGAGATCAAATCCGATGACGAGGGTGATGAGAA |
| | | | CCTGCAAGACACGAAATCTTCGGAGGACAAGAAATT |
| | | | AGATGACGACAAGAAGGATATCAAATCAATTACTAG |
| | | | CAATAATGACGATGAGGACCTGACACCAGAGCAGAA |
| | | | GGCAGAGCGTGAGAAGGAGCGGAGGATGGCCAACAA |
| | | | TGCCCGAGAGCGTCTGCGGGTCCGTGACATCAACGA |
| | | | GGCTTTCAAAGAGCTCGGCCGCATGGTGCAGCTCCA |
| | | | CCTCAAGAGTGACAAGCCCCAGACCAAGCTCGTGAT |
| | | | CCTCCACCAGGCGGTGGCCGTCATCCTCAGTCTGGA |
| | | | GCAGCAAGTCCGAGAAAGGAATCTGAATCCGAAAGC |
| | | | TGCGTGTCTGAAAAGAAGGGAGGAAGAGAAGGTGTC |
| | | | CTCGGAGCCTCCCCCTCTCTCCTTGGCCGGCGCACA |
| | | | CCCTGGAATGGGAGACGCATCGAATCACATGGGACA |
| | | | GATGTAAAAGGGTCCAAGTTGCCACATTGCTTCATT |
| | | | AAAACAAGAGACCACTTCCTTAACAGCTGTATTATC |
| | | | TTAAACCCACATAAACACTTCTCCTTAACCCCCATT |
| | | | TTTGTAATATAAGACAAGTCTGAGTAGTTATGAATC |
| | | | GCAGACGCAAGAGGTTTCAGCATTCCCAATTATCAA |
| | | | AAAACAGAAAAACAAAAAAAAGAAAGAAAAAAGTGC |
| | | | AACTTGAGGGACGACTTTCTTTAACATATCATTCAG |
| | | | AATGTGCAAAGCAGTATGTACAGGCTGAGACACAGC |
| | | | CCAGAGACTGAACGGC |
| S000050 | F36 | 163 | AAAAAAAAGAAAAAAAAAGGCACAAAAAAGTGGAAA |
| | | | CTTTTCCCTGTCCATTCCATCAAGTCCTGAAAAATC |
| | | | AAAATGGATTTAGAGAAAAATTATCCGACTCCTCGG |
| | | | ACCAGCAGGACAGGACATGGAGGAGTGAATCAGCTT |
| | | | GGGGGGGTTTTTGTGAATGGACGGCCACTCCCGGAT |
| | | | GTAGTCCGCCAGAGGATAGTGGAACTTGCTCATCAA |
| | | | GGTGTCAGGCCCTGCGACATCTCCAGGCAGCTTCGG |
| | | | GTCAGCCATGGTTGTGTCAGCAAAATTCTTGGCAGG |
| | | | TATTATGAGACAGGAAGCATCAAGCCTGGGGTAATT |
| | | | GGAGGATCCAAACCAAAGGTCGCCACACCCAAAGTG |
| | | | GTGGAAAAAATCGCTGAATATAAACGCCAAAATCCC |
| | | | ACCATGTTTGCCTGGGAGATCAGGGACCGGCTGCTG |
| | | | GCAGAGCGGGTGTGTGACAATGACACCGTGCCTAGC |
| | | | GTCAGTTCCATCAACAGGATCATCCGGACAAAAGTA |
| | | | CAGCAGCCACCCAACCAACCAGTCCCAGCTTCCAGT |
| | | | CACAGCATAGTGTCCACTGGCTCGGTGACGCAGGTG |
| | | | TCCTCGGTGAGCACGGATTCGGCCGGCTCGTCGTAC |
| | | | TCCATCAGCGGCATCCTGGGCATCACGTCCCCCAGC |
| | | | GCCGACACCAACAAGCGCAAGAGAGACGAAGGTATT |
| | | | CAGGAGTCTCCGGTGCCGAACGGCCACTCGCTTCCG |
| | | | GGCAGAGACTTCCTCCGGAAGCAGATGCGGGGAGAC |
| | | | TTGTTCACACAGCAGCAGCTGGAGGTGCTGGACCGC |
| | | | GTGTTTGAGAGGCAGCACTACTCAGACATCTTCACC |
| | | | ACCACAGAGCCCATCAAGCCCGAGCAGACCACAGAG |
| | | | TATTCAGCCATGGCCTCGCTGGCTGGTGGGCTGGAC |
| | | | GACATGAAGGCCAATCTGGCCAGCCCCACCCCTGCT |
| | | | GACATCGGGAGCAGTGTGCCAGGCCCGCAGTCCTAC |
| | | | CCCATTGTGACAGGCCGTGACTTGGCGAGCACGACC |
| | | | CTCCCCGGGTACCCTCCACACGTCCCCCCCGCTGGA |
| | | | CAGGGCAGCTACTCAGCACCGACGCTGACAGGGATG |
| | | | GTGCCTGGGAGTGAGTTTTCCGGGAGTCCCTACAGC |
| | | | CACCCTCAGTATTCCTCGTACAACGACTCCTGGAGG |
| | | | TTCCCCAACCCGGGGCTGCTTGGCTCCCCCTACTAT |
| | | | TATAGCGCTGCCGCCCGAGGAGCCGCCCCACCTGCA |
| | | | GCCGCCACTGCCTATGACCGTCACTGACCCTTGGAG |
| | | | CCAGGCGGGCACCAAACACTGATGGCACCTATTGAG |
| | | | GGTGACAGCCACCCAGCCCTCCTGAAGATAGCCAGA |
| | | | GAGCCCATGAGACCGTCCCCCAGCATCCCCCACTTG |
| | | | CCTGAAGCTCCCCTCTTCCTCTCTTCCTCCAGGGAC |
| | | | TCTGGGGCCCTTTGGTGGGGCCGTTGGACTTCTGGA |
| | | | TGCTTGTCTATTTCTAAAAGCCAATCTATGAGCTTC |
| | | | TCCCGATGGCCACTGGGTCTCTGCAAACCAATAGAC |
| | | | TGTCCTGCAAATAACCGCAGCCCCAGCCCAGCCTGC |
| | | | CTGTCCTCCAGCTGTCTGACTATCCATCCATCATAA |
| | | | CCACCCCAGCCTGGGAAGGAGAGCTTGCTTTTGTTG |
| | | | CTTCAGCAGCACCCATGTAAATACCTTCTTGCTTTT |
| | | | CTGTGGGCCTGAAGGTCCGACTGAGAAGACTGCTCC |
| | | | ACCCATGATGCATCTCGCACTCTTGGTGCATCACCG |
| | | | GACATCTTAGACCTATGGCAGAGCATCCTCTCTGCC |
| | | | CTGGGTGACCCTGGCAGGTGCGCTCAGAGCTGTCCT |
| | | | CAAGATGGAGGATGCTGCCCTTGGGCCCCAGCCTCC |
| | | | TGCTCATCCCTCCTTCTTTAGTATCTTTACGAGGAG |
| | | | TCTCACTGGGCTGGTTGTGCTGCAGGCTCCCCCTGA |
| | | | GGCCCCTCTCCAAGAGGAGCACACTTTGGGGAGATG |
| | | | TCCTGGTTTCCTGCCTCCATTTCTCTGGGACCGATG |
| | | | CAGTATCAGCAGCTCTTTTCCAGATCAAAGAACTCA |
| | | | AAGAAAACTGTCTGGGAGATTCCTCAGCTACTTTTC |
| | | | CGAAGCAGAATGTCATCCGAGGTATTGATTACATTG |
| | | | TGGACTTTGAATGTGAGGGCTGGATGGGACGCAGGA |
| | | | GATCATCTGATCCCAGCCAAGGAGGGGCCTGAGGCT |
| | | | CTCCCTACTCCCTCAGCCCCTGGAACGGTGTTTTCT |
| | | | GAGGCATGCCCAGGTTCAGGTCACTTCGGACACCTG |
| | | | CCATGGACACTTCACCCACCCTCCAGGACCCCAGCA |
| | | | AGTGGATTCTGGGCAAGCCTGTTCCGGTGATGTAGA |
| | | | CAATAATTAACACAGAGGACTTTCCCCCACACCCAG |
| | | | ATCACAAACAGCCTACAGCCAGAACTTCTGAGCATC |
| | | | CTCTCGGGGCAGACCCTCCCCGTCCTCGTGGAGCTT |
| | | | AGCAGGCAGCTGGGCATGGAGGTGCTGGGGCTGGGG |
| | | | CAGATGCCTAATTTCGCACAATGCATGCCCACCTGT |
| | | | TGATGTAAGGGGCCGCGATGGTCAGGGCCACGGCCA |
| | | | AGGGCGACGGGAACTTGGAGAGGGAGCTTGGAGAAC |
| | | | TCACTGTGGGCTAGGGTGGTCAGAGGAAGCCAGCAG |
| | | | GGAAGATCTGGGGGACAGAGGAAGGCCTCCTGAGGG |
| | | | AGGGGCAGGAGAGCAGTGAGGAGCTGCTGTGTGACC |
| | | | TGGGAGTGATTTTGACATGGGGGTGCCAGGTGCCAT |
| | | | CATCTCTTTACCTGGGGCCTTAATTCCTTGCATAGT |
| | | | CTCTCTTGTCAAGTCAGAACAGCCAGGTAGAGCCCT |
| | | | TGTCCAAACCTGGGCTGAATGACAGTGATGAGAGGG |
| | | | GGCTTGGCCTTCTTAGGTGACAATGTCCCCCATATC |
| | | | TGTATGTCACCAGGATGGCAGAGAGCCAGGGCAGAG |
| | | | AGAGACTGGACTTGGGATCAGCAGGCCAGGCAGGTC |
| | | | TTGTCCTGGTCCTGGCCACATGTCTTTGCTGTGGGA |
| | | | CCTCAGACAAAACCCTGCACCTCTTTGAGCCTTGGC |
| | | | TGCCTTGGTGCAGCAGGGTCATCTGTAGGGCCACCC |
| | | | CACAGCTCTTTCCTTCCCCTCCTCTCTCCAGGGAGC |
| | | | CGGGGCTGTGAGAGGATCATCTGGGGCAGGCCCTCC |
| | | | ACTTCCAAGCAAGCAGATGGGGGTGGGCACCTGAGG |
| | | | CCCAATAATATTTGGACCAAGTGGGAAACAAGAACA |
| | | | CTCGGAGGGGCGGGAATCAGAAGAGCCTGGAAAAAG |
| | | | ACCTAGCCCAACTTCCCTTGTGGGAAACTGAGGCCC |
| | | | AGCTTGGGGAAGGCCAGGACCATGCAGGGAGAAAAA |
| | | | G |
| S000056 | F37 | 164 | ATGGAGACCGAACCGCCTCACAACGAGCCCATCCCC |
| | | | GTCGAGAATGATGGCGAGGCCTGTGGACCCCCAGAG |
| | | | GTCTCCAGACCCAACTTTCAGGTCCTCAACCCGGCA |
| | | | TTCAGGGAAGCTGGAGCCCATGGAAGCTACAGCCCA |
| | | | CCTCCTGAGGAAGCAATGCCCTTCGAGGCTGAACAG |
| | | | CCCAGCTTGGGAGGCTTCTGGCCTACACTGGAGCAG |
| | | | CCTGGATTCCCCAGTGGGGTCCATGCAGGCCTTGCC |
| | | | AKGSTYSGSCCAGCACTCATGGAGCCCGGAGCCTTC |
| | | | AGTGGTGCCAGACCAGGCCTGGGAGGATACAGCCCT |
| | | | CCACCAGAAGAAGCTATGCCCTTTGAGTTTGACCAG |
| | | | CCTGCCCAGAGAGGCTGCAGTCAACTTCTCTTACAG |
| | | | GTCCCAGACCTTGCTCCAGGAGGCCCAGGTGCTGCA |
| | | | GGGGTCCCCGGAGCTCCTCCCGAGGAGCCCCAAGCC |
| | | | CTCAGGCCTGCAAAGGCTGGCTCCAGAGGAGGCTAC |
| | | | AGCCCTCCCCCTGAGGAGACTATGCCATTTGAGCTT |
| | | | GATGGAGAAGGATTTGGGGACGACAGCCCACCCCCG |
| | | | GGGCTTTCCCGAGTTATCGCACAAGTCGACGGCAGC |
| | | | AGCCAGTTCGCGGCAGTCGCGGCCTCGAGTGCGGTC |
| | | | CGCCTCACTCCCGCCGCGAACGCGCCTCCCCTCTGG |
| | | | GTCCCAGGCGCCATCGGCAGCCCATCCCAAGAGGCT |
| | | | GTCAGACCTCCTTCTAACTTCACGGGCAGCAGCCCC |
| | | | TGGATGGAGATCTCCGGACCCCCGTTCGAGATTGGC |
| | | | AGCGCCCCCGCTGGGGTCGACGACACTCCCGTCAAC |
| | | | ATGGACAGCCCCCCAATCGCGCTTGACGGCCCGCCC |
| | | | ATCAAGGTCTCCGGAGCCCCAGATAAGAGAGAGCGA |
| | | | GCAGAGAGACCCCCAGTTGAGGAGGAAGCAGCAGAG |
| | | | ATGGAAGGAGCCGCTGATGCCGCGGAGGGAGGAAAA |
| | | | GTACCCTCTCCGGGGTACGGATCCCCTGCCGCCGGG |
| | | | GCAGCCTCAGCGGATACCGCTGCCAGGGCAGCCCCT |
| | | | GCAGCCCCAGCCGATCCTGACTCCGGGGCAACCCCA |
| | | | GAAGATCCCGACTCCGGGACAGCACCAGCCGATCCT |
| | | | GACTCCGGGGCATTCGCAGCCGATCCCGACTCCGGG |
| | | | GCAGCCCCTGCCGCCCCAGCCGATCCCGACTCCGGG |
| | | | GCGGCCCCTGACGCCCCAGCCGATCCCGACTCCGGG |
| | | | GCGGCCCCTGACGCCCCAGCCGATCCAGATGCCGGG |
| | | | GCGGCCCCTGAGGCTCCCGCCGCCCCTGCGGCTGCT |
| | | | GAGACCCGGGCAGCCCATGTCGCCCCAGCTGCGCCA |
| | | | GACGCAGGGGCTCCCACTGCCCCAGCCGCTTCTGCC |
| | | | ACCCGGGCAGCCCAAGTCCGCCGGGCGGCCTCTGCA |
| | | | GCCCCTGCCTCCGGGGCCAGACGCAAGATCCATCTC |
| | | | AGACCCCCCAGCCCCGAGATCCAGGCTGCCGATCCG |
| | | | CCTACTCCGCGGCCTACTCGCGCGTCTGCCTGGCGG |
| | | | GGCAAGTCCGAGAGCAGCCGCGGCCGCCGCGTGTAC |
| | | | TACGATGAAGGGGTGGCCAGCAGCGACGATGACTCC |
| | | | AGCGGAGACGAGTCCGACGATGGGACCTCCGGATGC |
| | | | CTCCGCTGGTTTCAGCATCGGCGAAATCGCCGCCGC |
| | | | CGAAAGCCCCAGCGCAACTTACTCCGCAACTTTCTC |
| | | | GTGCAAGCCTTCGGGGGCTGCTTCGGTCGATCTGAG |
| | | | AGTCCCCAGCCCAAAGCCTCGCGCTCTCTCAAGGTC |
| | | | AAGAAGGTACCCCTGGCGGAGAAGCGCAGACAGATG |
| | | | CGCAAAGAAGCCCTGGAGAAGCGGGCCCAGAAGCGC |
| | | | GCAGAGAAGAAACGCAGTAAGCTCATCGACAAACAA |
| | | | CTCCAGGACGAAAAGATGGGCTACATGTGTACGCAC |
| | | | CGCCTGCTGCTTCTAG |
| S000058 | F38 | 165 | CTGCAGCTTCTAGGACCCGGTTTCTTTTACTGATTT |
| | | | AAAAACAAAACAAAAAAAAATAAAAAAGTTGTGCCT |
| | | | GAAATGAATCTTGTTTTTTTTTTATAAGTAGCCGCC |
| | | | TGGTTACTGTGTCCTGTAAAATACAGACATTGACCC |
| | | | TTGGTGTAGCTTCTGTTCAACTTTATATCACGGGAA |
| | | | TGGATGGGTCTGATTTCTTGGCCCTCTTCTTGAATT |
| | | | GGCCATATACAGGGTCCCTGGCCAGTGGACTGAAGG |
| | | | CTTTGTCTAAGATGACAAGGGTCAGCTCAGGGGATG |
| | | | TGGGGGAGGGCGGTTTTATCTTCCCCCTTGTCGTTT |
| | | | GAGGTTTTGATCTCTGGGTAAAGAGGCCGTTTATCT |
| | | | TTGTAAACACGAAACATTTTTGCTTTCTCCAGTTTT |
| | | | CTGTTAATGGCGAAAGAATGGAAGCGAATAAAGTTT |
| | | | TACTGATTTTTGAGACACTAGCACCTAGCGCTTTCA |
| | | | TTATTGAAACGTCCCGTGTGGGAGGGGCGGGTCTGG |
| | | | GTGCGGCTGCCGCATGACTCGTGGTTCGGAGGCCCA |
| | | | CGTGGCCGGGGCGGGGACTCAGGCGCCTGGCAGCCG |
| | | | ACTGATTACGTAGCGGGCGGGGCCGGAAGTGCCGCT |
| | | | CCTTGGTGGGGGCTGTTCATGGCGGTTCCGGGGTCT |
| | | | CCAACATTTTTCCCGGTCTGTGGTCCTAAATCTGTC |
| | | | CAAAGCAGAGGCAGTGGAGCTTGAGGTTCTTGCTGG |
| | | | TGTGAAATGACTGAGTACAAACTGGTGGTGGTTGGA |
| | | | GCAGGTGGTGTTGGGAAAAGCGCACTGACAATCCAG |
| | | | CTAATCCAGAACCACTTTGTAGATGAATATGATCCC |
| | | | ACCATAGAGGATTCTTACAGAAAACAAGTGGTTATA |
| | | | GATGGTGAAACCTGTTTGTTGGACATACTGGATACA |
| | | | GCTGGACAAGAAGAGTACAGTGCCATGAGAGACCAA |
| | | | TACATGAGGACAGGCGAAGGCTTCCTCTGTGTATTT |
| | | | GCCATCAATAATAGCAAGTCATTTGCGGATATTAAC |
| | | | CTCTACAGGGAGCAGATTAAGCGAGTAAAAGACTCG |
| | | | GATGATGTACCTATGGTGCTAGTGGGAAACAAGTGT |
| | | | GATTTGCCAACAAGGACAGTTGATACAAAACAAGCC |
| | | | CACGAACTGGCCAAGAGTTACGGGATTCCATTCATT |
| | | | GAAACCTCAGCCAAGACCAGACAGGGTGTTGAAGAT |
| | | | GCTTTTTACACACTGGTAAGAGAAATACGCCAGTAC |
| | | | CGAATGAAAAAACTCAACAGCAGTGATGATGGGACT |
| | | | CAGGGTTGTATGGGATTGCCATGTGTGGTGATGTAA |
| | | | CAAGATACTTTTAAAGTTTTGTCAGAAAAGAGCCAC |
| | | | TTTCAAGCTGCACTGACACCCTGGTCCTGACTTCCT |
| | | | GGAGGAGAAGTATTCCTGTTGCTGTCTTCAGTCTCA |
| | | | CAGAGAAGCTCCTGCTACTTCCCCAGCTCTCAGTAG |
| | | | TTTAGTACAATAATCTCTATTTGAGAAGTTCTCAGA |
| | | | ATAACTACCTCCTCACTTGGCTGTCTGACCAGAGAA |
| | | | TGCACCTCTTGTTACTCCCTGTTATTTTTCTGCCCT |
| | | | GGGTTCTTCCACAGCACAAACACACCTCAACACACC |
| | | | TCTGCCACCCCAGGTTTTTCATCTGAAAAGCAGTTC |
| | | | ATGTCTGAAACAGAGAACCAAACCGCAAACGTGAAA |
| | | | TTCTATTGAAAACAGTGTCTTGAGCTCTAAAGTAGC |
| | | | AACTGCTGGTGATTTTTTTTTTCTTTTTACTGTTGA |
| | | | ACTTAGAACTATGCCTAATTTTTGGAGAAATGTCAT |
| | | | AAATTACTGTTTTGCCAAGAATATAGTTATTATTGC |
| | | | TGTTTGGTTTGTTTATAATGTTATCGGCTCTATTCT |
| | | | CTAAACTGGCATCTGCTCTAGATTCATAAATACAAA |
| | | | AATGAATACTGAATTTTGAGTCTATCCTAGTCTTCA |
| | | | CAACTTTGACGTAATTAAATCCAACTTTTCACAGTG |
| | | | AAGTGCCTTTTTCCTAGAAGTGGTTTGTAGACTCCT |
| | | | TTATAATATTTCAGTGGAATAGATGTCTCAAAAATC |
| | | | CTTATGCATGAAATGAATGTCTGAGATACGTCTGTC |
| | | | ACTTATCTACCATTGAAGGAAAGCTATATCTATTTG |
| | | | AGAGCAGATGCCATTTTGTACATGTATGAAATTGGT |
| | | | TTTCCAGAGGCCTGTTTTGGGGCTTTCCCAGGAGAA |
| | | | AGATGAAACTGAAAGCATATGAATAATTTCACTTAA |
| | | | TAATTTTTACCTAATCTCCACTTTTTTCATAGGTTA |
| | | | CTACCTATACAATGTATGTAATTTGTTTCCCCTAGC |
| | | | TTACTGATAAACCTAATATTCAATGAACTTCCATTT |
| | | | GTATTCAAATTTGTGTCATACCAGAAAGCTCTACAT |
| | | | TTGCAGATGTTCAAATATTGTAAAACTTTGGTGCAT |
| | | | TGTTATTTAATAGCTGTGATCAGGATTTTCAAACCT |
| | | | CAAATATAGTATATTAACAAATT |
| S000072 | F39 | 166 | TTGGAGCTGCCGCCGCCGGGACTCCCGTCCCAGCAG |
| | | | GACATGGATTTGATTGACATACTTTGGAGGCAAGAT |
| | | | ATAGATCTTGGAGTAAGTCGAGAAGTATTTGACTTC |
| | | | AGTCAGCGACGGAAAGAGTATGAGCTGGAAAAACAG |
| | | | AAAAAACTTGAAAAGGAAAGACAAGAACAACTCCAA |
| | | | AAGGAGCAAGAGAAAGCCTTTTTCACTCAGTTACAA |
| | | | CTAGATGAAGAGACAGGTGAATTTCTCCCAATTCAG |
| | | | CCAGCCCAGCACACCCAGTCAGAAACCAGTGGATCT |
| | | | GCCAACTACTCCCAGGTTGCCCACATTCCCAAATCA |
| | | | GATGCTTTGTACTTTGATGACTGCATGCAGCTTTTG |
| | | | GCGCAGACATTCCCGTTTGTAGATGACAATGAGGTT |
| | | | TCTTCGGCTACGTTTCAGTCACTTGTTCCTGATATT |
| | | | CCCGGTCACATCGAGAGCCCAGTCTTCATTGCTACT |
| | | | AATCAGGCTCAGTCACCTGAAACTTCTGTTGCTCAG |
| | | | GTAGCCCCTGTTGATTTAGACGGTATGCAACAGGAC |
| | | | ATTGAGCAAGTTTGGGAGGAGCTATTATCCATTCCT |
| | | | GAGTTACAGTGTCTTAATATTGAAAATGACAAGCTG |
| | | | GTTGAGACTACCATGGTTCCAAGTCCAGAAGCCAAA |
| | | | CTGACAGAAGTTGACAATTATCATTTTTACTCATCT |
| | | | ATACCCTCAATGGAAAAAGAAGTAGGTAACTGTAGT |
| | | | CCACATTTTCTTAATGCTTTTGAGGATTCCTTCAGC |
| | | | AGCATCCTCTCCACAGAAGACCCCAACCAGTTGACA |
| | | | GTGAACTCATTAAATTCAGATGCCACAGTCAACACA |
| | | | GATTTTGGTGATGAATTTTATTCTGCTTTCATAGCT |
| | | | GAGCCCAGTATCAGCAACAGCATGCCCTCACCTGCT |
| | | | ACTTTAAGCCATTCACTCTCTGAACTTCTAAATGGG |
| | | | CCCATTGATGTTTCTGATCTATCACTTTGCAAAGCT |
| | | | TTCAACCAAAACCACCCTGAAAGCACAGCAGAATTC |
| | | | AATGATTCTGACTCCGGCATTTCACTAAACACAAGT |
| | | | CCCAGTGTGGCATCACCAGAACACTCAGTGGAATCT |
| | | | TCCAGCTATGGAGACACACTACTTGGCCTCAGTGAT |
| | | | TCTGAAGTGGAAGAGCTAGATAGTGCCCCTGGAAGT |
| | | | GTCAAACAGAATGGTCCTAAAACACCAGTACATTCT |
| | | | TCTGGGGATATGGTACAACCCTTGTCACCATCTCAG |
| | | | GGGCAGAGCACTCACGTGCATGATGCCCAATGTGAG |
| | | | AACACACCAGAGAAAGAATTGCCTGTAAGTCCTGGT |
| | | | CATCGGAAAACCCCATTCACAAAAGACAAACATTCA |
| | | | AGCCGCTTGGAGGCTCATCTCACAAGAGATGAACTT |
| | | | AGGGCAAAAGCTCTCCATATCCCATTCCCTGTAGAA |
| | | | AAAATCATTAACCTCCCTGTTGTTGACTTCAACGAA |
| | | | ATGATGTCCAAAGAGCAGTTCAATGAAGCTCAACTT |
| | | | GCATTAATTCGGGATATACGTAGGAGGGGTAAGAAT |
| | | | AAAGTGGCTGCTCAGAATTGCAGAAAAAGAAAACTG |
| | | | GAAAATATAGTAGAACTAGAGCAAGATTTAGATCAT |
| | | | TTGAAAGATGAAAAAGAAAAATTGCTCAAAGAAAAA |
| | | | GGAGAAAATGACAAAAGCCTTCACCTAGTGAAAAAA |
| | | | CAACTCAGCACCTTATATCTCGAAGTTTTCAGCATG |
| | | | CTACGTGATGAAGATGGAAAACCTTATTCTCCTAGT |
| | | | GAATACTCCCTGCAGCAAACAAGAGATGGCAATGTT |
| | | | TTCCTTGTTCCCAAAAGTAAGAAGCCAGATGTTAAG |
| | | | AAAAACTAGATTTAGGAGGATTTGACCTTTTCTGAG |
| | | | CTAGTTTTTTTGTACTATTATACTAAAAGCTCCTAC |
| | | | TGTGATGTGAAATGCTCATACTTTATAAGTAATTCT |
| | | | ATGCAAAATCATAGCCAAAACTAGTATAGAAAATAA |
| | | | TACGAAACTTTAAAAAGCATTGGAGTGTCAGTATGT |
| | | | TGAATCAGTAGTTTCACTTTAACTGTAAACAATTTC |
| | | | TTAGGACACCATTTGGGCTAGTTTCTGTGTAAGTGT |
| | | | AAATACTACAAAAACTTATTTATACTGTTCTTATGT |
| | | | CATTTGTTATATTCATAGATTTATATGATGATATGA |
| | | | CATCTGGCTAAAAAGAAATTATTGCAAAACTAACCA |
| | | | CGATGTACTTTTTTATAAATACTGTATGGACAAAAA |
| | | | ATGGCATTTTTTATAATTAAATTGTTTAGCTCTGGC |
| | | | AAAAAAAAAAAATTTTTTAAGAGCTGGTACTAATAA |
| | | | AGGATTATTATGACTGTT |
| S000083 | F40 | 167 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAAGACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCCACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTGCTTTTTAAAGTTGATTT |
| S000087 | F41 | 168 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTGCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGAGTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCGACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCCTAATTTTTTTTAT |
| | | | TTAAGTACATTTTGCTTTTTAAAGTTGATTT |
| S000090 | F42 | 169 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACGTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCCACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTGCTTTTTAAAGTTGATTT |
| S000098 | F43 | 170 | TCGGAGACCACATTGCCTCGTGTCCAACTATCCATT |
| | | | ACCAAGAAGAAATCTATTCGTTTGAGCCTGAGACAC |
| | | | TCTTTGAGGTAAAAAATTAGAATGAAAGAACCTTTG |
| | | | GATGGTGAATGTGGCAAAGCAGTGGTACCACAGCAG |
| | | | GAGCTTCTGGACAAAATTAAAGAAGAACCAGACAAT |
| | | | GCTCAAGAGTATGGATGTGTCCAACAGCCAAAAACT |
| | | | CAAGAAAGTAAATTGAAAATTGGTGGTGTGTCTTCA |
| | | | GTTAATGAGAGACCTATTGCCCAGCAGTTGAACCCA |
| | | | GGCTTTCAGCTTTCTTTTGCATCATCTGGCCCAAGT |
| | | | GTGTTGCTTCCTTCAGTTCCAGCTGTTGCTATTAAG |
| | | | GTTTTTTGTTCTGGTTGTAAAAAAATGCTTTATAAG |
| | | | GGCCAAACTGCATATCATAAGACAGGATCTACTCAG |
| | | | CTCTTCTGCTCCACACGATGCATCACCAGACATTCT |
| | | | TCACCTGCCTGCCTGCCACCTCCTCCCAAGAAAACC |
| | | | TGCACAAACTGCTCGAAAGACATTTTAAATCCTAAG |
| | | | GATGTGATCACAACTCGCTTTGAGAATTCCTATCCT |
| | | | AGCAAAGATTTCTGCAGCCAATCATGCTTGTCATCT |
| | | | TATGAGCTAAAGAAAAAACCTGTTGTTACCATATAT |
| | | | ACCAAAAGCATTTCAACTAAGTGCAGTATGTGTCAG |
| | | | AAGAATGCTGATACTCGATTTGAAGTTAAATATCAA |
| | | | AATGTGGTACATGGTCTTTGTAGTGATGCCTGTTTT |
| | | | TCAAAATTTCACTCTACAAACAACCTCACCATGAAC |
| | | | TGTTGTGAGAACTGTGGGAGCTATTGCTATAGTAGC |
| | | | TCTGGTCCTTGCCAATCCCAGAAGGTTTTTAGTTCA |
| | | | ACAAGTGTCACGGCATACAAGCAGAATTCTGCCCAA |
| | | | ATTCCTCCATATGCCCTGGGGAAGTCATTGAGGCCC |
| | | | TCAGCTGAAATGATTGAGACTACAAATGATTCAGGA |
| | | | AAAACAGAGCTTTTCTGCTCTATTAATTGCTTATCT |
| | | | GCTTACAGAGTTAAGACTGTTACTTCTTCAGGTGTC |
| | | | CAGGTTTCATGTCATAGTTGTAAAACCTCAGCAATC |
| | | | CCTCAGTATCACCTAGCCATGTCAAATGGAACTATA |
| | | | TACAGCTTCTGCAGCTCCAGTTGTGTGGTTGCTTTC |
| | | | CAGAATGTATTTAGCAAGCCAAAAGGAACAAACTCT |
| | | | TCGGCGGTGCCCCTGTCTCAGGGCCAAGTGGTTGTA |
| | | | AGCCCGCCCTCCTCCAGGTCAGCAGTGTCAATAGGA |
| | | | GGAGGTAACACCTCTGCCGTTTCCCCCAGCTCCATC |
| | | | CGTGGCTCTGCTGCAGCCAGCCTCCAACCTCTTGGT |
| | | | GAACAATCCCAGCAAGTTGCTTTAACCCATACAGTT |
| | | | GTTAAACTCAAGTGTCAGCACTGTAACCATCTATTT |
| | | | GCCACAAAACCAGAACTTCTTTTTTACAAGGGTAAA |
| | | | ATGTTTCTGTTTTGTGGCAAGAATTGCTCTGATGAA |
| | | | TACAAGAAGAAAAATAAAGTTGTGGCAATGTGTGAC |
| | | | TACTGTAAACTGCAGAAAATTATAAAGGAGACTGTG |
| | | | CGATTCTCAGGGGTTGATAAGCCATTCTGTAGTGAA |
| | | | GTTTGCAAATTCCTCTCTGCCCGTGACTTTGGAGAA |
| | | | CGATGGGGAAACTACTGTAAGATGTGCAGCTACTGT |
| | | | TCACAGACATCCCCAAATTTGGTAGAAAATCGATTG |
| | | | GAGGGCAAGTTAGAAGAGTTTTGTTGTGAAGATTGT |
| | | | ATGTCCAAATTTACAGTTCTGTTTTATCAGATGGCC |
| | | | AAGTGTGATGGTTGTAAACGACAGGGTAAACTAAGC |
| | | | GAGTCCATAAAGTGGCGAGGCAACATTAAACATTTC |
| | | | TGTAACCTATTTTGTGTCTTGGAGTTTTGTCATCAG |
| | | | CAAATTATGAATGACTGTCTTCCACAAAATAAAGTA |
| | | | AATATTTCTAAAGCAAAAACTGCTGTGACGGAGCTC |
| | | | CCTTCTGCAAGGACAGATACAACACCAGTTATAACC |
| | | | AGTGTGATGTCATTGGCAAAAATACCTGCTACCTTA |
| | | | TCTACAGGGAACACTAACAGTGTTTTAAAAGGTGCA |
| | | | GTTACTAAAGAGGCAGCAAAGATCATTCAAGATGAA |
| | | | AGTACACAGGAAGATGCTATGAAATTTCCATCTTCC |
| | | | CAATCTTCCCAGCCTTCCAGGCTTTTAAAGAACAAA |
| | | | GGCATATCATGCAAACCCGTCACACAGACCAAGGCC |
| | | | ACTTCTTGCAAACCACATACACAGCACAAAGAATGT |
| | | | CAGACAGAATGCCCTGTTCGTGCAGTTTGCTGAGGT |
| | | | GTTCCCGCTGAAGTATTTGGCTACCAGCCAGATCCC |
| | | | CTGAACTACCAAATAGCTGTGGGCTTTCTGGAACTG |
| | | | CTGGCTGGGTTGCTGCTGGTCATGGGCCCACCGATG |
| | | | CTGCAAGAGATCAGTAACT |
| S000104 | F44 | 171 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCCACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAAAGCACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTGCTTTTTAAAGTTGATTT |
| S000106 | F45 | 172 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTGCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCCACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTGCTTTTTAAAGTTGATTT |
| S000107 | F46 | 173 | GGGGGCAGAGGGAGCGAGCGGGGGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCGCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCGCGCAG |
| | | | GGCAGCCCCGAGCCCCTGGTGCTCCATGAGGAGACA |
| | | | CCGCCCACCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | TCTAACAGAAATGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | GAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTGCTTTTTAAAGTTGATTT |
| S000114 | F47 | 174 | GCATCCCGGCATCTGCACGTGGTTATGCTGCCGGAG |
| | | | TTTGGGCCGCCACTGTAGGAAAAGTAACTTCAGCTG |
| | | | CAGCCCCAAAGCGAGTGAGCCGAGCCGGAGCCATGG |
| | | | AGGGCCAGAGCGTGGAGGAGCTGCTCGCAAAGGCAG |
| | | | AGCAGGACGAGGCAGAGAAGTTGCAACGCATCACGG |
| | | | TGCACAAGGAGCTGGAGCTGCAGTTTGACCTGGGCA |
| | | | ACCTGCTGGCGTCGGACCGGAACCCCCCGACCGGGC |
| | | | TGCGGTGCGCCGGACCCACGCCGGAGGCCGAGCTAC |
| | | | AGGCCCTGGCGCGGGACAACACGCAACTGCTCATCA |
| | | | ACCAGCTGTGGCAGCTGCCCACGGAGCGCGTGGAAG |
| | | | AGGCGATAGTGGCGCGGCTGCCGGAGCCCACCACAC |
| | | | GCCTGCCGCGAGAGAAGCCTCTGCCCCGACCGCGGC |
| | | | CACTTACACGCTGGCAGCAGTTCGCGCGCCTCAAGG |
| | | | GCATCCGTCCCAAGAAGAAGACCAACCTGGTGTGGG |
| | | | ACGAGGTGAGTGGCCAGTGGCGGCGGCGCTGGGGCT |
| | | | ACCAGCGCGCCCGGGACGACACCAAAGAATGGCTGA |
| | | | TTGAGGTGCCCGGCAATGCCGACCCCTTGGAGGACC |
| | | | AGTTCGCCAAGCGGATTCAGGCCAAGAAGGAAAGGG |
| | | | TGGCCAAGAACGAGCTGAACCGGCTGCGTAACCTGG |
| | | | CCCGCCGCGCACAAGATGCAGCTGCCCAGCGCGGCG |
| | | | GCTTGCACCCTACCGGACACCAGAGTAAGGAGGAGC |
| | | | TGGGCCGCGCCATGCAAGTGGCCAAGGTCTCCACCG |
| | | | CCTCTGTGGGGCGCTTTCAGGAGCGCCTCCCCAAGG |
| | | | AGAAGGTGCCCCGGGGCTCCGGCAAGAAAAGGAAGT |
| | | | TTCAACCCCTTTTCGGGGACTTTGCAGCCGAGAAAA |
| | | | AGAACCAGTTGGAGCTGCTTCGTGTCATGAACAGCA |
| | | | AGAAGCCTCAGCTGGATGTGACTAGGGCCACCAATA |
| | | | AGCAGATGAGGGAGGAGGACCAGGAGGAGGCCGCCA |
| | | | AGAGGAGGAAAATGAGCCAGAAGGGCAAGAGAAAGG |
| | | | GAGGCCGGCAGGGGCCTGGGGGCAAGAGGAAAGGGG |
| | | | GCCCGCCCAGCCAGGGAGGGAAGAGGAAAGGGGGCT |
| | | | TGGGAGGCAAGATGAATTCTGGGCCGCCTGGCTTGG |
| | | | GTGGCAAGAGAAPAGGAGGACAGCGCCCAGGAGGAA |
| | | | AGAGGAGGAAGTAATAGTTTCTAACTGTCGGACCCG |
| | | | TCTGTAAACCAAGGACTATGAATACTAAATGTTAAG |
| | | | TTCTAGGCAATTATACGGGGACTCAGAAGGACCTGG |
| | | | CCGCTGCCTTCATTGAGTTTAAAGGGACAGGATTGC |
| | | | CGTTCCGTCAAGAAAGTATGTAAGTGTTGGACTGCA |
| | | | CAAATTAATGTTTTTCCCACAACCGAGACTTTGGAG |
| | | | ATTAAGAACTTATTTGAGGATTTAAGAATTAGGGAA |
| | | | ATAATTTGGTGGAAACCGGGAATGAGTTCTATTCTT |
| | | | AAACAGCCTTTTTTTTTCTTTTTAATGTTGGATATA |
| | | | CGGCGAGGTAGAGTTGGCCATATTTCAGAGACTTAG |
| | | | ATTGACGTATATGTTTCTGCATTATTTTTACAACAA |
| | | | GTTTGTGTATCAGAGCGGGAGTTCGGGGGAGGGAAA |
| | | | GAAAACAAACAGTTTCAGAATTGAATAGGCAAGTGA |
| | | | CTGTTTTAAAGATTAAGTAATAAAGATGTCTTATCT |
| | | | AGTG |
| S000116 | F48 | 175 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTAGACGCTGGAGTTTTTTCGGGAAG |
| | | | TGGGAAAGCAGCCTCCCGCGACGATGCCCCTCAACG |
| | | | TTAGCTTCACCAACAGGAACTATGACCTCGACTACG |
| | | | ACTCGGTGCAGCCGTATTTCTACTGCGACGAGGAGG |
| | | | AGAACTTCTACCAGCAGCAGCAGCAGAGCGAGCTGC |
| | | | AGCCCCCGGCGCCCAGCGAGGATATCTGGAAGAAAT |
| | | | TCGAGCTGCTGCCCACCCCGCCCCTGTCCCCTAGCC |
| | | | GCCGCTCCGGGCTCTGCTCGCCCTCCTACGTTGCGG |
| | | | TCACACCCTTCTCCCTTCGGGGAGACAACGACGGCG |
| | | | GTGGCGGGAGCTTCTCCACGGCCGACCAGCTGGAGA |
| | | | TGGTGACCGAGCTGCTGGGAGGAGACATGGTGAACC |
| | | | AGAGTTTCATCTGCGACCCGGACGACGAGACCTTCA |
| | | | TCAAAAACATCATCATCCAGGACTGTATGTGGAGCG |
| | | | GCTTCTCGGCCGCCGCCAAGCTCGTCTCAGAGAAGC |
| | | | TGGCCTCCTACCAGGCTGCGCGCAAAGACAGCGGCA |
| | | | GCCCGAACCCCGCCCGCGGCCACAGCGTCTGCTCCA |
| | | | CCTCCAGCTTGTACCTGCAGGATCTGAGCGCCGCCG |
| | | | CCTCAGAGTGCATCGACCCCTCGGTGGTCTTCCCCT |
| | | | ACCCTCTCAACGACAGCAGCTCGCCCAAGTCCTGCG |
| | | | CCTCGCAAGACTCCAGCGCCTTCTCTCCGTCCTCGG |
| | | | ATTCTCTGCTCTCCTCGACGGAGTCCTCCCCGCAGG |
| | | | GCAGCCCCGAGCCCCTGGTGCTCCATGAGGAGACAC |
| | | | CGCCCACCACCAGCAGCGACTCTGAGGAGGAACAAG |
| | | | AAGATGAGGAAGAAATCGATGTTGTTTCTGTGGAAA |
| | | | AGAGGCAGGCTCCTGGCAAAAGGTCAGAGTCTGGAT |
| | | | CACCTTCTGCTGGAGGCCACAGCAAACCTCCTCACA |
| | | | GCCCACTGGTCCTCAAGAGGTGCCACGTCTCCACAC |
| | | | ATCAGCACAACTACGCAGCGCCTCCCTCCACTCGGA |
| | | | AGGACTATCCTGCTGCCAAGAGGGTGAAGTTGGACA |
| | | | GTGTCAGAGTCCTGAGACAGATCAGCAACAACCGAA |
| | | | AATGCACCAGCCCCAGGTCCTCGGACACCGAGGAGA |
| | | | ATGTCAAGAGGCGAACACACAACGTCTTGGAGCGCC |
| | | | AGAGGAGGAACGAGCTAAAACGGAGCTTTTTTGCCC |
| | | | TGCGTGACCAGATCCCGGAGTTGGAAAACAATGAAA |
| | | | AGGCCCCCAAGGTAGTTATCCTTAAAAAAGCCACAG |
| | | | CATACATCCTGTCCGTCCAAGCAGAGGAGCAAAAGC |
| | | | TCATTTCTGAAGAGGACTTGTTGCGGAAACGACGAG |
| | | | AACAGTTGAAACACAAACTTGAACAGCTACGGAACT |
| | | | CTTGTGCGTAAGGAAAAGTAAGGAAAACGATTCCTT |
| | | | CTAACAGAAATGTCCTGAGCAATCACCTATGAACTT |
| | | | GTTTCAAATGCATGATCAAATGCAACCTCACAACCT |
| | | | TGGCTGAGTCTTGAGACTGAAAGATTTAGCCATAAT |
| | | | GTAAACTGCCTCAAATTGGACTTTGGGCATAAAAGA |
| | | | ACTTTTTATGCTTACCATCTTTTTTTTTTCTTTAAC |
| | | | AGATTTGTATTTAAGAATTGTTTTTAAAAAATTTTA |
| | | | AGATTTACACAATGTTTCTCTGTAAATATTGCCATT |
| | | | AAATGTAAATAACTTTAATAAAAACGTTTATAGCAG |
| | | | TTACACGAATTTCAATCCTAGTATATAGTACCTAGT |
| | | | ATTATAGTGTACTATAAACCCTAATTTTTTTTATTT |
| | | | AAGTACATTTTGCTTTTTAAAGTTGATTT |
| S000118 | F49 | 176 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCAGCGGTCCGCAAGCCTTGCCGCATCCACGAAACT |
| | | | TTGCCCATACTGCGGGCGTACACTTTGCACTTGAAC |
| | | | TTACAACACCTGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGTC |
| | | | CTTGAAGCTCCTTAGACGCTGGAGTTTTTTCGGGAA |
| | | | GTGGGAAAGCAGCCTCCCGCGACGATGCCCCTCAAC |
| | | | GTTAGCTTCACCAACAGGAACTATGACCTCGACTAC |
| | | | GACTCGGTGCAGCCGTATTTCTACTGCGACGAGGAG |
| | | | GAGAACTTCTACCAGCAGCAGCAGCAGAGCGAGCTG |
| | | | CAGCCCCCGGCGCCCAGCGAGGATATCTGGAAGAAA |
| | | | TTCGAGCTGCTGCCCACCCCGCCCCTGTCCCCTAGC |
| | | | CGCCGCTCCGGGCTCTGCTCGCCCTCCTACGTTGCG |
| | | | GTCACACCCTTCTCCCTTCGGGGAGACAACGACGGC |
| | | | GGTGGCGGGAGCTTCTCCACGGCCGACCAGCTGGAG |
| | | | ATGGTGACCGAGCTGCTGGGAGGAGACATGGTGAAC |
| | | | CAGAGTTTCATCTGCGACCCGGACGACGAGACCTTC |
| | | | ATCAAAAACATCATCATCCAGGACTGTATGTGGAGC |
| | | | GGCTTCTCGGCCGCCGCCAAGCTCGTCTCAGAGAAG |
| | | | CTGGCCTCCTACCAGGCTGCGCGCAAAGACAGCGGC |
| | | | AGCCCGAACCCCGCCCGCGGCCACAGCGTCTGCTCC |
| | | | ACCTCCAGCTTGTACCTGCAGGATCTGAGCGCCGCC |
| | | | GCCTCAGAGTGCATCGACCCCTCGGTGGTCTTCCCC |
| | | | TACCCTCTCAACGACAGCAGCTCGCCCAAGTCCTGC |
| | | | GCCTCGCAAGACTCCAGCGCCTTCTCTCCGTCCTCG |
| | | | GATTCTCTGCTCTCCTCGACGGAGTCCTCCCCGCAG |
| | | | GGCAGCCCCGAGCCCCTGGTGCTCCATGAGGAGACA |
| | | | CCGCCCAQCACCAGCAGCGACTCTGAGGAGGAACAA |
| | | | GAAGATGAGGAAGAAATCGATGTTGTTTCTGTGGAA |
| | | | AAGAGGCAGGCTCCTGGCAAAAGGTCAGAGTCTGGA |
| | | | TCACCTTCTGCTGGAGGCCACAGCAAACCTCCTCAC |
| | | | AGCCCACTGGTCCTCAAGAGGTGCCACGTCTCCACA |
| | | | CATCAGCACAACTACGCAGCGCCTCCCTCCACTCGG |
| | | | AAGGACTATCCTGCTGCCAAGAGGGTCAAGTTGGAC |
| | | | AGTGTCAGAGTCCTGAGACAGATCAGCAACAACCGA |
| | | | AAATGCACCAGCCCCAGGTCCTCGGACACCGAGGAG |
| | | | AATGTCAAGAGGCGAACACACAACGTCTTGGAGCGC |
| | | | CAGAGGAGGAACGAGCTAAAACGGAGCTTTTTTGCC |
| | | | CTGCGTGACCAGATCCCGGAGTTGGAAAACAATGAA |
| | | | AAGGCCCCCAAGGTAGTTATCCTTAAAAAAGCCACA |
| | | | GCATACATCCTGTCCGTCCAAGCAGAGGAGCAAAAG |
| | | | CTCATTTCTGAAGAGGACTTGTTGCGGAAACGACGA |
| | | | GAACAGTTGAAACACAAACTTGAACAGCTACGGAAC |
| | | | TCTTGTGCGTAAGGAAAAGTAAGGAAAACGATTCCT |
| | | | CTAACAGAAATTGTCCTGAGCAATCACCTATGAACT |
| | | | TGTTTCAAATGCATGATCAAATGCAACCTCACAACC |
| | | | TTGGCTGAGTCTTGAGACTGAAAGATTTAGCCATAA |
| | | | TGTAAACTGCCTCAAATTGGACTTTGGGCATAAAAG |
| | | | AACTTTTTATGCTTACCATCTTTTTTTTTTCTTTAA |
| | | | CAGATTTGTATTTAAGAATTGTTTTTAAAAAATTTT |
| | | | AAGATTTACACAATGTTTCTCTGTAAATATTGCCAT |
| | | | TAAATGTAAATAACTTTAATAAAAACGTTTATAGCA |
| | | | GTTACACAGAATTTCAATCCTAGTATATAGTACCTA |
| | | | GTATTATAGGTACTATAAACCCTAATTTTTTTTATT |
| | | | TAAGTACATTTTTGCTTTTTAAAGTTGATTT |
| S000121 | F50 | 177 | GGGGGCAGAGGGAGCGAGCGGGCGGCCGCCTAGGGT |
| | | | GCAAGAGCCGGGCGAGCAGAGTTGCGCTGCGGGCGT |
| | | | CCTGGGAAGGGAGTTCCGGAGCCAACAGGGGGCTTC |
| | | | GCCTCTGGCCCAGCCCTTCCGGAGCCAACAGGGGAC |
| | | | TTCGCCTCTGGCCCAGCCCTCCCGCTGATCCCCCAG |
| | | | TCGCACTTGAACTTACAACACCCGAGCAAGGACGCG |
| | | | ACTCTCCCGACGCGGGCGTACACTTTGCACTTGAAC |
| | | | TTACAACACCCGAGCAAGGACGCGACTCTCCCGACG |
| | | | CGGGGAGACTATTCTGCCCATTTGGGGACACTTCCC |
| | | | CGCCGCTGCCAGGACCCGGTTCTCTGGAAGGCTGCC |
| | | | TTGAAGCTCCTTAGACGCTGGAGTTTTTTCGGGAAG |
| | | | TGGGAAAGCAGCCTCCCGCGACGATGCCCCTCAACG |
| | | | TTAGCTTCACCAACAGGAACTATGACCTCGACTACG |
| | | | ACTCGGTGCAGCCGTATTTCTACTGCGACGAGGAGG |
| | | | AGAACTTCTACCAGCAGCAGCAGCAGAGCGAGCTGC |
| | | | AGCCCCCGGCGCCCAGCGAGGATATCTGGAAGAAAT |
| | | | TCGAGCTGCTGCCCACCCCGCCCCTGTCCCCTAGCC |
| | | | GCCGCTCCGGGCTCTGCTCGCCCTCCTACGTTGCGG |
| | | | TCACACCCTTCTCCCTTCGGGGAGACAACGACGGCG |
| | | | GTGGCGGGAGCTTCTGCACGGCCGACCAGCTGGAGA |
| | | | TGGTGACCGAGCTGCTGGGAGGAGACATGGTGAACC |
| | | | AGAGTTTCATCTGCGACCCGGACGACGAGACCTTCA |
| | | | TCAAAAACATCATCATCCAGGACTGTATGTGGAGCG |
| | | | GCTTCTCGGCCGCCGCCAAGCTCGTCTCAGAGAAGC |
| | | | TGGCCTCCTACCAGGCTGCGCGCAAAGACAGCGGCA |
| | | | GCCCGAACCCCGCCCGCGGCCACAGCGTCTGCTCCA |
| | | | CCTCCAGCTTGTACCTGCAGGATCTGAGCGCCGCCG |
| | | | CCTCAGAGTGCATCGACCCCTCGGTGGTCTTCCCCT |
| | | | ACCCTCTCAACGACAGCAGCTCGCCCAAGTCCTGCG |
| | | | CCTCGCAAGACTCCAGCGCCTTCTCTCCGTCCTCGG |
| | | | ATTCTCTGCTCTCCTCGACGGAGTCCTCCCCGCAGG |
| | | | GCAGCCCCGAGCCCCTGGTGCTCCATGAGGAGACAC |
| | | | CGCCCACCACCAGCAGCGACTCTGAGGAGGAACAAG |
| | | | AAGATGAGGAAGAAATCGATGTTGTTTCTGTGGAAA |
| | | | AGAGGCAGGCTCCTGGCAAAAGGTCAGAGTCTGGAT |
| | | | CACCTTCTGCTGGAGGCCACAGCAAACCTCCTCACA |
| | | | GCCCACTGGTCCTCAAGAGGTGCCACGTCTCCACAC |
| | | | ATCAGCACAACTACGCAGCGCCTCCCTCCACTCGGA |
| | | | AGGACTATCCTGCTGCCAAGAGGGTCAAGTTGGACA |
| | | | GTGTCAGAGTCCTGAGACAGATCAGCAACAACCGAA |
| | | | AATGCACCAGCCCCAGGTCCTCGGACACCGAGGAGA |
| | | | ATGTCAAGAGGCGAACACACAACGTCTTGGAGCGCC |
| | | | AGAGGAGGAACGAGCTAAAACGGAGCTTTTTTGCCC |
| | | | TGCGTGACCAGATCCCGGAGTTGGAAAACAATGAAA |
| | | | AGGCCCCCAAGGTAGTTATCCTTAAAAAAGCCACAG |
| | | | CATACATCCTGTCCGTCCAAGCAGAGGAGCAAAAGC |
| | | | TCATTTCTGAAGAGGACTTGTTGCGGAAACGACGAG |
| | | | AACAGTTGAAACACAAACTTGAACAGCTACGGAACT |
| | | | CTTGTGCGTAAGGAAAAGTAAGGAAAACGATTCCTT |
| | | | CTAACAGAAATGTCCTGAGCAATCACCTATGAACTT |
| | | | GTTTCAAATGCATGATCAAATGCAACCTCACAACCT |
| | | | TGGCTGAGTCTTGAGACTGAAAGATTTAGCCATAAT |
| | | | GTAAACTGCCTCAAATTGGACTTTGGGCATAAAAGA |
| | | | ACTTTTTATGCTTACCATCTTTTTTTTTTCTTTAAC |
| | | | AGATTTGTATTTAAGAATTGTTTTTAAAAAATTTTA |
| | | | AGATTTACACAATGTTTCTCTGTAAATATTGCCATT |
| | | | AAATGTAAATAACTTTAATAAAAACGTTTATAGCAG |
| | | | TTACACAGAATTTCAATCCTAGTATATAGTACCTAG |
| | | | TATTATAGGTACTATAAACCCTAATTTTTTTTATTT |
| | | | AAGTACATTTTGCTTTTTAAAGTTGATTT |

A Pik3r1 nucleic acid sequence of the invention is depicted in Table 4 as SEQ ID NO. 178. The nucleic acid sequence shown is from mouse. SEQ ID NO: 179 (Table 5) depicts the amino acid sequence encoded by SEQ ID NO: 178. SEQ ID NO: 178 and SEQ ID NO: 179 are from mouse.

TABLE 4

SEQ.
ID

| | |
|---|---|
| NO. | MOUSE SEQUENCE |

| | |
|---|---|
| 178 | GGCACGAGCC GAGTTGGAGG AAGCAGCGGC AGCGGCAGCG |
| | GCAGCGGTAG CGGTGAGGAC GGCTGTGCAG CCAAGGAACC |
| | GGGACAGCGA AGCGACGGCA GGTCGCAGCT GGATCGCAGG |
| | AGCCTGGGAG CTGGGAGCTT GAGAGGCCGC TGAAGCCCAG |
| | GCTGGGCAGA GGAAGGAAGC GAGCCGACCC GGAGGTGAAG |
| | CTGAGAGTGG AGCGTGGCAG TAAAATCAGA CGACAGATGG |
| | ACAGTGTGAC AGGAACGTGA GAGAGGATTG GGCCTCGCTG |
| | CGAGAGTCAG CCTGGAGTCA AGGTGTTGAC AAGTTGCTGA |
| | GAAGGACACG TGGGAGGACG GTGGCGCGCG GAGGGAGAGC |
| | CCTGTCTTCA GTCACCCCGT TGATGGAGGA CAGATGGACA |
| | GCAGCCGGAC GGCCAGTCAC CTCTCTTAAA CCTTTGGATA |
| | GTGGTCCTTT GTGCTCTGCT GGACACCTGT TGGGGATTTT |
| | AGCCCATTCT CTGAACTCAC TTTCTCTTAA AACGTAAACT |
| | CGGACGGCAG TGTGCGAGCC AGCTCCTCTG TGGCAGGGCA |
| | CTAGAGCTGC AGACATGAGT GCAGAGGGCT ACCAGTACAG |
| | AGCACTGTAC GACTACAAGA AGGAGCGAGA GGAAGACATT |
| | GACCTACACC TGGGGGACAT ACTGACTGTG AATAAAGGCT |
| | CCTTAGTGGC ACTTGGATTC AGTGATGGCC AGGAAGCCCG |
| | GCCTGAAGAT ATTGGCTGGT TAAATGGCTA CAATGAAACC |
| | ACTGGGGAGA GGGGAGACTT TCCAGGAACT TACGTTGAAT |
| | ACATTGGAAG GAAAAGAATT TCACCCCCTA CTCCCAAGCC |
| | TCGGCCCCCT CGACCGCTTC CTGTTGCTCC GGGTTCTTCA |
| | AAAACTGAAG CTGACACGGA GCAGCAAGCG TTGCCCCTTC |
| | CTGACCTGGC CGAGCAGTTT GCCCCTCCTG ATGTTGCCCC |
| | GCCTCTCCTT ATAAAGCTCC TGGAAGCCAT TGAGAAGAAA |
| | GGACTGGAAT GTTCGACTCT ATACAGAACA CAAAGCTCCA |
| | GCAACCCTGC AGAATTACGA CAGCTTCTTG ATTGTGATGC |
| | CGCGTCAGTG GACTTGGAGA TGATCGACGT ACACGTCTTA |
| | GCAGATGCTT TCAAACGCTA TCTCGCCGAC TTACCAAATC |
| | CTGTCATTCC TGTAGCTGTT TACAATGAGA TGATGTCTTT |
| | AGCCCAAGAA CTACAGAGCC CTGAAGACTG CATCCAGCTG |
| | TTGAAGAAGC TCATTAGATT GCCTAATATA CCTCATCAGT |
| | GTTGGCTTAC GCTTCAGTAT TTGCTCAAGC ATTTTTTCAA |
| | GCTCTCTCAA GCCTCCAGCA AAAACCTTTT GAATGCAAGA |
| | GTCCTCTCTG AGATTTTCAG CCCCGTGCTT TTCAGATTTC |
| | CAGCCGCCAG CTCTGATAAT ACTGAACACC TCATAAAAGC |
| | GATAGAGATT TTAATCTCAA CGGAATGGAA TGAGAGACAG |
| | CCAGCACCAG CACTGCCCCC CAAACCACCC AAGCCCACTA |
| | CTGTAGCCAA CAACAGCATG AACAACAATA TGTCCTTGCA |
| | GGATGCTGAA TGGTACTGGG GAGACATCTG AAGGGAAGAA |
| | GTGAATGAAA AACTCCGAGA CACTGCTGAT GGGACCTTTT |
| | TGGTACGAGA CGCATCTACT AAAATGCACG GCGATTACAC |
| | TCTTACACCT AGGAAAGGAG GAAATAACAA ATTAATCAAA |
| | ATCTTTCACC GTGATGGAAA ATATGGCTTC TCTGATCCAT |
| | TAACCTTCAA CTCTGTGGTT GAGTTAATAA ACCACTACCG |
| | GAATGAGTCT TTAGCTCAGT ACAACCCCAA GCTGGATGTG |
| | AAGTTGCTCT ACCCAGTGTC CAAATACCAG CAGGATCAAG |
| | TTGTCAAAGA AGATAATATT GAAGCTGTAG GGAAAAAATT |
| | ACATGAATAT AATACTCAAT TTCAAGAAAA AAGTCGGGAA |
| | TATGATAGAT TATATGAGGA GTACACCCGT ACTTCCCAGG |
| | AAATCCAAAT GAAAAGAACG GCTATCGAAG CATTTAATGA |
| | AACCATAAAA ATATTTGAAG AACAATGCCA AACCCAGGAG |
| | CGGTACAGCA AAGAATACAT AGAGAAGTTT AAACGCGAAG |
| | GCAACGAGAA AGAAATTCAA AGGATTATGC ATAACCATGA |
| | TAAGCTGAAG TCGCGTATCA GTGAGATCAT TGACAGTAGG |
| | AGGAGGTTGG AAGAAGACTT GAAGAAGCAG GCAGCTGAGT |
| | ACCGAGAGAT CGACAAACGC ATGAACAGTA TTAAGCCGGA |
| | CCTCATCCAG TTGAGAAAGA CAAGAGACCA ATACTTGATG |
| | TGGCTGACGC AGAAAGGTGT GCGGCAGAAG AAGCTGAACG |
| | AGTGGCTGGG GAATGAAAAT ACCGAAGATC AATACTCCCT |
| | GGTAGAAGAT GATGAGGATT TGCCCCACCA TGACGAGAAG |
| | ACGTGGAATG TCGGGAGCAG CAACCGAAAC AAAGCGGAGA |
| | ACCTATTGCG AGGGAAGCGA GACGGCACTT TCCTTGTCCG |
| | GGAGAGCAGT AAGCAGGGCT GCTATGCCTG CTCCGTAGTG |
| | GTAGACGGCG AAGTCAAGCA TTGCGTCATT AACAAGACTG |
| | CCACCGGCTA TGGCTTTGCC GAGCCCTACA ACCTGTACAG |
| | CTCCCTGAAG GAGCTGGTGC TACATTATCA ACACACCTCC |
| | CTCGTGCAGC ACAATGACTC CCTCAATGTC ACACTAGCAT |
| | ACCCAGTATA TGCACAACAG AGGCGATGAA GCGCTGCCCT |
| | CGGATCCAGT TCCTCACCTT CAAGCCACCC AAGGCCTCTG |
| | AGAAGCAAAG GGCTCCTCTC CAGCCCGACC TGTGAACTGA |
| | GCTGCAGAAA TGAAGCCGGC TGTCTGCACA TGGGACTAGA |
| | GCTTTCTTGG ACAAAAAGAA GTCGGGGAAG ACACGCAGCC |
| | TCGGACTGTT GGATGACCAG ACGTTTCTAA CCTTATCCTC |
| | TTTCTTTCTT TCTTTCTTTC TTTCTTTCTT TCTTTCTTTC |
| | TTTCTTTCTT TCTTTCTTTC TTTCTAATTT AAAGCCACAA |
| | CACACAACCA ACACACAGAG AGAAAGAAAT GCAAAAATCT |
| | CTCCGTGCAG GGACAAAGAG GCCTTTAACC ATGGTGCTTG |
| | TTAACGCTTT CTGAAGCTTT ACCAGCTACA AGTTGGGACT |
| | TTGGAGACCA GAAGGTAGAC AGGGCCGAAG AGCCTGCGCC |
| | TGGGGCCGCT TGGTCCAGCC TGGTGTAGCC TGGGTGTCGC |
| | TGGGTGTGGT GAACCCAGAC ACATCACACT GTGGATTATT |
| | TCCTTTTTAA AAGAGCGAAT GATATGTATC AGAGAGCCGC |
| | GTCTGCTCAC GCAGGACACT TTGAGAGAAC ATTGATGCAG |
| | TCTGTTCGGA GGAAAAATGA AACACCAGAA AACGTTTTTG |
| | TTTAAACTTA TCAAGTCAGC AACCAACAAC CCACCAACAG |
| | AAAAAAAAAA AAAA |

TABLE 5
MOUSE SEQUENCE

| | |
|---|---|
| 179 | MSAEGYQYRALYDYKKEREEDIDLHLGDILTVNKGSLVALGFSDGQE |
| | ARPEDIGWLNGYNETTGERGDFPGTYVEYIGRKRISPPTPKPRPPRP |
| | LPVAPGSSKTEADTEQQALPLPDLAEQFAPPDVAPPLLIKLLEAIEK |
| | KGLECSTLYRTQSSSNPAELRQLLDCDAASVDLEMIDVHVLADAFKR |
| | YLADLPNPVIPVAVYNEMMSLAQELQSPEDCIQLLKKLIRLPNIPHQ |
| | CWLTLQYLLKHFFKLSQASSKNLLNARVLSEIFSPVLFRFPAASSDN |
| | TEHLIKAIEILISTEWNERQPAPALPPKPPKPTTVANNSMNNNMSLQ |
| | DAEWYWGDISREEVNKLRDTADGTFLVRDASTKMHGDYTLTPRKGGN |
| | NKLIKIFHRDGKYGFSDPLTFNSVVELINHYRNESLAQYNPKLDVKL |
| | LYPVSKYQQDQVVKEDNIEAVGKKLHEYNTQFQEKSREYDRLYEEYT |
| | RTSQEIQMKRTAIEAFNETIKIFEEQCQTQERYSKEYIEKFKREGNE |
| | KEIQRIMHNHDKLKSRISEIIDSRRRLEEDLKKQAAEYREIDKRMNS |
| | IKPDLIQLRKTRDQYLMWLTQKGVRQKKLNEWLGNENTEDQYSLVED |
| | DEDLPHHDEKTWNVGSSNRNKAENLLRGKRDGTFLVRESSKQGCYAC |
| | SVVVDGEVKHCVINKTATGYGFAEPYNLYSSLKELVLHYQHTSLVQH |
| | NDSLNVTLAYPVYAQQRR |

Also suitable for use in the present invention is the sequence provided in Genbank Accession No. U50413 and AAC52847.

Table 6 (SEQ ID NO: 180) depicts the nucleotide sequence of human Pik3r1. Table 7 (SEQ ID NO:181) depicts the amino acid sequence of human Pik3r1.

TABLE 6

HUMAN

SEQ

| | |
|---|---|
| ID # | SEQUENCE |

| | |
|---|---|
| 180 | TACAACCAGG CTCAACTGTT GCATGGTAGC AGATTTGCAA |
| | ACATGAGTGC TGAGGGGTAC CAGTACAGAG CGCTGTATGA |
| | TTATAAAAAG GAAAGAGAAG AAGATATTGA CTTGCACTTG |
| | GGTGACATAT TGACTGTGAA TAAAGGGTCC TTAGTAGCTC |
| | TTGGATTCAG TGATGGACAG GAAGCCAGGC CTGAAGAAAT |
| | TGGCTGGTTA AATGGCTATA ATGAAACCAC AGGGGAAAGG |
| | GGGGACTT1C CGGGAACTTA CGTAGAATAT ATTGGAAGGA |
| | AAAAAATCTC GCCTCCCACA CCAAAGCCCC GGCCACCTCG |
| | GCCTCTTCCT GTTGCACCAG GTTCTTCGAA AACTGAAGCA |
| | GATGTTGAAC AACAAGCTTT GACTCTCCCG GATCTTGCAG |
| | AGCAGTTTGC CCCTCCTGAC ATTGCCCCGC CTCTTCTTAT |
| | CAAGCTCGTG GAAGCCATTG AAAAGAAAGG TCTGGAATGT |
| | TCAACTCTAT ACAGAACACA GAGCTCCAGC AACCTGGCAG |
| | AATTACGACA GCTTCTTGAT TGTGATACAC CCTCCGTGGA |
| | CTTGGAAATG ATCGATGTGC ACGTTTTGGC TGACGCTTTC |
| | AAACGCTATC TCCTGGACTT ACCAAATCCT GTCATTCCAG |
| | CAGCCGTTTA CAGTGAAATG ATTTCTTTAG CTCCAGAAGT |
| | ACAAAGCTCC GAAGAATATA TTCAGCTATT GAAGAAGCTT |
| | ATTAGGTCGC CTAGCATACC TCATCAGTAT TGGCTTACGC |
| | TTCAGTATTT GTTAAAACAT TTCTTCAAGC TCTCTCAAAC |
| | CTCCAGCAAA AATCTGTTGA ATGCAAGAGT ACTCTCTGAA |
| | ATTTTCAGCC CTATGCTTTT CAGATTCTCA GCAGCCAGCT |
| | CTGATAATAC TGAAAACCTC ATAAAAGTTA TAGAAATTTT |
| | AATCTCAACT GAATGGAATG AACGACAGCC TGCACCAGCA |
| | CTGGCTCCTA AACCACCAAA ACCTACTACT GTAGCCAACA |
| | ACGGTATGAA TAACAATATG TCCTTACAAA ATGCTGAATG |
| | GTACTGGGGA GATATCTCGA GGGAAGAAGT GAATGAAAAA |
| | CTTCGAGATA CAGCAGACGG GACC1TTTTG GTACGAGATG |
| | CGTCTACTAA AATGCATGGT GATTATACTC TTACACTAAG |
| | GAAAGGGGGA AATAACAAAT TAATCAAAAT ATTTCATCGA |
| | GATGGGAAAT ATGGCTTCTC TGACCCATTA ACCTTCAGTT |
| | CTGTGGTTGA ATTAATAAAC CACTACCGGA ATGAATCTCT |
| | AGCTCAGTAT AATCCCAAAT TGGATGTGAA ATTACTTTAT |
| | CCAGTATCCA AATACCAACA GGATCAAGTT GTCAAAGAAG |
| | ATAATATTGA AGCTGTAGGG AAAAAATTAC ATGAATATAA |
| | CACTCAGTTT CAAGAAAAAA GTCGAGAATA TGATAGATTA |
| | TATGAAGAAT ATACCCGCAC ATCCCAGGAA ATCCAAATGA |
| | AAAGGACAGC TATTGAAGCA TTTAATGAAA CCATAAAAAT |
| | ATTTGAAGAA CAGTGCCAGA CCCAAGAGCG GTACAGCAAA |
| | GAATACATAG AAAAGTTTAA ACGTGAAGGC AATGAGAAAG |
| | AAATACAAAG GATTATGCAT AATTATGATA AGTTGAAGTC |
| | TCGAATCAGT GAAATTATTG ACAGTAGAAG AAGATTGGAA |
| | GAAGACTTGA AGAAGCAGGC AGCTGAGTAT CGAGAAATTG |
| | ACAAACGTAT GAACAGCATT AAACCAGACC TTATCCAGCT |
| | GAGAAAGACG AGAGACCAAT ACTTGATGTG GTTGACTCAA |
| | AAAGGTGTTC GGCAAAAGAA GTTGAACGAG TGGTTGGGCA |
| | ATGAAAACAC TGAAGACCAA TATTCACTGG TGGAAGATGA |
| | TGAAGATTTG CCCCATCATG ATGAGAAGAC ATGGAATGTT |
| | GGAAGCAGCA ACCGAAACAA AGCTGAAAAC CTGTTGCGAG |
| | GGAAGCGAGA TGGCACTTTT CTTGTCCGGG AGAGCAGTAA |
| | ACAGGGCTGC TATGCCTGCT CTGTAGTGGT GGACGGCGAA |
| | GTAAAGCATT GTGTCATAAA CAAAACAGCA ACTGGCTATG |
| | GCTTTGCCGA GCCCTATAAC TTGTACAGCT CTCTGAAAGA |
| | ACTGGTGCTA CATTACCAAC ACACCTCCCT TGTGCAGCAC |
| | AACGACTCCC TCAATGTCAC ACTAGCCTAC CCAGTATATG |
| | CACAGCAGAG GCGATGAAGC GCTTACTCTT TGATCCTTCT |
| | CCTGAAGTTC AGCCACCCTG AGGCCTCTGG AAAGCAAAGG |
| | GCTCCTCTCC AGTCTGATCT GTGAATTGAG CTGCAGAAAC |
| | GAAGCCATCT TTCTTTGGAT GGGACTAGAG CTTTCTTTCA |
| | CAAAAAAGAA GTAGGGGAAG ACATGCAGCC TAAGGCTGTA |
| | TGATGACCAC ACGTTCCTAA GCTGGAGTGC TTATCCCTTC |
| | TTTTTCTTTT TTTCTTTGGT TTAATTTAAA GCCACAACCA |
| | CATACAACAC AAAGAGAAAA AGAAATGCAA AAATCTCTGC |
| | GTGCAGGGAC AAAGAGGCCT TTAACCATGG TGCTTGTTAA |
| | TGCTTTTTGA AGCTTTACCA GCTGAAAGTT GGGACTCTGG |
| | AGAGCGGAGG AGAGAGAGGC AGAAGAACCC TGGCCTGAGA |
| | AGGTTTGGTC CAGCCTGGTT TAGCCTGGAT GTTGCTGTGC |
| | ACGGTGGACC CAGACACATC GCACTGTGGA TTATTTCATT |
| | TTGTAACAAA TGAACGATAT GTAGCAGAAA GGCACGTCCA |
| | CTCACAAGGG ACGCTTTGGG AGAATGTCAG TTCATGTATG |
| | TTCAGAAGAA ATTCTGTCAT AGAAAGTGCC AGAAAGTGTT |
| | TAACTTGTCA AAAAACAAAA ACCCAGCAAC AGAAAAATGG |
| | AGTTTGGAAA ACAGGACTTA AAATGACATT CAGTATATAA |
| | AATATGTACA TAATATTGGA TGACTAACTA TCAAATAGAT |
| | GGATTTGTAT CAATACCAAA TAGCTTCTGT TTTGTTTTGC |
| | TGAAGGCTAA ATTCACAGCG CTATGCAATT CTTAATTTTC |
| | ATTAAGTTGT TATTTCAGTT TTAAATGTAC CTTCAGAATA |
| | AGCTTCCCCA CCCCAGTTTT TGTTGCTTGA AAATATTGTT |
| | GTCCCGGATT TTTGTTAATA TTCATTTTTG TTATCCTTTT |
| | TTAAAAATAA ATGTACAGGA TGCCAGTAAA AAAAAAAATG |
| | GCTTCAGAAT TAAAACTATG AAATATTTTA CAGTTTTTCT |
| | TGTACAGAGT ACTTGCTGTT AGCCCAAGGT TAAAAAGTTC |
| | ATAACAGATT TTTTTTGGAC TGTTTTGTTG GGCAGTGCCT |
| | GATAAGCTTC AAAGCTGCTT TATTCAATAA AAAAAAAACC |
| | CGAATTCACT GG |

TABLE 7
HUMAN SEQUENCE

| | |
|---|---|
| 181 | MSAEGYQYRA LYDYKKEREE DIDLHLGDIL TVNKGSLVAL |
| | GFSDGQEARP EEIGWLNGYN ETTGERGDFP GTYVEYIGRK |
| | KISPPTPKPR PPRPLPVAPG SSKTEADVEQ QALTLPDLAE |
| | QFAPPDIAPP LLIKLVEAIE KKGLECSTLY RTQSSSNLAE |
| | LRQLLDCDTP SVDLEMIDVH VLADAFKRYL LDLPNPVIPA |
| | AVYSEMISLA PEVQSSEEYI QLLKKLIRSP SIPHQYWLTL |
| | QYLLKHFFKL SQTSSKNLLN ARVLSEIFSP MLFRFSAASS |
| | DNTENLIKVI EILISTEWNE RQPAPALPPK PPKPTTVANN |
| | GMNNNMSLQN AEWYWGDISR EEVNEKLRDT ADGTFLVRDA |
| | STKMHGDYTL TLRKGGNNKL IKIFHRDGKY GFSDPLTFSS |
| | VVELINHYRN ESLAQYNPKL DVKLLYPVSK YQQDQVVKED |
| | NIEAVGKKLH EYNTQFQEKS REYDRLYEEY TRTSQEIQMK |
| | RTAIEAFNET IKIFEEQCQT QERYSKEYIE KFKREGNEKE |
| | IQRIMHNYDK LKSRISEIID SRRRLEEDLK KQAAEYREID |
| | DRMNSIKPDL IQLRKTRDQY LMWLTQKGVR QKKLNEWLGN |
| | ENTEDQYSLV EDDEDLPHHD EKTWNVGSSN RNKAENLLRG |
| | KRDGTFLVRE SSKQGCYACS VVVDGEVKHC VINKTATGYG |
| | FAEPYNLYSS LKELVLHYQH TSLVQHNDSL NVTLAYPVYA |
| | QQRR |

Also suitable for use in the present invention is the sequence provided in Genbank Accession No. M61906 and A38748.

A GNAS nucleic acid sequence of the invention is depicted in Table 8 as SEQ ID NO. 182. The nucleic acid sequence shown is from mouse.

TABLE 8

SEQ.
ID

| | |
|---|---|
| TAG # | NO. |

| | | |
|---|---|---|
| S00056 | 182 | GACGGTGATGCAGTAGAAATAAAGGTCTCAGCAGTGCAC |
| | | TGCAGAAAATCAAGCAAAGCCCCCTTAGGAGTTATTCAT |
| | | GTTTGCCGCTTTCGTGCAAATAGGGGAGGGGGCTTAAGG |
| | | CTTACCGGAAGACCCCCCACCTAGCTCAGGTCTTGTACT |
| | | TCTGTCTTCTGGGTAAAGGCAAAAGGAGATTTGGGGTGT |
| | | AGTTGATGGCCCATTTAGGGTGGTCTCGCAGACTAGAAA |
| | | ACCTGAAATGCACTTAAC |

A contig assembled from the mouse EST database by the National Center for Biotechnology Information (NCBI) having homology with all or parts of the GNAS nucleic acid sequence of the invention is depicted in Table 9 as SEQ ID NO. 183. SEQ ID NO. 184 represents the amino acid sequence of a protein encoded by SEQ ID NO. 183 and corresponds to mouse G protein Xl_{α}_{^{s}}.

TABLE 9

MOUSE

| | | |
|---|---|---|
| SAGRES | REF | SEQ |

| | | | |
|---|---|---|---|
| TAG # | # | ID # | SEQUENCE |

| | | | |
|---|---|---|---|
| S000056 | F12 | 183 | GTTGAGCGCGAAGCAGCCGAGATGGAAGGAAGCCCT |
| | | | ACCACCGCCACTGCGGTGGAAGGAAAAGTCCCCTCT |
| | | | CCGGAGAGAGGGGACGGATCTTCCACCCAGCCTGAA |
| | | | GCAATGGATGCCAAGCCAGCCCCTGCTGCCCAAGCC |
| | | | GTCTCTACCGGATCTGATGCTGGAGCTCCTACGGAT |
| | | | TCCGCGATGCTCACAGATAGCCAGAGCGATGCCGGA |
| | | | GAAGACGGGACAGCCCCAGGAACGCCTTCAGATCTC |
| | | | CAGTCGGATCCTGAAGAACTCGAAGAAGCCCCAGCT |
| | | | GTCCGCGCCGATCCTGACGGAGGGGCAGCCCCAGTC |
| | | | GCCCCAGCCACTCCTGCCGAGTCCGAGTCTGAAGGC |
| | | | AGCAGAGATCCAGCCGCCGAGCCAGCCTCCGAGGCA |
| | | | GTCCCTGCCACCACGGCCGAGTCTGCCTCCGGGGCA |
| | | | GCCCCTGTCACCCAGGTGGAGCCCGCAGCCGCGGCA |
| | | | GTCTCTGCCACCCTGGCGGAGCCTGCCGCCCGGGCA |
| | | | GCCCCTATCACCCCCAAGGAGCCCACTACCCGGGCA |
| | | | GTCCCCTCTGCTAGAGCCCATCCGGCCGCTGGAGCA |
| | | | GTCCCTGGCGCCCCAGCAATGTCAGCCTCTGCTAGG |
| | | | GCAGCTGCCGCTAGGGCAGCCTATGCAGGTCCACTG |
| | | | GTCTGGGGAGCCAGGTCACTCTCAGCTACTCCCGCC |
| | | | GCTCGGGCATCCCTTCCTGCCCGCGCAGCAGCTGCC |
| | | | GCCCGGGCAGCCTCTGCTGCCCGCGCAGTCGCTGCT |
| | | | GGCCGGTCAGCCTCTGCCGCGCCCAGCAGGGCCCAT |
| | | | CTTAGACCCCCCAGCCCCGAGATCCAGGTTGCTGAC |
| | | | CCGCCTACTCCGCGGCCTCCTCCGCGGCCGACTGCC |
| | | | TGGCCTGACAAGTACGAGCGGGGCCGAAGCTGCTGC |
| | | | AGGTACGAGGCATCGTCTGGCATCTGCGAGATCGAG |
| | | | TCCTCCAGTGATGAGTCGGAAGAAGGGGCCACCGGC |
| | | | TGCTTCCAGTGGCTTCTGCGGCGAAACCGCCGCCCT |
| | | | GGCCTGCCCCGGAGCCACACGGTCGGGAGCAACCCA |
| | | | GTCCGCAACTTCTTCACCCGAGCCTTCGGAAGCTGC |
| | | | TTCGGTCTATCCGAGTGTACCCGATCACGATCCCTC |
| | | | AGCCCCGGGAAGGCCAAGGATCCTATGGAGGAGAGG |
| | | | CGCAAACAGATGCGCAAAGAAGCCATTGAGATGCGA |
| | | | GAGCAGAAGCGCGCAGATAAGAAACGCAGCAAGCTC |
| | | | ATCGACAAGCAACTGGAGGAGGAGAAGATGGACTAC |
| | | | ATGTGTACACACCGCCTGCTGCTTCTAGGTGCTGGA |
| | | | GAGTCTGGCAAAAGCACCATTGTGAAGCAGATGAGG |
| | | | ATCCTGCATGTTAATGGGTTTAACGGAGATAGTGAG |
| | | | AAGGCCACTAAAGTGCAGGACATCAAAAACAACCTG |
| | | | AAGGAGGCCATTGAAACCATTGTGGCCGCCATGAGC |
| | | | AACCTGGTGCCCCCTGTGGAGCTGGCCAACCCTGAG |
| | | | AACCAGTTCAGAGTGGACTACATTCTGAGCGTGATG |
| | | | AACGTGCCGAACTTTGACTTCCCACCTGAATTCTAT |
| | | | GAGCATGCCAAGGCTCTGTGGGAGGATGAGGGAGTG |
| | | | CGTGCCTGCTACGAGCGCTCCAATGAGTACCAGCTG |
| | | | ATTGACTGTGCCCAGTACTTCCTGGACAAGATTGAT |
| | | | GTGATCAAGCAGGCCGACTACGTGCCAAGTGACCAG |
| | | | GACCTGCTTCGCTGCCGTGTCCTGACCTCTGGAATC |
| | | | TTTGAGACCAAGTTCCAGGTGGACAAAGTCAACTTC |
| | | | CACATGTTCGATGTGGGCGGCCAGCGCGATGAGCGC |
| | | | CGCAAGTGGATCCAGTGCTTCAATGATGTGACTGCC |
| | | | ATCATCTTCGTGGTGGCCAGCAGCAGCTACAACATG |
| | | | GTCATTCGGGAGGACAACCAGACTAACCGCCTGCAG |
| | | | GAGGCTCTGAACCTCTTCAAGAGCATCTGGAACAAC |
| | | | AGATGGCTGCGCACCATCTCTGTGAGGCTGTTCCTC |
| | | | AACAAGCAAGACCTGCTTGCTGAGAAAGTCCTCGCT |
| | | | GGCAAATCGAAGATTGAGGACTACTTTCCAGAGTTC |
| | | | GCTCGCTACACCACTCCTGAGGATGCGACTCCCGAG |
| | | | CCGGGAGAGGACCCACGCGTGACCCGGGCCAAGTAC |
| | | | TTCATTCGGGATGAGTTTCTGAGAATCAGCACTGCT |
| | | | AGTGGAGATGGGCGCCACTACTGCTACCCTCACTTT |
| | | | ACCTGCGCCGTGGACACTGAGAACATCCGCCGTGTC |
| | | | TTCAACGACTGCCGTGACATCATCCAGCGCATGCAT |
| | | | CTCCGCCAATACGAGCTGCTCTAAGAAGGGAACACC |
| | | | CAAATTTAATTCAGCCTTAAGCACAATTAATTAAGA |
| | | | GTGAAACGTAATTGTACAAGCAGTTGGTCACCCACC |
| | | | ATAGGGCATGATCAACACCGCAACCTTTCCTTTTTC |
| | | | CCCCAGTGATTCTGAAAAACCCCTCTTCCCTTCAGC |
| | | | TTGCTTAGATGTTCCAAATTTAGTAAGCTTAAGGCG |
| | | | GCCTACAGAAGAAAAAGAAAAAAAAGGCCACAAAAG |
| | | | TTCCCTCTCACTTTCAGTAAATAAAATAAAAGCAGC |
| | | | AACAGAAATAAAGAAATAAATGAAATTCAAAATGAA |
| | | | ATAAATATTGTGTTGTGCAGCATTAAAAAATCAATA |
| | | | AAAATCAAAAATGAGCAAAAAAAAAAA |
| | | 184 | MEGSPTTATAVEGKVPSPERGDGSSTQPEAMDAKPA |
| | | | PAAQAVSTGSDAGAPTDSAMLTDSQSDAGEDGTAPG |
| | | | TPSDLQSDPEELEEAPAVRADPDGGAAPVAPATPAE |
| | | | SESEGSRDPAAEPASEAVPATTAESASGAAPVTQVE |
| | | | PAAAAVSATLAEPAARAAPITPKEPTTRAVPSARAH |
| | | | PAAGAVPGAPAMSASARAAAARAAYAGPLVWGARSL |
| | | | SATPAARASLPARAAAAARAASAARAVAAGRSASAA |
| | | | PSRAHLRPPSPEIQVADPPTPRPPPRPTAWPDKYER |
| | | | GRSCCRYEASSGICEIESSSDESEEGATGCFQWLLR |
| | | | RNRRPGLPRSHTVGSNPVRNFFTRAFGSCFGLSECT |
| | | | RSRSLSPGKAKDPMEERRKQMRKEAIEMREQKRADK |
| | | | KRSKLIDKQLEEEKMDYMCTHRLLLLGAGESGKSTI |
| | | | VKQMRILHVNGFNGDSEKATKVQDIKNNLKEAIETI |
| | | | VAAMSNLVPPVELANPENQFRVDYILSVMNVPNFDF |
| | | | PPEFYEHAKALWEDEGVRACYERSNEYQLIDCAQYF |
| | | | LDKIDVIKQADYVPSDQDLLRCRVLTSGIFETKFQV |
| | | | DKVNFHMFDVGGQRDERRKWIQCFNDVTAIIFVVAS |
| | | | SSYNMVIREDNQTNRLQEALNLFKSIWNNRWLRTIS |
| | | | VILFLNKQDLLAEKVLAGKSKIEDYFPEFARYTTPE |
| | | | DATPEPGEDPRVTRAKYFIRDEFLRISTASGDGRHY |
| | | | CYPHFTCAVDTENIRRVFNDCRDIIQRMHLRQYELL |

Also suitable for use in the present invention is Genbank Accession No. AF116268.

A contig assembled from the human EST database by the NCBI having homology with all or parts of the GNAS nucleic acid sequence of the invention is depicted in Table 10 as SEQ ID NO. 185. SEQ ID NO. 186 represents the amino acid sequence of a protein encoded by SEQ ID NO. 185 and corresponds to human G protein Xl_{α}_{^{s}}.

TABLE 10

HUMAN

| | | |
|---|---|---|
| SAGRES | REF | SEQ |

| | | | |
|---|---|---|---|
| TAG # | # | ID # | SEQUENCE |

| | | | |
|---|---|---|---|
| S000056 | F37 | 185 | ATGGAGACCGAACCGCCTCACAACGAGCCCATCC |
| | | | CCGTCGAGAATGATGGCGAGGCCTGTGGACCCCC |
| | | | AGAGGTCTCCAGACCCAACTTTCAGGTCCTCAAC |
| | | | CCGGCATTCAGGGAAGCTGGAGCCCATGGAAGCT |
| | | | ACAGCCCACCTCCTGAGGAAGCAATGCCCTTCGA |
| | | | GGCTGAACAGCCCAGCTTGGGAGGCTTCTGGCCT |
| | | | ACACTGGAGCAGCCTGGATTCCCCAGTGGGGTCC |
| | | | ATGCAGGCCTTGCCAKGSTYSGSCCAGCACTCAT |
| | | | GGAGCCCGGAGCCTTCAGTGGTGCCAGACCAGGC |
| | | | CTGGGAGGATACAGCCCTCCACCAGAAGAAGCTA |
| | | | TGCCCTTTGAGTTTGACCAGCCTGCCCAGAGAGG |
| | | | CTGCAGTCAACTTCTCTTACAGGTCCCAGACCTT |
| | | | GCTCCAGGAGGCCCAGGTGCTGCAGGGGTCCCCG |
| | | | GAGCTCCTCCCGAGGAGCCCCAAGCCCTCAGGCC |
| | | | TGCAAAGGCTGGCTCCAGAGGAGGCTACAGCCCT |
| | | | CCCCCTGAGGAGACTATGCCATTTGAGCTTGATG |
| | | | GAGAAGGATTTGGGGACGACAGCCCACCCCCGGG |
| | | | GCTTTCCCGAGTTATCGCACAAGTCGACGGCAGC |
| | | | AGCCAGTTCGCGGCAGTCGCGGCCTCGAGTGCGG |
| | | | TCCGCCTCACTCCCGCCGCGAACGCGCCTCCCCT |
| | | | CTGGGTCCCAGGCGCCATCGGCAGCCCATCCCAA |
| | | | GAGGCTGTCAGACCTCCTTCTAACTTCACGGGCA |
| | | | GCAGCCCCTGGATGGAGATCTCCGGACCCCCGTT |
| | | | CGAGATTGGCAGCGCCCCCGGTGGGGTCGACGAC |
| | | | ACTCCCGTCAACATGGACAGCCCCCCAATCGCGC |
| | | | TTGACGGCCCGCCCATCAAGGTCTCCGGAGCCCC |
| | | | AGATAAGAGAGAGCGAGCAGAGAGACCCCCAGTT |
| | | | GAGGAGGAAGCAGCAGAGATGGAAGGAGCCGCTG |
| | | | ATGCCGCGGAGGGAGGAAAAGTACCCTCTCCGGG |
| | | | GTACGGATCCCCTGCCGCCGGGGCAGCCTCAGCG |
| | | | GATACCGCTGCCAGGGCAGCCCCTGCAGCCCCAG |
| | | | CCGATCCTGACTCCGGGGCAACCCCAGAAGATCC |
| | | | CGACTCCGGGACAGCACCAGCCGATCCTGACTCC |
| | | | GGGGCATTCGCAGCCGATCCCGACTCCGGGGCAG |
| | | | CCCCTGCCGCCCCAGCCGATCCCGACTCCGGGGC |
| | | | GGCCCCTGACGCCCCAGCCGATCCCGACTCCGGG |
| | | | GCGGCCCCTGACGCCCCAGCCGATCCAGATGCCG |
| | | | GGGCGGCCCCTGAGGCTCCCGCCGCCCCTGCGGC |
| | | | TGCTGAGACCCGGGCAGCCCATGTCGCCCCAGCT |
| | | | GCGCCAGACGCAGGGGCTCCCACTGCCCCAGCCG |
| | | | CTTCTGCCACCCGGGCAGCCCAAGTCCGCCGGGC |
| | | | GGCCTCTGCAGCCCCTGCCTCCGGGGCCAGACGC |
| | | | AAGATCCATCTCAGACCCCCCAGCCCCGAGATCC |
| | | | AGGCTGCCGATCCGCCTACTCCGCGGCCTACTCG |
| | | | CGCGTCTGCCTGGCGGGGCAAGTCCGAGAGCAGC |
| | | | CGCGGCCGCCGCGTGTACTACGATGAAGGGGTGG |
| | | | CCAGCAGCGACGATGACTCCAGCGGAGACGAGTC |
| | | | CGACGATGGGACCTCCGGATGCCTCCGCTGGTTT |
| | | | CAGCATCGGCGAAATCGCCGCCGCCGAAAGCCCC |
| | | | AGCGCAACTTACTCCGCAACTTTCTCGTGCAAGC |
| | | | CTTCGGGGGCTGCTTCGGTCGATCTGAGAGTCCC |
| | | | CAGCCCAAAGCCTCGCGCTCTCTCAAGGTCAAGA |
| | | | AGGTACCCCTGGCGGAGAAGCGCAGACAGATGCG |
| | | | CAAAGAAGCCCTGGAGAAGCGGGCCCAGAAGCGC |
| | | | GCAGAGAAGAAACGCAGTAAGCTCATCGACAAAC |
| | | | AACTCCAGGACGAAAAGATGGGCTACATGTGTAC |
| | | | GCACCGCCTGCTGCTTCTAG |
| | | 186 | MEISGPPFEIGSAPAGVDDTPVNMDSPPIALDGP |
| | | | PIKVSGAPDKRERAERPPVEEEAAEMEGAADAAE |
| | | | GGKVPSPGYGSPAAGAASADTAARAAPAAPADPD |
| | | | SGATPEDPDSGTAPADPDSGAFAADPDSGAAPAA |
| | | | PADPDSGAAPDAPADPDSGAAPDAPADPDAGAAP |
| | | | EAPAAPAAETRAAHVAPAAPDAGAPTAPAASATR |
| | | | AAQVRRAASAAPASGARRKIHLRPPSPEIQAADP |
| | | | PTPRPTRASAWRGKSESSRGRRVYYDEGVASSDD |
| | | | DSSGDESDDGTSGCLRWFQHRRNRRRRKPQRNLL |
| | | | RNFLVQAFGGCFGRSESPQPKASRSLKVKKVPLA |
| | | | EKRRQMRKEALEKRAQKRAEKKRSKLIDKQLQDE |
| | | | KMGYMCTHRLLLL |

Table 11 demonstrates the nucleic acid sequence (SEQ ID NO: 187) and amino acid sequence (SEQ ID NO: 188) of NESP55 from mouse. SEQ ID NO: 188 represents the protein encoded by SEQ ID NO: 187.

TABLE 11

MOUSE

| | | |
|---|---|---|
| SAGRES | REF | SEQ |

| | | | |
|---|---|---|---|
| TAG # | # | ID # | SEQUENCE |

| | |
|---|---|
| 187 | GAGAGGATCA GTGGAGGCAC CTCTCGGAGT |
| | CTTAGACTTC AGAGTCTGAG ACTTAGCGAG |
| | AGGAGCCTCG AGGAGACTCC TTCTCTCTTC |
| | TTTACCCATC CCTTTCTTTT ACTTACAGCC |
| | TCAAGCTGAG GCGCGGAGCT TTAGAAAGTT |
| | CGCAGTGGTT TGAAGTCCTT GCGCAGTGGG |
| | GCCACTCTCT GCAGAGCCAG AGGGTGAGTC |
| | GGCTTCTCGG TGAGCACCTA AGAGAATGGA |
| | TCGCAGGTCC CGGGCTCAGC AGTGGCGCCG |
| | AGCTCGCCAT AATTACAACG ACCTGTGCCC |
| | GCCCATAGGC CGCCGGGCTG CCACCGCTCT |
| | CCTCTGGCTC TCCTGCTCCA TTGCTCTCCT |
| | CCGCGCCCTA GCCTCTTCCA ACGCCCGCGC |
| | CCAGCAGCGT GCTGCCCATC GCCGGAGCTT |
| | CCTTAACGCC CACCACCGCT CCGCTGCCGC |
| | TGCAGCTGCC GCACAGGTAC TCCCTGAGTC |
| | CTCTGAATCT GAGTCTGATC ACGAGCACGA |
| | GGAGGTTGAG CCTGAGCTGG CCCGCCCCGA |
| | GTGCCTAGAG TACGATCAGG ACGACTACGA |
| | GACCGAGACC GATTCTGAGA CCGAGCCTGA |
| | GTCCGATATC GAATCCGAGA CCGAAATCGA |
| | GACCGAGCCA GAGACCGAGC CAGAAACCGA |
| | GCCAGAGACC GAGCCAGAGG ACGAGCGCGG |
| | CCCCCGGGGT GCCACCTTCA ACCAGTCACT |
| | CACTCAGCGT CTGCACGCTC TGAAGTTGCA |
| | GAGCGCCGAC GCCTCCCCGA GACGTGCGCA |
| | GCCCACCACT CAGGAGCCTG AGAGCGCAAG |
| | CGAGGGGGAG GAGCCCCAGC GAGGGCCCTT |
| | AGATCAGGAT CCTCGGGACC CCGAGGAGGA |
| | GCCAGAGGAG CGCAAGGAGG AAAACAGGCA |
| | GCCCCGCCGC TGCAAGACCA GGAGGCCAGC |
| | CCGCCGTCGC GACCAGTCCC CGGAGTCCCC |
| | TCCCAGAAAG GGGCCCATCC CCATCCGGCG |
| | TCACTAATGG GTGACTCCGT CCAGATTCTC |
| | CTTGTTTTCA TGGATAAAGG TGCTGGAGAG |
| | TCTGGCAAAA GCACCATTGT GAAGCAGATG |
| | AGGATCCTGC ATGTTAATGG GTTTAACGGA |
| | G |
| 188 | MDRRSRAQQWRRARHNYNDLCPPIGRRAATALLW |
| | LSCSIALLRALASSNARAQQRAAHRRSFLNAHHR |
| | SAAAAAAAQVLPESSESESDHEHEEVEPELARPE |
| | CLEYDQDDYETETDSETEPESDIESETEIETEPE |
| | TEPETEPETEPEDERGPRGATFNQSLTQRLHALK |
| | LQSADASPRRAQPTTQEPESASEGEEPQRGPLDQ |
| | DPRDPEEEPEERKEENRQPRRCKTRRPARRRDQS |
| | PPESPPRKGPIPIRRH |

Table 12 demonstrates the nucleic acid sequence (SEQ ID NO: 189) and amino acid-sequence (SEQ ID NO: 190) of NESP55 from human. SEQ ID NO: 190 represents the protein encoded by SEQ ID NO: 189.

TABLE 12

HUMAN

| | | |
|---|---|---|
| SAGRES | REF | SEQ |

| | | | |
|---|---|---|---|
| TAG # | # | ID # | SEQUENCE |

| | |
|---|---|
| 189 | CTCGCCTCAG TCTCCTCTGT CCTCTCCCAG |
| | GCAAGAGGAC CGGCGGAGGC ACCTCTCTCG |
| | AGTCTTAGGC TGCGGAATCT AAGACTCAGC |
| | GAGAGGAGCC CGGGAGGAGA CAGAACTTTC |
| | CCCTTTTTTC CCATCCCTTC TTCTTGCTCA |
| | GAGAGGCAAG CAAGGCGCGG AGCTTTAGAA |
| | AGTTCTTAAG TGGTCAGGAA GGTAGGTGCT |
| | TCCCTTTTTC TCCTCACAAG GAGGTGAGGC |
| | TGGGACCTCC GGGCCAGCTT CTCACCTCAT |
| | AGGGTGTACC TTTCCCGGCT CCAGCAGCCA |
| | ATGTGCTTCG GAGCCGCTCT CTGCAGAGCC |
| | AGAGGGCAGG CCGGCTTCTC GGTGTGTGCC |
| | TAAGAGGATG GATCGGAGGT CCCGGGCTCA |
| | GCAGTGGCGC CGAGCTCGCC ATAATTACAA |
| | CGACCTGTGC CCGCCCATAG GCCGCCGGGC |
| | AGCCACCGCG CTCCTCTGGC TCTCCTGCTC |
| | CATCGCGCTC CTCCGCGCCC TTGCCACCTC |
| | CAACGCCCGT GCCCAGCAGC GCGCGGCTGC |
| | CCAACAGCGC CGGAGCTTCC TTAACGCCCA |
| | CCACCGCTCC GGCGCCCAGG TATTCCCTGA |
| | GTCCCCCGAA TCGGAATCTG ACCACGAGCA |
| | CGAGGAGGCA GACCTTGAGC TGTCCCTCCC |
| | CGAGTGCCTA GAGTACGAGG AAGAGTTCGA |
| | CTACGAGACC GAGAGCGAGA CCGAGTCCGA |
| | AATCGAGTCC GAGACCGACT TCGAGACCGA |
| | GCCTGAGACC GCCCCCACCA CTGAGCCCGA |
| | GACCGAGCCT GAAGACGATC GCGGCCCGGT |
| | GGTGCCCAAG CACTCCACCT TCGGCCAGTC |
| | CCTCACCCAG CGTCTGCACG CTCTCAAGTT |
| | GCGAAGCCCC GACGCCTCCC CAAGTCGCGC |
| | GCCGCCCAGC ACTCAGGAGC CCCAGAGCCC |
| | CAGGGAAGGG GAGGAGCTCA AGCCCGAGGA |
| | CAAAGATCCA AGGGACCCCG AAGAGTCGAA |
| | GGAGCCCAAG GAGGAGAAGC AGCGGCGTCG |
| | CTGCAAGCCA AAGAAGCCCA CCCGCCGTGA |
| | CGCGTCCCCG GAGTCCCCTT CCAAAAAGGG |
| | ACCCATCCCC ATCCGGCGTC ACTAATGGAG |
| | GACGCCGTCC AGATTCTCCT TGTTTTCATG |
| | GATTCAGGTG CTGGAGAATC TGGTAAAAGC |
| | ACCATTGTGA AGCAGATGAG GATCCTGCAT |
| | GTTAATGGGT TTAATGGAGA GGGCGGCGAA |
| | GAGGACCCGC AGGCTGCAAG GAGCAACAGC |
| | GATGGCAGTG AGAAGGCAAC CAAAGTGCAG |
| | GACATCAAAA ACAACCTGAA AGAGGCGATT |
| | GAAACCATTG TGGCCGCCAT GAGCAACCTG |
| | GTGCCCCCCG TGGAGCTGGC CAACCCCGAG |
| | AACCAGTTCA GAGTGGACTA CATCCTGAGT |
| | GTGATGAACG TGCCTGACTT TGACTTCCCT |
| | CCCGAATTCT ATGAGCATGC CAAGGCTCTG |
| | TGGGAGGATG AAGGAGTGCG TGCCTGCTAC |
| | GAACGCTCCA ACGAGTACCA GCTGATTGAC |
| | TGTGCCCAGT ACTTCCTGGA CAAGATCGAC |
| | GTGATCAAGC AGGCTGACTA TGTGCCGAGC |
| | GATCAGGACC TGCTTCGCTG CCGTGTCCTG |
| | ACTTCTGGAA TCTTTGAGAC CAAGTTCCAG |
| | GTGGACAAAG TCAACTTCCA CATGTTTGAC |
| | GTGGGTGGCC AGCGCGATGA ACGCCGCAAG |
| | TGGATCCAGT GCTTCAACGA TGTGACTGCC |
| | ATCATCTTCG TGGTGGCCAG CAGCAGCTAC |
| | AACATGGTCA TCCGGGAGGA CAACCAGACC |
| | AACCGCCTGC AGGAGGCTCT GAACCTCTTC |
| | AAGAGCATCT GGAACAACAG ATGGCTGCGC |
| | ACCATCTCTG TGATCCTGTT CCTCAACAAG |
| | CAAGATCTGC TCGCTGAGAA AGTCCTTGCT |
| | GGGAAATCGA AGATTGAGGA CTACTTTCCA |
| | GAATTTGCTC GCTACACTAC TCCTGAGGAT |
| | GCTACTCCCG AGCCCGGAGA GGACCCACGC |
| | GTGACCCGGG CCAAGTACTT CATTCGAGAT |
| | GAGTTTCTGA GGATCAGCAC TGCCAGTGGA |
| | GATGGGCGTC ACTACTGCTA CCCTCATTTC |
| | ACCTGCGCTG TGGACACTGA GAACATCCGC |
| | CGTGTGTTCA ACGACTGCCG TGACATCATT |
| | CAGCGCATGC ACCTTCGTCA GTACGAGCTG |
| | CTCTAAGAAG GGAACCCCCA AATTTAATTA |
| | AAGCCTTAAG CACAATTAAT TAAAAGTGAA |
| | ACGTAATTGT ACAAGCAGTT AATCACCCAC |
| | CATAGGGCAT GATTAACAAA GCAACCTTTC |
| | CCTTCCCCCG AGTGATTTTG CGAAACCCCC |
| | TTTTCCCTTC AGCTTGCTTA GATGTTCCAA |
| | ATTTAGAAAG CTTAAGGCGG CCTACAGAAA |
| | AAGGAAAAAA GGCCACAAAA GTTCCCTCTC |
| | ACTTTCAGTA AAAATAAATA AAACAGCAGC |
| | AGCAAACAAA TAAAATGAAA TAAAAGAAAC |
| | AAATGAAATA AATATTGTGT TGTGCAGCAT |
| | TAAAAAAAAT CAAAATAAAA ATTAAATGTG |
| | AGCAAAGAAA AAAAAA GAGAGGATCA |
| | GTGGAGGCAC CTCTCGGAGT CTTAGACTTC |
| | AGAGTCTGAG ACTTAGCGAG AGGAGCCTCG |
| | AGGAGACTCC TTCTCTCTTC TTTACCCATC |
| | CCTTTCTTTT ACTTACAGCC TCAAGCTGAG |
| | GCGCGGAGCT TTAGAAAGTT CGCAGTGGTT |
| | TGAAGTCCTT GCGCAGTGGG GCCACTCTCT |
| | GCAGAGCCAG AGGGTGAGTC GGCTTCTCGG |
| | TGAGCACCTA AGAGAATGGA TCGCAGGTCC |
| | CGGGCTCAGC AGTGGCGCCG AGCTCGCCAT |
| | AATTACAACG ACCTGTGCCC GCCCATAGGC |
| | CGCCGGGCTG CCACCGCTCT CCTCTGGCTC |
| | TCCTGCTCCA TTGCTCTCCT CCGCGCCCTA |
| | GCCTCTTCCA ACGCCCGCGC CCAGCAGCGT |
| | GCTGCCCATC GCCGGAGCTT CCTTAACGCC |
| | CACCACCGCT CCGCTGCCGC TGCAGCTGCC |
| | GCAGAGGTAC TCCCTGAGTC CTCTGAATCT |
| | GAGTCTGATC ACGAGCACGA GGAGGTTGAG |
| | CCTGAGCTGG CCCGCCCCGA GTGCCTAGAG |
| | TACGATCAGG ACGACTACGA GACCGAGACC |
| | GATTCTGAGA CCGAGCCTGA GTCCGATATG |
| | GAATCCGAGA CCGAAATCGA GACCGAGCCA |
| | GAGACCGAGC CAGAAACCGA GCCAGAGACC |
| | GAGCCAGAGG ACGAGCGCGG CCCCCGGGGT |
| | GCCACCTTCA ACCAGTCACT CACTCAGCGT |
| | CTGCACGCTC TGAAGTTGCA GAGCGCCGAC |
| | GCCTCCCCGA GACGTGCGCA GCCCACCACT |
| | CAGGAGCCTG AGAGCGCAAG CGAGGGGGAG |
| | GAGCCCCAGC GAGGGCCCTT AGATCAGGAT |
| | CCTCGGGACC CCGAGGAGGA GCCAGAGGAG |
| | CGCAAGGAGG AAAACAGGCA GCCCCGCCGC |
| | TGCAAGACCA GGAGGCCAGC CCGCCGTCGC |
| | GACCAGTCCC CGGAGTCCCC TCCCAGAAAG |
| | GGGCCCATCC CCATCCGGCG TCACTAATGG |
| | GTGACTCCGT CCAGATTCTC CTTGTTTTCA |
| | TGGATAAAGG TGCTGGAGAG TCTGGCAAAA |
| | GCACCATTGT GAAGCAGATG AGGATCCTGC |
| | ATGTTAATGG GTVTAACGGA G |
| 190 | MDRRSRAQQWRRARHNYNDLCPPIGRRAATALLW |
| | LSCSIALLRALATSNARAQQRAAAQQRRSFLNAH |
| | HRSGAQVFPESPESESDHEHEEADLELSLPECLE |
| | YEEEFDYETESETESEIESETDFETEPETAPTTE |
| | PETEPEDDRGPVVPKHSTFGQSLTQRLHALKLRS |
| | PDASPSRAPPSTQEPQSPREGEELKPEDKDPRED |
| | PEESKEPKEEKQRRRCKPKKPTRRDASPESPSKK |
| | GPIPIRRH |

Table 13 demonstrates the nucleic acid sequence (SEQ ID NO: 191) and amino acid sequence (SEQ ID NO: 192) of GNAS1 from mouse. SEQ ID NO: 192 represents the protein encoded by SEQ ID NO: 191.

TABLE 13

MOUSE

| | | |
|---|---|---|
| SAGRES | REF | SEQ |

| | | | |
|---|---|---|---|
| TAG # | # | ID # | SEQUENCE |

| | |
|---|---|
| 191 | CCCCGCGCCC CGCCGCCGCA TGGGCTGCCT |
| | CGGCAACAGT AAGACCGAGG ACCAGCGCAA |
| | CGAGGAGAAG GCGCAGCGCG AGGCCAACAA |
| | AAAGATCGAG AAGCAGCTGC AGAAGGACAA |
| | GCAGGTCTAC CGGGCCACGC ACCGCCTGCT |
| | GCTGCTGGGT GCTGGAGAGT CTGGCAAAAG |
| | CACCATTGTG AAGCAGATGA GGATCCTGCA |
| | TGTTAATGGG TTTAACGGAG AGGGCGGCGA |
| | AGAGGACCCG CAGGCTGCAA GGAGCAACAG |
| | CGATGGTGAG AAGGCCACTA AAGTGCAGGA |
| | CATCAAAAAC AACCTGAAGG AGGCCATTGA |
| | AACCATTGTG GCCGCCATGA GCAACCTGGT |
| | GCCCCCTGTG GAGCTGGCCA ACCCTGAGAA |
| | CCAGTTCAGA GTGGACTACA TTCTGAGCGT |
| | GATGAACGTG CCCGACTTTG ACTTCCCACC |
| | TGAATTCTAT GAGCATGCCA AGGCTCTGTG |
| | GGAGGATGAG GGAGTGCGTG CCTGCTACGA |
| | GCGCTCCAAT GAGTACCAGC TGATTGACTG |
| | TGCCCAGTAC TTCCTGGACA AGATTGATGT |
| | GATCAAGCAG GCCGACTACG TGCCAAGTGA |
| | CCAGGACCTG CTTCGCTGCC GTGTCCTGAC |
| | CTCTGGAATC TTTGAGACCA AGTTCCAGGT |
| | GGACAAAGTC AACTTCCACA TGTTCGATGT |
| | GGGCGGCCAG CGCGATGAAC GCCGCAAGTG |
| | GATCCAGTGC TTCAATGATG TGACTGCCAT |
| | CATCTTCGTG GTGGCCAGCA GCAGCTACAA |
| | CATGGTCATT CGGGAGGACA ACCAGACTAA |
| | CCGCCTGCAG GAGGCTCTGA ACCTCTTCAA |
| | GAGCATCTGG AACAACAGAT GGCTGCGCAC |
| | CATCTCTGTG ATTCTCTTCC TCAACAAGCA |
| | AGACCTGCTT GCTGAGAAAG TCCTCGCTGG |
| | CAAATCGAAG ATTGAGGACT ACTTTCCAGA |
| | GTTCGCTCGC TACACCACTC CTGAGGATGC |
| | GACTCCCGAG CCGGGAGAGG ACCCACGCGT |
| | GACCCGGGCC AAGTACTTCA TTCGGGATGA |
| | GTTTCTGAGA ATCAGCACTG CTAGTGGAGA |
| | TGGGCGCCAC TACTGCTACC CTCACTTTAC |
| | CTGCGCCGTG GACACTGAGA ACATCCGCCG |
| | TGTCTTCAAC GACTGCCGTG ACATCATCCA |
| | GCGCATGCAT CTCCCCCAAT ACGAGCTGCT |
| | CTAAGAAGGG AACACCCAAA TTTAATTCAG |
| | CCTTAAGCAC AATTAATTAA GAGTGAAACG |
| | TAATTGTACA AGCAGTTGGT CACCCACCAT |
| | AGGGCATGAT CAACACCGCA ACCTTTCCTT |
| | TTTCCCCCAG TGATTCTGAA AAACCCCTCT |
| | TCCCTTCAGC TTGCTTAGAT GTTCCAAATT |
| | TAGAAGCTT |
| 192 | MGCLGNSKTEDQRNEEKAQREANKKIEKQLQKDK |
| | QVYRATHRLLLLGAGESGKSTIVKQMRILHVNGF |
| | NGEGGEEDPQAARSNSDGEKATKVQDIKNNLKEA |
| | IETIVAAMSNLVPPVELANPENQFRVDYILSVMN |
| | VPDFDFPPEFYEHAKALWEDEGVRACYERSNEYQ |
| | LIDCAQYFLDKIDVIKQADYVPSDQDLLRCRVLT |
| | SGIFETKFQVDKVNFHMFDVGGQRDERRKWIQCF |
| | NDVTAIIFVVASSSYNMVIREDNQTNRLQEALNL |
| | FKSIWNNRWLRTISVILFLNKQDLLAEKVLAGKS |
| | KIEDYFPEFARYTTPEDATPEPGEDPRVTRAKYF |
| | IRDEFLRISTASGDGRHYCYPHFTCAVDTENIRR |
| | VFNDCRDIIQRMHLPQYELL |

Table 14 demonstrates the nucleic acid sequence (SEQ ID NO: 193) and amino acid sequence (SEQ ID NO: 194) of GNAS1 from human. SEQ ID NO: 194 represents the protein encoded by SEQ ID NO: 193.

TABLE 14

HUMAN

| | | |
|---|---|---|
| SAGRES | REF | SEQ |

| | | | |
|---|---|---|---|
| TAG # | # | ID # | SEQUENCE |

| | |
|---|---|
| 193 | GCGGGCGTGC TGCCGCCGCT GCCGCCGCCG |
| | CCGCAGCCCG GCCGCGCCGC GCCGCCGCCG |
| | CCGCCGCCAT GGGCTGCCTC GGGAACAGTA |
| | AGACCGAGGA CCAGCGCAAC GAGGAGAAGG |
| | CGCAGCGTGA GGCCAACAAA AAGATCGAGA |
| | AGCAGCTGCA GAAGGACAAG CAGGTCTACC |
| | GGGCCACGCA CCGCCTGCTG CTGCTGGGTG |
| | CTGGAGAATC TGGTAAAAGC ACCATTGTGA |
| | AGCAGATGAG GATCCTGCAT GTTAATGGGT |
| | TTAATGGAGA GGGCGGCGAA GAGGACCCGC |
| | AGGCTGCAAG GAGCAACAGC GATGGTGAGA |
| | AGGCAACCAA AGTGCAGGAC ATCAAAAACA |
| | ACCTGAAAGA GGCGATTGAA ACCATTGTGG |
| | CCGCCATGAG CAACCTGGTG CCCCCCGTGG |
| | AGCTGGCCAA CCCCGAGAAC CAGTTCAGAG |
| | TGGACTACAT CCTGAGTGTG ATGAACGTGC |
| | CTGACTTTGA CTTCCCTCCC GAATTCTATG |
| | AGCATGCCAA GGCTCTGTGG GAGGATGAAG |
| | GAGTGCGTGC CTGCTACGAA CGCTCCAACG |
| | AGTACCAGCT GATTGACTGT GCCCAGTACT |
| | TCCTGGACAA GATCGACGTG ATCAAGCAGG |
| | CTGACTATGT GCCGAGCGAT CAGGACCTGC |
| | TTCGCTGCCG TGTCCTGACT TCTGGAATCT |
| | TTGAGACCAA GTTCCAGGTG GACAAAGTCA |
| | ACTTCCACAT GTTTGACGTG GGTGGCCAGC |
| | GCGATGAACG CCGCAAGTGG ATCCAGTGCT |
| | TCAACGATGT GACTGCCATC ATCTTCGTGG |
| | TGGCCAGCAG CAGCTACAAC ATGGTCATCC |
| | GGGAGGACAA CCAGACCAAC CGCCTGCAGG |
| | AGGCTCTGAA CCTCTTCAAG AGCATCTGGA |
| | ACAACAGATG GCTGCGCACC ATCTCTGTGA |
| | TCCTGTTCCT CAACAAGCAA GATCTGCTCG |
| | CTGAGAAAGT CCTTGCTGGG AAATCGAAGA |
| | TTGAGGACTA CTTTCCAGAA TTTGCTCGCT |
| | ACACTACTCC TGAGGATGCT ACTCCCGAGC |
| | CCGGAGAGGA CCCACGCGTG ACCCGGGCCA |
| | AGTACTTCAT TCGAGATGAG TTTCTGAGGA |
| | TCAGCACTGC CAGTGGAGAT GGGCGTCACT |
| | ACTGCTACCC TCATTTCACC TGCGCTGTGG |
| | ACACTGAGAA CATCCGCCGT GTGTTCAACG |
| | ACTGCCGTGA CATCATTCAG CGCATGCACC |
| | TTCGTCAGTA CGAGCTGCTC TAAGAAGGGA |
| | ACCCCCAAAT TTAATTAAAG CCTTAAGCAC |
| | AATTAATTAA AAGTGAAACG TAATTGTACA |
| | AGCAGTTAAT CACCGACCAT AGGGCATGAT |
| | TAACAAAGCA ACCTTTCCCT TCCCCCGAGT |
| | GATTTTGCGA AACCCCCTTT TCCCTTCAGC |
| | TTGCTTAGAT GTTCCAAATT TAGAAAGCTT |
| | AAGGCGGCCT ACAGAAAAAG GAAAAAAGGC |
| | CACAAAAGTT CCCTCTCACT TTCAGTAAAA |
| | ATAAATAAAA CAGCAGCAGC AAACAAATAA |
| | AATGAAATAA AAGAAACAAA TGAAATAAAT |
| | ATTGTGTTGT GCAGCATTAA AAAAAATCAA |
| | AATAAAAATT AAATGTGAGC |
| 194 | MGCLGNSKTEDQRNEEKAQREANKKIEKQLQKDK |
| | QVYRATHRLLLLGAGESGKSTIVKQMRILHVNGF |
| | NGEGGEEDPQAARSNSDGEKATKVQDIKNNLKEA |
| | IETIVAAMSNLVPPVELANPENQFRVDYILSVMN |
| | VPDFDFPPEFYEHAKALWEDEGVRACYERSNEYQ |
| | LIDCAQYFLDKIDVIDQADYVPSDQDLLRCRVLT |
| | SGIFETKFQVDKVNFHMFDVGGQRDERRKWIQCF |
| | NDVTAIIFVVASSSYNMVIREDNQTNRLQEALNL |
| | FKSIWNNRWLRTISVILFLNKQDLLAEKVLAGKS |
| | KIEDYFPEFARYTTPEDATPEPGEDPRVTRAKYF |
| | IRDEFLRISTASGDGRHYCYPHFTCAVDTENIRR |
| | VFNDCRDIIQRMHLRQYELL |

Also suitable for use in the present invention is Genbank Accession No. AJ224868.

A HIPK1 nucleic acid sequence of the invention is depicted in Table 15 as SEQ ID NO. 195. The nucleic acid sequence shown is from mouse.

TABLE 15

| | |
|---|---|
| TAG | SEQ ID |

| | | |
|---|---|---|
| # | NO. | SEQUENCE |

| | | |
|---|---|---|
| S00013 | 195 | CTCCGTNGGGAGCCANCNTGGACGGNGTGTGGGGAC |
| | | CGGTNTCCCAGTCNTCTCCGCAAANCGGTCTCCNAG |
| | | GTGGTTTAACCGGNGTTTGGTGGNGGTCGGGTTTCT |
| | | TACAGTTAGATGTCANCTCANCTAGTGTGACATCAC |
| | | CCCAAACCAGTGTGATTTTTCCCCCAACATCCCAAT |
| | | CACATCCCAGCGATTGGGCAGCGCAGGGAGACATTG |
| | | ACTACCTGGGGGATGACTCTGAGGGTTTAGAATTCT |
| | | CAGTTTTTACTTAAATTGTTTGCTGCCATGTCGATT |
| | | TCAGGGCAGCNAGGGGGNATTTAGATGCCTCCCTGT |
| | | CCTTNGA |

A contig assembled from the mouse EST database by the National Center for Biotechnology Information (NCBI) having homology with all or parts of a HIPK1 nucleic acid sequence of the invention is depicted in Table 16 as SEQ ID NO. 196. SEQ ID NO. 197 represents the amino acid sequence of a protein encoded by SEQ ID NO. 196.

TABLE 16

MOUSE

| | | |
|---|---|---|
| SAGRES | REF | SEQ |

| | | | |
|---|---|---|---|
| TAG # | # | ID # | SEQUENCE |

| | | | |
|---|---|---|---|
| S000013 | F3 | 196 | CCGCCACCAAACGCCGGTTAAACCACCTCGGAGA |
| | | | CTGCTGTGCGGAGAGGACTGGGAAACCGGTCCCC |
| | | | ACACACTGTCCACGCTGGCTCCCCACGGAGGCCC |
| | | | ACCCACACCCGCGGCCCGGGGCAAGATGCAGTGA |
| | | | TCTCAGCCCTCCCGCTCCTCCGCACTTCCGCCTC |
| | | | AGTATGGCCTCACAGCTGCAGGTGTTTCGCCCCC |
| | | | ATCAGTGTCGTCGAGTGCCTTCTGCAGTGCAAAG |
| | | | AAACTGAAAATAGAGCCCTCTGGCTGGGATGTTT |
| | | | CAGGACAGAGCAGCAACGACAAATACTATACCAC |
| | | | AGCAAAACCCTCCCAGCTACACAAGGGCAAGCCA |
| | | | GCTCCTCTCACCAGGTAGCAAATTTCAATCTTCC |
| | | | TGCTTACGACCAGGGCCTCCTTCTCCCAGCTCCT |
| | | | GCCGTGGAGCATATTGTGGTAACAGCTGCTGATA |
| | | | GCTCAGGCAGCGCCGCTACAGCAACCTTCCAAAG |
| | | | CAGCCAGACCCTGACTCACAGGAGCAACGTTCTT |
| | | | TGCTTGAGCCATATCAAAAATGTGGATTGAAGAG |
| | | | AAAGAGTGAGGAAGTGGAGAGCAACGGTAGCGTG |
| | | | CAGATCATAGAAGAACACCCCCCTCTCATGCTGC |
| | | | AGAACAGAACCGTGGTGGGTGCTGCTGCCACGAC |
| | | | CACCACTGTGACCACCAAGAGTAGCAGTTCCAGT |
| | | | GGAGAAGGGGATTACCAGCTGGTCCAGCATGAGA |
| | | | TCCTTTGCTCTATGACCAACAGCTATGAAGTCCT |
| | | | GGAGTTCCTAGGCCGGGGGACATTTGGACAGGTG |
| | | | GCAAAGTGCTGGAAGCGGAGCACCAAGGAAATGT |
| | | | GGCCATTAAGATCTTGAAGAACCACCCCTCCTAT |
| | | | GCCAGACAAGGACAGATTGAAGTGAGCATCCTTC |
| | | | CCGCCTAAGCAGTGAAAATGCTGATGAGTATAAC |
| | | | TTTGTCCGTTCTTATGAGTGTTCAGCACAAGAAT |
| | | | CATACCTGCCTTGTGTTTGAGATGTTGGAGCAGA |
| | | | ACTTGTACGATTTTCTAAAGCAGAACAAGTTTAG |
| | | | CCCACTGCCACTCAAGTACATAAGACCAATCTTG |
| | | | CAGCAGGTGGCCACAGCCCTGATGAAGCTGAAGA |
| | | | GTCTTGGTCTGATTCATGCTGACCTTAAACCTGA |
| | | | CATAATGCTAGTCGATCCAGTTCGCCAACCCTAC |
| | | | CGAGTGAAGGTCATTGACTTTGGTTCTGCTAGTC |
| | | | ATGTTTCCAAAGCCGTGTGTTCAACCTACCTGCA |
| | | | ATCACGCTACTACAGAGCTCCTGAAATATCCTTG |
| | | | GATTACCATTCTGTGAAGCTATTGACATGTGGTC |
| | | | ACTGGGCTGTGTAATAGCTGAGCTGTTCCTGGGA |
| | | | TGGCCTCTTTATTCCTGGTGCTTCAGAATACGAT |
| | | | CAGATTCGCTATATTCACAAACACAAGGCCTGCC |
| | | | AGCTGAGTATCTTCTCAGTGCCGGAACAAAAACA |
| | | | ACCAGGTTTTTTAACAGAGATCCTAATTTGGGGT |
| | | | ACCCACTGTGGAGGCTTAAGACACCTGAAGAACA |
| | | | TGAATTGGAAACTGGAATAAGTCAAAAGAAGCTC |
| | | | GGAAGTACATTTTTAACTGTTTAGATGACATGGC |
| | | | TCAGGTAAATATGTCTACAGACTTAGAGGGGACA |
| | | | GATATGTTAGCAGAGAAAGCAGATCGGAGAGAGT |
| | | | ATATTGATCTTCTAAAGAAAATGCTGACGATTGA |
| | | | TGCAGATAAGAGAATCACGCCTCTGAAGACTCTT |
| | | | AACCACCAATTTGTGACGATGAGTCACCTCCTGG |
| | | | ACTTTCCTCACAGCAGCCACGTTAAGTCCTGTTT |
| | | | CCAGAACATGGAGATCTGCAAGCGGAGGGTTCAC |
| | | | ATGTATGACACAGTGAGTCAGATCAAGAGTCCCT |
| | | | TCACTACACATGTCGCTCCAAATACAAGCACAAA |
| | | | TCTAACCATGAGCTTCAGCAACCAGCTCAACACA |
| | | | GTGCACAATCAGGCCAGTGTTCTAGCTTCCAGCT |
| | | | CTACTGCAGCAGCAGCTACCCTTTCTCTGGCTAA |
| | | | TTCAGATGTCTCGCTGCTAAACTACCAATCGGCT |
| | | | TTGTACCCATCGTCGGCAGCGCCAGTTCCTGGAG |
| | | | TTGCCCAGGAGGGTGTTTCCTTACAACCTGGAAC |
| | | | CACCCAGATCTGCACTCAGACAGATCCATTCCAG |
| | | | CAAACATTTATAGTATGCCCACCTGCTTTTCAGA |
| | | | CTGGACTACAAGCAACAACAAAGCATTCTGGATT |
| | | | CCCTGTGAGGATGGATAATGCTGTGCCAATTGTA |
| | | | CCCCAGGCGCCTGCTGCTCAGCGGCTGCAGATCC |
| | | | AGTCAGGAGTACTCACACAGGGAAGCTGTACACC |
| | | | ACTAATGGTAGCAACTCTCCACCCTCAAGTAGCC |
| | | | ACCATCACGCCGCAGTATGCGGTGCCCTTTACCC |
| | | | TGAGCTGCGCAGCAGGCCGGCCGGCGCTGGTTGA |
| | | | ACAGACTGCTGCTGTACTGCAAGCCTGGCCTGGA |
| | | | GGAACCCAACAAATTCTCCTGCCTTCAGCCTGGC |
| | | | AGCAGCTGCCCGGGGTAGCTCTGCACAACTCTGT |
| | | | CCAGCCTGCTGCAGTGATTCCAGAGGCCATGGGG |
| | | | AGCAGCCAACAGCTAGCTGACTGGAGGAATGCCC |
| | | | ACTCTCATGGCAACCAGTACAGCACTATTATGCA |
| | | | GCAGCCATCTTTGCTGACCAACCATGTGACCTTG |
| | | | GCCACTGCTCAGCCTCTGAATGTTGGTGTTGCCC |
| | | | ATGTGTCAGACAACAACAGTCTAGTTCCCTCCCT |
| | | | TCAAAGAAGAATAAGCAGTCTGCTCCAGTTTCAT |
| | | | CCAAATCCTCTCTGGAAGTCCTGCCTTCTCAAGT |
| | | | TTATTCTCTGGTTGGGAGTAGTCCTCTTCGTACC |
| | | | ACATCTTCTTATAATTCCCTAGTTCCTGTCCAAG |
| | | | ACCAGCATCAGCCAATCATCATTCCAGATACCCC |
| | | | CAGCCCTCCTGTGAGTGTCATCACTATCCGTAGT |
| | | | GACACTGATGAAGAAGAGGACAACAAATACAAGC |
| | | | CCAATAGCTCGAGCCTGAAGGCGAGGTCTAATGT |
| | | | CATCAGTTATGTCACTGTCAATGATTCTCCAGAC |
| | | | TCTGACTCCTCCCTGAGCAGCCCACATCCCACAG |
| | | | ACACTCTGAGTGCTCTGCGGGGCAACAGTGGGAC |
| | | | CCTTCTGGAGGGACCTGGCAGACCTGCAGCAGAT |
| | | | GGCATTGGCACCCGTACTATCATTGTGCCTCCTT |
| | | | TGAAAACACAGCTTGGCGACTGCACTGTAGCAAC |
| | | | ACAGGCCTCAGGTCTCCTTAGCAGTAAGACCAAG |
| | | | CCAGTGGCCTGAGTGAGTGGGCAGTCATCTGGAT |
| | | | GCTGTATCACTCCCACGGGGTACCGGGCTCAGCG |
| | | | AGGGGGAGCCAGCGCGGTGCAGCCACTCAACCTT |
| | | | AGCCAGAACCAGCAGTCATCGTCAGCTTCAACCT |
| | | | CGCAGGAAAGAAGCAGCAACCCTGCTCCCCGCAG |
| | | | ACAGCAGGCATTTGTGGCCCCGCTCTCCCAAGCC |
| | | | CCCTACGCCTTCCAGCATGGCAGCCCACTGCACT |
| | | | CGACGGGGCACCCACACTTGGCCCCAGCCCCTGC |
| | | | TCACCTGCCAAGCCAGCCTCACCTGTATACGTAC |
| | | | GCTGCCCCCACTTCTGCTGCTGCATTGGGCTCCA |
| | | | CCAGTTCCATTGCTCATCTGTTCTCCCCCCAGGG |
| | | | TTCCTCAAGGCATGCTGCAGCTTATACCACACAC |
| | | | CCTAGCACTCTGGTGCATCAGGTTCCTGTCAGTG |
| | | | TCGGGCCAGCCTCCTCACTTCTGCCAGTGTGGCC |
| | | | CCTGCTCAGTACCAACACCAGTTTGCCACTCAGT |
| | | | CCTACATCGGGTCTTCCCGAGGCTCAACAATTTA |
| | | | CACTGGATACCCGCTGAGTCCTACCAAGATCAGT |
| | | | CAGTATTCTTACTTGTAGTTGATGAGCACGAGGA |
| | | | GGGCTCCGTGGCTGCCTGCTAAGTAGCCCTGAGT |
| | | | TCTTAATGGGCTCTGGAGAGCACCTCCATTATCT |
| | | | CCTCTTGAAAGTTCCTAGCCAGCAGCGCGTTCTG |
| | | | CGGGGCCCACTGAAGCAGAAGGCTTTTCCCTGGG |
| | | | AACAGCTCTCGGTGTTGACTGCATTGTTGCAGTC |
| | | | TCCCAAGTCTGCCCTGTTTTTTTAATTCTTTATT |
| | | | CTTGTGACAGCATTTTTGGACGTTGGAAGAGCTC |
| | | | AGAAGCCCATCTTCTGCAGTTACCAAGGAAGAAA |
| | | | GATCGTTCTGAAGTTACCCTCTGTCATACATTTG |
| | | | GTCTCTTTGACTTGGTTTCTATAAATGTTTTTAA |
| | | | AATGAAGTAAAGCTCTTCTTTACGAGGGGAAATG |
| | | | CTGACTTGAAATCCTGTAGCAGATGAGAAAGAGT |
| | | | CATTACTTTTTGTTTGCTTAAAAAACTAAAACAC |
| | | | AAGACTCCTTGTCTTTTATTTTGAAAGCAGCTTA |
| | | | GCAAGGGTGTGCTTATGGCGTATGGAAACAGAAT |
| | | | GATTTCATTTTCATGTCGTGCTGTCCTTACTGGG |
| | | | CAGTTGTTAGAGTTTTAGTACAACGAGTCACTGA |
| | | | AACCTGTGCAGCTGCTGCTGAGCTGCTCGCAGAG |
| | | | CAGCACTGAACAGGCAGCCAGCGCTGCTGGGAAG |
| | | | GAAGGTGAGGGTGAGGACTGTGCCCACCAGGATT |
| | | | CATTCTAAATGAAGACCATGAGTTCAAGTCCTCC |
| | | | TCCTCTCTCTAGTTTAACTTAAATTCTCCTTATA |
| | | | GAAAAGCCAGTGAGGTGGTAAGTGTATGGTGGTG |
| | | | GTTTGCATACAATAGTATGCAAAATCTCTCTCTA |
| | | | GAATGAGATACTGGCACTGATAAACATTGCCTAA |
| | | | GATTTCTATGAATTTCAATAATACACGTCTGTGT |
| | | | TTTCCTCATCTCTCCCTTCTGTTTCATGTGACTT |
| | | | ATTTGAGGGGAAAACTAAAGAAAACTAAACCAGA |
| | | | TAAGTTGTGTATAGCTTTTATACTTTAAAGTAGC |
| | | | TTCCTTTTGTATGCCAACAGCAGAAATTGAAGCT |
| | | | CTTACTAAGACTTATGTAATAAGTGCATGTAGGA |
| | | | ATTGCAGAAAATATTTTAAAAGTTTATTACTGAA |
| | | | TTTAAAAATATTTTAGAAGTTTTGTAATGGTGGT |
| | | | GTTTTAATATTTTGCATAATTAAATATGTACATA |
| | | | TTGATTAGAAGAAATATAACAATTTTTCCTCTAA |
| | | | CCCAAAATGTTATTTGTAATCAAATGTGTAGTGA |
| | | | TTACACTTGAATTGTGTATTTAGTGTGTATCTGA |
| | | | TCCTCCAGTGTTACCCCGGAGATGGATTATGTCT |
| | | | CCATTGTATTTAAACCAAAATGAACTGATACTTG |
| | | | TTGGAATGTATGTGAACTAATTGCAATTCTATTA |
| | | | GAGCATATTACTGTAGTGCTGAGAGAGCAGGGGC |
| | | | ATTGCCTGCAGAGAGGAGACCTTGGGATTGTTTT |
| | | | GCACAGGTGTGTCTGGTGAGGAGTTGTCAGTGTG |
| | | | TGTCTTTTCCTTCCTCCTCTCCTCTCTCCCCTTA |
| | | | TTGTAGTGCCTTATATGATAATGTAGTGGTAATA |
| | | | GAGTTTACAGTGAGCTTGCCTTAGGATGACCAGC |
| | | | AAGCCCCAGTGACCCCAAGCTGTTCGCTGGGATT |
| | | | TAACAGAGCAGGTTGAGTAGCTGTGTTGTGTAAA |
| | | | ATGCGTTCGTGTTCTCAGTCTCCCTACCGACAGT |
| | | | GACAAGTCAAGCCGCAGCTTTCCTCCTTAACTGC |
| | | | CACCTCTGTCCCGTTCCATTTTGGATCTTCAGCT |
| | | | CAGTTCTCACAGAAGCATTCCCTAACGTGGCTCT |
| | | | CTCACTGTGCCTTGCTACCTGGCTGTGAGAGTTC |
| | | | AGGAAGCAGGCGAGAAGAGTGACGCCAGTGCTAA |
| | | | ATATGCATATTTGAAGGTTTGTGCATTACTTAGG |
| | | | GTGGGATTCCTTTTCTCTCCTCCATGTGATATGA |
| | | | TAGTCCTTTCTGCATAGCTGTCGTTTCCTGGTAA |
| | | | ACTTTGCTTGGTTTTTTTTTTTTTTGTTTGTTGT |
| | | | TTTTTTTTTAAAGCATGTAACAGATGTGTTTATA |
| | | | CCAAAGAGCCTGTTGTATTGCTAATATGTCCCAT |
| | | | ACTACGAGAAGGGTTTTGTAGAACTACTGGTGAC |
| | | | AAGAAGCTCACAGAAAGGTTTCTTAATTAGTGAC |
| | | | GAATATGAAAAAGCAAAAGCAAACCTCTTGAATC |
| | | | TGAACAATTCCTGAGGTTTCTTTGGGACAACATG |
| | | | TTGTTCTTGGGGCCCTGCACACTGTAAAATTGTC |
| | | | CTAGTATTCAACCCCTCCATGGATTTGGGTCAAG |
| | | | TTGAAGGTACTAGGGGTGGGGACATTCTTGCCCA |
| | | | TGAGGGATTTGTGGGGAGAAGGTTAACCCTAAGC |
| | | | TACAGAGTGGTCCACCTGAATTAAATTATATCAG |
| | | | AGTGGTAATTCTAGGATGGTTCTGTGTAGGTGGT |
| | | | GTCAGGAGGTGCAGGATGGAGATGGGAGATTTCA |
| | | | TGGAACCCGTTCAGGAAAGCTCTGAACCAGGTGG |
| | | | AACACCGAGGGGCTGTCAACGAACTTGGAGTTTC |
| | | | TTCATCATGGGGAGGAAGAGTTTCCAGGGCAGGG |
| | | | CAGGTAGTCAGTTTAGCCTGCCGGCAACGTGGTG |
| | | | TGTGTTGTCTTTTCTTTAATCATTATATTAAGCT |
| | | | GTGCGTTCAGCAGTCTGTTGGTTGAGATAACCAC |
| | | | GCATCATTGTGTAGTTTGTCACTAGTGTTATACC |
| | | | GTTTATGTCATTCTGTGTGTGATCTTTGTGTTTC |
| | | | CTTTCCCCCAAGCATTCTGGGTTTTTCCTATTTA |
| | | | AATACAGTTCTAGTTTCTAGGGCAAACATTTTTT |
| | | | TTAACCTTTTCTCTATAAGGGACAAGATTTATTG |
| | | | TTTTTATAGGAATGAGATGCAGGGAAAAAACAAA |
| | | | CCAACCCTGTCCCCACTCCTCACCTCCCTAATCC |
| | | | AATAAGCAGTTATTGAAGATGGGAGTCTTAAATT |
| | | | TATGGGAAAGAGGATGCCTAGGAGTTTGCATCGT |
| | | | TACCTGAGACATCTGGCTAGCAGTGTGACTTTAC |
| | | | AGACTTTGAGGTTGTCACTCTGCAAACTGACATT |
| | | | TCAGATTTTCCTAGATAACCCATCTGTGTCTGCT |
| | | | GAATGTGTATGCGCCAGACATAGTTTTACATTCA |
| | | | TTCTGGCCTGGGGCTTAACATTGACTGCTTGCCC |
| | | | TGATGGCATGGAGGAGAGCCCTACGAACATAGCG |
| | | | CTGACTAGGTCAGCATTGCCTGACCTTGGAACAG |
| | | | CTTAAGGCTTTAAACCTTCTCTTAGAACGTGCAT |
| | | | TTCCAGTTTCTCCCTTCCCAGGTGAGAGAGGAAC |
| | | | TGGAAGGGTTGCATAGGCACACACCAGGACACTT |
| | | | AGTCACTCCAGAGTCCCCAGTTGCAACTAGGAGG |
| | | | TGGTTACCCTGTTAACCCCAGGAAGAAGAACCGC |
| | | | ATTTCAAACAGTTCCGGCCATTGAGAGCCTGCTT |
| | | | TTGTGGTTGCTCATCCGTCATCATCCGCTAGAGG |
| | | | GGCTTAGCCAGGCCAGCACAGTACTGGCTGTCCT |
| | | | ATCTGCATTAGTATGCAGGAATTTACTAGTTGAG |
| | | | ATGGTTTGTTTTAGGATAGGAGATGAAATTGCCT |
| | | | TTCGGTGACAGGAATGGCCAAGCCTGCTTTGTGT |
| | | | TTTTTTTTAAATGATGGATGGTGCAGCATGTTTC |
| | | | CAAGTTTCCATGGTTGTTTGTTGCTAAAATTTAT |
| | | | ATAATGTGTGGTTTCAATTCAATTCAGCTTGAAA |
| | | | AATAATTTCACTATATGTAGCAGTACATTATATG |
| | | | TACATTATATGTAATGTTAGTATTTTTGCTTTGA |
| | | | ATCCTTGATATTGCAATGGAATTCCTAATTTATT |
| | | | AAATGTATTTGATATGCTAAAAAA |
| | | 197 | MASQLQVFSPPSVSSSAFCSAKKLKIEPSGWDVS |
| | | | GQSSNDKYYTHSKTLPATQGQASSSHQVANFNLP |
| | | | AYDQGLLLPAPAVEHIVVTAADSSGSAATATFQS |
| | | | SQTLTHRSNVSLLEPYQKCGLKRKSEEVESNGSV |
| | | | QIIEEHPPLMLQNRTVVGAAATTTTVTTKSSSSS |
| | | | GEGDYQLVQHEILCSMTNSYEVLEFLGRGTFGQV |
| | | | AKCWKRSTKEIVAIKILKNHPSYARQGQIEVSIL |
| | | | SRLSSENADEYNFVRSYECFQHKNHTCLVFEMLE |
| | | | QNLYDFLKQNKFSPLPLKYIRPILQQVATALMKL |
| | | | KSLGLIHADLKPENIMLVDPVRQPYRVKVIDFGS |
| | | | ASHVSKAVCSTYLQSRYYRAPEIILGLPFCEAID |
| | | | MWSLGCVIAELFLGWPLYPGASEYDQIRYISQTQ |
| | | | GLPAEYLLSAGTKTTRFFNRDPNLGYPLWRLKTP |
| | | | EEHELETGIKSKEARKYIFNCLDDMAQVNMSTDL |
| | | | EGTDMLAEKADRREYIDLLKKMLTIDADKRITPL |
| | | | KTLNHQFVTMSHLLDFPHSSHVKSCFQNMEICKR |
| | | | RVHMYDTVSQIKSPFTTHVAPNTSTNLTMSFSNQ |
| | | | LNTVHNQASVLASSSTAAAATLSLANSDVSLLNY |
| | | | QSALYPSSAAPVPGVAQQGVSLQPGTTQICTQTD |
| | | | PFQQTFIVCPPAFQTGLQATTKHSGFPVRMDNAV |
| | | | PIVPQAPAAPQLQIQSGVLTQGSCTPLMVATLHP |
| | | | QVATITPQYAVPFTLSCAAGRPALVEQTAAVLQA |
| | | | WPGGTQQILLPSAWQQLPGVALHNSVQPAAVIPE |
| | | | AMGSSQQLADWRNAHSHGNQYSTIMQQPSLLTNH |
| | | | VTLATAQPLNVGAHVVRQQQSSSLPSKKNKQSAP |
| | | | VSSKSSLEVLPSQVYSLVGSSPLRTTSSYNSLVP |
| | | | VQDQHQPIIIPDTPSPPVSVITIRSDTDEEEDNK |
| | | | YKPNSSSLKARSNVISYVTVNDSPDSDSSLSSPH |
| | | | PTDTLSALRGNSGTLLEGPGRPAADGIGTRTIIV |
| | | | PPLKTQLGDCTVATQASGLLSSKTKPVASVSGQS |
| | | | SGCCIPTTGYRAQRGGASAVQPLNLSQNQQSSSA |
| | | | STSQERSSNPAPRRQQAFVAPLSQAPYAFQHGSP |
| | | | LHSTGHPHLAPAPAHLPSQPHLYTYAAPTSAAAL |
| | | | GSTSSIAHLFSPQGSSRHAAAYTTHPSTLVHQVP |
| | | | VSVGPSLLTSASVAPAQYQHQFATQSYIGSSRGS |
| | | | TIYTGYPLSPTKISQYSYL |

Also suitable for use in the present invention is the sequence provided in Genbank Accession No. AF077658.

A contig assembled from the human EST database by the NCBI having homology with all or parts of a HIPK1 nucleic acid sequence of the invention is depicted in Table 17 as SEQ ID NO. 198. SEQ ID NO. 199 depicts the amino acid sequence of a open reading frame of SEQ ID NO. 198 which encodes the C-terminal portion of human HIPK1 protein.

TABLE 17

MOUSE

| | | |
|---|---|---|
| SAGRES | REF | SEQ |

| | | | |
|---|---|---|---|
| TAG # | # | ID # | SEQUENCE |

| | | | |
|---|---|---|---|
| S000013 | F30 | 198 | CACACCGCAGTATGCGGTGCCCTTTACTCTGAGC |
| | | | TGCGCAGCCGGCCGGCCGGCGCTGGTTGAACAGA |
| | | | CTGCCGCTGTACTGGCGTGGCCTGGAGGGACTCA |
| | | | GCAAATTCTCCTGCCTTCAACTTGGCAACAGTTG |
| | | | CCTGGGGTAGCTCTACACAACTCTGTCCAGCCCA |
| | | | CAGCAATGATTGCAGAGGCCATGGGGAGTGGACA |
| | | | GCAGCTAGCTGACTGGAGGAATGCCCACTCTCAT |
| | | | GGCAACCAGTACAGCACTATCATGCAGCAGCCAT |
| | | | CCTTGCTGACTAACCATGTGACATTGGCCACTGC |
| | | | TCAGCCTCTGAATGTTGGTGTTGCCCATGTTGTC |
| | | | AGACAACAACAATCCAGTTCCCTCCCTTCGAAGA |
| | | | AGAATAAGCAGTCAGCTCCAGTCTCAACCAAGTC |
| | | | CTCTCTAGATGTTCTGCCTTCCCAAGTCTATTCT |
| | | | CTGGTTGGGAGCAGTCCCCTCCGCACCACATCTT |
| | | | CTTATAATTCCTTGGTCCCTGTCCAAGATCAGCA |
| | | | TCAGCCCATCATCATTCCAGATACTCCCAGCCCT |
| | | | CCTGTGAGTGTCATCACTATCCGAAGTGACACTG |
| | | | ATGAGGAAGAGGACAACAAATACAAGCCCAGTAG |
| | | | CTCTGGACTGAAGCCAAGGTCTAATGTCATCAGT |
| | | | TATGTCACTGTCAATGATTCTCCAGACTCTGACT |
| | | | CTTCTTTGAGCAGCCCTTATTCCACTGATACCCT |
| | | | GAGTGCTCTCCGAGGCAATAGTGGATCCGTTTTG |
| | | | GAGGGGCCTGGCAGAGTTGTGGCAGATGGCACTG |
| | | | GCACCCGCACTATCATTGTGCCTCCACTGAAAAC |
| | | | TCAGCTTGGTGACTGCACTGTAGCAACCCAGGCC |
| | | | TCAGGTCTCCTGAGCAATAAGACTAAGCCAGTCG |
| | | | CTTCAGTGAGTGGGCAGTCATCTGGATGCTGTAT |
| | | | CACCCCCACAGGGTATCGAGCTCAACGCGGGGGG |
| | | | ACCAGTGCAGCACAACCACTCAATCTTAGCCAGA |
| | | | ACCAGCAGTCATCGGCGGCTCCAACCTCACAGGA |
| | | | GAGAAGCAGCAACCCAGCCCCCCGCAGGCAGCAG |
| | | | GCGTTTGTGGCCCCTCTCTCCCAAGCCCCCTACA |
| | | | CCTTCCAGCATGGCAGCCCGCTACACTCGACAGG |
| | | | GCACCCACACCTTGCCCCGGCCCCTGCTCACCTG |
| | | | CCAAGCCAGGCTCATCTGTATACGTATGCTGCCC |
| | | | CGACTTCTGCTGCTGCACTGGGCTCAACCAGCTC |
| | | | CATTGCTCATCTTTTCTCCCCACAGGGTTCCTCA |
| | | | AGGCATGCTGCAGCCTATACCACTCACCCTAGCA |
| | | | CTTTGGTGCACCAGGTCCCTGTCAGTGTTGGGCC |
| | | | CAGCCTCCTCACTTCTGCCAGCGTGGCCCCTGCT |
| | | | CAGTACCAACACCAGTTTGCCACCCAATCCTACA |
| | | | TTGGGTCTTCCCGAGGCTCAACAATTTACACTGG |
| | | | ATACCCGCTGAGTCCTACCAAGATCAGCCAGTAT |
| | | | TCCTACTTATAGTTGGTGAGCATGAGGGAGGAGG |
| | | | AATCATGGCTACCTTCTCCTGGCCCTGCGTTCTT |
| | | | AATATTGGGCTATGGAGAGATCCTCCTTTACCCT |
| | | | CTTGAAATTTCTTAGCCAGCAACTTGTTCTGCAG |
| | | | GGGCCCACTGAAGCAGAAGGTTTTTCTCTGGGGG |
| | | | AACCTGTCTCAGTGTTGACTGCATTGTTGTAGTC |
| | | | TTCCCAAAGTTTGCCCTATTTTTAAATTCATTAT |
| | | | TTTTGTGACAGTAATTTTGGTACTTGGAAGAGTT |
| | | | CAGATGCCCATCTTCTGCAGTTACCAAGGAAGAG |
| | | | AGATTGTTCTGAAGTTACCCTCTGAAAAATATTT |
| | | | TGTCTCTCTGACTTGATTTCTATAAATGCTTTTA |
| | | | AAAACAAGTGAAGCCCCTCTTTATTTCATTTTGT |
| | | | GTTATTGTGATTGCTGGTCAGGAAAAATGCTGAT |
| | | | AGAAGGAGTTGAAATCTGATGACAAAAAAAGAAA |
| | | | AATTACTTTTTGTTTGTTTATAAACTCAGACTTG |
| | | | CCTATTTTATTTTAAAAGCGGCTTACACAATCTC |
| | | | CCTTTTGTTTATTGGACATTTAAACTTACAGAGT |
| | | | TTCAGTTTTGTTTTAATGTCATATTATACTTAAT |
| | | | GGGCAATTGTTATTTTTGCAAAACTGGTTACGTA |
| | | | TTACTCTGTGTTACTATTGAGATTCTCTCAATTG |
| | | | CTCCTGTGTTTGTTATAAAGTAGTGTTTAAAAGG |
| | | | CAGCTCACCATTTGCTGGTAACTTAATGTGAGAG |
| | | | AATCCATATCTGCGTGAAAACACCAAGTATTCTT |
| | | | TTTAAATGAAGCACCATGAATTCTTTTTTAAATT |
| | | | ATTTTTTAAAAGTCTTTCTCTCTCTGATTCAGCT |
| | | | TAAATTTTTTTATCGAAAAAGCCATTAAGGTGGT |
| | | | TATTATTACATGGTGGTGGTGGTTTTATTATATG |
| | | | CAAAATCTCTGTCTATTATGAGATACTGGCATTG |
| | | | ATGAGCTTTGCCTAAAGATTAGTATGAATTTTCA |
| | | | GTAATACACCTCTGTTTTGCTCATCTCTCCCTTC |
| | | | TGTTTTATGTGATTTGTTTGGGGAGAAAGCTAAA |
| | | | AAAACCTGAAACCAGATAAGAACATTTCTTGTGT |
| | | | ATAGCTTTTATACTTCAAAGTAGCTTCCTTTGTA |
| | | | TGCCAGCAGCAAATTGAATGCTCTCTTATTAAGA |
| | | | CTTATATAATAAGTGCATGTAGGAATTGCAAAAA |
| | | | ATATTTTAAAAATTTATTACTGAATTTAAAAATA |
| | | | TTTTAGAAGTTTTGAACAAGCAATTTTTCCTGCT |
| | | | AACCCAAAATGTTATTTGTAATCAAATGTGTAGT |
| | | | GATTACACTTGAATTGTGTACTTAGTGTGTATGT |
| | | | GATCCTCCAGTGTTATCCCGGAGATGGATTGATG |
| | | | TCTCCATTGTATTTAAACCAAAATGAACTGATAC |
| | | | TTGTTGGAATGTATGTGAACTAATTGCAATTATA |
| | | | TTAGAGCATATTACTGTAGTGCTGAATGAGCAGG |
| | | | GGCATTGCCTGCAAGGAGAGGAGACCCTTGGAAT |
| | | | TGTTTTGCACAGGTGTGTCTGGTGAGGAGTTTTT |
| | | | CAGTGTGTGTCTCTTCCTTCCCTTTCTTCCTCCT |
| | | | TCCCTTATTGTAGTGCCTTATATGATAATGTAGT |
| | | | GGTTAATAGAGTTTACAGTGAGCTTGCCTTAGGA |
| | | | TGGACCAGCAAGCCCCCGTGGACCCTAAGTTGTT |
| | | | CACCGGGATTTATCAGAACAGGATTAGTAGCTGT |
| | | | ATTGTGTAATGCATTGTTCTCAGTTTCCCTGCCA |
| | | | ACATTGAAAAATAAAAACAGCAGCTTTTCTCCTT |
| | | | TACCACCACCTCTACCCCTTTCCATTTTGGATTC |
| | | | TCGGCTGAGTTCTCACAGAAGCATTTTCCCCATG |
| | | | TGGCTCTCTCACTGTGCGTTGCTACCTTGCTTCT |
| | | | GTGAGAATTCAGGAAGCAGGTGAGAGGAGTCAAG |
| | | | CCAATATTAAATATGCATTCTTTTAAAGTATGTG |
| | | | CAATCACTTTTAGAATGAATTTTTTTTTCCTTTT |
| | | | CCCATGTGGCAGTCCTTCCTGCACATAGTTGACA |
| | | | TTCCTAGTAAAATATTTGCTTGTTGAAAAAAACA |
| | | | TGTTAACAGATGTGTTTATACCAAAGAGCCTGTT |
| | | | GTATTGCTTACCATGTCCCCATACTATGAGGAGA |
| | | | AGTTTTGTGGTGCCGCTGGTGACAAGGAACTCAC |
| | | | AGAAAGGTTTCTTAGCTGGTGAAGAATATAGAGA |
| | | | AGGAACCAAAGCCTGTTGAGTCATTGAGGCTTTT |
| | | | GAGGTTTCTTTTTTAACAGCTTGTATAGTCTTGG |
| | | | GGCCCTTCAAGCTGTGAAATTGTCCTTGTACTCT |
| | | | CAGCTCCTGCATGGATCTGGGTCAAGTAGAAGGT |
| | | | ACTGGGGATGGGGACATTCCTGCCCATAAAGGAT |
| | | | TTGGGGAAAGAGATTCCTAATCCTAAAACAGGTG |
| | | | TGTTCCATCCGAATTGAAAATGATATATTTGAGA |
| | | | TATAATTTTAGGACTGGTTCTGTGTAGATAGAGA |
| | | | TGGTGTCAAGGAGGTGCAGGATGGAGATGGGAGA |
| | | | TTTCATGGAGCCTGGTCAGCCAGCTCTGTACCAG |
| | | | GTTGAACACCGAGGAGCTGTCAAAGTATTTGGAG |
| | | | TTTCTTCATTGTAAGGAGTAAGGGCTTCCAAGAT |
| | | | GGGGCAGGTAGTCCGTACAGCCTACCAGGAACAT |
| | | | GTTGTGTTTTCTTTATTTTTTAAAATCATTATAT |
| | | | TGAGTTGTGTTTTCAGCACTATATTGGTCAAGAT |
| | | | AGCCAAGCAGTTTGTATAATTTCTGTCACTAGTG |
| | | | TCATACAGTTTTCTGGTCAACATGTGTGATCTTT |
| | | | GTGTCTCCTTTTTGCCAAGCACATTCTGATTTTC |
| | | | TTGTTGGAACACAGGTCTAGTTTCTAAAGGACAA |
| | | | ATTTTTTGTTCCTTGTCTTTTTTCTGTAAGGGAC |
| | | | AAGATTTGTTGTTTTTGTAAGAAATGAGATGCAG |
| | | | GAAAGAAAACCAAATCCCATTCCTGCACCCCAGT |
| | | | CCAATAAGCAGATACCACTTAAGATAGGAGTCTA |
| | | | AACTCCACAGAAAAGGATAATACCAAGAGCTTGT |
| | | | ATTGTTACCTTAGTCACTTGCCTAGCAGTGTGTG |
| | | | GCTTTAAAAACTAGAGATTTTTCAGTCTTAGTCT |
| | | | GCAAACTGGCATTTCCGATTTTCCAGCATAAAAA |
| | | | TCCACCTGTGTCTGCTGAATGTGTATGTATGTGC |
| | | | TCACTGTGGCTTTAGATTCTGTCCCTGGGGTTAG |
| | | | CCCTGTTGGCCCTGACAGGAAGGGAGGAAGCCTG |
| | | | GTGAATTTAGTGAGCAGCTGGCCTGGGTCACAGT |
| | | | GACCTGACCTCAAACCAGCTTAAGGCTTTAAGTC |
| | | | CTCTCTCAGAACTTGGCATTTCCAACTTCTCCTT |
| | | | TCCGGGTGAGAGAAGAAGCGGAAGAAGGGTTCAG |
| | | | TGTAGCCACTCTGGGCTCATAGGGACACTTGGTC |
| | | | ACTCCAGAGTTTTTAATAGCTCCCAGGAGGTGAT |
| | | | ATTATTTTCAGTGCTCAGCTGAAATACCAACCCC |
| | | | AGGAATAAGAACTCCATTTCAAACAGTTCTGGCC |
| | | | ATTCTGAGCCTGCTTTTGTGATTGCTCATCCATT |
| | | | GTCCTCCACTAGAGGGGCTAAGCTTGACTGCCCT |
| | | | TAGCCAGGCAAGCACAGTAATGTGTGTTTTGTTC |
| | | | AGCATTATTATGCAAAAATTCACTAGTTGAGATG |
| | | | GTTTGTTTTAGGATAGGAAATGAAATTGCCTCTC |
| | | | AGTGACAGGAGTGGCCCGAGCCTGCTTCCTATTT |
| | | | TGATTTTTTTTTTTTTTAACTGATAGATGGTGCA |
| | | | GCATGTCTACATGGTTGTTTGTTGCTAAACTTTA |
| | | | TATAATGTGTGGTTTCAATTCAGCTTGAAAAATA |
| | | | ATCTCACTACATGTAGCAGTACATTATATGTACA |
| | | | TTATATGTAATGTTAGTATTTCTGCTTTGAATCC |
| | | | TTGATATTGCAATGGAATTCCTACTTTATTAAAT |
| | | | GTATTTGATATGCTAGTTATTGTGTGCGATTTAA |
| | | | ACTTTTTTTGCTTTCTCCCTTTTTTTGGTTGTGC |
| | | | GCTTTCTTTTACAACAAGCCTCTAGAAACAGATA |
| | | | GTTTCTGAGAATTACTGAGCTATGTTTGTAATGC |
| | | | AGATGTACTTAGGGAGTATGTAAAATAATCATTT |
| | | | TAACAAAAGAAATAGATATTTAAAATTTAATACT |
| | | | AACTATGGGAAAAGGGTCCATTGTGTAAAACATA |
| | | | GTTTATCTTTGGATTCAATGTTTGTCTTTGGTTT |
| | | | TACAAAGTAGCTTGTATTTTCAGTATTTTCTACA |
| | | | TAATATGGTAAAATGTAGAGCAATTGCAATGCAT |
| | | | CAATAAAATGGGTAAATTTTCTG |
| | | 199 | TPQYAVPFTLSCAAGRPALVEQTAAVLAWPGGTQ |
| | | | QILLPSTWQQLPGVALHNSVQPTAMIPEAMGSGQ |
| | | | QLADWRNAHSHGNQYSTIMQQPSLLTNHVTLATA |
| | | | QPLNVGVAHVVRQQQSSSLPSKKNKQSAPVSSKS |
| | | | SLDVLPSQVYSLVGSSPLRVISSYNSLVPVQDQH |
| | | | QPIIIPDTPSPPVSVITIRSDTDEEEDNKYKPSS |
| | | | SGLKPRSNVISYVTVNDSPDSDSSLSSPYSTDTL |
| | | | SALRGNSGSVLEGPGRVVADGTGTRTIIVPPLKT |
| | | | QLGDCTVATQASGLLSNKTKPVASVSGQSSGCCI |
| | | | TPTGYRAQRGGTSAAdPLNLSQNQQSSAAPTSQE |
| | | | RSSNPAPRRQQAFVAPLSQAPYTFQHGSPLHSTG |
| | | | HPHLAPAPAHLPSQAHLYTYAAPTSAAALGSTSS |
| | | | IAHLFSPQGSSRHAAAYTTHPSTLVHQVPVSVGP |
| | | | SLLTSASVAPAQYQHQFATQSYIGSSRGSTIYTG |
| | | | YPLSPTKSQYSYL |

The JAKI nucleic acid sequences of the invention are depicted in Tables 18 and 19. The nucleic acid sequence shown in Table 18 is from mouse. The nucleic acid sequence shown in Table 19 is from human. The nucleic acid sequence shown in Table 22 is Sagres Tag No. S00039. The JAKI amino acid sequences are shown in Tables 20 and 21. Table 20 shows the amino acid sequence from mouse and Table 21 shows the amino acid sequence from human.

TABLE 18

JAK1 Nucleotide Sequence from Mouse

| | |
|---|---|
| Sagres | Seq. |
| Tag | ID |

| | |
|---|---|
| No. | No. |

| | | |
|---|---|---|
| S00039 | 200 | CAGCCGCGGAGTAGCCGGCAGCCGCTGACGCGCCGCGGG |
| | | TCCGCCCCAGCCTCGCTCGTCCTTTCGGTGCCTCTCCTT |
| | | AGCCGCGGGTGTCCACGCCGGACCCTGCACGGCAGGCTG |
| | | AGTTGCCTGCCAGACTCCTGACCCAGATCGACCCTGCGC |
| | | CAAGGAGCCGCGCGGCCCGGCGCACACGGAAGTGATCAG |
| | | CTCTGAATGGGCTTTGGAAGGTAAGAAGAAAAATCCAGT |
| | | CTGCTTTCAGGACACTGGACAACCGAATAAATGCAGTAT |
| | | CTAAATATAAAAGAGGACTGCAATGCCATGGCGTTCTGT |
| | | GCTAAAATGAGGAGCTTCAAGAAGACTGAGGTGAAGCAG |
| | | GTGGTCCCTGAGCCTGGAGTGGAGGTGACTTTCTATCTG |
| | | TTGGACAGGGAGCCCCTCCGCCTGGGCAGCGGAGAGTAT |
| | | ACAGCCGAGGAGCTGTGCATCAGGGCCGCCCAGGAGTGC |
| | | AGTATCTCTCCTCTCTGTCACAACCTCTTCGCCCTGTAC |
| | | GATGAGAGCACCAAGCTCTGGTACGCTCCGAACCGAATC |
| | | ATCACTGTGGATGACAAAACGTCTCTCCGGCTCCACTAC |
| | | CGCATGAGGTTCTACTTTACCAACTGGCACGGAACCAAT |
| | | GACAACGAACAGTCTGTATGGCGACATTCTCCAAAGAAG |
| | | CAGAAAAACGGCTATGAGAAGAAAAGGGTTCCAGAAGCA |
| | | ACCCCACTCCTTGATGCCAGTTCACTGGAGTATCTGTTT |
| | | GCACAGGGACAGTATGATTTGATCAAATGCCTGGCTCCC |
| | | ATTCGGGACCCCAAGACGGAGCAAGACGGACATGATATT |
| | | GAAAATGAGTGCCTGGGCATGGCGGTCCTGGCCATCTCC |
| | | CACTATGCCATGATGAAGAAGATGCAGTTGCCGGAACTT |
| | | CCCAAAGACATCAGCTACAAGCGATATATTCCAGAAACA |
| | | TTGAATAAATCCATCAGACAGAGGAACCTTCTTACCAGG |
| | | ATGCGAATAAATAATGTTTTCAAGGATTTCTTGAAGGAA |
| | | TTTAACAACAAGACCATCTGTGACAGCAGTGTGCATGAC |
| | | CTGAAGGTGAAATACCTGGCTACCTTGGAAACTTCTACA |
| | | TTGACAAAACATTATGGAGCTGAAATATTGAGACTTCTA |
| | | TGCTACTGATTTCATCAGAAAATGAATTGAGTCGATGCC |
| | | ATTCGAATGACAGTGGCAATGTTCTCTATGAGGTCATGG |
| | | TGACTGGAAATCTCGGGATCCAGTGGCGGCAGAAACCAA |
| | | ATGTTGTTCCTGTTGAAAAGGAAAAAAATAAACTGAAGC |
| | | GGAAAAAACTGGAATATAATAAACACAAGAAGGATGATG |
| | | AGAGAAACAAACTCCGGGAAGAGTGGAACAATTTTTCCT |
| | | ATTTCCCTGAAATCACCCACATTGTAATAAAGGAGTCTG |
| | | TGGTCAGCATTAACAAACAGGACAACAAAAACATGGAAC |
| | | TCAAGCTCTCTTCTCGAGAGGAAGCCTTGTCCTTTGTGT |
| | | CCCTGGTGGATGGCTACTTCCGGCTCACTGCAGATGCCC |
| | | ACCATTACCTCTGTACTGATGTGGCTCCCCCACTGATTG |
| | | TCCACAATATACAGAACGGCTGCCACGGTCCAATCTGCA |
| | | CAGAATATGCCATCAATAAGCTGCGGCAGGAAGGGAGTG |
| | | AAGAGGGGATGTACGTGCTGAGGTGGAGCTGCACCGACT |
| | | TTGACAACATTCTTATGACTGTCACCTGCTTTGAAAAGT |
| | | CTGAGGTATTGGGTGGCCAGAAGCAGTTCAAGAACTTTC |
| | | AGATTGAGGTACAGAAGGGCCGCTACAGCCTGCATGGCT |
| | | CTATGGACCACTTTCCCAGCCTGCGAGACCTCATGAACC |
| | | ACCTCAAGAAGCAGATCCTGCGCACGGACAACATAAGCT |
| | | TTGTGCTGAAACGATGCTGTCAGCCTAAGCCTCGAGAAA |
| | | TCTCCAATCTGCTCGTAGCCACTAAGAAAGCCCAGGAGT |
| | | GGCAGCCTGTCTACTCCATGAGCCAGCTGAGCTTTGATC |
| | | GGATCCTTAAGAAAGATATTATACAAGGTGAGCACCTTG |
| | | GCAGAGGCACAAGAACACATATCTATTCTGGGACCCTGC |
| | | TGGACTACAAGGATGAGGAAGGAATTGCTGAAGAGAAGA |
| | | AGATAAAAGTGATCCTCAAAGTCCTAGACCCCAGCCACC |
| | | GGGACATCTCTCTGGCCTTCTTTGAGGCTGCTAGCATGA |
| | | TGAGACAGGTTTCCCACAAACATATAGTGTACCTCTACG |
| | | GCGTGTGTGTCCGAGATGTGGAAAATATCATGGTGGAAG |
| | | AGTTTGTGGAGGGGGGGCCGTTGGATCTCTTCATGCACC |
| | | GGAAAGTGATGCGCTTACTACCCCCTGGAAGTTCAAGGT |
| | | TGCCAAACAGCTGGCCAGTGCCCTGAGTTACTTGGAAGA |
| | | TAAAGACCTGGTTCATGGAAATGTGTGCACTAAAAACCT |
| | | CCTTCTGGCCCGTGAGGGCATTGACAGTGACATTGGCCC |
| | | GTTCATCAAGCTTAGTGACCTGGCATCCCAGTCTCTGTG |
| | | CTGACCAGGCAAGAGTGCATAGAGCGAATCCCCTGGATC |
| | | GCTCCTGAGTGTGTTGAAGACTCCAAGAACCTGAGTGTG |
| | | GCTGCTGACAAGTGGAGCTTTGGAACCACGCTCTGGGAA |
| | | ATCTGCTACAACGGAGAGATTCCTCTCAAAGACAAGACC |
| | | CTCATTGAGAAAGAGAGGTTTTATGAAAGCCGCTGCAGG |
| | | CCTGTGACTCCATCTTGCAAGGAGCTAGCTGACCTCATG |
| | | ACTCGCTGCATGAACTATGACCCCAACCAGAGACCCTTC |
| | | TTCCGAGCCATCATGAGGGACATTAACAAGCTGGAGGAG |
| | | CAGAATCCAGACATTGTTTCAGAAAAGCAGCCAACAACA |
| | | GAGGTGGACCCCACTCACTTTGAAAAGCGGTTCCTGAAG |
| | | AGGATTCGTGACTTGGGAGAGGGTCACTTTGGGAAGGTT |
| | | GAGCTCTGCAGATATGATCCTGAGGGAGACAACACAGGG |
| | | GAGCAGGTAGCTGTCAAGTCCCTGAAGCCTGAGAGTGGA |
| | | GGTAACCACATAGCTGATCTGAAGAAGGAGATAGAGATC |
| | | TTACGGAACCTCTACCATGAGAACATTGTGAAGTACAAA |
| | | GGAATCTGCATGGAAGACGGAGGCAATGGTATCAAGCTC |
| | | ATCATGGAGTTTCTGCCTTCGGGAAGCCTAAAGGAGTAT |
| | | CTGCCAAAGAATAAGAACAAAATCAACCTCAAACAGCAG |
| | | CTAAATATGCCATCCAGATTTGTAAGGGGATGGACTACT |
| | | TGGGTTCTCGGCAATACGTTCACCGGGACTTAGCAGCAA |
| | | GAAATGTCCTTGTTGAGAGTGAGCATCAAGTGAAGATCG |
| | | GAGACTTTGGTTTAACCAAAGCAAATTGAACCGATAAGG |
| | | AGTACTACACAGTCAAGGACGACCGGGACAGCCCAGTGT |
| | | TCTGGTACGCTCCGGAATGTTTAATCCAGTGTAAATTTT |
| | | ATATCGCCTCTGATGTCTGGTCTTTTGGAGTGACACTGC |
| | | ACGAGCTGCTCACTTACTGTGACTCAGATTTTAGTCCCA |
| | | TGGCCTTGTTCCTGAAAATGATAGGCCCAACTCATGGCC |
| | | AGATGACAGTGACACGGCTTGTGAAGACTCTGAAAGAAG |
| | | GAAAGCGTCTGCCATGTCCACCCAACTGTCCTGATGAGG |
| | | TTTATCAGCTTATGAGAAAATGCTGGGAATTCCAACCAT |
| | | CTAACCGGACAACTTTTCAGAACCTTATTGAAGGATTTG |
| | | AAGCACTTTTAAAATAAGAAGCATGAACAACATTTAAAT |
| | | TCCCATTTATCAAATCCTTCTCTCCCAAGCCATTTAAAA |
| | | ACGTTTTTTAAGTGAAAAGTTTGTATTCTGCCTCTAAAG |
| | | TTCCTCAACAAATACTCGAGTTACACATATGCATATGTC |
| | | ACACTGTCACTCAGTGTGTGGATATGCCTATGTCACACT |
| | | GTCACTCAGTGTGTGGAACTTTCTCTTTAAAGGTGTAAC |
| | | ATCTTAAATTTGGTGATGAATAGTGACAACCAAAAGACT |
| | | AGATTGTGCCTAAGCACTCCTTCTGGAACAACCGAATGA |
| | | TCAGCTGCATAGCAAAGGACTGTGCCGCTGGCATATTGA |
| | | TCTCAGATAAAACTTGTGGACTTGGCTGACACTCTCCCT |
| | | TGCCCTGAAATCTCAATGTCTATTCAGTGATAGTACAAG |
| | | CACGTAGATACCACTTAGTATACTATTGTTTCTATTAAA |
| | | AAAAAAAAAAA |

TABLE 19

JAK1 Nucleotide Sequence from Human

| | |
|---|---|
| Sagres | Seq. |
| Tag | ID |

| | |
|---|---|
| No. | No. |

| | | |
|---|---|---|
| S00039 | 201 | TCCAGTTTGCTTCTTGGAGAACACTGGACAGCTGAATAA |
| | | ATGCAGTATCTAAATATAAAAGAGGACTGCAATGCCATG |
| | | GCTTTCTGTGCTAAAATGAGGAGCTCCAAGAAGACTGAG |
| | | GTGAACCTGGAGGCCCCTGAGCCAGGGGTGGAAGTGATC |
| | | TTCTATCTGTCGGACAGGGAGCCCCTCCGGCTGGGCAGT |
| | | GGAGAGTACACAGCAGAGGAACTGTGCATCAGGGCTGCA |
| | | CAGGCATGCCGTATCTCTCCTCTTTGTCACAACCTCTTT |
| | | GCCCTGTATGACGAGAACACCAAGCTCTGGTATGCTCCA |
| | | AATCGCACCATCACCGTTGATGACAAGATGTCCCTCCGG |
| | | CTCCACTACCGGATGAGGTTCTATTTCACCAATTGGCAT |
| | | GGAACCAACGACAATGAGCAGTCAGTGTGGCGTCATTCT |
| | | CCAAAGAAGCAGAAAAATGGCTACGAGAAAAAAAAGATT |
| | | CCAGATGCAACCCCTCTCCTTGATGCCAGCTCACTGGAG |
| | | TATCTGTTTGCTCAGGGACAGTATGATTTGGTGAAATGC |
| | | CTGGCTCCTATTCGAGACCCCAAGACCGAGCAGGATGGA |
| | | CATGATATTGAGAACGAGTGTCTAGGGATGGCTGTCCTG |
| | | GCCATCTCACACTATGCCATGATGAAGAAGATGCAGTTG |
| | | CCAGAACTGCCCAAGGACATCAGGTAAAGCGATATATTC |
| | | CAGAAACATTGAATAAGTCCATCAGACAGAGGAACCTTC |
| | | TCACCAGGATGCGGATAAATAATGTTTTCAAGGATTTCC |
| | | TAAAGGAATTTAACAACAAGACCATTTGTGACAGCAGCG |
| | | TGTCCACGCATGACCTGAAGGTGAAATACTTGGCTACCT |
| | | TGGAAACTTTGACAAAACATTACGGTGCTGAAATATTTG |
| | | AGACTTCCATGTTACTGATTTCATCAGAAAATGAGATGA |
| | | ATTGGTTTCATTCGAATGACGGTGGAACGTTCTCTACTA |
| | | CGAAGTGATGGTGACTGGGAATCTTGGAATCCAGTGGAG |
| | | GCATAAACCAAATGTTGTTTCTGTTGAAAAGGAAAAAAA |
| | | TAAACTGAAGCGGAAAAAACTGGAAAATAAACACAAGAA |
| | | GGATGAGGAGAAAAACAAGATCCGGGAAGAGTGGAACAA |
| | | TTTTTCTTACTTCCCTGAAATCACTCACATTGTAATAAA |
| | | GGAGTCTGTGGTCAGCATTAACAAGCAGGACAACAAGAA |
| | | AATGGAACTGAAGCTCTCTTCCCACGAGGAGGCCTTGTC |
| | | CTTTGTGTCCCTGGTAGATGGCTACTTCCGGCTCACAGC |
| | | AGATGCCCATCATTACCTCTGCACCGACGTGGCCCCCCC |
| | | GTTGATCGTCCACAACATACAGAATGGCTGTCATGGTCC |
| | | AATCTGTACAGAATACGCCATCAATAAATTGCGGCAAGA |
| | | AGGAAGCGAGGAGGGGATGTACGTGCTGAGGTGGGCTGC |
| | | ACCGACTTTGACAACATCCTCATGACCGTCACCTGCTTT |
| | | GAGAAGTCTGAGCAGGTGCAGGGTGCCCAGAAGCAGTTC |
| | | AAGAACTTTCAGATCGAGGTGCAGAAGGGCCGCTACAGT |
| | | CTGCACGGTTCGGACCGCAGCTTCCCCAGCTTGGGAGAC |
| | | CTCATGAGCCACCTCAAGAAGCAGATCCTGCGCACGGAT |
| | | AACATCAGCTTCATGCTAAAACGCTGCTGCCAGCCCAAG |
| | | CCCCGAGAAATCTCCAACCTGCTGGTGGCTACTAAGAAA |
| | | GCCCAGGAGTGGCAGCCCGTCTACCCCATGAGCCAGCTG |
| | | AGTTTCGATCGGATCCTCAAGAAGGATCTGGTGCAGGGC |
| | | GAGCACCTTGGGAGAGGCACGAGAACACACATCTATTCT |
| | | GGGACCCTGATGGATTACAAGGATGACGAAGGAACTTCT |
| | | GAAGAGAAGAAGATAAAAGTGATCCTCAAAGTCTTAGAC |
| | | CCCAGCCACAGGGATATTTCCCTGGCCTTCTTCGAGGCA |
| | | GCCAGCATGATGAGACAGGTCTCCCACAAACACATCGTG |
| | | TACCTCTATGGCGTCTGTGTCCGCGACGTGGAGAATATC |
| | | ATGGTGGAAGAGTTTGTGGAAGGGGGTCCTCTGGATCTC |
| | | TTCATGCACCGGAAAAGCGATGTCCTTACCACACCATGG |
| | | AAATTCAAAGTTGCCAAACAGCTGGCCAGTGCCCTGAGC |
| | | TACTTGGAGGATAAAGACCTGGTCCATGGAAATGTGTGT |
| | | ACTAAAAACCTCCTCCTGGCCCGTGAGGGCATCGACAGT |
| | | GAGTGTGGCCCGTTCATCAAGCTCAGTGACCCCGGCATC |
| | | CCCATTACGGTGCTGTCTAGGCAAGAATGCATTGAACGA |
| | | ATCCCATGGATTGCTCCTGAGTGTGTTGAGGACTCCAAG |
| | | AAACCTGAGTGTGGCTGCTGACAAGTGGAGCTTTGGAAC |
| | | CACGCTCTGGGAAATCTGCTACAATGGCGAGATCCCCTT |
| | | GAAAGACAAGACGCTGATTGAGAAAGAGAGATTCTATGA |
| | | AAGCCGGTGCAGGCCAGTGACACCATCATGTAAGGAGCT |
| | | GGCTGACCTCATGACCCGCTGCATGAACTATGACCCCAA |
| | | TCAGAGGCCTTTCTTCCGAGCCATCATGAGAGACATTAA |
| | | TAAGCTTGAAGAGCAGAATCCAGATATTGTTTCAGAAAA |
| | | AAAACCAGCAACTGAAGTGGACCCCACACATTTTGAAAA |
| | | GCGTTCCTAAAGAGGATCCGTGACTTGGGAGAGGGCCAC |
| | | TTTGGGAAGGTTGAGCTCTGCAGGTATGACCCCGAAGGG |
| | | GACAATACAGGGGAGCAGGTGGCTGTTAATCTCTGAAGC |
| | | CTGAGAGTGGAGGTAACCACATAGCTGATCTGAAAAAGG |
| | | AAATCGAGATCTTAAGGAACCTCTATCATGAGAACATTG |
| | | TGAAGTACAAAGGAATCTGCACAGAAGACGAGGAAATGG |
| | | TATTAAGCTCATCATGGAATTTCTGCCTTCGGGAAGCCT |
| | | TAAGGAATATCTTCCAAAGAATAAGAACAAAATAAACCT |
| | | CAAACAGCAGCTAAAATATGCCGTTCAGATTTGTAAGGG |
| | | GATGGACTATTTGGGTTCTCGGCAATACGTTCACCGGGA |
| | | CTTGGCAGCAAGAAATGTCCTTGTTGAGAGTGAACACCA |
| | | AGTGAAAATTGGAGACTTCGGTTTAACCAAAGCAATTGA |
| | | AACCGATAAGGAGTATTACACCGTCAAGGATGACCGGGA |
| | | CAGCCCTGTGTTTGGTATGCTCCAGAATGTTTAATGCAA |
| | | TCTAAATTTTATATTGCCTCTGACGTCTGGTCTTTTGGA |
| | | GTCACTCTGCATGAGCTGCTGACTTACTGTGATTCAGAT |
| | | TCTAGTCCCATGGCTTTGTTCCTGAAAATGATAGGCCCA |
| | | ACCCATGGCCAGATGACAGTCACAAGACTTGTGAATACG |
| | | TTAAAAGAAGGAAAACGCCTGCCGTGCCCACCTAACTGT |
| | | CCAGATGAGGTTTATCAACTTATGAGGAAATGCTGGGAA |
| | | TTCCAACCATCCAATCGGACAAGCTTTCAGAACCTTATT |
| | | GAAGGATTTGAAGCACTTTTAAAATAAGAAGCATGAATA |
| | | ACATTTAAATTCCACAGATTATCAA |

TABLE 20

Amino Acid Sequence from Mouse

| | |
|---|---|
| Sagres | Seq ID |

| | |
|---|---|
| Tag No. | No. |

| | | |
|---|---|---|
| S00039 | 202 | MQYLNIKEDCNAMAFCAKMRSFKKTEVKQVVPEPGV |
| | | EVTFYLLDREPLRLGSGEYTAEELCIRAAQECSISP |
| | | LCHNLFALYDESTKLWYAPNRIITVDDKTSLRLHYR |
| | | MRFYFTNWHGTNDNEQSVWRHSPKKQKNGYEKKRVP |
| | | EATPLLDASSLEYLFAQGQYDLIKCLAPIRDPKTEQ |
| | | DGHDIENECLGMAVLAISHYAMMKKMQLPELPKDIS |
| | | YKRYIPETLNKSIRQRNLLTRMRINNVFKDFLKEFN |
| | | NKTICDSSVHDLKVKYLATLETSTLTKHYGAEIFET |
| | | SMLLISSENELSRCHSNDSGNVLYEVMVTGNGIQWR |
| | | QKPNVVPVEKEKNKLKRKKLEYNKHKKDDERNKLRE |
| | | EWNNFSYFPEITHIVIKESVVSINKQDNKNMELKLS |
| | | SREEALSFVSLVDGYFRLTADAHHYLCTDVAPPLIV |
| | | HNIQNGCHGPICTEYAINKLRQEGSEEGMYVLRWSC |
| | | TDFDNILMTVTCFEKSEVLGGQKQFKNFQIEVQKGR |
| | | YSLHGSMDHFPSLRDLMNHLKKQILRTDNISFVLKR |
| | | CCQPKPREISNLLVATKKAQEWQPVYSMSQLSFDRI |
| | | LKKDIIQGEHLGRGTRTHIYSGTLLDYKDEEGIAEE |
| | | KKIKVILKVLDPSHRDISLAFFEAASMMRQVSHKHI |
| | | YLYGVCVRDVENIMVEEFVEGGPLDLFMHRKSDALT |
| | | TPWKFKVAKQLASALSYLEDKDLVHGNVCTKNLLLA |
| | | REGIDSDIGPFIKLSDPGIPVSVLTRQECIERIPWI |
| | | APECVEDSKNLSVAADKWSFGTTLWEICYNGEIPLK |
| | | DKTLIEKERFYESRCRPVTPSCKELADLMTRCMNYD |
| | | PNQRPFFRAIMRDINKLEEQNPDIVSEKQPTTEVDP |
| | | THFEKRFLKRIRDLGEGHFGKVELCRYDPEGDNTGE |
| | | QVAVKSLKPESGGNHIADLKKEIEILRNLYHENIVK |
| | | YKGICMEDGGNGIKLIMEFLPSGSLKEYLPKNKNKI |
| | | NLKQQLKYAIQICKGMDYLGSRQYVHRDLAARNVLV |
| | | ESEHQVKIGDFGLTKAIETDKEYYTVKDDRDSPVFW |
| | | YAPECLIQCKFYIASDVWSFGVTLHELLTYCDSDFS |
| | | PMALFLKMIGPTHGQMTVTRLVKTLKEGKRLPCPPN |
| | | CPDEVYQLMRKCWEFQPSNRTTFQNLIEGFEALLK |

TABLE 21

Amino Acid Sequence from Human

| | |
|---|---|
| Sagres | Seq. ID |

| | |
|---|---|
| Tag No. | No. |

| | | |
|---|---|---|
| S00039 | 203 | MQYLNIKEDCNAMAFCAKMRSSKKTEVNLEAPEP |
| | | GVEVIFYLSDREPLRLGSGEYTAEELCIRAAQAC |
| | | RISPLCHNLFALYDENTKLWYAPNRTITVDDKMS |
| | | LRLHYRMRFYFTNWHGTNDNEQSVWRHSPKKQKN |
| | | GYEKKKIPDATPLLDASSLEYLFAQGQYDLVKCL |
| | | APIRDPKTEQDGHDIENECLGMAVLAISHYAMMK |
| | | KMQLPELPKDISYKRYIPETLNKSIRQRNLLTRM |
| | | RINNVFKDFLKEFNNKTICDSSVSTHDLKVKYLA |
| | | TLETLTKHYGAEIFETSMLLISSENEMNWFHSND |
| | | GGNVLYYEVMVTGNLGIQWRHKPNVVSVEKEKNK |
| | | LKRKKLENKHKKDEEKNKIREEWNNFSYFPEITH |
| | | IVIKESVVSINKQDNKKMELKLSSHEEALSFVSL |
| | | VDGYFRLTADAHHYLCTDVAPPLIVHNIQNGCHG |
| | | PICTEYAINKLRQEGSEEMGYVLRWSCTDFDNIL |
| | | MTVTCFEKSEQVQGAQKQFKNFQIEVQKGRYSLH |
| | | GSDRSFPSLGDLMSHLKKQILRTDNISFMLKRCC |
| | | QPKPREISNLLVATKKAQEWQPVYPMSQLSFDRI |
| | | LKKDLVQGEHLGRGTRTHIYSGTLMDYKDDEGTS |
| | | EEKKIKVILKVLDPSHRDISLAFFEAASMMRQVS |
| | | HKHIVYLYGVCVRDVENIMVEEFVEGGPLDLFMH |
| | | RKSDVLTTPWKFKVAKQLASALSYLEDKDLVHGN |
| | | VCTKNLLLAREGIDSECGPFIKLSDPGIPITVLS |
| | | RQECIERIPWIAPECVDSKNLSVAADKWSFGTTL |
| | | WEICYNGEIPLKDKTLIEKEFYESRCRPVTPSCK |
| | | ELADLMTRCMNYDPNQRPFFRAIMRDINKLEEQN |
| | | PDIVSEKKPATEVDPTHFEDRFLKRIRDLGEGHF |
| | | GKVELCRYDPEGDNTGEQVAVKSLKPESGGNHIA |
| | | DLKKEIEILRNLYHENIVKYKGICTEDGGNGIKL |
| | | IMEFLPSGSLKEYLPKNKNKINLKQQLKYAVQIC |
| | | KGMDYLGSRQYVHRDLAARNVLVESEHQVKIGDF |
| | | GLTKAIETDKEYYTVKDDRDSPVFWYAPECLMQS |
| | | KFYIASDVWSFGVTLHELLTYCDSDSSPMALFLK |
| | | MIGPTHGMQTVTRLVNTLKEGKRLPCPPNCPDEV |
| | | YQLMRKCWEFQPSNRTSFQNLIEGFEALLK |

TABLE 22

Sagres Tag No. S00039 Nucleotide Sequence

| | |
|---|---|
| Sagres | Seq ID |

| | |
|---|---|
| Tag No. | No. |

| | | |
|---|---|---|
| S00039 | 204 | ACAAGACTTTGAAAAGCGGTTCCTGAAGAGGATTCG |
| | | TGACTTGGGAGAGGGTCACTTTGGGAAGGTTGAGCT |
| | | CTGCAGATATGATCCTGAGGGAGACAACACAGGGGA |
| | | GCAGGTGCTGTCAAGTCCCTGAAGCCTGAGAGTGGA |
| | | GGTAACCACATAGCTGATCTGAAGAAGGAGATAGAG |
| | | ATCTTACGGAACCTCTACCATGAGAACATTGTGAAG |
| | | TACAAAGGAATCTGCATGGAAGACGGAGGCAATGGT |
| | | ATCAAGCTCATCATGGAGTTTCTGCCTTCGGGAAGC |
| | | CTAAAGGAGTATCTGCCAAAGAATAAGAACAAAATC |
| | | AACCTCAAACAGCAGCTAAAAATATGCCATCCAGAA |
| | | TTGTAAGGGGATGGACTACTTGGGTTCTCGGCAATA |
| | | AGTTCACCGGGACTTAGCAGCCAGAATGTCCTTGTT |
| | | GAGAGTGAGCATCCAGTTGAGATTGGAGACCTTGGG |
| | | TTAACCCAAGCCATTTGAAACGATTAGGAGTACTTA |
| | | CACAGTTCAGGACCACCGGGAAAAGCCAGTGTTCCG |
| | | GTACGCTCCGGAATGTTTAATCCAGTGTTAATTTTA |
| | | AAACGCCTCCGATGTCCGGTCCTTTGGAGTGACACT |
| | | GCACGAGCTGCTCAATTACTGTGACTCCGAATTTAG |
| | | TCCCATGGCCTTGGTCCCGAAAAGGTAAGCCCAACT |
| | | CCAGGCCAGAAGACAATTGAAGGCCTGTGGATCACT |
| | | GAAAGAAGGAAAGCCCTGGCATGTCCACCCAATGTC |
| | | CTGATGAAGTTAACAGCCTATGGGAAAATTCCTGGA |
| | | ATTCGANCTACTAACCGAACAATTTTCGGAACCTAT |
| | | GGAAGAGTTTAAGCCCCTTTAAATAGAAGCCTGGCA |
| | | CACTTTAATCCCCATTTCAAATCTTTCTCCAAGCCT |
| | | TTAAAAAGGTTTAAAGGAAAGTTGAATCGGGCCTAA |
| | | GTCCCAAAAAACCGCGGTACAATTGCAATTCACGGG |
| | | TCC |

The Neurogranin nucleic acid and amino acid sequences of the invention are depicted in Tables 23, 24, 25, 26 and 27. The nucleic acid sequence shown in Table 23 is from mouse. The nucleic acid sequence shown in Table 24 is from human. The amino acid sequence shown in Table 25 is from mouse. The amino acid sequence shown in Table 26 is from human. The sequence of Sagres Tag No. S00092 is shown in Table 27.

TABLE 23

Neurogranin Nucleic Acid Sequence from Mouse

| | |
|---|---|
| Sagres | Seq. ID |

| | |
|---|---|
| Tag No. | No. |

| | | |
|---|---|---|
| S00092 | 205 | GTTGGTCCTCGCTCCAGTTCTCCCCGCCCACCCT |
| | | GCAGAAAGTGTCTTCTGATTGGCTTCGAGGCCGC |
| | | AGGGCTCAGGTTACATTCGCAAGAGTTGCGGAGC |
| | | GCGGGAGACCGGACCCAAGAGGAGAGAGGCTGGT |
| | | TCTGCAAGGATTCTGCGCTGGTCGGGGAGTGCCC |
| | | GACAGCCCCTGAGCTGCCACCCAGCATCGTACAA |
| | | ACCCACCCCCGCTCTGCGCCAGGCTCCACCCCAG |
| | | CCAAGGACCCTCAACACCGGCAATGGACTGCTGC |
| | | ACGGAGAGCGCCTGCTCCAAGCCAGACGACGATA |
| | | TTCTTGACATCCCGCTGGATGATCCCGGAGCCAA |
| | | CGCCGCTGCAGCCAAAATCCAGGCGAGTTTCCGG |
| | | GGCCACATGGCGAGGAAGAAGATAAAGAGCGGAG |
| | | AGTGTGGCCGGAAGGGACCGGGCCCCGGGGGACC |
| | | AGGCGGAGCTGGGGGCGCCCGGGGAGGCGCGGGC |
| | | GGCGGCCCCAGCGGAGACTAGGCCAGAGCTGAAC |
| | | GTTTTAGAAGTTCCAGAGGAGAGTCGGATGCCGC |
| | | GTCCCCTTCGCAGTGACAAGACTTCCCTACTGTG |
| | | TTTGTGAGCCCCTCCTTCCCACCAACCAGCCAGC |
| | | TTCAGGAGCCCCCCCCCTCCCCCCGCCGCGTCCC |
| | | AGAGACTCCCTCTCCCAGGCTGGCTTCGTCTTGG |
| | | GCGTAGCAAGTCCGTGCCCTTTTTAGCTCTTCAG |
| | | TCTAAC721GTGGTCTCCTTTTGCCTTTTCTCCC |
| | | ACCCTCGTCCCAAACCCATACTCCAAAATGTCCT |
| | | TTTGCTTCACGCCCACCTGTCCACGCGCCCAGCA |
| | | TGCAGCTCTGCCTCCGCAGCCTCGGTGCGCTTCG |
| | | CTGCGCGTACTTGCAGAGGGCGCCCAATGCGTCG |
| | | CCCAAATACTCTCAAAAAAAGAAAGAAAAAAAGA |
| | | AAAAGAAAGAAAGAAAAAAAAAGCAACCACCAAG |
| | | TCCTTCGTTCTGTGGGCAACGAAAGGGGGCGCCC |
| | | GCGTCTTTCCACCCTAGCCTAACCTCAACCTCCT |
| | | AAACCTGGGGCTAGGAAAGAGGGGAGGAGGTTTT |
| | | CATGGTTATCTGATAATTTCCCTTGCTCAAATGG |
| | | AAAGTGAAGTCCTATCCCATACCTGCCTGTCACC |
| | | CTCTTTTTTCTTGAAAACGCACCCTGAGAGCAGC |
| | | CCCTCCCGCTCTTCTTTGTTTATGCAAAAGCCTC |
| | | CTGAGCGCCTGGAGGCTCCGGCAGGAGGAGACTT |
| | | CCGCAGCCCCGCCCCATGATAGCCTCTCCCCCGT |
| | | TGGGCTCCTCGGGTTGTGGCTGGAAGGCTTTTAA |
| | | TCTCTGCGTGTGCATGTTACCATACTGGGTTGGA |
| | | ATGTGAATAATAAAGAGGAATGTCGAAGTGT |

TABLE 24

Neurogranin Nucleic Acid Sequence from Human

| | |
|---|---|
| Sagres | Seq. ID |

| | |
|---|---|
| Tag No. | No. |

| | | |
|---|---|---|
| S00092 | 206 | GGCACGAGGCGCCAGCCTTCGTCCCCGCAGAGGA |
| | | CCCCCCGACACCAGCATGGACTGCTGCACCGAGA |
| | | ACGCCTGCTCCAAGCCGGACGACGACATTCTAGA |
| | | CATCCCGCTGGACGATCCCGGCGCCAACGCGGCC |
| | | GCCGCCAAAATCCAGGCGAGTTTTCGGGGCCACA |
| | | TGGCGCGGAAGAAGATAAAGAGCGGAGAGCGCGG |
| | | CCGGAAGGGCCCGGGCCCTGGGGGGCCTGGCGGA |
| | | GCTGGGGTGGCCCGGGGAGGCGCGGGCGGCGGCC |
| | | CCAGCGGAGACTAGGCCAGAAGAACTGAGCATTT |
| | | TCAAAGTTCCCGAGGAGAGATGGATGCCGCGTCC |
| | | CCTTCGCAGCGACGAGACTTCCCTGCCGTGTTTG |
| | | TGACCCCCTCCTGCCCAGCAACCTGCCAGCTACA |
| | | GGAGCCCCCTGCGTCCCAGAGACTCCCTCACCCA |
| | | GGCAGGCTCCGTCGCGGAGTCGCTGAGTCCGTGC |
| | | CCTTTTAGTTAGTTCTGCAGTCTAGTATGGTCCC |
| | | CATTTGCCCTTCCACTCCACCCCACCCTAAACCA |
| | | TGCGCTCCCAATCTTCCTTCTTTTGCTTCTCGCC |
| | | CACCTCTTCCCGCACCCAGCATGCAGCTCTGCCT |
| | | CCGCAGCCTCAGTGCGCTTTCCTGCGCGCACTGC |
| | | GGAGGGCGCCCTAAGCGTCACCCAAGCACACTCA |
| | | CTTAAAGAAAAAACGAGTTCTTTCGTTCTGTGCG |
| | | CAGCTAAAAGGGGCGCCCTACATCTCCGTGCCAC |
| | | TCCCGCCCCAGCCTAGCCCCAAGACTTGGATCCG |
| | | GGGCGAGATGAAGGGAAGAGGGTTGTTTTGGTTT |
| | | CGGACGACCCTTGCTCTGACCGGAAGAGAAGTCC |
| | | CTATCCCACACCTGCCTGTGCACGTTCCCTCCCC |
| | | TTTCCCCAGCGCACTGTTGAGGGCAGCCTCTCCA |
| | | GCTCTCTTGTTTATGCAAACGCCGAGCGCCTGGG |
| | | AGGCTCGGTAGGAGGAGTCTTCCACGGCCCCGCC |
| | | CCGCCCCTGTCGGTCCCGCCCTCCCCCCCGCCGG |
| | | GCTCCTGGGGCTGTGGCCGAAAGGTTTCTGATCT |
| | | CCGTGTGTGCATGTGACTGTGCTGGGTTGGAATG |
| | | TGAACAATAAAGAGGAATGTCCAAGTGAAAAAAA |
| | | AAAAAAAAAAAAA |

TABLE 25

Neurogranin Nucleic Acid Sequence from Mouse

| | |
|---|---|
| Sagres | Seq. ID |

| | |
|---|---|
| Tag No. | No. |

| | | |
|---|---|---|
| S00092 | 207 | MDCCTESACSKPDDDILDIPLDDPGANAAAAKIQ |
| | | ASFRGHMARKKIKSGECGRKGPGPGGPGGAGGAR |
| | | GGAGGGPSGD |

TABLE 26

Neurogranin Amino Acid Sequence from Human

| | |
|---|---|
| Sagres | Seq. ID |

| | |
|---|---|
| Tag No. | No. |

| | | |
|---|---|---|
| S00092 | 208 | MDCCTENACSKPDDDILDIPLDDPGANAAAAKIQ |
| | | ASFRGHMARKKIKSGERGRKGPGPGGPGGAGVAR |
| | | GGAGGGPSGD |

TABLE 27

Sagres Tag No. S00092 Nucleic Acid Sequence

| | |
|---|---|
| Sagres | Seq. ID |

| | |
|---|---|
| Tag No. | No. |

| | | |
|---|---|---|
| S00092 | 209 | GTCAAAATACTGAGAATTAGAGGCTATTGGATGC |
| | | CAAGTCATAGAGAGGACACATATATACCAATACT |
| | | TCCAAGGCTCAGGAAACATCATGGAAGAAGGGGT |
| | | AGGAAGAATTTAANAACCAGAAGAAGGGGGGTGA |
| | | GGTATGGAATGATGATTTCCAGTCATGACTTGGC |
| | | TATTGAGTTAACAACAGCTGGATCACCTGCACAA |
| | | GATCTCCACAAGAGTGGGCCCATTAACACTCTAT |
| | | CATGGAAAGAGGAGGGGCNTATGAGGTACCACCC |
| | | CACCCTGAAGATTTATACACAATTAATANTTGGT |
| | | GAGGTAGGGAGAGACATTTACTTTAGGGGTGCAG |
| | | TCACTAGTACAGTGCCTAC |

The Nrf2 nucleic acid sequences of the invention are depicted in Tables 28 through 31.

A Nrf2 nucleic acid sequence of the invention is depicted in Table 28 as SEQ ID NO. 210. The nucleic acid sequence shown is from mouse.

TABLE 28

MOUSE

SEQ

| | |
|---|---|
| ID # | SEQUENCE |

| | |
|---|---|
| 210 | TGCTCCATGCCCTTGTCCTCGCTCTGGCCCTTGCCTCTTGCCCTA |
| | GCCTTTTCTCCGCCTCTAAGTTCTTGTCCCGTCCCTAGGTCCTTG |
| | TTCCAGGGGGTGGGGGCGGGGCGGACTAAGGCTGGCCTGCCACTC |
| | CAGCGAGCAGGCTATCTCCTTAGTTCTCGCTGCTCGGACTAGCCA |
| | TTGCCGCCGCCTCACCTCTGCTGCAAGTAGCCTCGCCGTCGGGGA |
| | GCCCTACCACACGGTCCGCCCTCAGCATGATGGACTTGGAGTTGC |
| | CACCGCCAGACTACAGTCCCAGCAGGACATGGATTTGATTGACAT |
| | CCTTTGGAGGCAAGACATAGATCTTGGAGTAAGTCGAGAAGTGTT |
| | TGACTTTAGTCAGCGACAGAAGGACTATGAGCTGGAAAAACAGAA |
| | AAAACTCGAAAAGGAAAGACAAGAGCAACTCCAGAAGGAACAGGA |
| | GAAGGCCTTTTTTGCTCAGTTTCAACTGGATGAAGAAACAGGAGA |
| | ATTCCTCCCAATTCAGCCGGCCCAGCACATCCAGACAGACACCAG |
| | TGGATCCGCCAGCTACTCCCAGGTTGCCCACATTCCCAAACAAGA |
| | TGCCTTGTACTTTGAAGACTGTATGCAGCTTTTGGCAGAGACATT |
| | CCCATTTGTAGATGACCATGAGTCGCTTGCCCTGGATATCCCCAG |
| | CCACGCTGAAAGTTCAGTCTTCACTGCCCCTCATCAGGCCCAGTC |
| | CCTCAATAGCTCTCTGGAGGCAGCCATGACTGATTTAAGCAGCAT |
| | AGAGCAGGACATGGAGCAAGTTTGGCAGGAGCTATTTTCCATTCC |
| | CGAATTACAGTGTCTTAATACCGAAACAAGCAGCTGGCTGATACT |
| | ACCGCTGTTCCCAGCCCAGAAGCCACACTGACAGAAATGGACAGC |
| | AATTACCATTTTTACTCATCGATCTCCTCGCTGGAAAAAGAAGTG |
| | GGCAACTGTGGTCCACATTTCCTTCATGGTTTTGAGGATTCTTTC |
| | AGCAGCATCCTCTCCACTGATGATGCCAGCCAGCTGACCTCCTTA |
| | GACTCAAATCCCACCTTAAACACAGATTTTGGCGATGAATTTTAT |
| | TCTGCTTTCATAGCAGAGCCCAGTGACGGTGGCAGCATGCCTTCC |
| | TCCGCTGCCATCAGTCAGTCACTCTCTGAACTCCTGGACGGGACT |
| | ATTGAAGGCTGTGACCTGTCACTGTGTAAAGCTTTCAACCCGAAG |
| | CACGCTGAAGGCACAATGGAATTCAATGACTCTGACTCTGGCATT |
| | TCACTGAACACGAGTCCCAGCCGAGCGTCCCCAGAGCACTCGTGG |
| | AGTCTTCCATTTACGGAGACCCACCGCCTGGGTTCAGTGACTCGG |
| | AAATGGAGGAGCTAGATAGTGCCCCTGGAAGTGTCAAACAGAACG |
| | GCCCTAAAGCACAGCCAGCACATTCTCCTGGAGACACAGTACAGC |
| | CTCTGTCACCAGCTCAAGGGCACAGTGCTCCTATGCGTGAATCCC |
| | AATGTGAAAATACAACAAAAAAAGAAGTTCCCGTGAGTCCTGGTC |
| | ATCAAAAAGCCCCATTCACAAAAGACAAACATTCAAGCCGCTTAG |
| | AGGCTCATCTCACACGAGATGAGCTTAGGGCAAAAGCTCTCCATA |
| | TTCCATTCCCTGTCGAAAAAATCATTAACCTCCCTGTTGATGACT |
| | TCAATGAAATGATGTCCAAGGAGCAATTCAATGAAGCTCAGCTCG |
| | CATTGATCCGAGATATACGCAGGAGAGGTAAGAATAAAGTCGCCG |
| | CCCAGAACTGTAGGAAAAGGAAGCTGGAGAACATTGTCGAGCTGG |
| | AGCAAGACTTGGGCCACTTAAAAGACGAGAGAGAAAAACTACTCA |
| | GAGAAAAGGGAGAAAACGACAGAAACCTCCATCTACTGAAAAGGC |
| | GGCTCAGCACCTTGTATCTTGAAGTCTTCAGCATGTTACGTGATG |
| | AGGATGGAAAGCCTTACTCTCCCAGTGAATACTCTCTGCAGCAAA |
| | CCAGAGATGGCAATGTGTTCCTTGTTCCCAAAAGCAAGAAGCCAG |
| | ATACAAAGAAAAACTAGGTTCGGGAGGATGGAGCCTTTTCTGAGC |
| | TAGTGTTTGTTTTGTACTGCTAAAACTTCCTACTGTGATGTGAAA |
| | TGCAGAAACACTTTATAAGTAACTATGCAGAATTATAGCCAAAGC |
| | TAGTATAGCAATAATATGAAACTTTACAAAGCATTAAAGTCTCAA |
| | TGTTGAATCAGTTTCATTTTAACTCTCAAGTTAATTCTTAGGCAC |
| | CATTTGGGAGAGTTTCTGTTTAAGTGTAAATACTACAGAACTTAT |
| | TATACTGTTCTCACTTGTTACAGTCATAGACTTATATGACATCTG |
| | GCTAAAAGCAAACTATTGAAAACTAACCAGACCACTATACTTTTT |
| | TATATACTGTATGAACAGGAAATGACATTTTTATATTAATTGTTT |
| | AGCTCATAAAAATTAAGGAGCTAGCACTAATAAAAGAATATCATG |
| | ACT |

SEQ ID NO. 211 (in Table 29) represents the amino acid sequence of a protein encoded by SEQ ID NO. 210.

TABLE 29

MOUSE

SEQ

| | |
|---|---|
| ID # | SEQUENCE |

| | |
|---|---|
| 211 | MDLIDILWRQDIDLGVSREVFDFSQRQKDYELEKQKKLEKERQEQ |
| | QKEQEKAFFAQFQLDEETGEFLPIQPAQHIQTDTSGSASYSQVAH |
| | IPKQDALYFEDCMQLLAETFPFVDDHESLALDIPSHAESSVFTAP |
| | HQAQSLNSSLEAAMTDLSSIEQDMEQVWQELFSIPELQCLNTENK |
| | QLADTTAVPSPEATLTEMDSNYHFYSSISSLEKEVGNCGPHFLHG |
| | FEDSFSSILSTDDASQLTSLDSNPTLNTDFGDEFYSAFIAEPSDG |
| | GSMPSSAAISQSLSELLDGTIEGCDLSLCKAFNPKHAEGTMEFND |
| | SDSGISLNTSPSRASPEHSVESSIYGDPPPGFSDSEMEELDSAPG |
| | SVKQNGPKAQPAHSPGDTVQPLSPAQGHSAPMRESQCENTTKKEV |
| | PVSPGHQKAPFTKDKHSSRLEAHLTRDELRAKALHIPFPVEKIIN |
| | LPVDDFNEMMSKEQFNEAQLALIRDIRRRGKNKVAAQNCRKRKLE |
| | NIVELEQDLGHLKDEREKLLREKGENDRNLHLLKRRLSTLYLEVF |
| | SMLRDEDGKPYSPSEYSLQQTRKGNVFLVPKSKKPDTKKN |

Table 30 (SEQ ID NO: 212) depicts a human Nrf2 nucleic acid sequence of the invention.

TABLE 30

HUMAN

SEQ

| | |
|---|---|
| ID # | SEQUENCE |

| | |
|---|---|
| 212 | TTGGAGCTGCCGCCGCCGGGACTCCCGTCCCAGCAGGACATGGAT |
| | TTGATTGACATACTTTGGAGGCAAGATATAGATCTTGGAGTAAGT |
| | CGAGAAGTATTTGACTTCAGTCAGCGACGGAAAGAGTATGAGCTG |
| | GAAAAACAGAAAAAACTTGAAAAGGAAAGACAAGAACAACTCCAA |
| | AAGGAGCAAGAGAAAGCCTTTTTCACTCAGTTACAACTAGATGAA |
| | GAGACAGGTGAATTTCTCCCAATTCAGCCAGCCCAGCACACCCAG |
| | TCAGAAACCAGTGGATCTGCCAACTACTCCCAGGTTGCCCACATT |
| | CCCAAATCAGATGCTTTGTACTTTGATGACTGCATGCAGCTTTTG |
| | GCGCAGACATTCCCGTTTGTAGATGACAATGAGGTTTCTTCGGCT |
| | ACGTTTCAGTCACTTGTTCCTGATATTCCCGGTCACATCGAGAGC |
| | CCAGTCTTCATTGCTACTAATCAGGCTCAGTCACCTGAAACTTCT |
| | GTTGCTCAGGTAGCCCCTGTTGATTTAGACGGTATGCAACAGGAC |
| | ATTGAGCAAGTTTGGGAGGAGCTATTATCCATTCCTGAGTTACAG |
| | TGTCTTAATATTGAAAATGACAAGCTGGTTGAGACTACCATGGTT |
| | CCAAGTCCAGAAGCCAAACTGACAGAAGTTGACAATTATCATTTT |
| | TACTCATCTATACCCTCAATGGAAAAAGAAGTAGGTAACTGTAGT |
| | CCACATTTTCTTAATGCTTTTGAGGATTCCTTCAGCAGCATCCTC |
| | TCCACAGAAGACCCCAACCAGTTGACAGTGAACTCATTAAATTCA |
| | GATGCCACAGTCAACACAGATTTTGGTGATGAATTTTATTCTGCT |
| | TTCATAGCTGAGCCCAGTATCAGCAACAGCATGCCCTCACCTGCT |
| | ACTTTAAGCCATTCACTCTCTGAACTTCTAAATGGGCCCATTGAT |
| | GTTTCTGATCTATCACTTTGCAAAGCTTTCAACCAAAACCACCCT |
| | GAAAGCACAGCAGAATTCAATGATTCTGACTCCGGCATTTCACTA |
| | AACACAAGTCCCAGTGTGGCATCACCAGAACACTCAGTGGAATCT |
| | TCCAGCTATGGAGACACACTACTTGGCCTCAGTGATTCTGAAGTG |
| | GAAGAGCTAGATAGTGCCCCTGGAAGTGTCAAACAGAATGGTCCT |
| | AAAACACCAGTACATTCTTCTGGGGATATGGTACAACCCTTGTCA |
| | CCATCTCAGGGGCAGAGCACTCACGTGCATGATGCCCAATGTGAG |
| | AACACACCAGAGAAAGAATTGCCTGTAAGTCCTGGTCATCGGAAA |
| | ACCCCATTCACAAAAGACAAACATTCAAGCCGCTTGGAGGCTCAT |
| | CTCACAAGAGATGAACTTAGGGCAAAAGCTCTCCATATCCCATTC |
| | CCTGTAGAAAAAATCATTAACCTCCCTGTTGTTGACTTCAACGAA |
| | ATGATGTCCAAAGAGCAGTTCAATGAAGCTCAACTTGCATTAATT |
| | CGGGATATACGTAGGAGGGGTAAGAATAAAGTGGCTGCTCAGAAT |
| | TGCAGAAAAAGAAAACTGGAAAATATAGTAGAACTAGAGCAAGAT |
| | TTAGATCATTTGAAAGATGAAAAAGAAAAATTGCTCAAAGAAAAA |
| | GGAGAAAATGACAAAAGCCTTCACCTACTGAAAAAACAACTCAGC |
| | ACCTTATATCTCGAAGTTTTCAGCATGCTACGTGATGAAGATGGA |
| | AAACCTTATTCTCCTAGTGAATACTCCCTGCAGCAAACAAGAGAT |
| | GGCAATGTTTTCCTTGTTCCCAAAAGTAAGAAGCCAGATGTTAAG |
| | AAAAACTAGATTTAGGAGGATTTGACCTTTTCTGAGCTAGTTTTT |
| | TTGTACTATTATACTAAAAGCTCCTACTGTGATGTGAAATGCTCA |
| | TACTTTATAAGTAATTCTATGCAAAATCATAGCCAAAACTAGTAT |
| | AGAAAATAATACGAAACTTTAAAAAGCATTGGAGTGTCAGTATGT |
| | TGAATCAGTAGTTTCACTTTAACTGTAAACAATTTCTTAGGACAC |
| | CATTTGGGCTAGTTTCTGTGTAAGTGTAAATACTACAAAAACTTA |
| | TTTATACTGTTCTTATGTCATTTGTTATATTCATAGATTTATATG |
| | ATGATATGACATCTGGCTAAAAAGAAATTATTGCAAAACTAACCA |
| | CGATGTACTTTTTTATAAATACTGTATGGACAAAAAATGGCATTT |
| | TTTATAATTAAATTGTTTAGCTCTGGCAAAAAAAAAAAATTTTTT |
| | AAGAGCTGGTACTAATAAAGGATTATTATGACTGTTAAAAAAAAA |
| | AAAAAAAAA |

Table 31 (SEQ ID NO: 213 depicts the amino acid sequence encoded by the nucleic acid sequence of SEQ ID NO: 212).

TABLE 31

HUMAN

SEQ

| | |
|---|---|
| ID # | SEQUENCE |

| | |
|---|---|
| 213 | MDLIDILWRQDIDLGVSREVFDFSQRRKEYELEKQKKLEKERQEQ |
| | LQKEQEKAFFTQLQLDEETGEFLPIQPAQHTQSETSGSANYSQVA |
| | HIPKSDALYFDDCMQLLAQTFPFVDDNEVSSATFQSLVPDIPGHI |
| | ESPVFIATNQAQSPETSVAQVAPVDLDGMQQDIEQVWEELLSIPE |
| | LQCLNIENDKLVETTMVPSPEAKLTEVDNYHFYSSIPSMEKEVGN |
| | CSPHFLNAFEDSFSSILSTEDPNQLTVNSLNSDATVNTDFGDEFY |
| | SAFIAEPSISNSMPSPATLSHSLSELLNGPIDVSDLSLCKAFNQN |
| | HPESTAEFNDSDSGISLNTSPSVASPEHSVESSSYGDTLLGLSDS |
| | EVEELDSAPGSVKQNGPKTPVHSSGDMVQPLSPSQGQSTHVHDAQ |
| | CENTPEKELPVSPGHRKTPFTKDKHSSRLEAHLTRDELRAKALHI |
| | PFPVEKIINLPVVDFNEMMSKEQFNEAQLALIRDIRRRGKNKVAA |
| | QNCRKRKLENIVELEQDLDHLKDEKEKLLKEKGENDKSLHLLKKQ |
| | LSTLYLEVFSMLRDEDGKPYSPSEYSLQQTRDGNVFLVPKSKKPD |
| | VKKN |

All accession numbers cited herein are incorporated by reference in their entirety. All references cited herein are expressly incorporated in their entirety by reference.

What is claimed is:

1. A method of diagnosing cancer in a patient comprising detecting the presence of differential expression of HIPK1 in a patient sample, wherein the presence of differential expression of HIPK1 in said sample is indicative of a patient who has cancer.

2. The method of claim 1 wherein the cancer is lymphoma or leukemia.

3. The method of claim 1 wherein the differential expression is downregulation of HIPK1 expression as compared to a control.

4. A method of diagnosing cancer comprising:
(a) measuring a level of a HIPK1 mRNA in a first sample, said first sample comprising a first tissue type of a first individual; and
(b) comparing the level of HIPK1 mRNA in (a) to:
(1) a level of the HIPK1 mRNA in a second sample, said second sample comprising a normal tissue type of said first individual, or
(2) a level of the HIPK1 mRNA in a third sample, said third sample comprising a normal tissue type from an unaffected individual;
wherein a decrease of at least 50% between the level of HIPK1 mRNA in (a) and the level of the HIPK1 mRNA in the second sample or the third sample indicates that the first individual has or is predisposed to cancer.

5. The method of claim 4 wherein the HIPK1 mRNA has a nucleotide sequence of SEQ ID NO:198.

6. The method of claim 4 wherein the cancer is lymphoma or leukemia.

7. A method of diagnosing cancer comprising:
(a) measuring a level of HIPK1 gene expression in a first sample, said first sample comprising a first tissue type of a first individual; and
(b) comparing the level of HIPK1 gene expression in (a) to:
(1) a level of HIPK1 gene expression in a second sample, said second sample comprising a normal tissue type of said first individual, or
(2) a level of HIPK1 gene expression in a third sample, said third sample comprising a normal tissue type from an unaffected individual;
wherein a decrease of at least about 50% between the level of HIPK1 gene expression in (a) and the level of HIPK1 gene expression in the second sample or the third sample indicates that the first individual has or is predisposed to cancer.

8. The method of claim 7 wherein the HIPK1 gene encodes a protein having a sequence of SEQ ID NO:198.

9. The method of claim 7 wherein the cancer is lymphoma or leukemia.

10. The method of claim 4 or claim 7 wherein the decrease between the level of HIPK1 gene expression in (a) and the level of the HIPK1 gene expression in the second sample or the third sample is at least 100%.

11. The method of claim 7 wherein the level of HIPK1 gene expression is determined by measuring HIPK1 mRNA (SEQ ID NO: 198).

12. A method of screening for anti-cancer activity comprising:
(a) contacting a cell that expresses HIPK1 with a candidate anti-cancer agent; and
(b) detecting a difference of at least about 50% between the level of HIPK1 gene expression in the cell in the presence and in the absence of the candidate anti-cancer agent, wherein a difference between the level of HIPK1 gene expression in the cell in the presence and in the absence of the candidate anti-cancer agent of at least 50% indicates that the candidate anti-cancer agent has anti-cancer activity.

13. The method of claim 12 wherein a difference of at least 100% between the level of HIPK1 gene expression in the cell in the presence and in the absence of the candidate anti-cancer agent indicates that the candidate anti-cancer agent has anti-cancer activity.

14. The method of claim 12 wherein the candidate anti-cancer agent is an antibody, small organic compound, small inorganic compound, or polynucleotide.

15. The method of claim 12 wherein the candidate anti-cancer agent is a monoclonal antibody.

16. The method of claim 12 wherein the candidate anti-cancer agent is a human or humanized antibody.

17. The method of claim 14 wherein the polynucleotide is an antisense oligonucleotide.

18. The method of claim 9 wherein the cancer is lymphoma or leukemia.
